(19) United States
(12) Patent Application Publication
(10) Pub. No.: US 2026/0014146 A1
(43) Pub. Date: Jan. 15, 2026

(54) CDK2 INHIBITORS AND METHODS OF USING THE SAME

(71) Applicant: Cedilla Therapeutics, Inc., Foxboro, MA (US)

(72) Inventors: Louise Clare KIRMAN, Foxboro, MA (US); Carl Eric SCHWARTZ, Foxboro, MA (US); Thomas P. BLAISDELL, Foxboro, MA (US)

(21) Appl. No.: 18/995,327

(22) PCT Filed: Jul. 28, 2023
(86) PCT No.: PCT/US2023/071250

§ 371 Date: Jan. 16, 2025

Related U.S. Application Data
(60) Provisional application No. 63/393,711, filed on Jul. 29, 2022.

(51) Int. Cl. A61K 31/498; A61K 31/4155; A61K 31/416; A61K 31/422; A61K 31/427; A61K 31/428; A61K 31/437; A61K 31/438; A61K 31/4439; A61K 31/454; A61K 31/4545; A61K 31/506; A61K 31/519; A61P 35/00; C07D 487/10; C07D 519/00

(52) U.S. Cl. CPC A61K 31/498; A61K 31/4155; A61K 31/416; A61K 31/422; A61K 31/427; A61K 31/428; A61K 31/437; A61K 31/438; A61K 31/4439; A61K 31/454; A61K 31/4545; A61K 31/506; A61K 31/519; A61P 35/00; C07D 487/10; C07D 519/00

(57) ABSTRACT

The present disclosure provides compounds, compositions thereof, and methods of using the same for the inhibition of CDK2, and the treatment of CDK2 related diseases and disorders.

### CROSS REFERENCE TO RELATED APPLICATIONS

This application claims the benefit of priority to U.S. Provisional Application No. 63/393,711, filed Jul. 29, 2022, the entire contents of which is herein incorporated by reference.

### FIELD

The present disclosure relates generally to Cyclin-dependent kinase 2 (CDK2) inhibiting chemical compounds and uses thereof in the inhibition of the activity of CDK2. The disclosure also provides pharmaceutically acceptable compositions comprising compounds disclosed herein and methods of using said compounds and compositions in the treatment of various disorders related to CDK2 activity.

### BACKGROUND

Cell cycle dysregulation, including uncontrolled cell growth, impaired cell differentiation and abnormal apoptosis have been shown to be caused by over activity of Cyclin-dependent kinases (CDKs). CDKs are important serine/threonine protein kinases that become active when combined with a specific cyclin partner. There are various subtypes of CDKs, each having a different role during the cell cycle, with varying levels of activity during each of the phases. CDK1, CDK2, CDK4 and CDK6 have been found to be specifically important subtypes, where over activity of one or more of these subtypes may lead to dysregulation of the cell cycle and the development of a variety of cancers. The S phase of the cell cycle is responsible for DNA replication and is the phase where aberrant DNA replication may occur. The CDK2/cyclin E complex is required for the cell cycle transition from the G1 phase to the S phase and the CDK2/cyclin A complex is required for the cell cycle transition from the S phase to the G2 phase. Therefore, selective inhibition of the CDK2/cyclin E and/or CDK2/cyclin A complexes can prevent aberrant DNA replication and can be used to treat certain cancers.

Accordingly, there is a need for the development of compounds capable of inhibiting the activity of CDK2/cyclin complexes, and pharmaceutical compositions thereof, for the prevention, and treatment of CDK2 related diseases or disorders.

### SUMMARY

The present disclosure is based at least in part on the identification of compounds that bind and inhibit Cyclin-dependent kinase 2 (CDK2) and/or CDK2/cyclin complexes and methods of using the same to treat diseases associated with CDK2 activity. Disclosed herein is a compound according to Formula I or a pharmaceutically acceptable salt thereof:

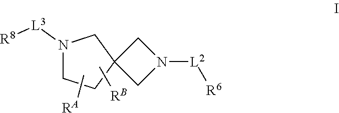

wherein each variable is as defined and described herein.

Compounds of the present disclosure, and pharmaceutically acceptable compositions thereof, are useful for treating a variety of diseases, disorders or conditions, associated with CDK2 activity. Such diseases, disorders, or conditions include those described herein.

### DETAILED DESCRIPTION OF CERTAIN EMBODIMENTS

### 1. General Description of Compounds of the Disclosure

The present disclosure provides compounds capable of inhibiting Cyclin-dependent kinase 2 (CDK2) and/or CDK2/cyclin complexes.

In some embodiments, provided herein are compounds according to Formula I:

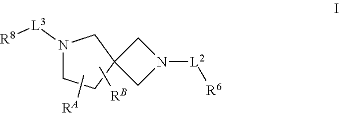

or a pharmaceutically acceptable salt thereof, wherein:
R^{A} is

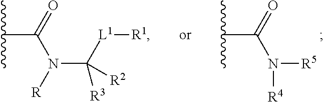

R^{B} is a hydrogen, an optionally substituted C₁₋₆ aliphatic group, —OR, —NR₂, or a halogen;
L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—:
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
R² is hydrogen, an optionally substituted C₁₋₆ aliphatic group, —C₁₋₆ alkylene-OR, —C₁₋₃alkylene-O—C₁₋₃ alkylene-R—C(O)OR, —C(O)NR₂, or an optionally substituted cyclic group selected from phenyl and a 5-6 membered monocyclic heteroaromatic ring (having 1-4 heteroatoms independently selected from nitrogen, oxygen, and sulfur); and
R³ is hydrogen; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and
R⁵ is hydrogen; or
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur), or two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or the two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
wherein one or both of L² and L³ is a covalent bond.

Overexpression of CDK2 is associated with abnormal regulation of the cell-cycle. The cyclin E/CDK2 complex plays an important role in regulation of the G1/S transition, histone biosynthesis and centrosome duplication. Progressive phosphorylation of retinoblastoma (Rb) by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al., Nat. Rev. Drug. Discov. 2015; 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Mol. Cancer Ther. (2012) 11:1488-99; Herrera-Abreu et al., Cancer Res. (2016) 76:2301-2313). Cyclin E amplification also reportedly contributes to trastuzumab resistance in HER2+ breast cancer. (Scaltriti et al., Proc Natl Acad Sci. (2011) 108:3761-6). Cyclin E overexpression has also been reported to play a role in basal-like and triple negative breast cancer (TNBC), as well as inflammatory breast cancer. (Elsawaf & Sinn, Breast Care (2011) 6:273-278; Alexander et al., Oncotarget (2017) 8:14897-14911.)

Amplification or overexpression of cyclin E1 (CCNE1) is also associated with poor outcomes in ovarian, gastric, endometrial and other cancers. (Nakayama et al., Gene amplification CCNE1 is related to poor survival and potential therapeutic target in ovarian cancer, Cancer (2010) 116:2621-34; Etemadmoghadam et al., Clin Cancer Res (2013) 19:5960-71: Au-Yeung et al., Clin. Cancer Res. (2017) 23:1862-1874; Ayhan et al., Modern Pathology (2017) 30:297-303; Ooi et al., Hum Pathol. (2017) 61:58-67; Noske et al., Oncotarget (2017) 8:14794-14805).

There remains a need in the art for CDK inhibitors, especially selective CDK2 inhibitors, which may be useful for the treatment of cancer or other proliferative diseases or conditions. In particular, CDK2 inhibitors may be useful in treating CCNE1 or CCNE2 amplified tumors.

### 2. Compounds and Definitions

Compounds of this present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry.” Thomas Sorrell, University Science Books, Sausalito: 2005, and “March's Advanced Organic Chemistry: Reactions Mechanisms and Structure,” 8^{th} Ed.: Smith, M. B., John Wiley & Sons, New York: 2019, the entire contents of which are hereby incorporated by reference.

The term “aliphatic” or “aliphatic group.” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments. “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused, bridged, or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

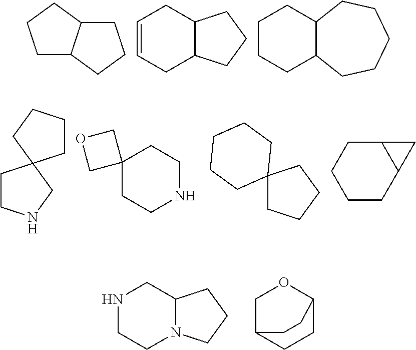

Exemplary bridged bicyclics, contemplated as falling under the scope of a “bicycle” or “bicyclic ring” include:

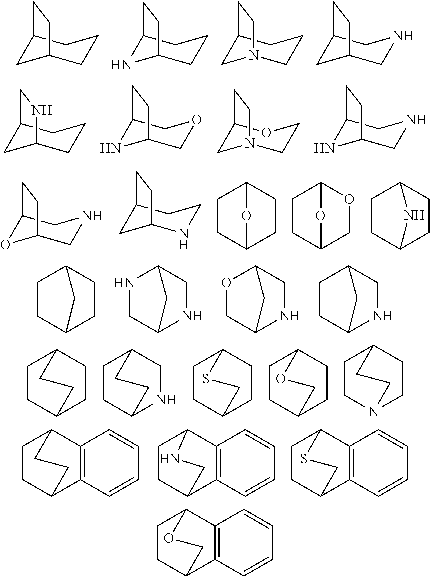

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl.” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring.” In certain embodiments of the present disclosure. “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl.” or “heteroaralkoxy.” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety.” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Covalent substituents on a substitutable carbon atom of an “optionally substituted” group are independently halogen: —(CH₂)₀₋₆R^{∘}; —(CH₂)₀₋₆OR^{∘}; —O(CH₂)₀₋₆R^{∘}, —O—(CH₂)₀₋₆C(O)OR^{∘}; —(CH₂)₀₋₆CH(OR^{∘})₂; —(CH₂)₀₋₆SR^{∘}; —(CH₂)₀₋₆Ph, which Ph may be substituted with R^{∘}; —(CH₂)₀₋₄₆O(CH₂)₀₋₁Ph which Ph may be substituted with R^{∘}; —CH═CHPh, which Ph may be substituted with R^{∘}; —(CH₂)₀₋₆O(CH₂)₀₋₁-pyridyl which pyridyl may be substituted with R^{∘}; —NO₂; —CN; —N₃; —(CH₂)₀₋₆N(R^{∘})₂; —(CH₂)₀₋₆N(R^{∘})C(O)R^{∘}); —N(R^{∘})C(S)R^{∘}); —(CH₂)₀₋₆N(R^{∘})C(O)NR^{∘})₂; —N(R^{∘})C(S)NR^{∘})₂; —(CH)₀₋₆N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₆C(O)R^{∘}; —C(S)R^{∘}; —(CH₂)₀₋₆C(O)OR^{∘}; —(CH₂)₀₋₆C(O)SR^{∘}; —(CH₂)₀₋₆C(O)OSiR^{∘}₃; —(CH₂)₀₋₆OC(O)R^{∘}; —OC(O)(CH₂)₀₋₆SR^{∘}, —(CH₂)₀₋₆SC(O)R^{∘}; —(CH₂)₀₋₆C(O)NR^{∘}₂; —C(S)NR^{∘}₂; —C(S)SR^{∘}; —SC(S)SR^{∘}, —(CH₂)₀₋₆OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₆SSR^{∘}; —(CH₂)₀₋₆SR^{∘}; —(CH₂)₀₋₆S(O)R^{∘}; —(CH₂)₀₋₆S(O)₂OR^{∘}; —(CH)₀₋₆OS(O)R^{∘}; —S(O)₂NR^{∘}₂; —(CH₂)₀₋₆S(O)R^{∘}; —N(R^{∘})S(O)NR^{∘}₂; —N(R^{∘})S(O)R^{∘}; —N(OR^{∘})R^{∘}; —C(NH)NR^{∘}₂; —P(O)R^{∘}; —P(O)R^{∘}2; —P(O)(OR^{∘})₂; —OP(O)(R^{∘})OR^{∘}; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{∘}, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, (haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}2, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur). Suitable divalent substituents on a saturated carbon atom of R^{∘} include ═O and ═S.

Suitable divalent substituents on a saturated carbon atom of an “optionally substituted” group include the following: ═O, ═S, ═NNR*2, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include: —O(CR*₂)₂₋₃O—, wherein each independent occurrence of R* is selected from hydrogen. C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{∘}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}₂, —C(O)R^{†}, —C(O)OR^{†}, —C(O)C(O)R^{†}, C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of R^{†}, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

“One or more instances” or “one or more” as referencing substitutions, as used herein, refers to, for example, 1, 2, 3, 4, 5, 6, 7, etc. instances of substitution of functional groups, which may each be independently selected, on a chemical moiety to which “one or more” instances of substitution refers. It is to be understood that any “optionally substituted” moiety, may be substituted with “one or more” optional substituents each independently selected from those optional substituents as described herein.

As used herein, the term “provided compound” or “compound of the present disclosure” refers to any genus, subgenus, and/or species set forth herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise stated, structures depicted herein are also meant to include all isomeric (e.g., enantiomeric, diastereomeric, and geometric (or conformational)) forms of the structure: for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

As used herein, the term “inhibitor” is defined as a compound that binds to and/or inhibits CDK2 with measurable affinity. In certain embodiments, an inhibitor has an IC₅₀ and/or binding constant of less than about 5 μM, less than about 1 μM, less than about 500 nM, less than about 100 nM, less than about 10 nM, or less than about 1 nM, when measured in an appropriate assay.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily or degratorily active metabolite or residue thereof.

As used herein, the term “inhibitorily active metabolite or residue thereof” means that a metabolite or residue thereof is also an inhibitor of a CDK2 protein, or a mutant thereof.

### 3. Description of Exemplary Embodiments

In certain embodiments, the present disclosure provides inhibitors of CDK2 activity. In some embodiments, the inhibitors of CDK2 include compounds of Formula I:

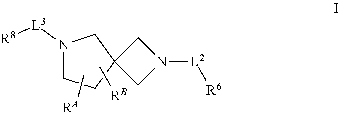

or a pharmaceutically acceptable salt thereof, wherein:
R^{A} is

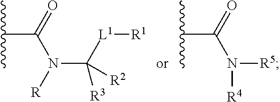

R^{B} is a hydrogen, an optionally substituted C₁₋₆ aliphatic group, —OR, —NR₂, or a halogen;
L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
R² is hydrogen, an optionally substituted C₁₋₆ aliphatic group, —C₁₋₆ alkylene-OR—C₁₋₃ alkylene-O—C₁₋₃alkylene-R—C(O)OR, —C(O)NR₂, or an optionally substituted cyclic group selected from phenyl and a 5-6 membered monocyclic heteroaromatic ring (having 1-4 heteroatoms independently selected from nitrogen, oxygen, and sulfur); and
R³ is hydrogen; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and
R⁵ is hydrogen; or
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur), or two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur), or the two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
wherein one or both of L² and L³ is a covalent bond.

As defined generally above, R^{A} is

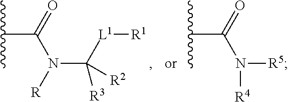

In some embodiments, R^{A} is

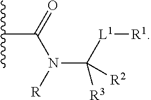

In some embodiments, R^{A} is

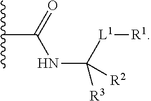

In some embodiments, R^{A} is

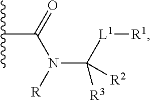

wherein the R group shown is an optionally substituted C₁₋₆ aliphatic group. In some embodiments, R^{A} is

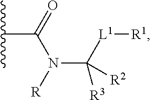

wherein the R group shown is an optionally substituted methyl group. In some embodiments, R^{A} is

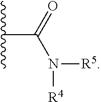

In some embodiments, R^{A} is selected from those depicted in the compounds of Table 7, below.

As defined generally above, R^{B} is a hydrogen, an optionally substituted C₁₋₆ aliphatic group, —OR, —NR₂, or a halogen. In some embodiments, R^{B} is a hydrogen. In some embodiments, R^{B} is an optionally substituted C₁₋₆ aliphatic group or a halogen. In some embodiments, R^{B} is an optionally substituted C₁₋₆ aliphatic group. In some embodiments, R^{B} is an optionally substituted methyl group. In some embodiments, R^{B} is a methyl group. In some embodiments, R^{B} is —OR. In some embodiments. R^{B} is —NR₂. In some embodiments, R^{B} is a halogen. In some embodiments, R^{B} is a F. In some embodiments, R^{B} is selected from those depicted in the compounds of Table 7, below.

In some embodiments, R^{A} and R^{B} are geminally attached to the same carbon.

As defined generally above, L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L¹ is a covalent bond. In some embodiments, L¹ is a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is a saturated or unsaturated, straight or branched, optionally substituted bivalent C₁₋₄ hydrocarbon chain, wherein 0-2 methylene units of L are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is a saturated or unsaturated, straight or branched, optionally substituted bivalent C₁₋₄ hydrocarbon chain. In some embodiments, L¹ is a saturated or unsaturated, straight or branched, optionally substituted bivalent C₁₋₄ hydrocarbon chain, wherein 1 or 2 methylene units of L¹ are replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is a saturated, straight or branched, optionally substituted bivalent C₁₋₄ hydrocarbon chain. In some embodiments, L¹ is a partially unsaturated, straight or branched, optionally substituted bivalent C₁₋₄ hydrocarbon chain. In some embodiments. L¹ is a saturated, straight, optionally substituted bivalent C₁₋₄ hydrocarbon chain, wherein 1-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is an optionally substituted straight or branched C₁₋₄ alkylene chain, wherein 1-2 methylene units of L¹ are independently replaced by —O—, —NR—, —S—, —C(O)O—, —C(O)—, —S(O)₂—, or —NRC(O)—. In some embodiments, L¹ is an optionally substituted straight or branched C₁₋₄ alkylene chain, wherein 1-2 methylene units of L¹ are independently replaced by —O—, —NR—, —C(O)O—, —C(O)—, or —NRC(O)—. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is an optionally substituted straight or branched C₁₋₄ alkylene chain, wherein 1-2 methylene units of L¹ are independently replaced by —O—. In some embodiments, L¹ is an optionally substituted straight or branched C₁₋₄ alkylene chain, wherein 1-2 methylene units of L¹ are independently replaced by —S—. In some embodiments, L¹ is an optionally substituted straight or branched C₁₋₄ alkylene chain, wherein 1-2 methylene units of L¹ are independently replaced by —S(O)₂—. In some embodiments. L¹ is an optionally substituted straight or branched C₁₋₄ alkylene chain, wherein 1-2 methylene units of L¹ are independently replaced by —NR—. In some embodiments, L¹ is an optionally substituted straight or branched C₁₋₄ alkylene chain, wherein 1-2 methylene units of L¹ are independently replaced by —C(O)O—. In some embodiments, L¹ is an optionally substituted straight or branched C₁₋₄ alkylene chain, wherein 1-2 methylene units of L¹ are independently replaced by —NRC(O)—. In some embodiments, L¹ is an unsubstituted straight chain C₁₋₄ alkynylene. In some embodiments, L¹ is selected from those depicted in the compounds of Table 7, below.

In some embodiments, L¹ is a covalent bond,

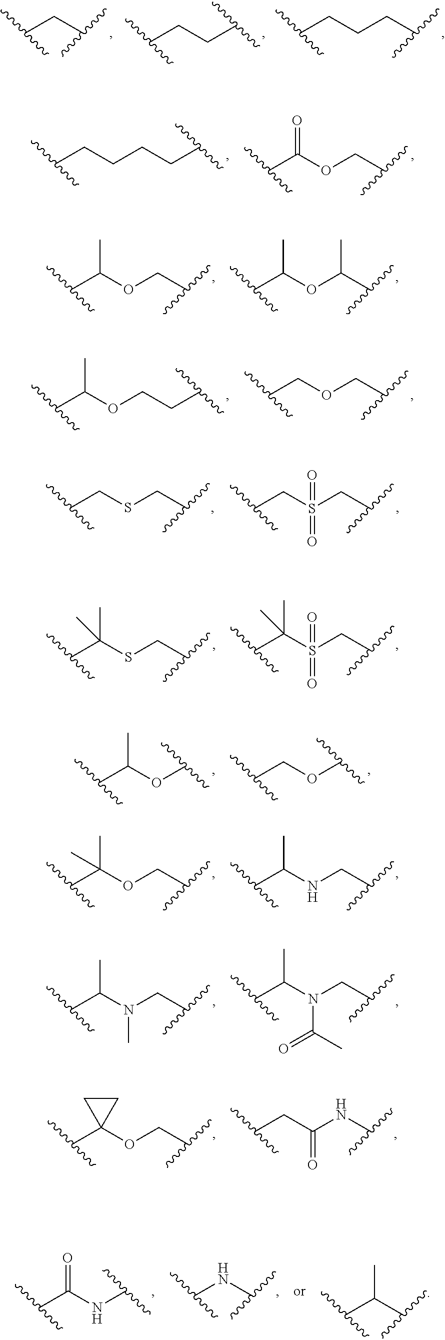

In some embodiments, L¹ is

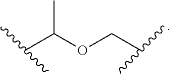 [SMILES: *COC(*)C]

As defined generally above, R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur).

In some embodiments, R¹ is hydrogen. In some embodiments, R¹ is an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur).

In some embodiments, R¹ is an optionally substituted C₁₋₆ aliphatic group. In some embodiments, R¹ is methyl. In some embodiments, R¹ is ethyl. In some embodiments, R¹ is isopropyl.

In some embodiments, R¹ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R¹ is an optionally substituted cyclic group selected from a 3-8 membered saturated or partially unsaturated monocyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 3-8 membered saturated or partially unsaturated monocyclic heterocyclic ring (having 1-2 heteroatoms independently selected from nitrogen, oxygen, and sulfur). In some embodiments, R¹ is an optionally substituted 5-6 membered monocyclic heteroaromatic ring (having 1-4 heteroatoms independently selected from nitrogen, oxygen, and sulfur). In some embodiments, R¹ is an optionally substituted 8-10 membered bicyclic heteroaromatic ring (having 1-5 heteroatoms independently selected from nitrogen, oxygen, and sulfur). In some embodiments, R¹ is an optionally substituted cyclic group selected from phenyl, cyclohexyl, cyclopentyl, cyclobutyl, cyclopropyl, cycloheptyl, oxazolyl, pyridinyl, pyridazinyl, 1,3,4-oxadiazolyl, 1,2,3-triazolyl, pyrazolyl, and tetrahydropyranyl. In some embodiments, R¹ is optionally substituted phenyl. In some embodiments, R¹ is optionally substituted cyclohexyl. In some embodiments, R¹ is selected from those depicted in the compounds of Table 7, below.

As defined generally above, R² is hydrogen, an optionally substituted C₁₋₆ aliphatic group, —C₁₋₆ alkylene-OR, —C₁₋₃ alkylene-O—C₁₋₃ alkylene-R—C(O)OR, —C(O)NR₂, or an optionally substituted cyclic group selected from phenyl and a 5-6 membered monocyclic heteroaromatic ring (having 1-4 heteroatoms independently selected from nitrogen, oxygen, and sulfur); and R³ is hydrogen; or R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur). In some embodiments, R² is hydrogen. In some embodiments, R³ is hydrogen. In some embodiments, R² is an optionally substituted C₁₋₆ aliphatic group, C₁₋₆ alkylene-OR C₁₋₃ alkylene-O—C₁₋₃ alkylene-R, —C(O)OR, or C(O)NR₂.

In some embodiments, R² is hydrogen, an optionally substituted C₁₋₆ aliphatic group, —C₁₋₆ alkylene-OR—C₁₋₃ alkylene-O—C₁₋₃ alkylene-R—C(O)OR, or —C(O)NR₂; and R³ is hydrogen. In some embodiments, R² is hydrogen, methyl, —CH₂OR—CH₂OCH₂R—C(O)OR, or —C(O)NR₂; and R³ is hydrogen. In some embodiments, R² is hydrogen. In some embodiments, R² is an optionally substituted C₁₋₆ aliphatic group. In some embodiments, R² is methyl. In some embodiments, R² is —C₁₋₆ alkylene-OR. In some embodiments, R² is CH₂OR. In some embodiments, R² is —CH₂OCH₂R. In some embodiments, R² is —C(O)OR. In some embodiments, R² is —C(O)NR₂. In some embodiments, R² is —C(O)NR₂, wherein the two R groups, taken together with the intervening nitrogen atom, form an optionally substituted 4-7 membered saturated, partially unsaturated, or heteroaryl ring (having 0-3 heteroatoms, in addition to the nitrogen, independently selected from nitrogen, oxygen, and sulfur). In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur). In some embodiments, R² is —C(O)NR₂, wherein the two R groups, taken together with the intervening nitrogen atom, form an optionally substituted 4-7 membered saturated ring, selected from a piperidinyl, morpholinyl, piperazinyl, azetindinyl, pyrrolidinyl, azaspiro[3.3]heptanyl, and diazaspiro[3.3]heptanyl.

In some embodiments, R² is selected from those depicted in the compounds of Table 7, below.

In some embodiments, R³ is hydrogen and R² is hydrogen or a substituent in Table 1:

TABLE 1
Exemplary R² substituents

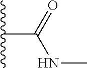 [SMILES: *C(=O)NC]

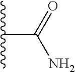 [SMILES: *C(N)=O]

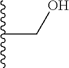 [SMILES: *CO]

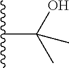 [SMILES: *C(C)(C)O]

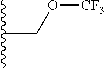 [SMILES: *COC(F)(F)F]

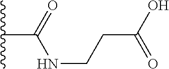 [SMILES: *C(=O)NCCC(=O)O]

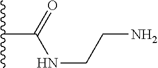 [SMILES: *C(=O)NCCN]

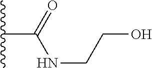 [SMILES: *C(=O)NCCO]

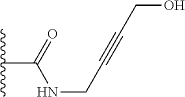 [SMILES: *C(=O)NCC#CCO]

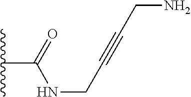 [SMILES: *C(=O)NCC#CCN]

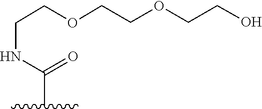 [SMILES: *C(=O)NCCOCCOCCO]

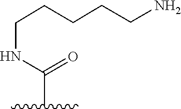 [SMILES: *C(=O)NCCCCCN]

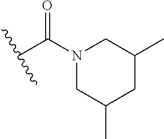 [SMILES: *C(=O)N1CC(C)CC(C)C1]

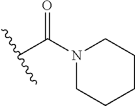 [SMILES: *C(=O)N1CCCCC1]

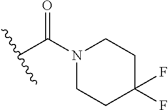 [SMILES: *C(=O)N1CCC(F)(F)CC1]

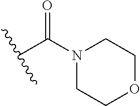 [SMILES: *C(=O)N1CCOCC1]

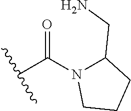 [SMILES: *C(=O)N1CCCC1CN]

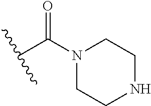 [SMILES: *C(=O)N1CCNCC1]

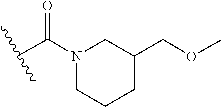 [SMILES: *C(=O)N1CCCC(COC)C1]

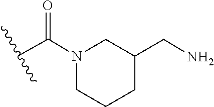 [SMILES: *C(=O)N1CCCC(CN)C1]

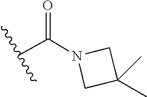 [SMILES: *C(=O)N1CC(C)(C)C1]

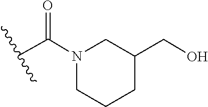 [SMILES: *C(=O)N1CCCC(CO)C1]

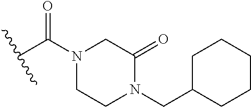 [SMILES: *C(=O)N1CCN(CC2CCCCC2)C(=O)C1]

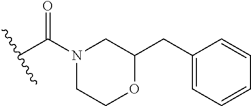 [SMILES: *C(=O)N1CCOC(Cc2ccccc2)C1]

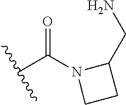 [SMILES: *C(=O)N1CCC1CN]

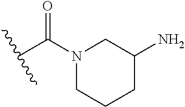 [SMILES: *C(=O)N1CCCC(N)C1]

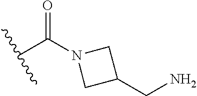 [SMILES: *C(=O)N1CC(CN)C1]

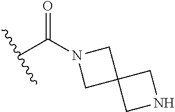 [SMILES: *C(=O)N1CC2(CNC2)C1]

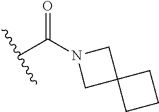 [SMILES: *C(=O)N1CC2(CCC2)C1]

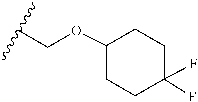 [SMILES: *COC1CCC(F)(F)CC1]

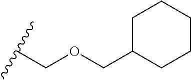 [SMILES: *COCC1CCCCC1]

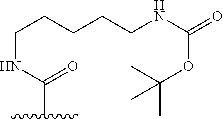 [SMILES: *C(=O)NCCCCCNC(=O)OC(C)(C)C]

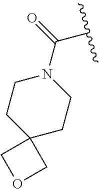 [SMILES: *C(=O)N1CCC2(CC1)COC2]

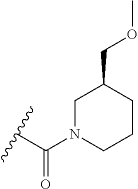 [SMILES: *C(=O)N1CCC[C@H](COC)C1]

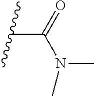 [SMILES: *C(=O)N(C)C]

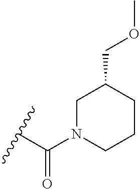 [SMILES: *C(=O)N1CCC[C@@H](COC)C1]

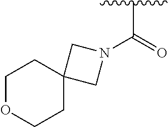 [SMILES: *C(=O)N1CC2(CCOCC2)C1]

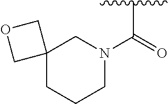 [SMILES: *C(=O)N1CCCC2(COC2)C1]

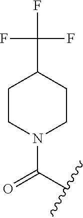 [SMILES: *C(=O)N1CCC(C(F)(F)F)CC1]

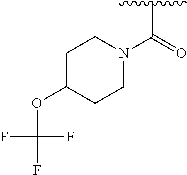 [SMILES: *C(=O)N1CCC(OC(F)(F)F)CC1]

In some embodiments, R³ is hydrogen and R² is

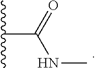 [SMILES: *C(=O)NC]

In some embodiments, R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur). In some embodiments, R² and R³ together with the intervening carbon atom form an optionally substituted 3-7 membered saturated or partially unsaturated carbocyclic ring. In some embodiments, R² and R³ together with the intervening carbon atom form an optionally substituted 3-7 membered saturated carbocyclic ring. In some embodiments, R² and R³ together with the intervening carbon atom form an optionally substituted 3-7 membered saturated or partially unsaturated heterocyclic ring (having 1-2 heteroatoms independently selected from nitrogen, oxygen, and sulfur). In some embodiments, R² and R³ together with the intervening carbon atom form an optionally substituted 3-7 membered saturated heterocyclic ring (having 1-2 heteroatoms independently selected from nitrogen, oxygen, and sulfur). In some embodiments, R² and R³ together with the intervening carbon atom form an optionally substituted oxetanyl, cyclopropyl, cyclobutyl, cyclopentyl, tetrahydropyranyl, piperidinyl, piperazinyl, morpholinyl, pyrrolidinyl, or 1,4-oxazepanyl. In some embodiments, R² and R³ form a cyclic group selected from those depicted in the compounds of Table 7, below.

As defined generally above, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), and R⁵ is hydrogen; or R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, independently selected from nitrogen, oxygen, and sulfur).

In some embodiments, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), and R⁵ is hydrogen. In some embodiments, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur). In some embodiments, R⁴ is an optionally substituted 3-8 membered saturated or partially unsaturated monocyclic carbocyclic ring. In some embodiments, R⁴ is an optionally substituted 7-12 membered saturated or partially unsaturated bicyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 3-8 membered saturated or partially unsaturated monocyclic heterocyclic ring (having 1-2 heteroatoms independently selected from nitrogen, oxygen, and sulfur). In some embodiments, R⁴ is an optionally substituted 5-6 membered monocyclic heteroaromatic ring (having 1-4 heteroatoms independently selected from nitrogen, oxygen, and sulfur). In some embodiments. R⁴ is an optionally substituted cyclic group selected from phenyl, piperidinyl, tetrahydropyranyl, 1,4-oxazepanyl, oxazolyl, cyclobutyl, cyclopentyl, or pyrrolidinyl. In some embodiments, R⁴ is selected from those depicted in the compounds of Table 7, below.

In some embodiments, R⁴ and R⁵ together with the intervening nitrogen atom form an optionally substituted 4-7 membered saturated, or partially unsaturated heterocyclic ring (having 0)-2 heteroatoms, in addition to the nitrogen, independently selected from nitrogen, oxygen, and sulfur), or an optionally substituted heteroaryl ring (having 0)-3 heteroatoms, in addition to the nitrogen, independently selected from nitrogen, oxygen, and sulfur). In some embodiments, R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur). In some embodiments, R⁴ and R⁵ together with the intervening nitrogen atom form an optionally substituted heteroaryl ring (having 0-3 heteroatoms, in addition to the nitrogen, independently selected from nitrogen, oxygen, and sulfur). In some embodiments, R⁴ and R⁵ together with the intervening nitrogen atom form an optionally substituted cyclic group selected from piperindinyl, piperazinyl, morpholinyl, and pyrrolidinyl. In some embodiments, R⁴ and R⁵ together with the intervening nitrogen atom form a substituted cyclic group, wherein the cyclic group is substituted with a group selected from —C₁₋₆ alkylene-phenyl, —O—C₁₋₄ alkylene-phenyl, —C₁₋₄ alkylene-cyclohexyl, and —O—C₁₋₆ alkylene-cyclohexyl. In some embodiments, R⁴ and R⁵ form a cyclic group selected from those depicted in the compounds of Table 7, below.

In some embodiments, R^{A} is a substituent of Table 2:

TABLE 2
Exemplary R^{A} substituents

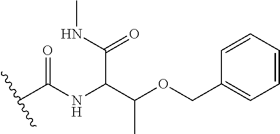 [SMILES: *C(=O)NC(C(=O)NC)C(C)OCc1ccccc1]

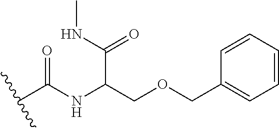 [SMILES: *C(=O)NC(COCc1ccccc1)C(=O)NC]

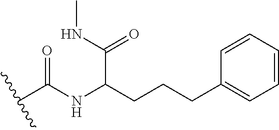 [SMILES: *C(=O)NC(CCCc1ccccc1)C(=O)NC]

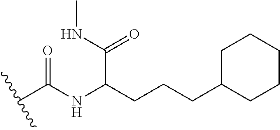 [SMILES: *C(=O)NC(CCCC1CCCCC1)C(=O)NC]

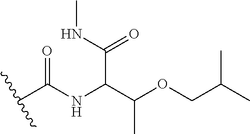 [SMILES: *C(=O)NC(C(=O)NC)C(C)OCC(C)C]

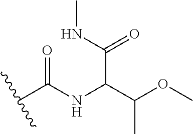 [SMILES: *C(=O)NC(C(=O)NC)C(C)OC]

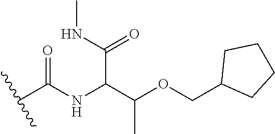 [SMILES: *C(=O)NC(C(=O)NC)C(C)OCC1CCCC1]

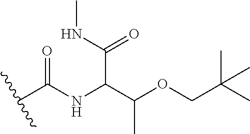 [SMILES: *C(=O)NC(C(=O)NC)C(C)OCC(C)(C)C]

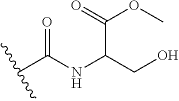 [SMILES: *C(=O)NC(CO)C(=O)OC]

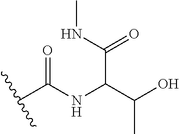 [SMILES: *C(=O)NC(C(=O)NC)C(C)O]

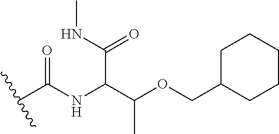 [SMILES: *C(=O)NC(C(=O)NC)C(C)OCC1CCCCC1]

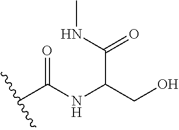 [SMILES: *C(=O)NC(CO)C(=O)NC]

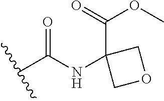 [SMILES: *C(=O)NC1(C(=O)OC)COC1]

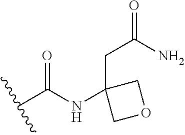 [SMILES: *C(=O)NC1(CC(N)=O)COC1]

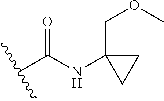 [SMILES: *C(=O)NC1(COC)CC1]

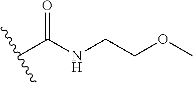 [SMILES: *C(=O)NCCOC]

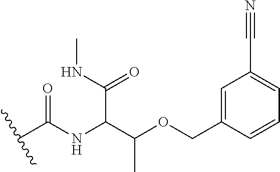 [SMILES: *C(=O)NC(C(=O)NC)C(C)OCc1cccc(C#N)c1]

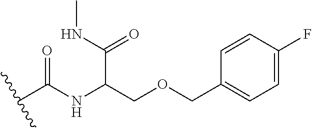 [SMILES: *C(=O)NC(COCc1ccc(F)cc1)C(=O)NC]

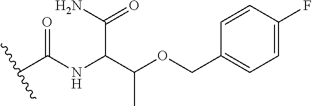 [SMILES: *C(=O)NC(C(N)=O)C(C)OCc1ccc(F)cc1]

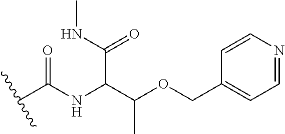 [SMILES: *C(=O)NC(C(=O)NC)C(C)OCc1ccncc1]

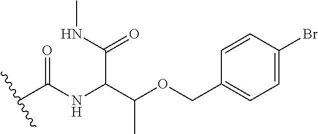 [SMILES: *C(=O)NC(C(=O)NC)C(C)OCc1ccc(Br)cc1]

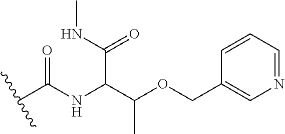 [SMILES: *C(=O)NC(C(=O)NC)C(C)OCc1cccnc1]

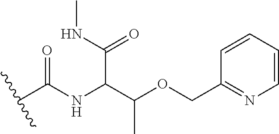 [SMILES: *C(=O)NC(C(=O)NC)C(C)OCc1ccccn1]

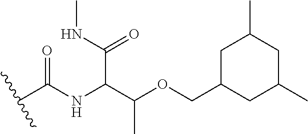 [SMILES: *C(=O)NC(C(=O)NC)C(C)OCC1CC(C)CC(C)C1]

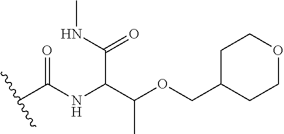 [SMILES: *C(=O)NC(C(=O)NC)C(C)OCC1CCOCC1]

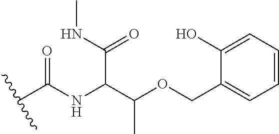 [SMILES: *C(=O)NC(C(=O)NC)C(C)OCc1ccccc1O]

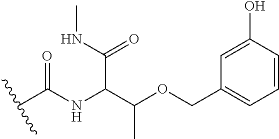 [SMILES: *C(=O)NC(C(=O)NC)C(C)OCc1cccc(O)c1]

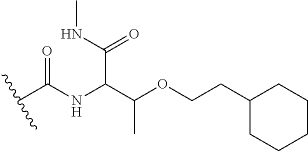 [SMILES: *C(=O)NC(C(=O)NC)C(C)OCCC1CCCCC1]

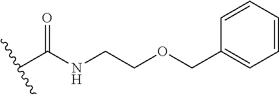 [SMILES: *C(=O)NCCOCc1ccccc1]

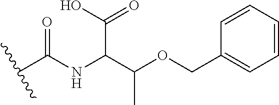 [SMILES: *C(=O)NC(C(=O)O)C(C)OCc1ccccc1]

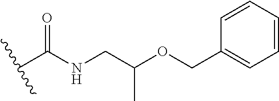 [SMILES: *C(=O)NCC(C)OCc1ccccc1]

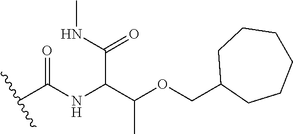 [SMILES: *C(=O)NC(C(=O)NC)C(C)OCC1CCCCCC1]

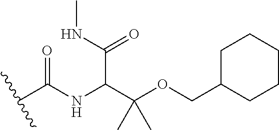 [SMILES: *C(=O)NC(C(=O)NC)C(C)(C)OCC1CCCCC1]

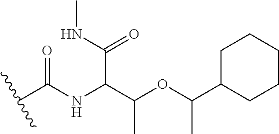 [SMILES: *C(=O)NC(C(=O)NC)C(C)OC(C)C1CCCCC1]

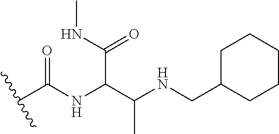 [SMILES: *C(=O)NC(C(=O)NC)C(C)NCC1CCCCC1]

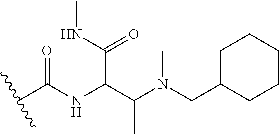 [SMILES: *C(=O)NC(C(=O)NC)C(C)N(C)CC1CCCCC1]

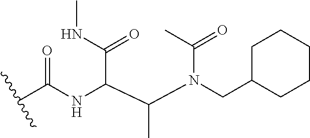 [SMILES: *C(=O)NC(C(=O)NC)C(C)N(CC1CCCCC1)C(C)=O]

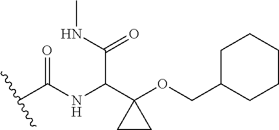 [SMILES: *C(=O)NC(C(=O)NC)C1(OCC2CCCCC2)CC1]

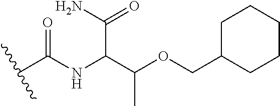 [SMILES: *C(=O)NC(C(N)=O)C(C)OCC1CCCCC1]

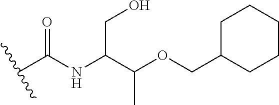 [SMILES: *C(=O)NC(CO)C(C)OCC1CCCCC1]

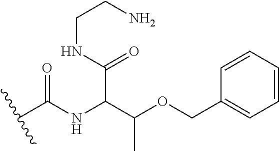 [SMILES: *C(=O)NC(C(=O)NCCN)C(C)OCc1ccccc1]

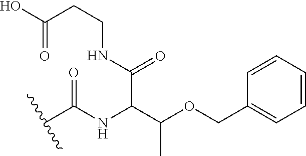 [SMILES: *C(=O)NC(C(=O)NCCC(=O)O)C(C)OCc1ccccc1]

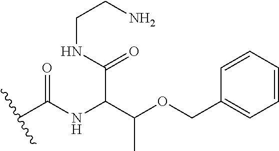 [SMILES: *C(=O)NC(C(=O)NCCN)C(C)OCc1ccccc1]

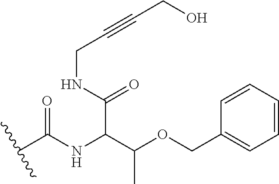 [SMILES: *C(=O)NC(C(=O)NCC#CCO)C(C)OCc1ccccc1]

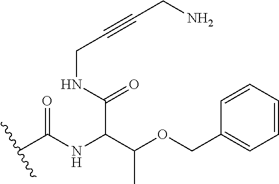 [SMILES: *C(=O)NC(C(=O)NCC#CCN)C(C)OCc1ccccc1]

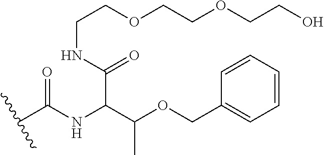 [SMILES: *C(=O)NC(C(=O)NCCOCCOCCO)C(C)OCc1ccccc1]

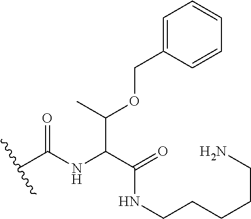 [SMILES: *C(=O)NC(C(=O)NCCCCCN)C(C)OCc1ccccc1]

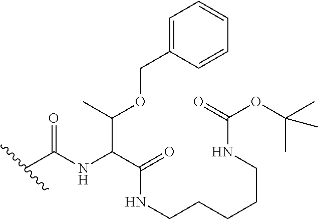 [SMILES: *C(=O)NC(C(=O)NCCCCCNC(=O)OC(C)(C)C)C(C)OCc1ccccc1]

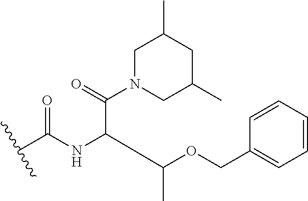 [SMILES: *C(=O)NC(C(=O)N1CC(C)CC(C)C1)C(C)OCc1ccccc1]

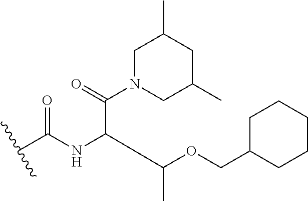 [SMILES: *C(=O)NC(C(=O)N1CC(C)CC(C)C1)C(C)OCC1CCCCC1]

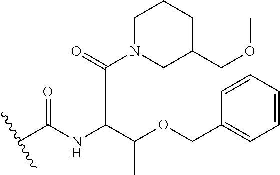 [SMILES: *C(=O)NC(C(=O)N1CCCC(COC)C1)C(C)OCc1ccccc1]

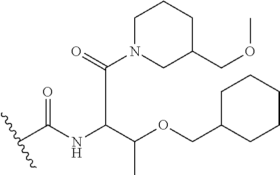 [SMILES: *C(=O)NC(C(=O)N1CCCC(COC)C1)C(C)OCC1CCCCC1]

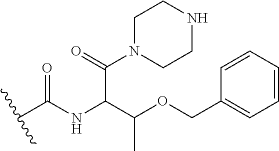 [SMILES: *C(=O)NC(C(=O)N1CCNCC1)C(C)OCc1ccccc1]

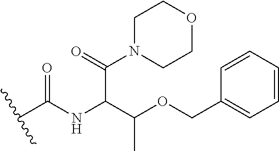 [SMILES: *C(=O)NC(C(=O)N1CCOCC1)C(C)OCc1ccccc1]

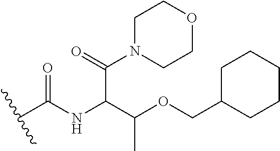 [SMILES: *C(=O)NC(C(=O)N1CCOCC1)C(C)OCC1CCCCC1]

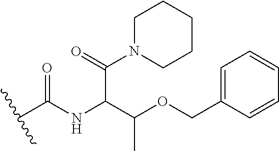 [SMILES: *C(=O)NC(C(=O)N1CCCCC1)C(C)OCc1ccccc1]

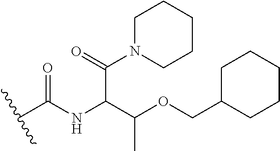 [SMILES: *C(=O)NC(C(=O)N1CCCCC1)C(C)OCC1CCCCC1]

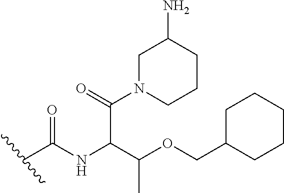 [SMILES: *C(=O)NC(C(=O)N1CCCC(N)C1)C(C)OCC1CCCCC1]

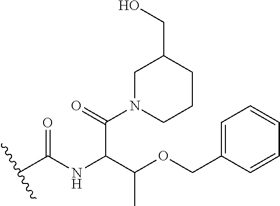 [SMILES: *C(=O)NC(C(=O)N1CCCC(CO)C1)C(C)OCc1ccccc1]

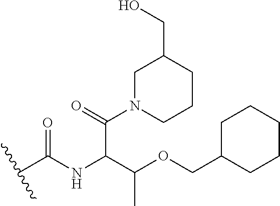 [SMILES: *C(=O)NC(C(=O)N1CCCC(CO)C1)C(C)OCC1CCCCC1]

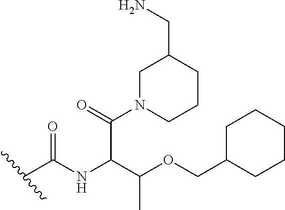 [SMILES: *C(=O)NC(C(=O)N1CCCC(CN)C1)C(C)OCC1CCCCC1]

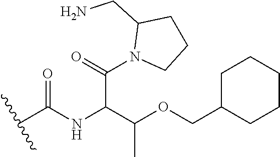 [SMILES: *C(=O)NC(C(=O)N1CCCC1CN)C(C)OCC1CCCCC1]

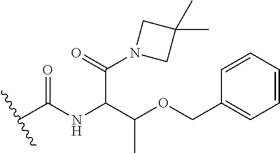

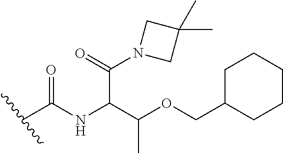 [SMILES: *C(=O)NC(C(=O)N1CC(C)(C)C1)C(C)OCC1CCCCC1]

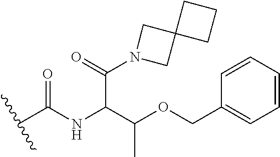 [SMILES: *C(=O)NC(C(=O)N1CC2(CCC2)C1)C(C)OCc1ccccc1]

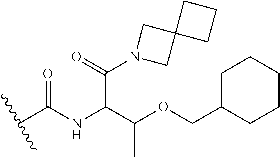 [SMILES: *C(=O)NC(C(=O)N1CC2(CCC2)C1)C(C)OCC1CCCCC1]

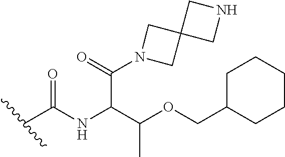 [SMILES: *C(=O)NC(C(=O)N1CC2(CNC2)C1)C(C)OCC1CCCCC1]

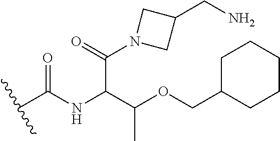 [SMILES: *C(=O)NC(C(=O)N1CC(CN)C1)C(C)OCC1CCCCC1]

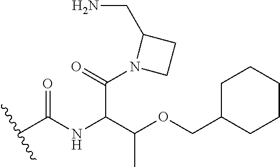 [SMILES: *C(=O)NC(C(=O)N1CCC1CN)C(C)OCC1CCCCC1]

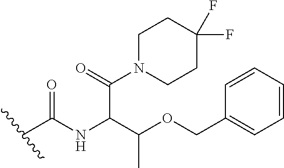 [SMILES: *C(=O)NC(C(=O)N1CCC(F)(F)CC1)C(C)OCc1ccccc1]

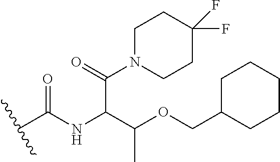 [SMILES: *C(=O)NC(C(=O)N1CCC(F)(F)CC1)C(C)OCC1CCCCC1]

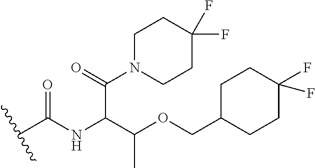 [SMILES: *C(=O)NC(C(=O)N1CCC(F)(F)CC1)C(C)OCC1CCC(F)(F)CC1]

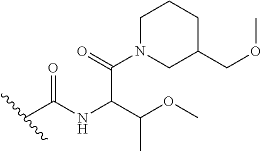 [SMILES: *C(=O)NC(C(=O)N1CCCC(COC)C1)C(C)OC]

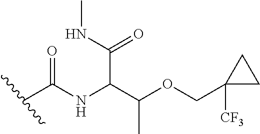 [SMILES: *C(=O)NC(C(=O)NC)C(C)OCC1(C(F)(F)F)CC1]

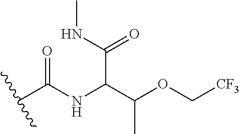 [SMILES: *C(=O)NC(C(=O)NC)C(C)OCC(F)(F)F]

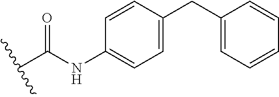 [SMILES: *C(=O)Nc1ccc(Cc2ccccc2)cc1]

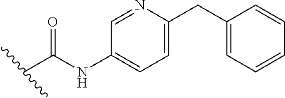 [SMILES: *C(=O)Nc1ccc(Cc2ccccc2)nc1]

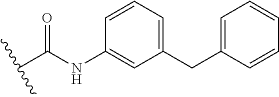 [SMILES: *C(=O)Nc1cccc(Cc2ccccc2)c1]

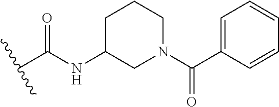 [SMILES: *C(=O)NC1CCCN(C(=O)c2ccccc2)C1]

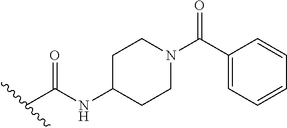 [SMILES: *C(=O)NC1CCN(C(=O)c2ccccc2)CC1]

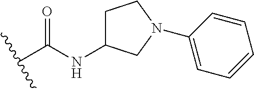 [SMILES: *C(=O)NC1CCN(c2ccccc2)C1]

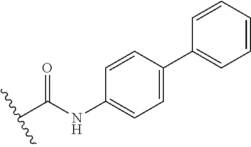 [SMILES: *C(=O)Nc1ccc(-c2ccccc2)cc1]

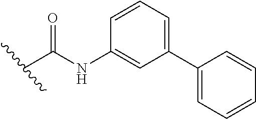 [SMILES: *C(=O)Nc1cccc(-c2ccccc2)c1]

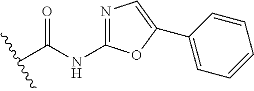 [SMILES: *C(=O)Nc1ncc(-c2ccccc2)o1]

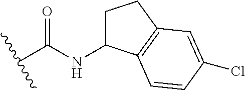 [SMILES: *C(=O)NC1CCc2cc(Cl)ccc21]

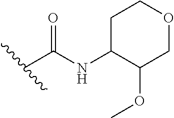 [SMILES: *C(=O)NC1CCOCC1OC]

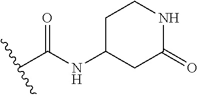 [SMILES: *C(=O)NC1CCNC(=O)C1]

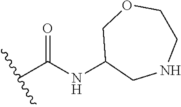 [SMILES: *C(=O)NC1CNCCOC1]

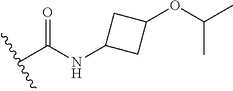 [SMILES: *C(=O)NC1CC(OC(C)C)C1]

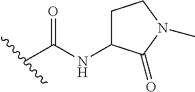 [SMILES: *C(=O)NC1CCN(C)C1=O]

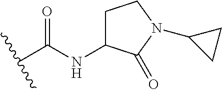 [SMILES: *C(=O)NC1CCN(C2CC2)C1=O]

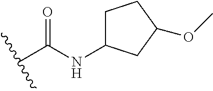 [SMILES: *C(=O)NC1CCC(OC)C1]

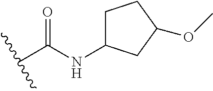 [SMILES: *C(=O)NC1CCC(OC)C1]

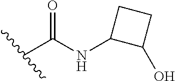 [SMILES: *C(=O)NC1CCC1O]

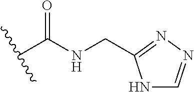 [SMILES: *C(=O)NCc1nnc[nH]1]

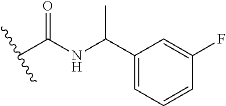 [SMILES: *C(=O)NC(C)c1cccc(F)c1]

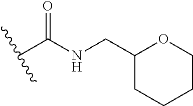 [SMILES: *C(=O)NCC1CCCCO1]

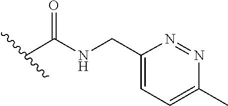 [SMILES: *C(=O)NCc1ccc(C)nn1]

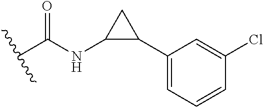 [SMILES: *C(=O)NC1CC1c1cccc(Cl)c1]

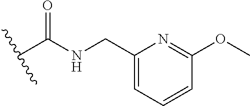 [SMILES: *C(=O)NCc1cccc(OC)n1]

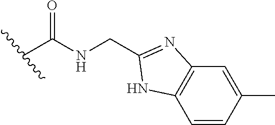 [SMILES: *C(=O)NCc1nc2cc(C)ccc2[nH]1]

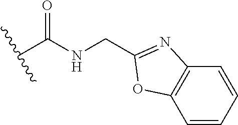 [SMILES: *C(=O)NCc1nc2ccccc2o1]

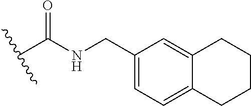 [SMILES: *C(=O)NCc1ccc2c(c1)CCCC2]

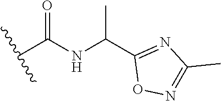 [SMILES: *C(=O)NC(C)c1nc(C)no1]

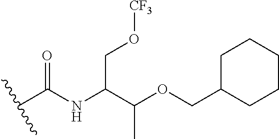 [SMILES: *C(=O)NC(COC(F)(F)F)C(C)OCC1CCCCC1]

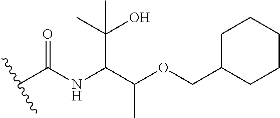 [SMILES: *C(=O)NC(C(C)OCC1CCCCC1)C(C)(C)O]

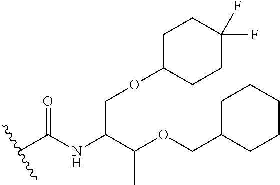 [SMILES: *C(=O)NC(COC1CCC(F)(F)CC1)C(C)OCC1CCCCC1]

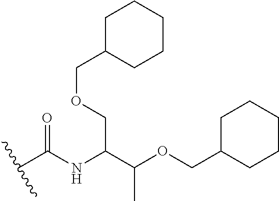 [SMILES: *C(=O)NC(COCC1CCCCC1)C(C)OCC1CCCCC1]

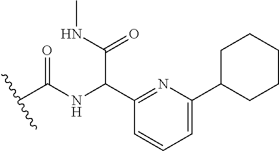 [SMILES: *C(=O)NC(C(=O)NC)c1cccc(C2CCCCC2)n1]

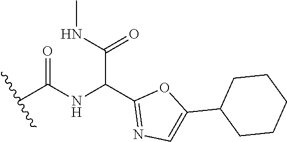 [SMILES: *C(=O)NC(C(=O)NC)c1ncc(C2CCCCC2)o1]

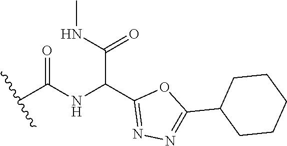 [SMILES: *C(=O)NC(C(=O)NC)c1nnc(C2CCCCC2)o1]

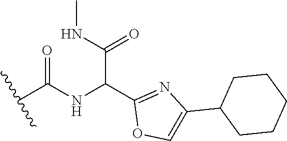 [SMILES: *C(=O)NC(C(=O)NC)c1nc(C2CCCCC2)co1]

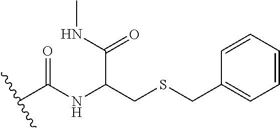 [SMILES: *C(=O)NC(CSCc1ccccc1)C(=O)NC]

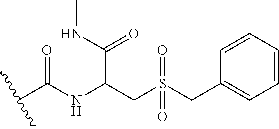 [SMILES: *C(=O)NC(CS(=O)(=O)Cc1ccccc1)C(=O)NC]

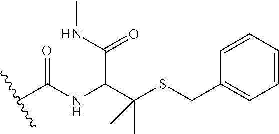 [SMILES: *C(=O)NC(C(=O)NC)C(C)(C)SCc1ccccc1]

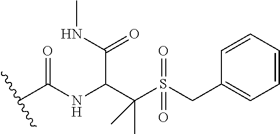 [SMILES: *C(=O)NC(C(=O)NC)C(C)(C)S(=O)(=O)Cc1ccccc1]

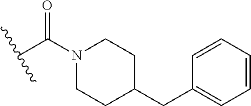 [SMILES: *C(=O)N1CCC(Cc2ccccc2)CC1]

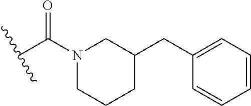 [SMILES: *C(=O)N1CCCC(Cc2ccccc2)C1]

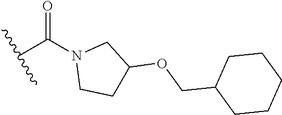 [SMILES: *C(=O)N1CCC(OCC2CCCCC2)C1]

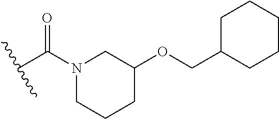 [SMILES: *C(=O)N1CCCC(OCC2CCCCC2)C1]

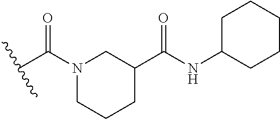 [SMILES: *C(=O)N1CCCC(C(=O)NC2CCCCC2)C1]

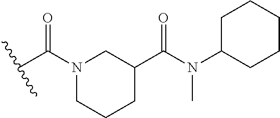 [SMILES: *C(=O)N1CCCC(C(=O)N(C)C2CCCCC2)C1]

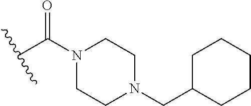 [SMILES: *C(=O)N1CCN(CC2CCCCC2)CC1]

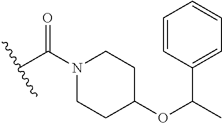 [SMILES: *C(=O)N1CCC(OC(C)c2ccccc2)CC1]

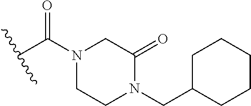 [SMILES: *C(=O)N1CCN(CC2CCCCC2)C(=O)C1]

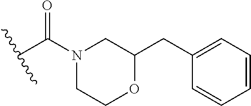 [SMILES: *C(=O)N1CCOC(Cc2ccccc2)C1]

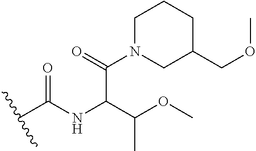 [SMILES: *C(=O)NC(C(=O)N1CCCC(COC)C1)C(C)OC]

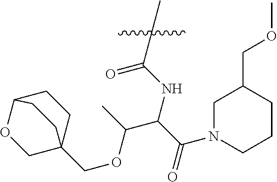 [SMILES: *C(=O)NC(C(=O)N1CCCC(COC)C1)C(C)OCC12CCC(CC1)OC2]

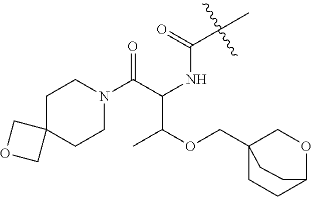 [SMILES: *C(=O)NC(C(=O)N1CCC2(CC1)COC2)C(C)OCC12CCC(CC1)OC2]

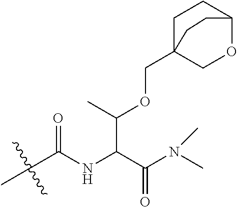 [SMILES: *C(=O)NC(C(=O)N(C)C)C(C)OCC12CCC(CC1)OC2]

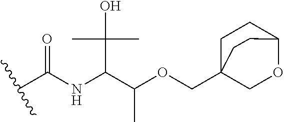 [SMILES: *C(=O)NC(C(C)OCC12CCC(CC1)OC2)C(C)(C)O]

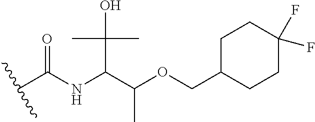 [SMILES: *C(=O)NC(C(C)OCC1CCC(F)(F)CC1)C(C)(C)O]

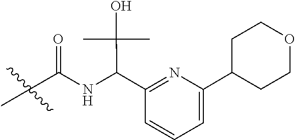 [SMILES: *C(=O)NC(c1cccc(C2CCOCC2)n1)C(C)(C)O]

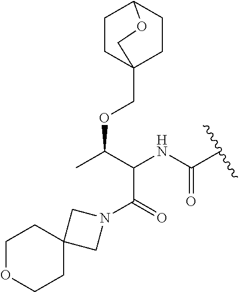 [SMILES: *C(=O)NC(C(=O)N1CC2(CCOCC2)C1)[C@@H](C)OCC12CCC(CC1)OC2]

As defined generally above, L¹ is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L² is a covalent bond. In some embodiments, L² is a saturated or unsaturated, straight or branched, optionally substituted bivalent C₁₋₄ hydrocarbon chain, wherein 0-2 methylene units of L² are independently replaced by —C(O)O—, —C(O)—, or —C(O)NR—. In some embodiments, L¹ is a C₁₋₄ alkylene chain, wherein 1-2 methylene units of L² are independently replaced by —C(O)O—, —C(O)—, or —C(O)NR—. In some embodiments, L² is C₁₋₄ alkylene chain, wherein 1 methylene unit of L² is replaced by —C(O)O—, —C(O)—, or —C(O)NR—. In some embodiments, L² is a saturated optionally substituted bivalent C₁₋₄ hydrocarbon chain. In some embodiments, L² is a saturated bivalent C₁₋₄ hydrocarbon chain, substituted on a single methylene unit by two substituents, which together with the intervening carbon atom form a 3-7 membered carbocyclic ring or heterocyclic ring (having 1-2 heteroatoms independently selected from nitrogen, oxygen, and sulfur). In some embodiments, L² is

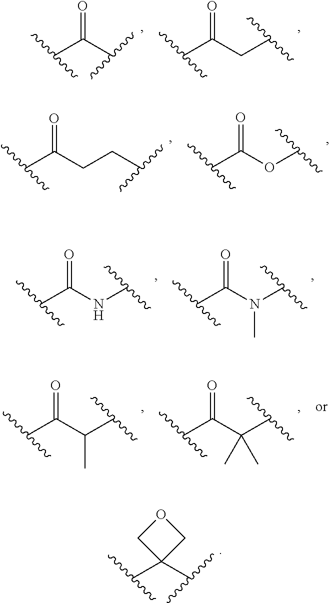

In some embodiments, L² is

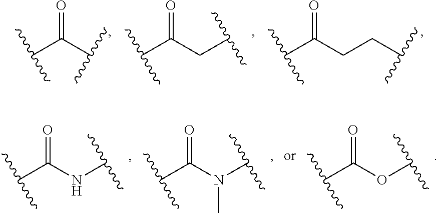

In some embodiments, L² is

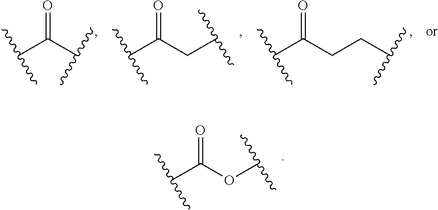

In some embodiments, L² is

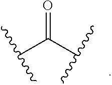 [SMILES: *C(*)=O]

In some embodiments, L² is a saturated, straight or branched, optionally substituted bivalent C₁₋₄ hydrocarbon chain. In some embodiments, L² is methylene. In some embodiments, L² is —S(O)₂—. In some embodiments, L² is selected from those depicted in the compounds of Table 7, below.

As defined generally above, R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷.

In some embodiments, R⁶ is an optionally substituted C₁₋₆ aliphatic group. In some embodiments, R⁶ is an optionally substituted methyl, ethyl, isopropyl, or tert-butyl group.

In some embodiments, R⁶ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷. In some embodiments, R⁶ is a 3-8 membered saturated or partially unsaturated monocyclic carbocyclic ring, optionally substituted with one or more instances of R⁷. In some embodiments, R⁶ is a phenyl group, optionally substituted with one or more instances of R⁷. In some embodiments, R⁶ is a cyclic group selected from cyclopropyl, cyclobutyl, cyclohexyl and phenyl, wherein the cyclic group is optionally substituted with one or more instances of R⁷. In some embodiments, R⁶ is a cyclopropyl group, optionally substituted with one or more instances of R⁷. In some embodiments, R⁶ is selected from those depicted in the compounds of Table 7, below.

As defined generally above, each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy. In some embodiments, each instance of R⁷ is independently halogen, —OR, —CN, an optionally substituted C₁₋₆ aliphatic group, an optionally substituted C₁₋₆ aliphatic-Cy group, or Cy. In some embodiments, each instance of R⁷ is independently —F, methyl, ethyl, isopropyl, isobutyl, —CN, optionally substituted phenyl, optionally substituted benzyl, —CF₃, —CH₂OH, —CH₂OCH₃, —CH₂CH₂OCH₃, —CH₂CH₂F, cyclopropyl or —CH₂-(cyclopropyl). In some embodiments, each instance of R⁷ is independently a C₁₋₆ aliphatic group.

In some embodiments, there are 0 instances of R⁷. In some embodiments, there is 1 instance of R⁷. In some embodiments, there are 2 instances of R⁷. In some embodiments, there are 3 instances of R⁷. In some embodiments, there are 4 instances of R⁷.

In some embodiments, -L²-R⁶, wherein R⁶ is optionally substituted with one or more instances of R⁷, is a substituent of Table 3 or Table 4. In some embodiments, the -L²-R⁶ of Table 3 or Table 4 is shown with the one or more instance of R⁷. Also contemplated are embodiments wherein the -L²-R⁶ of Table 3 or Table 4 is further substituted with one or more instances of R⁷ which are not shown in Table 3 or Table 4.

TABLE 3
Exemplary —L²—R⁶ substituents

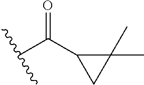 [SMILES: *C(=O)C1CC1(C)C]

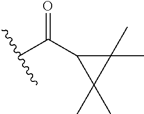 [SMILES: *C(=O)C1C(C)(C)C1(C)C]

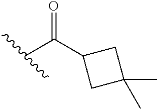 [SMILES: *C(=O)C1CC(C)(C)C1]

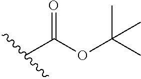 [SMILES: *C(=O)OC(C)(C)C]

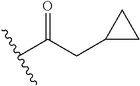 [SMILES: *C(=O)CC1CC1]

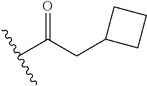 [SMILES: *C(=O)CC1CCC1]

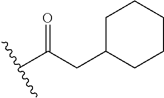 [SMILES: *C(=O)CC1CCCCC1]

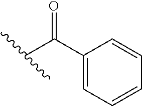 [SMILES: *C(=O)c1ccccc1]

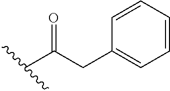 [SMILES: *C(=O)Cc1ccccc1]

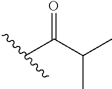 [SMILES: *C(=O)C(C)C]

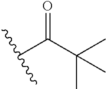 [SMILES: *C(=O)C(C)(C)C]

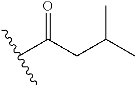 [SMILES: *C(=O)CC(C)C]

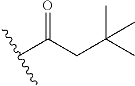 [SMILES: *C(=O)CC(C)(C)C]

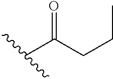 [SMILES: *C(=O)CCC]

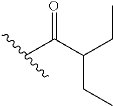 [SMILES: *C(=O)C(CC)CC]

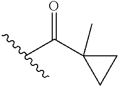 [SMILES: *C(=O)C1(C)CC1]

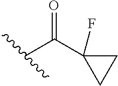 [SMILES: *C(=O)C1(F)CC1]

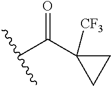 [SMILES: *C(=O)C1(C(F)(F)F)CC1]

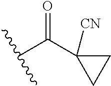 [SMILES: *C(=O)C1(C#N)CC1]

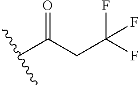 [SMILES: *C(=O)CC(F)(F)F]

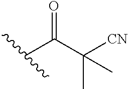 [SMILES: *C(=O)C(C)(C)C#N]

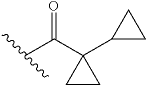 [SMILES: *C(=O)C1(C2CC2)CC1]

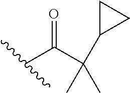 [SMILES: *C(=O)C(C)(C)C1CC1]

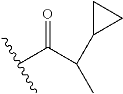 [SMILES: *C(=O)C(C)C1CC1]

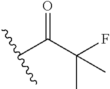 [SMILES: *C(=O)C(C)(C)F]

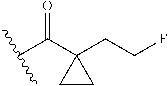 [SMILES: *C(=O)C1(CCF)CC1]

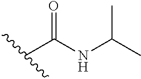 [SMILES: *C(=O)NC(C)C]

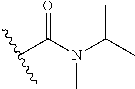 [SMILES: *C(=O)N(C)C(C)C]

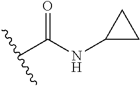 [SMILES: *C(=O)NC1CC1]

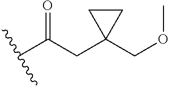 [SMILES: *C(=O)CC1(COC)CC1]

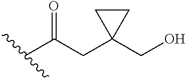 [SMILES: *C(=O)CC1(CO)CC1]

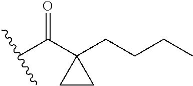 [SMILES: *C(=O)C1(CCCC)CC1]

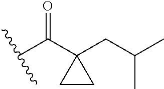 [SMILES: *C(=O)C1(CC(C)C)CC1]

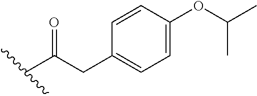 [SMILES: *C(=O)Cc1ccc(OC(C)C)cc1]

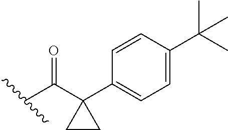 [SMILES: *C(=O)C1(c2ccc(C(C)(C)C)cc2)CC1]

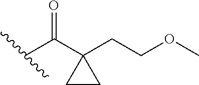 [SMILES: *C(=O)C1(CCOC)CC1]

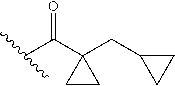 [SMILES: *C(=O)C1(CC2CC2)CC1]

TABLE 4
Exemplary —L₂—R⁶ or R⁶ substituents

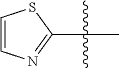 [SMILES: *c1nccs1]

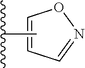

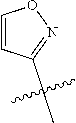 [SMILES: *c1ccon1]

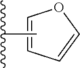

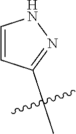 [SMILES: *c1cc[nH]n1]

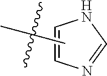

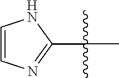 [SMILES: *c1ncc[nH]1]

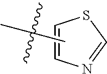

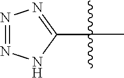 [SMILES: *c1nnn[nH]1]

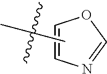

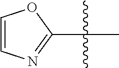 [SMILES: *c1ncco1]

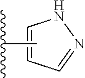

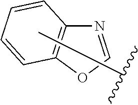

In some embodiments, -L²-R⁶ is

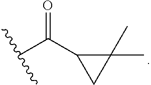 [SMILES: *C(=O)C1CC1(C)C]

In some embodiments, -L²-R⁶ is

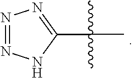 [SMILES: *c1nnn[nH]1]

In some embodiments, -L²-R⁶ is

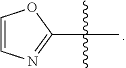 [SMILES: *c1ncco1]

In some embodiments, -L²-R⁶ is

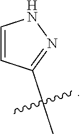 [SMILES: *c1cc[nH]n1]

As defined generally above, L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L³ is a covalent bond. In some embodiments, L³ is a saturated or unsaturated, straight or branched, optionally substituted bivalent C₁₋₄ hydrocarbon chain, wherein 0-2 methylene units of L³ are independently replaced by —S(O)₂—, —C(O)NR—, or —C(O)—. In some embodiments, L³ is a C₁₋₄ alkylene chain, wherein 1-2 methylene units of L³ are independently replaced by —S(O)₂—, —C(O)NR— or —C(O)—. In some embodiments. L³ is C₁₋₄ alkylene chain, wherein 1 methylene unit of L³ is replaced by —S(O)₂—, —C(O)NR—, or —C(O)—. In some embodiments, L³ is a saturated or unsaturated, straight or branched, optionally substituted bivalent C₁₋₄ alkylene chain, wherein 0-2 methylene units of L³ are independently replaced by —C(O)O— or —C(O)—. In some embodiments, L³ is a C₁₋₄ alkylene chain, wherein 1-2 methylene units of L³ are independently replaced by —C(O)O— or —C(O)—. In some embodiments, L³ is C₁₋₄ alkylene chain, wherein 1 methylene unit of L³ is replaced by —C(O)O— or —C(O)—. In some embodiments, L³ is a saturated optionally substituted bivalent C₁₋₄ hydrocarbon chain. In some embodiments, L¹ is a saturated bivalent C₁₋₄ hydrocarbon chain, substituted on a single methylene unit by two substituents, which together with the intervening carbon atom (the single methylene unit) form a 3-7 membered carbocyclic ring or heterocyclic ring (having 1-2 heteroatoms independently selected from nitrogen, oxygen, and sulfur). In some embodiments, L³ is

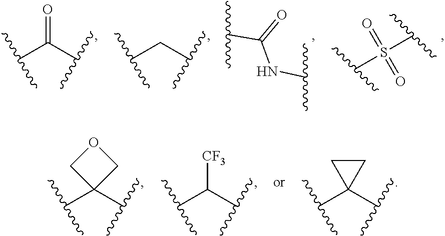

In some embodiments, L³ is

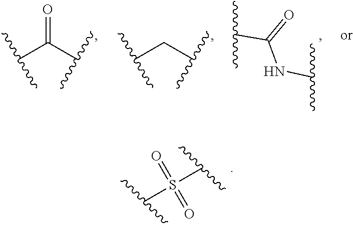

In some embodiments, L³ is

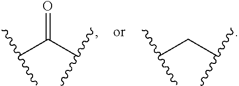

In some embodiments. L³ is selected from those depicted in the compounds of Table 7, below.

As defined generally above, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a cyclic group selected from a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a 3-8 membered saturated or partially unsaturated monocyclic heterocyclic ring (having 1-2 heteroatoms independently selected from nitrogen, oxygen, and sulfur), optionally substituted with one or more instances of R⁹. In some embodiments, R⁸ is a 5-6 membered monocyclic heteroaromatic ring (having 1-4 heteroatoms independently selected from nitrogen, oxygen, and sulfur), optionally substituted with one or more instances of R⁹. In some embodiments, R⁸ is an 8-10 membered bicyclic heteroaromatic ring (having 1-5 heteroatoms independently selected from nitrogen, oxygen, and sulfur), optionally substituted with one or more instances of R⁹. In some embodiments, R⁸ is a cyclic group selected from pyrazolyl, oxazolyl, thiazolyl, pyrrolidinyl, tetrahydropyranyl, pyridinyl, imidazolyl, indolyl, 1,2,4-triazolyl, 1,2,4-thiadiazolyl, piperidinyl, and indazolyl, wherein the cyclic group is optionally substituted with one or more instances of R⁹. In some embodiments, R⁸ is a pyrazolyl or thiazolyl group, optionally substituted with one or more instances of R⁹. In some embodiments, R⁸ is a pyrazolyl or thiazolyl group.

In some embodiments, R⁸ is a cyclic group selected from an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments. R⁸ is an 8-10 membered bicyclic heteroaromatic ring (having 1-5 heteroatoms independently selected from nitrogen, oxygen, and sulfur), optionally substituted with one or more instances of R⁹, and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L³ is a covalent bond and R⁸ is a 5-6 membered heteroaryl optionally substituted with one or more instances of R⁹.

In some embodiments, R⁸ is selected from those depicted in the compounds of Table 7, below.

As defined generally above, each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy. In some embodiments, there are 0 instances of R⁹. In some embodiments, there is 1 instance of R⁹. In some embodiments, there are 2 instances of R⁹. In some embodiments, there are 3 instances of R⁹.

In some embodiments, each instance of R⁹ is independently halogen, an optionally substituted C₁₋₆ aliphatic group, an optionally substituted C₁₋₆ aliphatic-Cy group, or Cy. In some embodiments, each instance of R⁹ is independently an optionally substituted C₁₋₆ aliphatic-Cy group, wherein the Cy is an optionally substituted group selected from phenyl, cyclohexyl, pyridinyl, piperidinyl, cyclopropyl, or tetrahydropyranyl. In some embodiments, R⁹ is a benzylic group. In some embodiments, each instance of R⁹ is independently halogen or an optionally substituted C₁₋₆ aliphatic group. In some embodiments, R⁹ is selected from those depicted in the compounds of Table 7, below.

In some embodiments, -L³-R⁸, wherein R⁸ is optionally substituted with one or more instances of R⁹, is a substituent of Table 5 or Table 6. In some embodiments, the -L³-R⁸ of Table 5 or Table 6 is shown with the one or more instance of R⁹. Also contemplated are embodiments wherein the -L³-R⁸ of Table 5 or Table 6 is further substituted with one or more instances of R⁹ which are not shown in Table 5 or Table 6.

In some embodiments, -L³-R⁸, wherein R⁸ is optionally substituted with one or more instances of R⁹, is a substituent of Table 5 or Table 6, and L¹ is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, the -L′-R⁸ of Table 5 or Table 6 is shown with the one or more instance of R⁹, and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

Also contemplated are embodiments wherein the -L³-R⁸ of Table 5 or Table 6 is further substituted with one or more instances of R′ which are not shown in Table 5 or Table 6, and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

TABLE 5
Exemplary —L³—R⁸ substituents

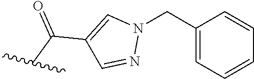 [SMILES: *C(=O)c1cnn(Cc2ccccc2)c1]

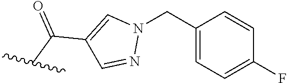 [SMILES: *C(=O)c1cnn(Cc2ccc(F)cc2)c1]

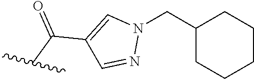 [SMILES: *C(=O)c1cnn(CC2CCCCC2)c1]

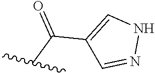 [SMILES: *C(=O)c1cn[nH]c1]

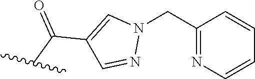 [SMILES: *C(=O)c1cnn(Cc2ccccn2)c1]

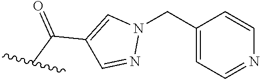 [SMILES: *C(=O)c1cnn(Cc2ccncc2)c1]

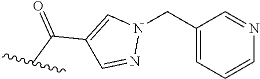 [SMILES: *C(=O)c1cnn(Cc2cccnc2)c1]

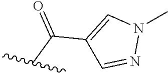 [SMILES: *C(=O)c1cnn(C)c1]

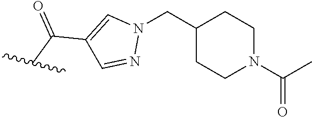 [SMILES: *C(=O)c1cnn(CC2CCN(C(C)=O)CC2)c1]

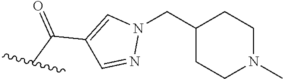 [SMILES: *C(=O)c1cnn(CC2CCN(C)CC2)c1]

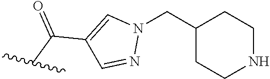 [SMILES: *C(=O)c1cnn(CC2CCNCC2)c1]

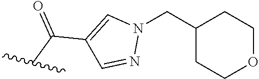 [SMILES: *C(=O)c1cnn(CC2CCOCC2)c1]

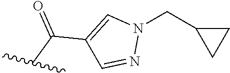 [SMILES: *C(=O)c1cnn(CC2CC2)c1]

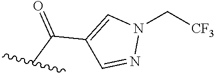 [SMILES: *C(=O)c1cnn(CC(F)(F)F)c1]

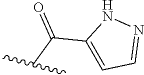 [SMILES: *C(=O)c1ccn[nH]1]

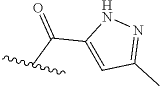 [SMILES: *C(=O)c1cc(C)n[nH]1]

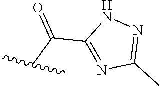 [SMILES: *C(=O)c1nc(C)n[nH]1]

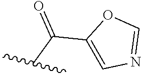 [SMILES: *C(=O)c1cnco1]

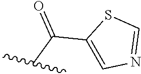 [SMILES: *C(=O)c1cncs1]

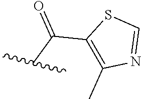 [SMILES: *C(=O)c1scnc1C]

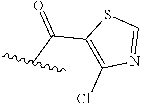 [SMILES: *C(=O)c1scnc1Cl]

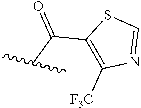 [SMILES: *C(=O)c1scnc1C(F)(F)F]

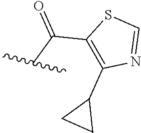 [SMILES: *C(=O)c1scnc1C1CC1]

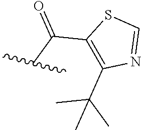 [SMILES: *C(=O)c1scnc1C(C)(C)C]

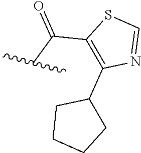 [SMILES: *C(=O)c1scnc1C1CCCC1]

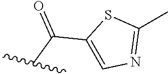 [SMILES: *C(=O)c1cnc(C)s1]

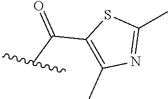 [SMILES: *C(=O)c1sc(C)nc1C]

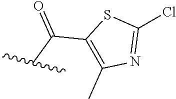 [SMILES: *C(=O)c1sc(Cl)nc1C]

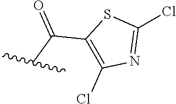 [SMILES: *C(=O)c1sc(Cl)nc1Cl]

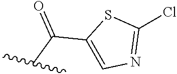 [SMILES: *C(=O)c1cnc(Cl)s1]

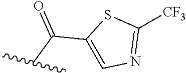 [SMILES: *C(=O)c1cnc(C(F)(F)F)s1]

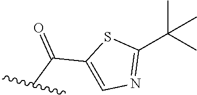 [SMILES: *C(=O)c1cnc(C(C)(C)C)s1]

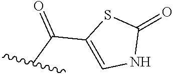 [SMILES: *C(=O)c1c[nH]c(=O)s1]

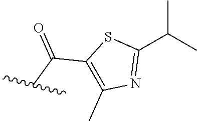 [SMILES: *C(=O)c1sc(C(C)C)nc1C]

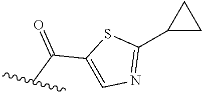 [SMILES: *C(=O)c1cnc(C2CC2)s1]

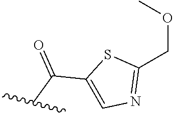 [SMILES: *C(=O)c1cnc(COC)s1]

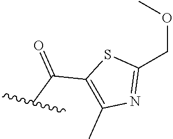 [SMILES: *C(=O)c1sc(COC)nc1C]

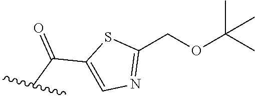 [SMILES: *C(=O)c1cnc(COC(C)(C)C)s1]

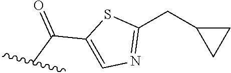 [SMILES: *C(=O)c1cnc(CC2CC2)s1]

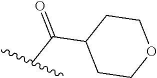 [SMILES: *C(=O)C1CCOCC1]

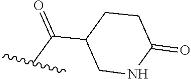 [SMILES: *C(=O)C1CCC(=O)NC1]

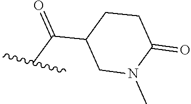 [SMILES: *C(=O)C1CCC(=O)N(C)C1]

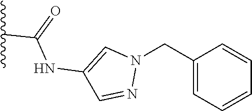 [SMILES: *C(=O)Nc1cnn(Cc2ccccc2)c1]

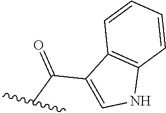 [SMILES: *C(=O)c1c[nH]c2ccccc12]

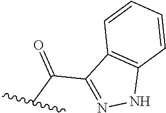 [SMILES: *C(=O)c1n[nH]c2ccccc12]

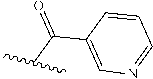 [SMILES: *C(=O)c1cccnc1]

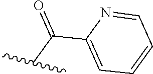 [SMILES: *C(=O)c1ccccn1]

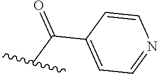 [SMILES: *C(=O)c1ccncc1]

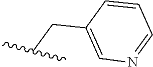 [SMILES: *Cc1cccnc1]

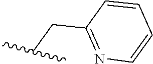 [SMILES: *Cc1ccccn1]

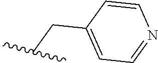 [SMILES: *Cc1ccncc1]

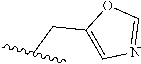 [SMILES: *Cc1cnco1]

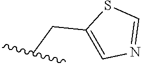 [SMILES: *Cc1cncs1]

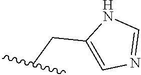 [SMILES: *Cc1cnc[nH]1]

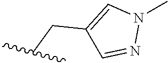 [SMILES: *Cc1cnn(C)c1]

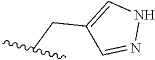 [SMILES: *Cc1cn[nH]c1]

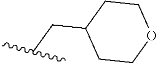 [SMILES: *CC1CCOCC1]

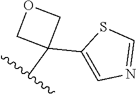 [SMILES: *C1(c2cncs2)COC1]

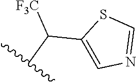 [SMILES: *C(c1cncs1)C(F)(F)F]

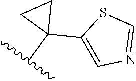 [SMILES: *C1(c2cncs2)CC1]

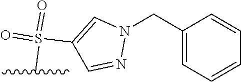 [SMILES: *S(=O)(=O)c1cnn(Cc2ccccc2)c1]

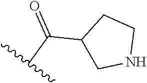 [SMILES: *C(=O)C1CCNC1]

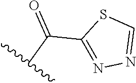 [SMILES: *C(=O)c1nncs1]

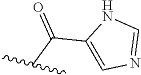 [SMILES: *C(=O)c1cnc[nH]1]

TABLE 6
Exemplary —L³—R⁸ or R⁸ substituents, wherein R⁸
is optionally substituted with one
or more instances of R⁹, wherein the one or more
R⁹ is or is not pictured in Table 6

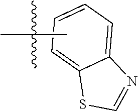

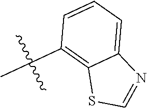 [SMILES: *c1cccc2ncsc12]

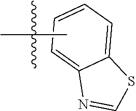

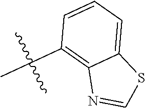 [SMILES: *c1cccc2scnc12]

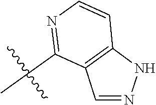 [SMILES: *c1nccc2[nH]ncc12]

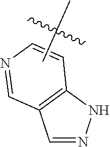

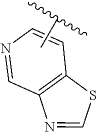

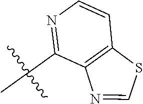 [SMILES: *c1nccc2scnc12]

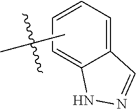

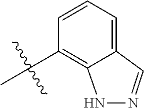 [SMILES: *c1cccc2cn[nH]c12]

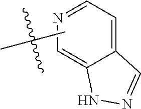

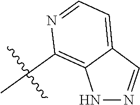 [SMILES: *c1nccc2cn[nH]c12]

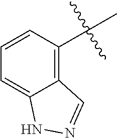 [SMILES: *c1cccc2[nH]ncc12]

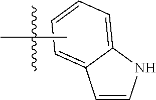

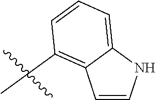 [SMILES: *c1cccc2[nH]ccc12]

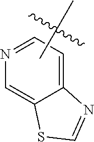

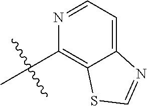 [SMILES: *c1nccc2ncsc12]

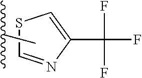

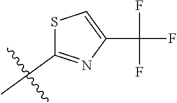 [SMILES: *c1nc(C(F)(F)F)cs1]

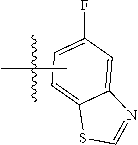

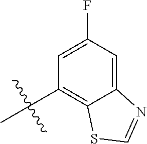 [SMILES: *c1cc(F)cc2ncsc12]

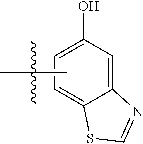

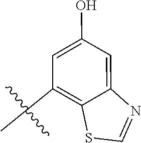 [SMILES: *c1cc(O)cc2ncsc12]

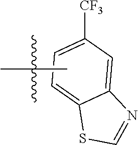

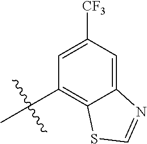 [SMILES: *c1cc(C(F)(F)F)cc2ncsc12]

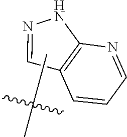

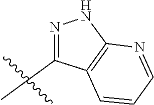 [SMILES: *c1n[nH]c2ncccc12]

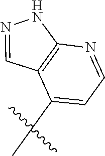 [SMILES: *c1ccnc2[nH]ncc12]

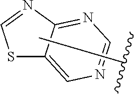

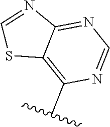 [SMILES: *c1ncnc2ncsc12]

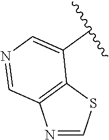 [SMILES: *c1cncc2ncsc12]

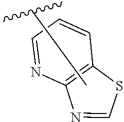

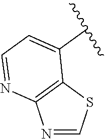 [SMILES: *c1ccnc2ncsc12]

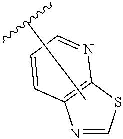

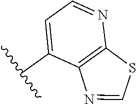 [SMILES: *c1ccnc2scnc12]

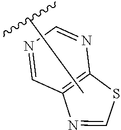

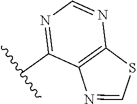 [SMILES: *c1ncnc2scnc12]

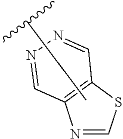

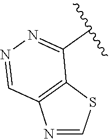 [SMILES: *c1nncc2ncsc12]

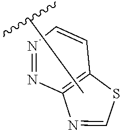

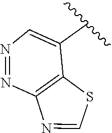 [SMILES: *c1cnnc2ncsc12]

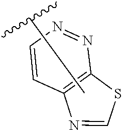

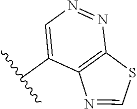 [SMILES: *c1cnnc2scnc12]

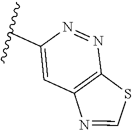 [SMILES: *c1cc2ncsc2nn1]

In some embodiments, -L³-R⁸ is

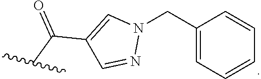 [SMILES: *C(=O)c1cnn(Cc2ccccc2)c1]

As defined generally above, each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur). In some embodiments, each Cy is independently a 3-8 membered saturated or partially unsaturated monocyclic carbocyclic ring or phenyl. In some embodiments, each Cy is independently an optionally substituted cyclic group selected from a 3-8 membered saturated or partially unsaturated monocyclic carbocyclic ring. In some embodiments. Cy is phenyl. In some embodiments, each Cy is independently a 3-8 membered saturated or partially unsaturated monocyclic heterocyclic ring (having 1-2 heteroatoms independently selected from nitrogen, oxygen, and sulfur) or a 5-6 membered monocyclic heteroaromatic ring (having 1-4 heteroatoms independently selected from nitrogen, oxygen, and sulfur). In some embodiments, each Cy is independently a 3-8 membered saturated or partially unsaturated monocyclic heterocyclic ring (having 1-2 heteroatoms independently selected from nitrogen, oxygen, and sulfur). In some embodiments, each Cy is independently a 5-6 membered monocyclic heteroaromatic ring (having 1-4 heteroatoms independently selected from nitrogen, oxygen, and sulfur).

As defined generally above, each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur), or two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur), or the two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur).

In some embodiments, R is hydrogen. In some embodiments, each R is independently an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur). In some embodiments, each R is independently an optionally substituted C₁₋₆ aliphatic group. In some embodiments, each R is independently an optionally substituted phenyl. In some embodiments, each R is independently an optionally substituted 3-7 membered saturated or partially unsaturated carbocyclic ring. In some embodiments, each R is independently an optionally substituted 3-7 membered saturated or partially unsaturated heterocyclic ring (having 1-2 heteroatoms independently selected from nitrogen, oxygen, and sulfur). In some embodiments, each R is independently an optionally substituted 5-6 membered heteroaryl ring (having 1-4 heteroatoms independently selected from nitrogen, oxygen, and sulfur).

In some embodiments, two R groups on the same nitrogen are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur); or two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments, two R groups on the same nitrogen are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur). In some embodiments, two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur). In some embodiments, two R groups on the same nitrogen are taken together with the nitrogen atom to form an optionally substituted moiety selected from the group consisting of

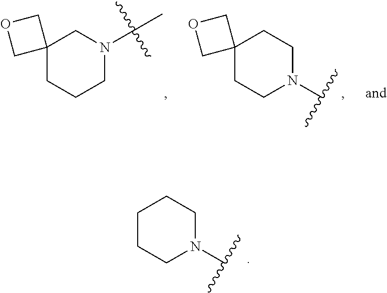 [SMILES: *N1CCCC2(COC2)C1 ; *N1CCC2(CC1)COC2 ; *N1CCCCC1]

In some embodiments, two R groups on the same nitrogen atom are taken together with the nitrogen atom to form

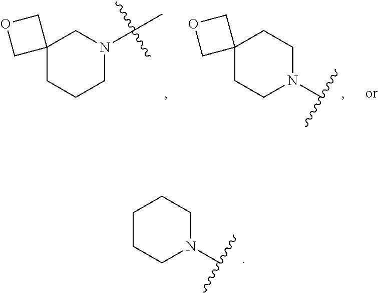 [SMILES: *N1CCCC2(COC2)C1 ; *N1CCC2(CC1)COC2 ; *N1CCCCC1]

In some embodiments, two R groups on the same nitrogen are taken together with the nitrogen atom to form optionally substituted

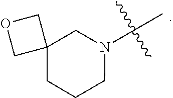 [SMILES: *N1CCCC2(COC2)C1]

In some embodiments, two R groups on the same nitrogen atom are taken together with the nitrogen atom to form

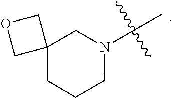 [SMILES: *N1CCCC2(COC2)C1]

In some embodiments, two R groups on the same nitrogen atom are taken together with the nitrogen atom to form optionally substituted

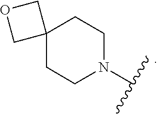 [SMILES: *N1CCC2(CC1)COC2]

In some embodiments, two R groups on the same nitrogen atom are taken together with the nitrogen atom to form

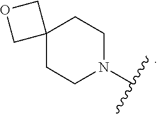 [SMILES: *N1CCC2(CC1)COC2]

In some embodiments, two R groups on the same nitrogen atom taken together with the nitrogen atom to form optionally substituted

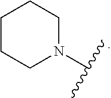 [SMILES: *N1CCCCC1]

In some embodiments, two R groups on the same nitrogen atom are taken together with the nitrogen atom to form

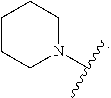 [SMILES: *N1CCCCC1]

As defined generally above, one or both of L¹ and L³ is a covalent bond. In certain embodiments, L² is a covalent bond and L³ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In certain embodiments, L³ is a covalent bond and L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In certain embodiments, L² and L³ are both a covalent bond.

In some embodiments, the compound of Formula I is a compound of Formula IIA:

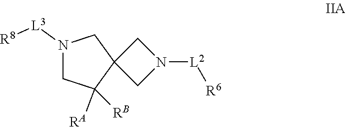

or a pharmaceutically acceptable salt thereof, wherein R^{A}, R^{B}, L², R⁶, L³ and R⁸, and their constituent groups, are each as defined and described herein. In some embodiments, R^{A}, R^{B}, L², R⁶, L³ and R⁸, and their constituent groups, are each as defined and described in Formula I. In some embodiments, R^{A} is a substituent from Table 2. In some embodiments, -L²-R⁶ is a substituent from Table 3 or Table 4. In some embodiments, -L³-R⁸ is a substituent from Table 5 or Table 6. In some embodiments, R^{A} is a substituent from Table 2, and -L²-R⁶ is a substituent from Table 3 or Table 4. In some embodiments, R^{A} is a substituent from Table 2, and -L³-R⁸ is a substituent from Table 5 or Table 6. In some embodiments, -L²-R⁶ is a substituent from Table 3 or Table 4, and -L³-R⁸ is a substituent from Table 5 or Table 6. In some embodiments, R^{A} is a substituent from Table 2, -L²-R⁶ is a substituent from Table 3 or Table 4, and -L³-R⁸ is a substituent from Table 5 or Table 6.

In some embodiments, the compound of Formula I is a compound of Formula IIB:

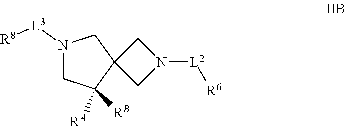

or a pharmaceutically acceptable salt thereof, wherein R^{A}, R^{B}, L², R⁶, L³ and R⁸, and their constituent groups, are each as defined and described herein. In some embodiments, R^{A}, R^{B}, L², R⁶, L³ and R⁸, and their constituent groups, are each as defined and described in Formula I. In some embodiments, R^{A} is a substituent from Table 2. In some embodiments, -L²-R⁶ is a substituent from Table 3 or Table 4. In some embodiments, -L³-R⁸ is a substituent from Table 5 or Table 6. In some embodiments, R^{A} is a substituent from Table 2, and -L²-R⁶ is a substituent from Table 3 or Table 4. In some embodiments, R^{A} is a substituent from Table 2, and -L³-R⁸ is a substituent from Table 5 or Table 6. In some embodiments, -L²-R⁶ is a substituent from Table 3 or Table 4, and -L³-R⁸ is a substituent from Table 5 or Table 6. In some embodiments, R^{A} is a substituent from Table 2, -L²-R⁶ is a substituent from Table 3 or Table 4, and -L³-R⁸ is a substituent from Table 5 or Table 6.

In some embodiments, the compound of Formula I is a compound of Formula IIB:

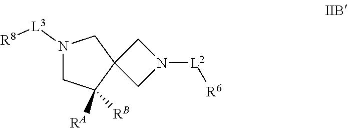

or a pharmaceutically acceptable salt thereof, wherein R^{A}, R^{B}, L², R⁶, L³ and R⁸, and their constituent groups, are each as defined and described herein. In some embodiments, R^{A}, R^{B}, L², R⁶, L³ and R⁸, and their constituent groups, are each as defined and described in Formula I. In some embodiments, R^{A} is a substituent from Table 2. In some embodiments, -L²-R⁶ is a substituent from Table 3 or Table 4. In some embodiments, -L³-R⁸ is a substituent from Table 5 or Table 6. In some embodiments, R^{A} is a substituent from Table 2, and -L²-R⁶ is a substituent from Table 3 or Table 4. In some embodiments, R^{A} is a substituent from Table 2, and -L³-R⁸ is a substituent from Table 5 or Table 6. In some embodiments, -L²-R⁶ is a substituent from Table 3 or Table 4, and -L³-R⁸ is a substituent from Table 5 or Table 6. In some embodiments, R^{A} is a substituent from Table 2, -L²-R⁶ is a substituent from Table 3 or Table 4, and -L³-R⁸ is a substituent from Table 5 or Table 6.

In some embodiments, the compound of Formula I is a compound of Formula II:

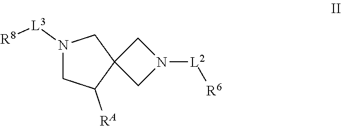

or a pharmaceutically acceptable salt thereof, wherein R^{A}, L², R⁶, L³ and R⁸, and their constituent groups, are each as defined and described herein. In some embodiments, R^{A}, L², R⁶, L³ and R⁸, are as described in Formula I. In some embodiments, R^{A} is a substituent from Table 2. In some embodiments, -L²-R⁶ is a substituent from Table 3 or Table 4. In some embodiments, -L³-R⁸ is a substituent from Table 5 or Table 6. In some embodiments, R^{A} is a substituent from Table 2, and -L²-R⁶ is a substituent from Table 3 or Table 4. In some embodiments, R^{A} is a substituent from Table 2, and -L³-R⁸ is a substituent from Table 5 or Table 6. In some embodiments, -L²-R⁶ is a substituent from Table 3 or Table 4, and -L³-R⁸ is a substituent from Table 5 or Table 6. In some embodiments, R^{A} is a substituent from Table 2, -L²-R⁶ is a substituent from Table 3 or Table 4, and -L³-R⁸ is a substituent from Table 5 or Table 6.

In some embodiments, the compound of Formula I is a compound of Formula IIIa:

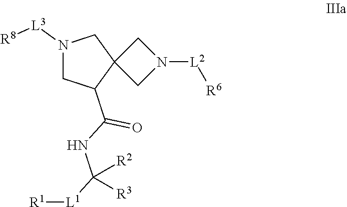

or a pharmaceutically acceptable salt thereof, wherein L¹, R¹. R², R³, L², R⁶, L¹ and R⁸, and their constituent groups, are each as defined and described herein. In some embodiments, R¹ is phenyl. In some embodiments, R¹ is cyclohexyl. In some embodiments, R² is a substituent from Table 2. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, L² is a methylene. In some embodiments. L² is a covalent bond. In some embodiments, L³ is a methylene. In some embodiments, L³ is a covalent bond. In some embodiments, L¹ is a —C(O)—. In some embodiments, L³ is a —C(O)—. In some embodiments, both L¹ and L³ are a covalent bond. In some embodiments, -L²-R⁶ is a substituent from Table 3 or Table 4. In some embodiments, -L³-R⁸ is a substituent from Table 5 or Table 6.

In some embodiments, the compound of Formula I is a compound of Formula IIIb:

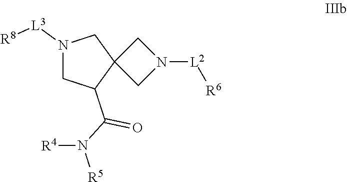

or a pharmaceutically acceptable salt thereof, wherein R⁴, R⁵, L², R⁶, L³ and R⁸, and their constituent groups, are each as defined and described herein. In some embodiments, L¹ is a methylene. In some embodiments, L² is a covalent bond. In some embodiments, L³ is a methylene. In some embodiments, L³ is a covalent bond. In some embodiments, L² is a —C(O)—. In some embodiments, L³ is a —C(O)—. In some embodiments, both L² and L³ are a covalent bond. In some embodiments, -L²-R⁶ is a substituent from Table 3 or Table 4. In some embodiments, -L³-R⁸ is a substituent from Table 5 or Table 6.

In some embodiments, the compound of Formula I is a compound of Formula IVa:

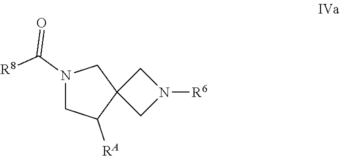

or a pharmaceutically acceptable salt thereof, wherein R^{A}, R⁶, and R⁸, and their constituent groups, are each as defined and described herein. In some embodiments, R^{A} is a substituent from Table 2. In some embodiments, R⁶ is a substituent from Table 4.

In some embodiments, the compound of Formula I is a compound of Formula IVb:

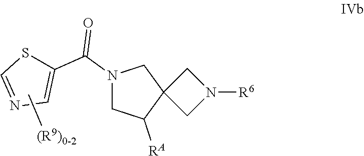

or a pharmaceutically acceptable salt thereof, wherein R^{A}, R⁶, and R⁹, and their constituent groups, are each as defined and described herein. In some embodiments, the thiazolyl group is not substituted with R⁹. In some embodiments, R^{A} is a substituent from Table 2. In some embodiments, R⁶ is a substituent from Table 4.

In some embodiments, the compound of Formula I is a compound of Formula IVc:

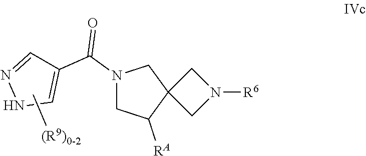

or a pharmaceutically acceptable salt thereof, wherein R^{A}, R⁶, and R⁹, and their constituent groups, are each as defined and described herein. In some embodiments, the pyrazolyl group is not substituted with R⁹. In some embodiments, the pyrazolyl group is substituted with one instance of R⁹, which is a benzyl group. In some embodiments, R^{A} is a substituent from Table 2. In some embodiments, R⁶ is a substituent from Table 4.

In some embodiments, the compound of Formula I is a compound of Formula Va:

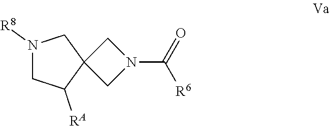

or a pharmaceutically acceptable salt thereof, wherein R^{A}, R⁶, and R⁸, and their constituent groups, are each as defined and described herein. In some embodiments, R⁶ is an optionally substituted cyclopropyl group. In some embodiments, R^{A} is a substituent from Table 2. In some embodiments, R⁸ is a substituent from Table 6.

In some embodiments, the compound of Formula I is a compound of Formula VIa:

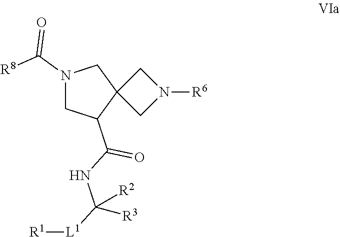

or a pharmaceutically acceptable salt thereof, wherein L¹, R¹, R², R³, R⁶, and R⁸, and their constituent groups, are each as defined and described herein. In some embodiments, R⁶ is a substituent from Table 4. In some embodiments, R¹ is phenyl. In some embodiments, R¹ is cyclohexyl. In some embodiments, R² is a substituent from Table 2. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, R⁶ is an optionally substituted 5-6 membered monocyclic heteroaromatic ring (having 1-4 heteroatoms independently selected from nitrogen, oxygen, and sulfur).

In some embodiments, the compound of Formula I is a compound of Formula VIb:

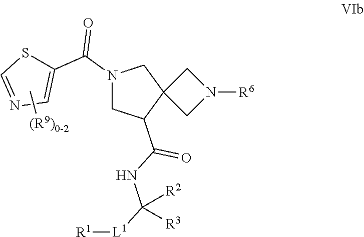

or a pharmaceutically acceptable salt thereof, wherein L¹, R¹, R², R³, R⁶, and R⁹, and their constituent groups, are each as defined and described herein. In some embodiments, R⁶ is a substituent from Table 4. In some embodiments, R¹ is phenyl. In some embodiments, R¹ is cyclohexyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, R² is a substituent from Table 2. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, R⁶ is an optionally substituted 5-6 membered monocyclic heteroaromatic ring (having 1-4 heteroatoms independently selected from nitrogen, oxygen, and sulfur). In some embodiments, the thiazolyl group is not substituted with R⁹.

In some embodiments, the compound of Formula I is a compound of Formula VIc:

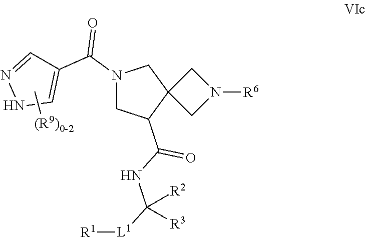

or a pharmaceutically acceptable salt thereof, wherein L¹, R¹, R², R³, R⁶, and R⁹, and their constituent groups, are each as defined and described herein. In some embodiments, R⁶ is a substituent from Table 4. In some embodiments, R¹ is phenyl. In some embodiments, R¹ is cyclohexyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, R² is a substituent from Table 2. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, R⁶ is an optionally substituted 5-6 membered monocyclic heteroaromatic ring (having 1-4 heteroatoms independently selected from nitrogen, oxygen, and sulfur). In some embodiments, the pyrazolyl group is not substituted with R⁹. In some embodiments, the pyrazolyl group is substituted with one instance of R⁹, which is a benzyl group.

In some embodiments, the compound of Formula I is a compound of Formula Via′:

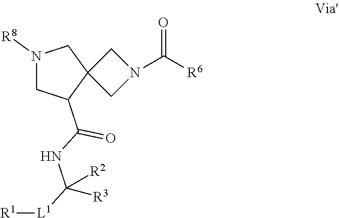

or a pharmaceutically acceptable salt thereof, wherein L¹, R¹, R², R³, R⁶, and R⁸, and their constituent groups, are each as defined and described herein. In some embodiments, R⁸ is a substituent from Table 6. In some embodiments, R¹ is phenyl. In some embodiments, R¹ is cyclohexyl. In some embodiments, R² is a substituent from Table 2. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, R⁶ is an optionally substituted cyclopropyl group. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur).

In some embodiments, the compound of Formula I is a compound of Formula VIb′:

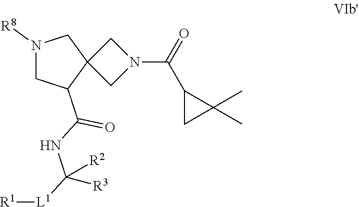

or a pharmaceutically acceptable salt thereof, wherein L¹, R¹, R², R³, R⁸, and R⁹, and their constituent groups, are each as defined and described herein. In some embodiments, R⁸ is a substituent from Table 6. In some embodiments, R¹ is phenyl. In some embodiments, R¹ is cyclohexyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, R² is a substituent from Table 2. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur).

In some embodiments, the compound of Formula I is a compound of Formula VId:

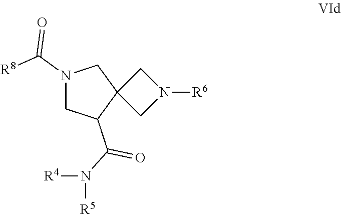

or a pharmaceutically acceptable salt thereof, wherein R⁴, R⁵, R⁶, and R⁸, and their constituent groups, are each as defined and described herein. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur). In some embodiments, R⁸ is a pyrazolyl or thiazolyl group, optionally substituted with one or more instances of R⁹. In some embodiments, R⁸ is a pyrazolyl or thiazolyl group.

In some embodiments, the compound of Formula I is a compound of Formula VIe:

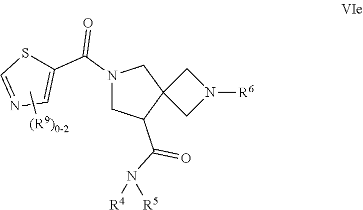

or a pharmaceutically acceptable salt thereof, wherein R⁴, R⁵, R⁶, and R⁹, and their constituent groups, are each as defined and described herein. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur). In some embodiments, the thiazolyl group is not substituted with R⁹.

In some embodiments, the compound of Formula I is a compound of Formula VIf:

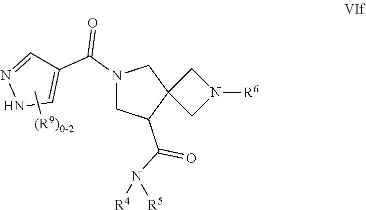

or a pharmaceutically acceptable salt thereof, wherein R⁴, R⁵, R⁶, and R⁹, and their constituent groups, are each as defined and described herein. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur). In some embodiments, the pyrazolyl group is not substituted with R⁹. In some embodiments, the pyrazolyl group is substituted with one instance of R⁹, which is a benzyl group.

In some embodiments, the compound of Formula I is a compound of Formula VId′:

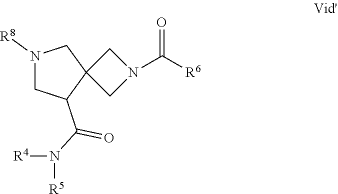

or a pharmaceutically acceptable salt thereof, wherein R⁴, R⁵, R⁶, and R⁸, and their constituent groups, are each as defined and described herein. In some embodiments, R⁸ is a substituent from Table 6. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur). In some embodiments, R⁶ is an optionally substituted cyclopropyl.

In some embodiments, the compound of Formula I is a compound of Formula VIe′:

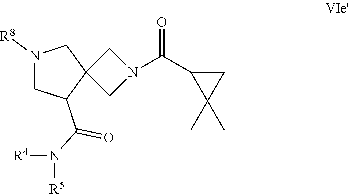

or a pharmaceutically acceptable salt thereof, wherein R⁴, R⁵, and R⁸, and their constituent groups, are each as defined and described herein. In some embodiments, R⁸ is a substituent from Table 6. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur).

In some embodiments, the compound of Formula I is a compound of Formula VIIa:

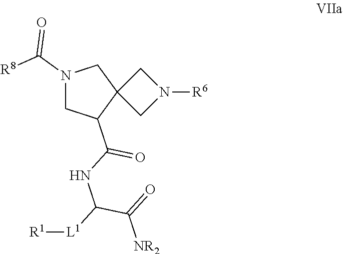

or a pharmaceutically acceptable salt thereof, wherein L¹, R¹, R, R⁶, and R⁸, and their constituent groups, are each as defined and described herein. In some embodiments, R′ is phenyl. In some embodiments, R′ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur). In some embodiments, R⁸ is a pyrazolyl or thiazolyl group, optionally substituted with one or more instances of R⁹. In some embodiments, R⁸ is a pyrazolyl or thiazolyl group.

In some embodiments, the compound of Formula I is a compound of Formula VIIb:

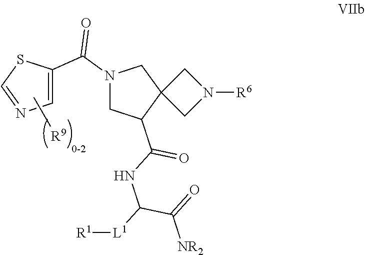

or a pharmaceutically acceptable salt thereof, wherein L¹, R¹, R, R¹, and R⁹, and their constituent groups, are each as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur). In some embodiments, R⁶ is a pyrazolyl or thiazolyl group, optionally substituted with one or more instances of R⁷. In some embodiments, R⁶ is a pyrazolyl or thiazolyl group. In some embodiments, the thiazolyl group is not substituted with R⁷.

In some embodiments, the compound of Formula I is a compound of Formula VIIc:

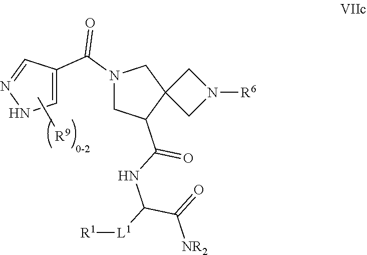

or a pharmaceutically acceptable salt thereof, wherein L¹, R¹, R, R⁶, and R⁹, and their constituent groups, are each as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur). In some embodiments, R⁶ is a pyrazolyl or thiazolyl group, optionally substituted with one or more instances of R⁷. In some embodiments, R⁶ is a pyrazolyl or thiazolyl group. In some embodiments, the pyrazolyl group is not substituted with R⁷. In some embodiments, the pyrazolyl group is substituted with one instance of R⁷, which is a benzyl group.

In some embodiments, the compound of Formula I is a compound of Formula VIIa′:

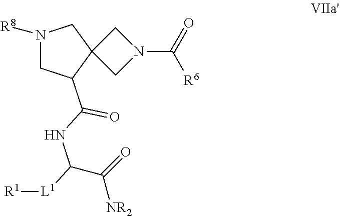

or a pharmaceutically acceptable salt thereof, wherein L¹, R¹, R, R⁶, and R⁸, and their constituent groups, are each as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, R⁸ is a substituent from Table 6. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur). In some embodiments, R⁶ is an optionally substituted cyclopropyl.

In some embodiments, the compound of Formula I is a compound of Formula VIIb′:

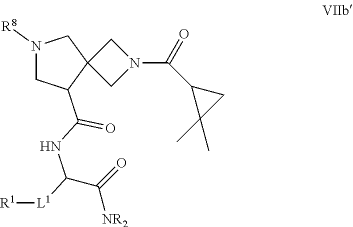

or a pharmaceutically acceptable salt thereof, wherein L¹, R¹, R, and R⁸, and their constituent groups, are each as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, R⁸ is a substituent from Table 6. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur).

In some embodiments, the compound of Formula I is a compound of Formula VIIIa:

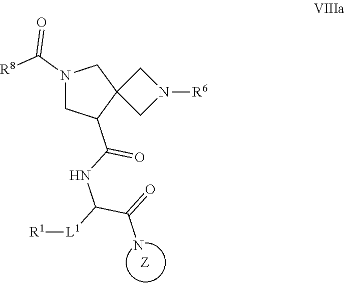

or a pharmaceutically acceptable salt thereof, wherein L¹, R¹, R⁶, and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments. Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur). In some embodiments, R⁸ is a pyrazolyl or thiazolyl group, optionally substituted with one or more instances of R⁹. In some embodiments, R⁸ is a pyrazolyl or thiazolyl group.

In some embodiments, the compound of Formula I is a compound of Formula VIIIb:

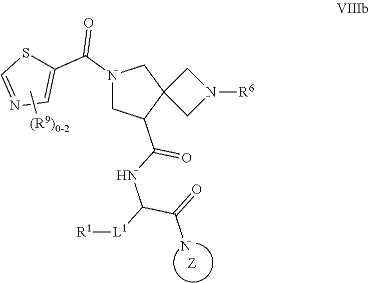

or a pharmaceutically acceptable salt thereof, wherein L¹, R¹, R⁶, and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, the thiazolyl group is not substituted with R⁹. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur).

In some embodiments, the compound of Formula I is a compound of Formula VIIIc:

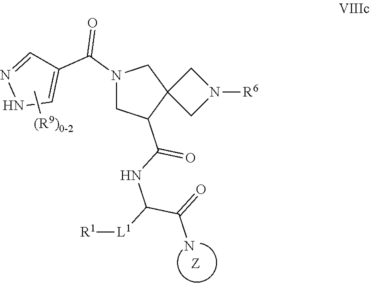

or a pharmaceutically acceptable salt thereof, wherein L¹, R¹, R⁶, and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, the pyrazolyl group is not substituted with R⁹. In some embodiments, the pyrazolyl group is substituted with one instance of R⁹, which is a benzyl group. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur).

In some embodiments, the compound of Formula I is a compound of Formula VIIIa′:

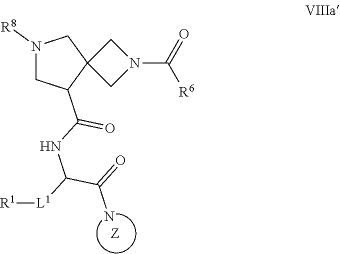

or a pharmaceutically acceptable salt thereof, wherein L¹, R¹, R⁶, and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁸ is a substituent from Table 6. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur). In some embodiments, R⁶ is an optionally substituted cyclopropyl.

In some embodiments, the compound of Formula I is a compound of Formula VIIIb′:

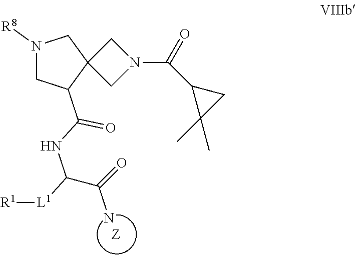

or a pharmaceutically acceptable salt thereof, wherein L¹, R¹, and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁸ is a substituent from Table 6. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur).

In some embodiments, the compound of Formula I is a compound of Formula IXa:

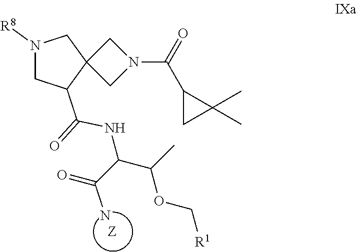

or a pharmaceutically acceptable salt thereof, wherein R¹ and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R¹ is 2-oxabicyclo[2.2.2]octyl. In some embodiments, R⁸ is a substituent from Table 6. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur).

In some embodiments, the compound of Formula I is a compound of Formula IXa*:

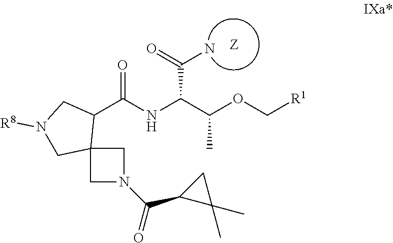

or a pharmaceutically acceptable salt thereof, wherein R¹ and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁸ is a substituent from Table 6. In some embodiments, R⁸ is an optionally substituted 5-6 membered monocyclic heteroaromatic ring (having 1-4 heteroatoms independently selected from nitrogen, oxygen, and sulfur) or an optionally substituted 8-10 membered bicyclic heteroaromatic ring (having 1-5 heteroatoms independently selected from nitrogen, oxygen, and sulfur).

In some embodiments, the compound of Formula I is a compound of Formula IXb:

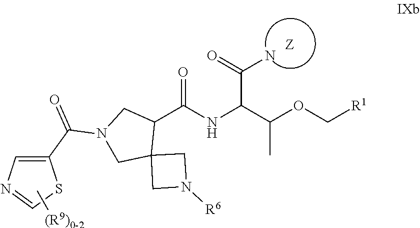

or a pharmaceutically acceptable salt thereof, wherein R¹, R⁶ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, the thiazolyl group is not substituted with R⁹. In some embodiments, the thiazolyl group is substituted with one instance of R⁹, which is a benzyl group. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur).

In some embodiments, the compound of Formula I is a compound of Formula IXb*:

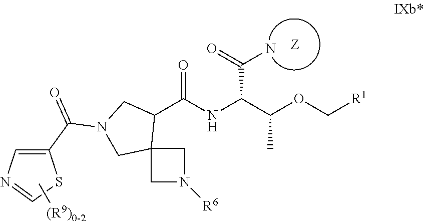

or a pharmaceutically acceptable salt thereof, wherein R¹, R⁶ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, the thiazolyl group is not substituted with R⁹. In some embodiments, the thiazolyl group is substituted with one instance of R⁹, which is a benzyl group. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur).

In some embodiments, the compound of Formula I is a compound of Formula IXc:

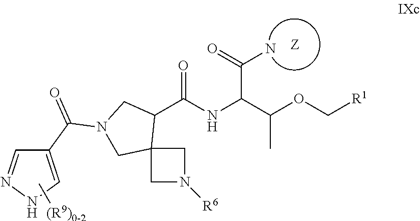

or a pharmaceutically acceptable salt thereof, wherein R¹, R⁶ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, the pyrazolyl group is not substituted with R⁹. In some embodiments, the pyrazolyl group is substituted with one instance of R⁹, which is a benzyl group. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur).

In some embodiments, the compound of Formula I is a compound of Formula IXc*:

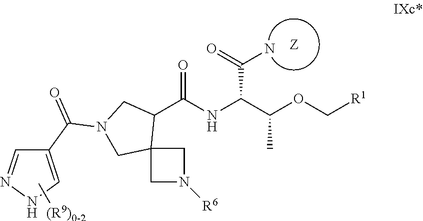

or a pharmaceutically acceptable salt thereof, wherein R¹, R⁶ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is 2-oxabicyclo[2.2.2]octyl. In some embodiments, the pyrazolyl group is not substituted with R⁹. In some embodiments, the pyrazolyl group is substituted with one instance of R⁹, which is a benzyl group. In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, R⁶ is a substituent from Table 4. In some embodiments, R⁶ is an optionally substituted 5-6 membered monocyclic heteroaromatic ring (having 1-4 heteroatoms independently selected from nitrogen, oxygen, and sulfur).

Exemplary compounds of the present disclosure are set forth in Table 7, below.

TABLE 7
Exemplary Compounds

| Compound No. | Structure |
|---|---|
| I-1 | 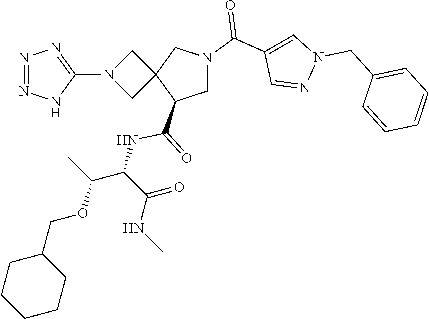 [SMILES: CNC(=O)[C@@H](NC(=O)[C@@H]1CN(C(=O)c2cnn(Cc3ccccc3)c2)CC12CN(c1nnn[nH]1)C2)[C@@H](C)OCC1CCCCC1] |
| I-2 | 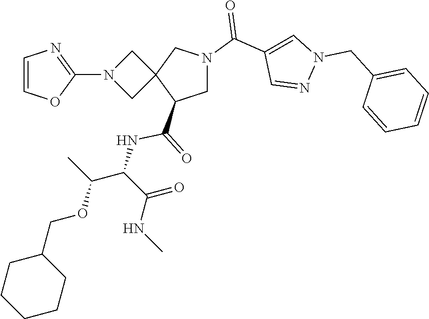 [SMILES: CNC(=O)[C@@H](NC(=O)[C@@H]1CN(C(=O)c2cnn(Cc3ccccc3)c2)CC12CN(c1ncco1)C2)[C@@H](C)OCC1CCCCC1] |
| I-3 | 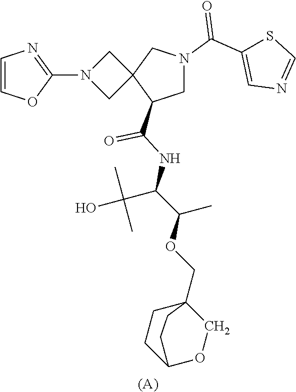 |
| | 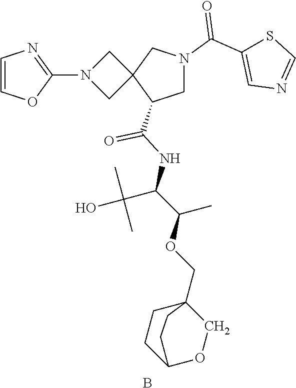 |
| I-4 | 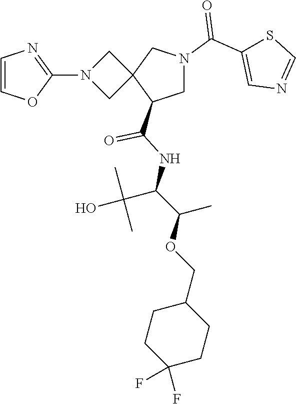 [SMILES: C[C@@H](OCC1CCC(F)(F)CC1)[C@H](NC(=O)[C@@H]1CN(C(=O)c2cncs2)CC12CN(c1ncco1)C2)C(C)(C)O] |
| I-5 | 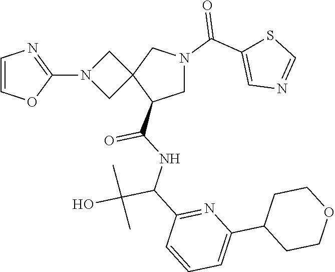 [SMILES: CC(C)(O)C(NC(=O)[C@@H]1CN(C(=O)c2cncs2)CC12CN(c1ncco1)C2)c1cccc(C2CCOCC2)n1] |
| I-6 | 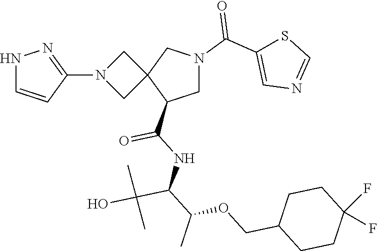 [SMILES: C[C@@H](OCC1CCC(F)(F)CC1)[C@H](NC(=O)[C@@H]1CN(C(=O)c2cncs2)CC12CN(c1cc[nH]n1)C2)C(C)(C)O] |
| I-7 | 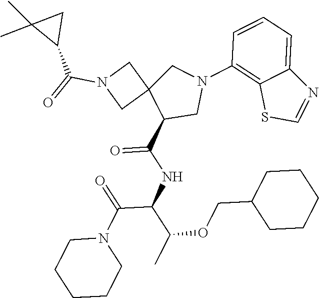 [SMILES: C[C@@H](OCC1CCCCC1)[C@H](NC(=O)[C@@H]1CN(c2cccc3ncsc23)CC12CN(C(=O)[C@H]1CC1(C)C)C2)C(=O)N1CCCCC1] |
| I-8 | 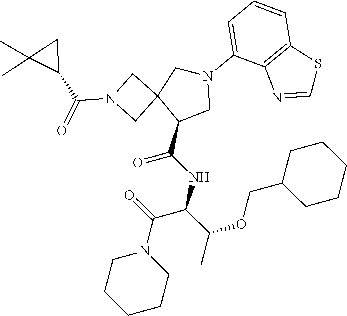 [SMILES: C[C@@H](OCC1CCCCC1)[C@H](NC(=O)[C@@H]1CN(c2cccc3scnc23)CC12CN(C(=O)[C@H]1CC1(C)C)C2)C(=O)N1CCCCC1] |
| I-9 | 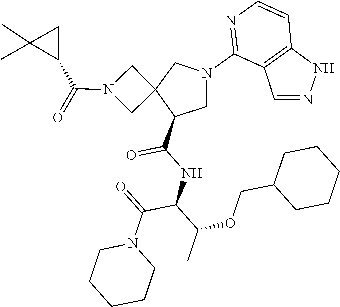 [SMILES: C[C@@H](OCC1CCCCC1)[C@H](NC(=O)[C@@H]1CN(c2nccc3[nH]ncc23)CC12CN(C(=O)[C@H]1CC1(C)C)C2)C(=O)N1CCCCC1] |
| I-10 | 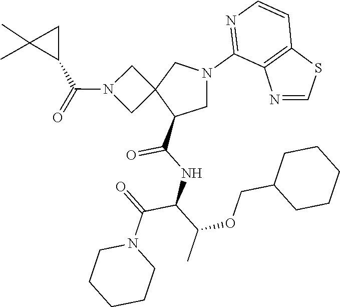 [SMILES: C[C@@H](OCC1CCCCC1)[C@H](NC(=O)[C@@H]1CN(c2nccc3scnc23)CC12CN(C(=O)[C@H]1CC1(C)C)C2)C(=O)N1CCCCC1] |
| I-11 | 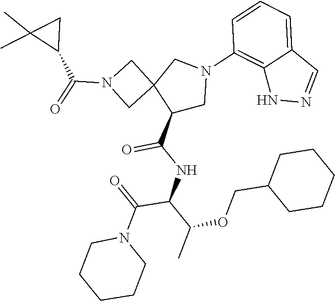 [SMILES: C[C@@H](OCC1CCCCC1)[C@H](NC(=O)[C@@H]1CN(c2cccc3cn[nH]c23)CC12CN(C(=O)[C@H]1CC1(C)C)C2)C(=O)N1CCCCC1] |
| I-12 | 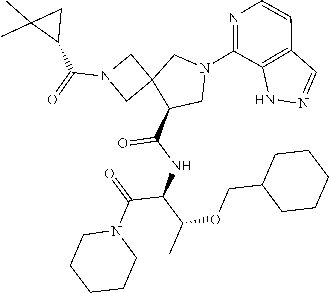 [SMILES: C[C@@H](OCC1CCCCC1)[C@H](NC(=O)[C@@H]1CN(c2nccc3cn[nH]c23)CC12CN(C(=O)[C@H]1CC1(C)C)C2)C(=O)N1CCCCC1] |
| I-13 | 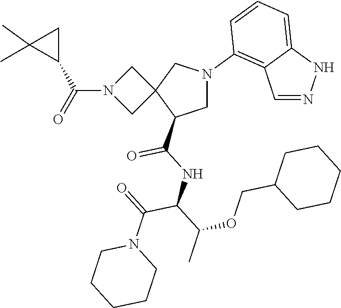 [SMILES: C[C@@H](OCC1CCCCC1)[C@H](NC(=O)[C@@H]1CN(c2cccc3[nH]ncc23)CC12CN(C(=O)[C@H]1CC1(C)C)C2)C(=O)N1CCCCC1] |
| I-14 | 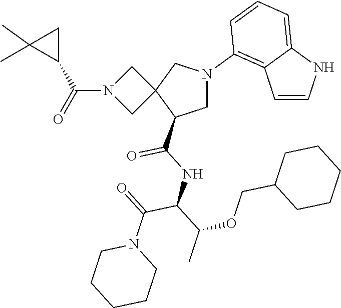 [SMILES: C[C@@H](OCC1CCCCC1)[C@H](NC(=O)[C@@H]1CN(c2cccc3[nH]ccc23)CC12CN(C(=O)[C@H]1CC1(C)C)C2)C(=O)N1CCCCC1] |
| I-15 | 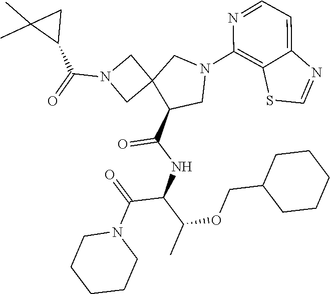 [SMILES: C[C@@H](OCC1CCCCC1)[C@H](NC(=O)[C@@H]1CN(c2nccc3ncsc23)CC12CN(C(=O)[C@H]1CC1(C)C)C2)C(=O)N1CCCCC1] |
| I-16 | 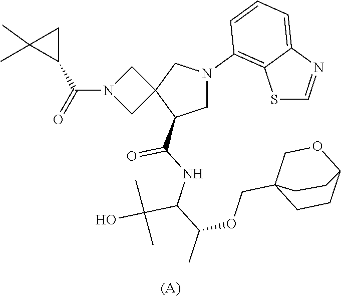 |
| | 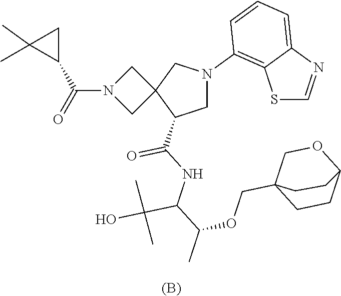 |
| I-17 | 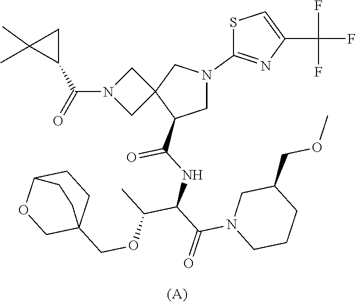 |
| | 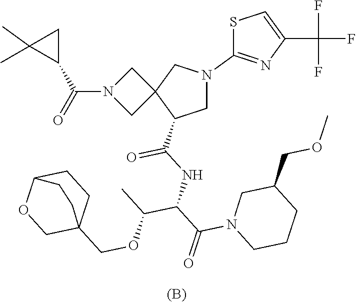 |
| I-18 | 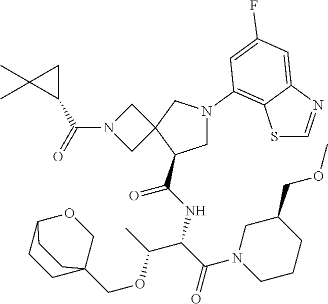 |
| | (A) |
| | 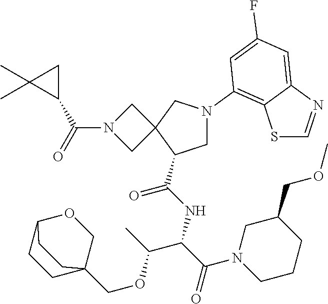 |
| | (B) |
| I-19 | 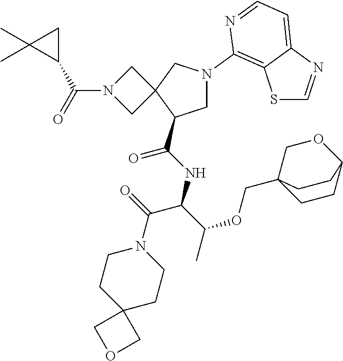 [SMILES: C[C@@H](OCC12CCC(CC1)OC2)[C@H](NC(=O)[C@@H]1CN(c2nccc3ncsc23)CC12CN(C(=O)[C@H]1CC1(C)C)C2)C(=O)N1CCC2(CC1)COC2] |
| I-20 | 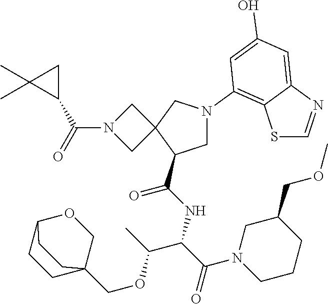 |
| I-21 | 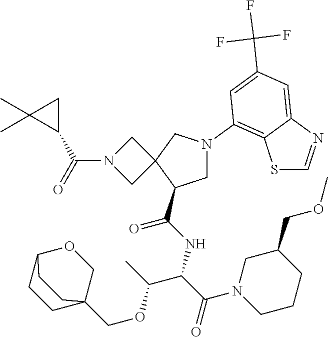 |
| I-22 | 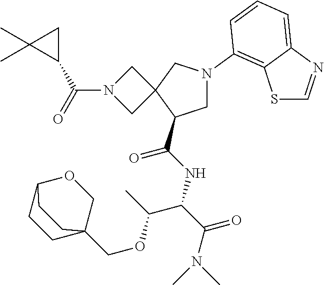 [SMILES: C[C@@H](OCC12CCC(CC1)OC2)[C@H](NC(=O)[C@@H]1CN(c2cccc3ncsc23)CC12CN(C(=O)[C@H]1CC1(C)C)C2)C(=O)N(C)C] |
| I-23 | 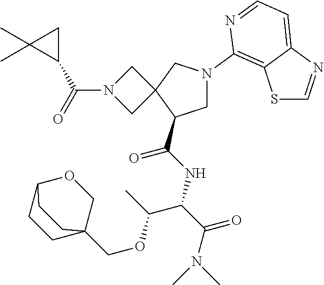 [SMILES: C[C@@H](OCC12CCC(CC1)OC2)[C@H](NC(=O)[C@@H]1CN(c2nccc3ncsc23)CC12CN(C(=O)[C@H]1CC1(C)C)C2)C(=O)N(C)C] |
| I-24 | 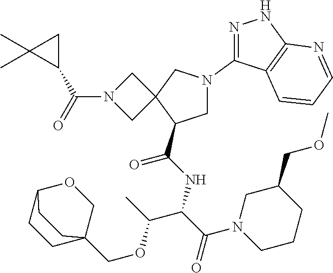 [SMILES: COC[C@H]1CCCN(C(=O)[C@@H](NC(=O)[C@@H]2CN(c3n[nH]c4ncccc34)CC23CN(C(=O)[C@H]2CC2(C)C)C3)[C@@H](C)OCC23CCC(CC2)OC3)C1] |
| I-25 | 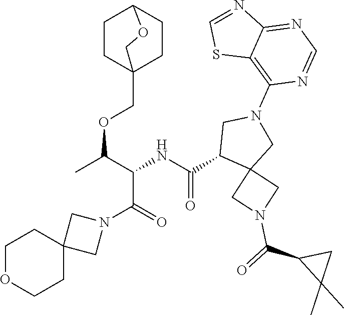 |
| I-26 | 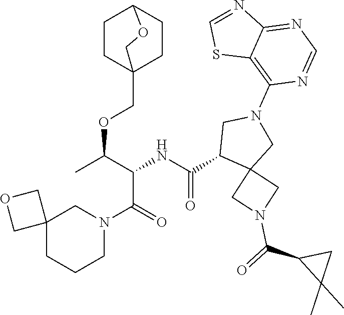 [SMILES: C[C@@H](OCC12CCC(CC1)OC2)[C@H](NC(=O)[C@@H]1CN(c2ncnc3ncsc23)CC12CN(C(=O)[C@H]1CC1(C)C)C2)C(=O)N1CCCC2(COC2)C1] |
| | (A) |
| | 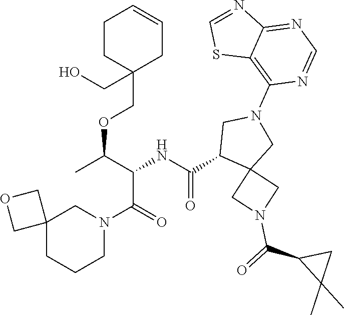 [SMILES: C[C@@H](OCC1(CO)CC=CCC1)[C@H](NC(=O)[C@@H]1CN(c2ncnc3ncsc23)CC12CN(C(=O)[C@H]1CC1(C)C)C2)C(=O)N1CCCC2(COC2)C1] |
| | (B) |
| I-27 | 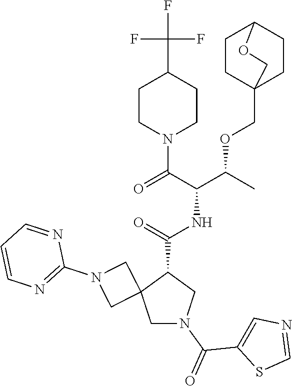 [SMILES: C[C@@H](OCC12CCC(CC1)OC2)[C@H](NC(=O)[C@@H]1CN(C(=O)c2cncs2)CC12CN(c1ncccn1)C2)C(=O)N1CCC(C(F)(F)F)CC1] |
| | (A) |
| | 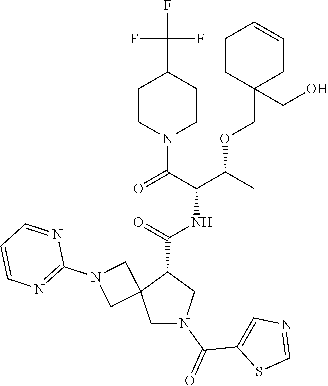 [SMILES: C[C@@H](OCC1(CO)CC=CCC1)[C@H](NC(=O)[C@@H]1CN(C(=O)c2cncs2)CC12CN(c1ncccn1)C2)C(=O)N1CCC(C(F)(F)F)CC1] |
| | (B) |
| I-28 | 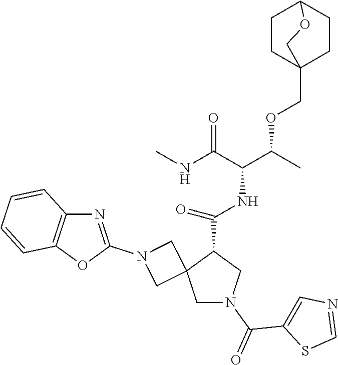 [SMILES: CNC(=O)[C@@H](NC(=O)[C@@H]1CN(C(=O)c2cncs2)CC12CN(c1nc3ccccc3o1)C2)[C@@H](C)OCC12CCC(CC1)OC2] |
| | (A) |
| | 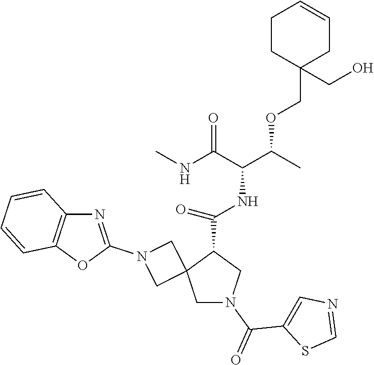 [SMILES: CNC(=O)[C@@H](NC(=O)[C@@H]1CN(C(=O)c2cncs2)CC12CN(c1nc3ccccc3o1)C2)[C@@H](C)OCC1(CO)CC=CCC1] |
| | (B) |
| I-29 | 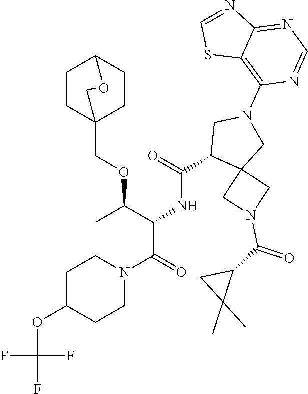 [SMILES: C[C@@H](OCC12CCC(CC1)OC2)[C@H](NC(=O)[C@@H]1CN(c2ncnc3ncsc23)CC12CN(C(=O)[C@H]1CC1(C)C)C2)C(=O)N1CCC(OC(F)(F)F)CC1] |
| I-30 | 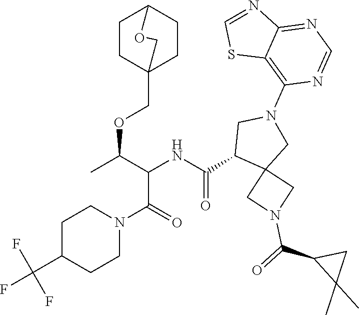 [SMILES: C[C@@H](OCC12CCC(CC1)OC2)C(NC(=O)[C@@H]1CN(c2ncnc3ncsc23)CC12CN(C(=O)[C@H]1CC1(C)C)C2)C(=O)N1CCC(C(F)(F)F)CC1] |
| I-31 | 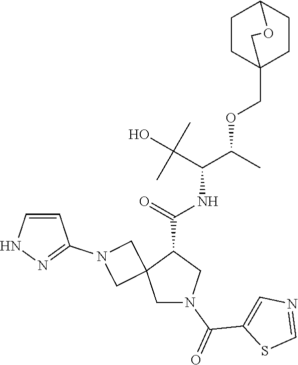 [SMILES: C[C@@H](OCC12CCC(CC1)OC2)[C@H](NC(=O)[C@@H]1CN(C(=O)c2cncs2)CC12CN(c1cc[nH]n1)C2)C(C)(C)O] |
| I-32 | 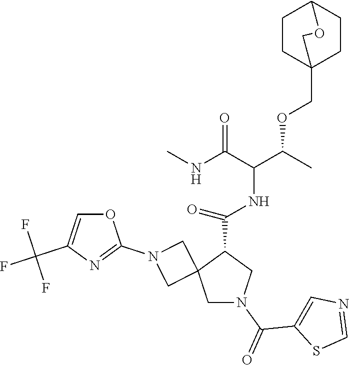 [SMILES: CNC(=O)C(NC(=O)[C@@H]1CN(C(=O)c2cncs2)CC12CN(c1nc(C(F)(F)F)co1)C2)[C@@H](C)OCC12CCC(CC1)OC2] |
| I-33 | 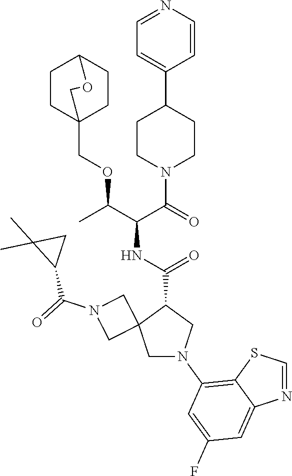 [SMILES: C[C@@H](OCC12CCC(CC1)OC2)[C@H](NC(=O)[C@@H]1CN(c2cc(F)cc3ncsc23)CC12CN(C(=O)[C@H]1CC1(C)C)C2)C(=O)N1CCC(c2ccncc2)CC1] |
| I-34 | 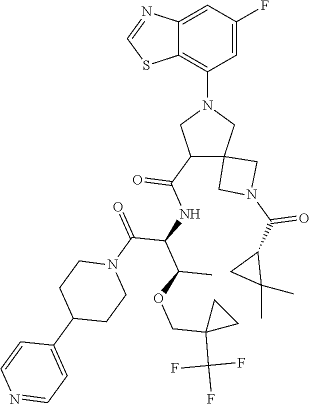 [SMILES: C[C@@H](OCC1(C(F)(F)F)CC1)[C@H](NC(=O)C1CN(c2cc(F)cc3ncsc23)CC12CN(C(=O)[C@H]1CC1(C)C)C2)C(=O)N1CCC(c2ccncc2)CC1] |
| I-35 | 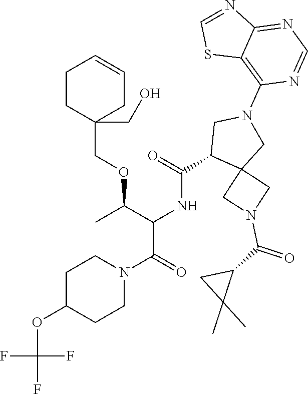 [SMILES: C[C@@H](OCC1(CO)CC=CCC1)C(NC(=O)[C@@H]1CN(c2ncnc3ncsc23)CC12CN(C(=O)[C@H]1CC1(C)C)C2)C(=O)N1CCC(OC(F)(F)F)CC1] |
| I-36 | 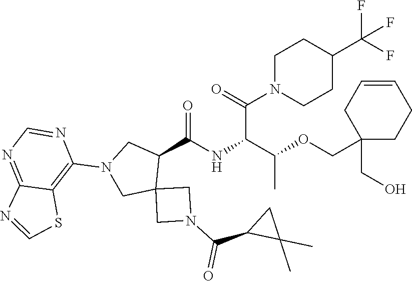 [SMILES: C[C@@H](OCC1(CO)CC=CCC1)[C@H](NC(=O)[C@@H]1CN(c2ncnc3ncsc23)CC12CN(C(=O)[C@H]1CC1(C)C)C2)C(=O)N1CCC(C(F)(F)F)CC1] |
| I-37 | 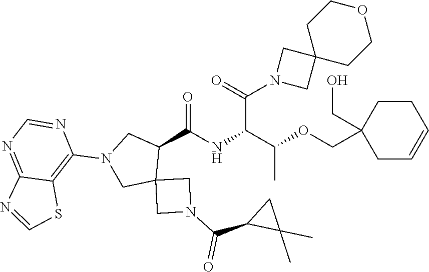 [SMILES: C[C@@H](OCC1(CO)CC=CCC1)[C@H](NC(=O)[C@@H]1CN(c2ncnc3ncsc23)CC12CN(C(=O)[C@H]1CC1(C)C)C2)C(=O)N1CC2(CCOCC2)C1] |
| I-38 | 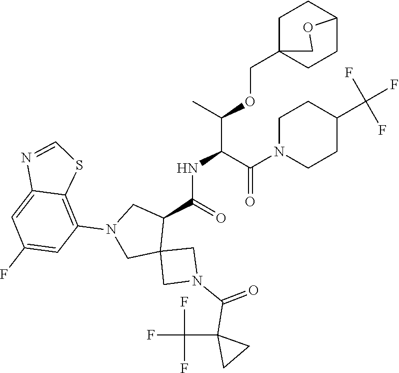 [SMILES: C[C@@H](OCC12CCC(CC1)OC2)[C@H](NC(=O)[C@@H]1CN(c2cc(F)cc3ncsc23)CC12CN(C(=O)C1(C(F)(F)F)CC1)C2)C(=O)N1CCC(C(F)(F)F)CC1] |
| I-39 | 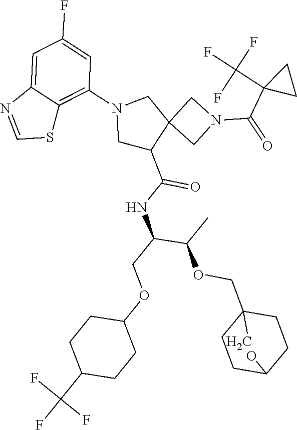 |
| I-40 | 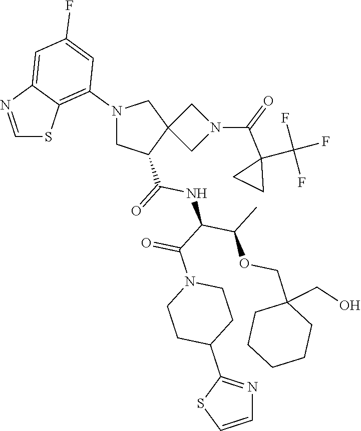 [SMILES: C[C@@H](OCC1(CO)CCCCC1)[C@H](NC(=O)[C@@H]1CN(c2cc(F)cc3ncsc23)CC12CN(C(=O)C1(C(F)(F)F)CC1)C2)C(=O)N1CCC(c2nccs2)CC1] |
| I-41 | 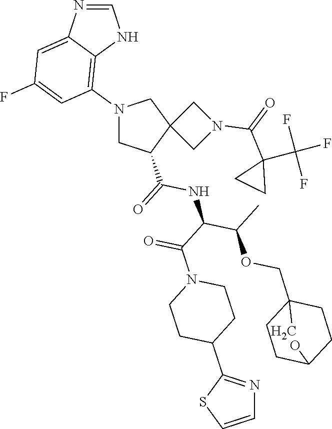 |
| I-42 | 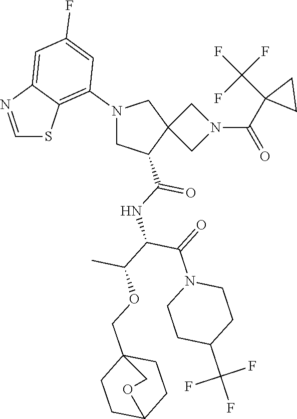 [SMILES: C[C@@H](OCC12CCC(CC1)OC2)[C@H](NC(=O)[C@@H]1CN(c2cc(F)cc3ncsc23)CC12CN(C(=O)C1(C(F)(F)F)CC1)C2)C(=O)N1CCC(C(F)(F)F)CC1] |
| I-43 | 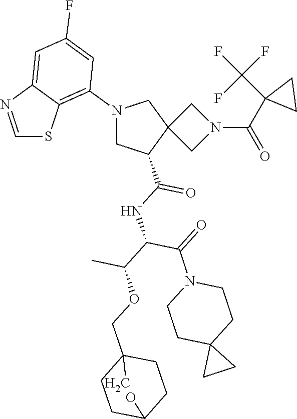 |
| I-44 | 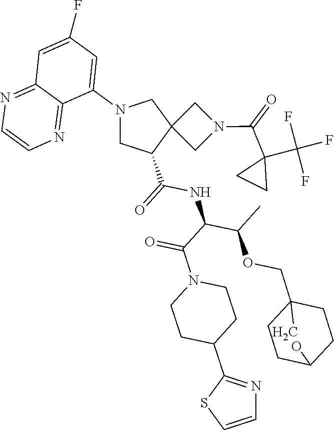 |
| I-45 | 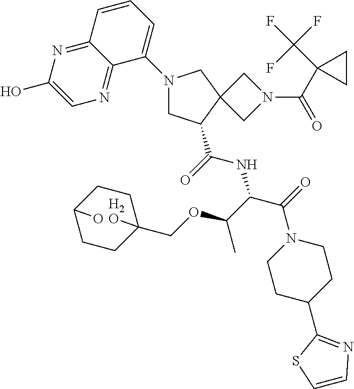 |
| I-46 | 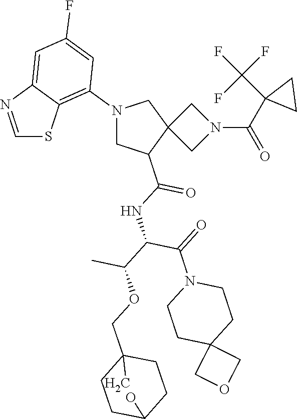 |
| I-47 | 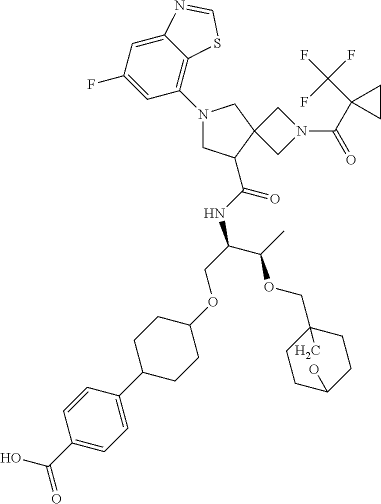 |
| I-48 | 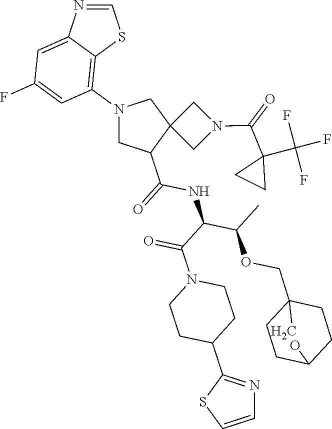 |
| I-49 | 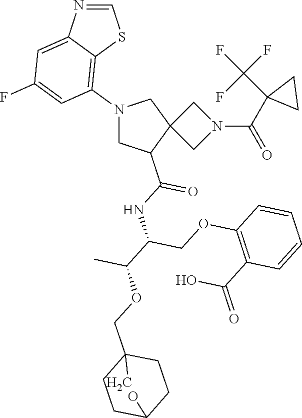 |
| I-50 | 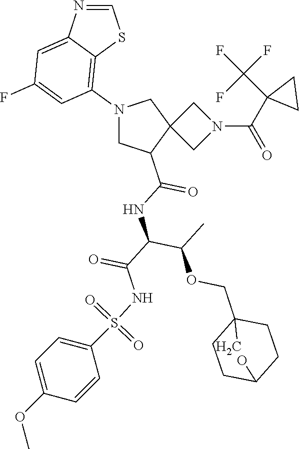 |
| I-51 | 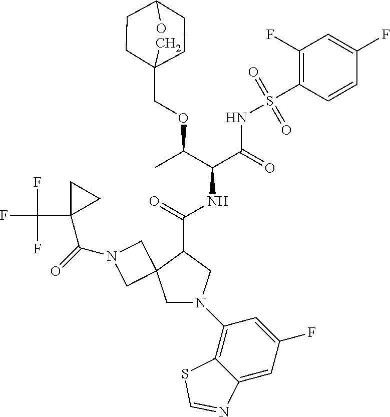 |
| I-52 | 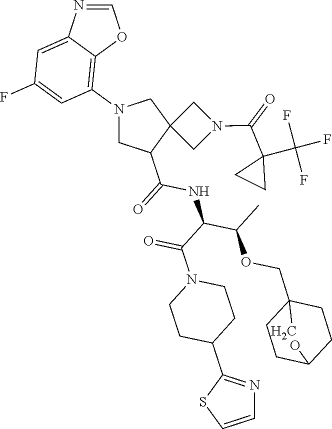 |
| I-53 | 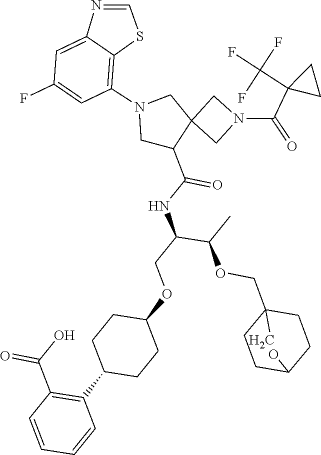 |
| I-54 | 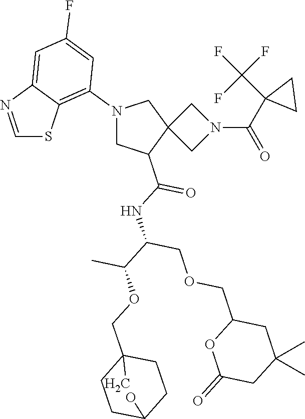 |
| I-55 | 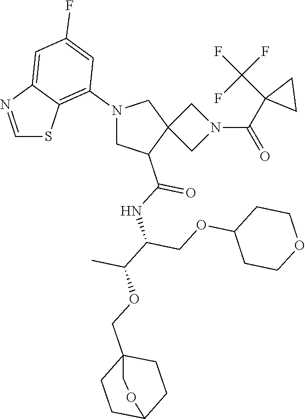 [SMILES: C[C@@H](OCC12CCC(CC1)OC2)[C@@H](COC1CCOCC1)NC(=O)C1CN(c2cc(F)cc3ncsc23)CC12CN(C(=O)C1(C(F)(F)F)CC1)C2] |
| I-56 | 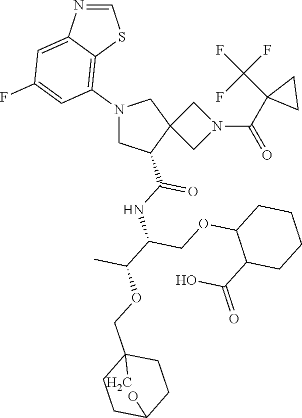 |
| I-57 | 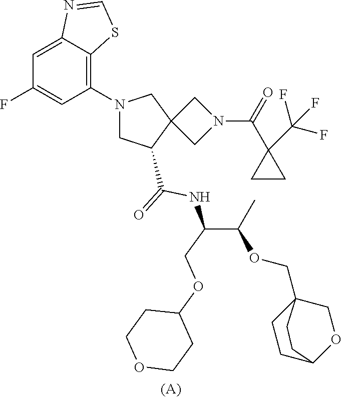 |
| | 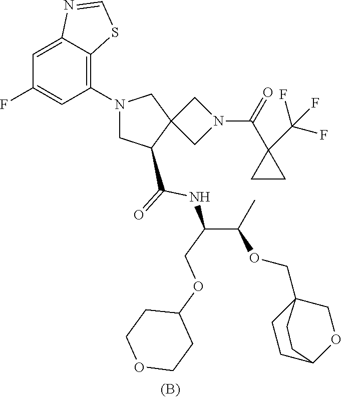 |
| I-58 | 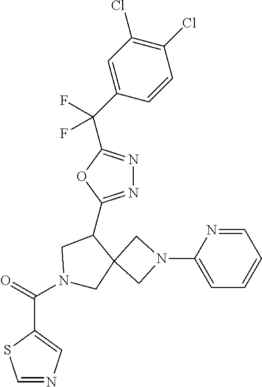 [SMILES: O=C(c1cncs1)N1CC(c2nnc(C(F)(F)c3ccc(Cl)c(Cl)c3)o2)C2(C1)CN(c1ccccn1)C2] |
| I-59 | 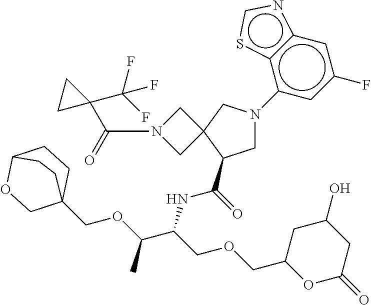 |
| I-60 | 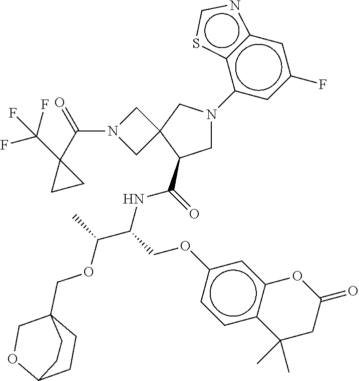 |
| I-61 | 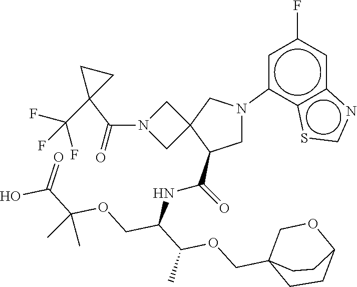 |
| I-62 | 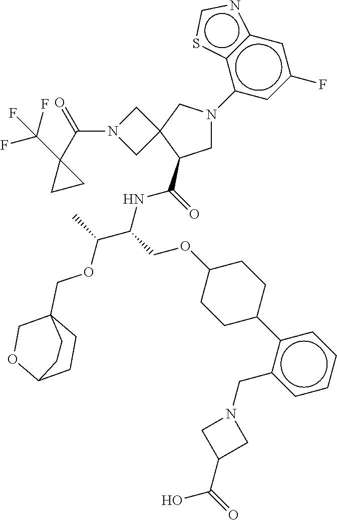 |
| I-63 | 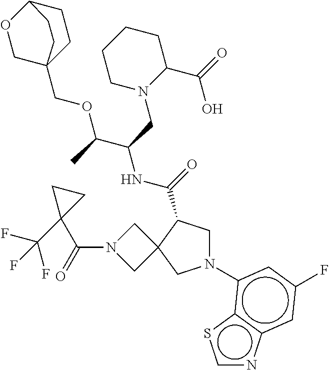 |
| I-64 | 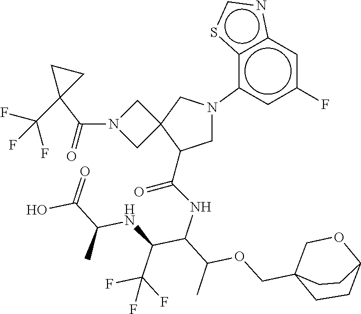 |
| I-65 | 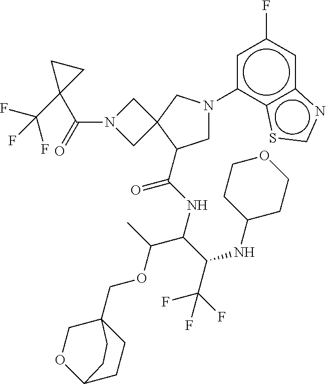 |
| I-66 | 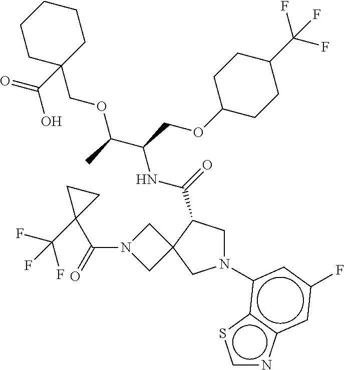 |
| I-67 | 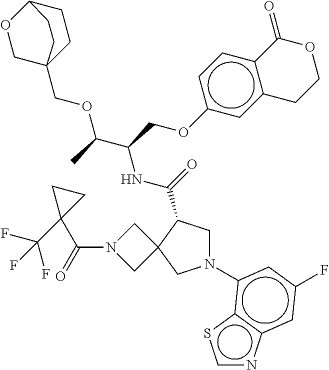 |
| I-68 | 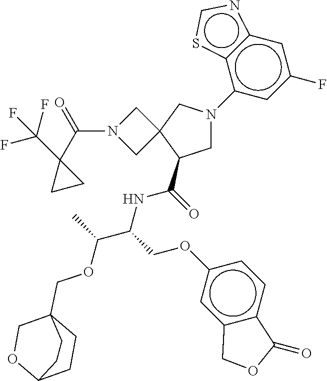 |
| I-69 | 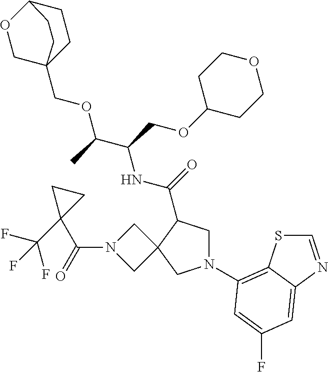 [SMILES: C[C@@H](OCC12CCC(CC1)OC2)[C@@H](COC1CCOCC1)NC(=O)C1CN(c2cc(F)cc3ncsc23)CC12CN(C(=O)C1(C(F)(F)F)CC1)C2] |
| I-70 | 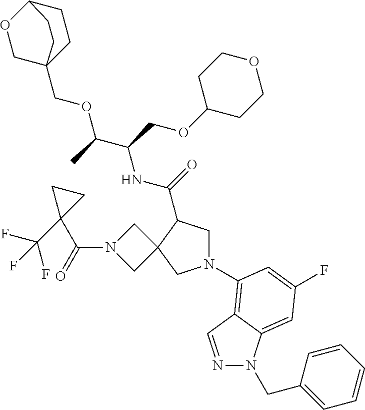 [SMILES: C[C@@H](OCC12CCC(CC1)OC2)[C@@H](COC1CCOCC1)NC(=O)C1CN(c2cc(F)cc3c2cnn3Cc2ccccc2)CC12CN(C(=O)C1(C(F)(F)F)CC1)C2] |
| I-71 | 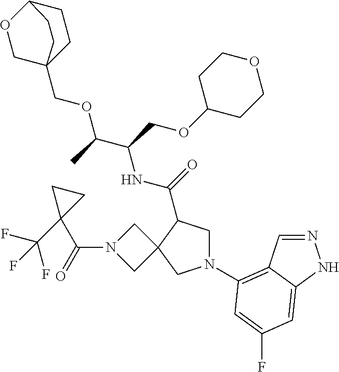 [SMILES: C[C@@H](OCC12CCC(CC1)OC2)[C@@H](COC1CCOCC1)NC(=O)C1CN(c2cc(F)cc3[nH]ncc23)CC12CN(C(=O)C1(C(F)(F)F)CC1)C2] |
| I-72 | 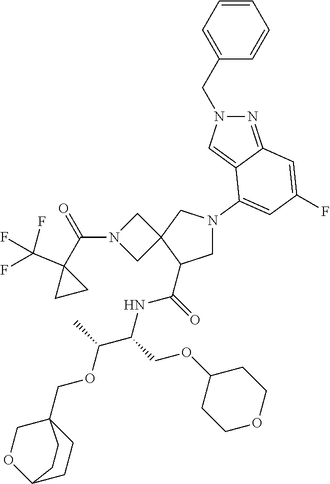 [SMILES: C[C@@H](OCC12CCC(CC1)OC2)[C@@H](COC1CCOCC1)NC(=O)C1CN(c2cc(F)cc3nn(Cc4ccccc4)cc23)CC12CN(C(=O)C1(C(F)(F)F)CC1)C2] |

The present disclosure contemplates any and all enantiomers, diastereomers and conformation isomers of a compound shown herein.

In some embodiments, the present disclosure provides a compound set forth in Table 7, above, or a pharmaceutically acceptable salt thereof. In some embodiments, the disclosure provides a compound set forth in Table 7, above, or a pharmaceutically acceptable salt thereof, and any enantiomers, diastereomers, or conformation isomers thereof.

In some embodiments, the present disclosure provides a pharmaceutical composition comprising a compound of the present disclosure, or a pharmaceutically acceptable salt thereof, together with a pharmaceutically acceptable carrier, excipient, vehicle, adjuvant or diluent. In some embodiments, the present disclosure provides a pharmaceutical composition comprising a compound set forth in Table 7 above, or a pharmaceutically acceptable salt thereof, together with a pharmaceutically acceptable carrier, excipient, vehicle, adjuvant or diluent. In some embodiments, the pharmaceutical composition further comprises an additional therapeutic agent.

In some embodiments, the present disclosure provides a complex comprising a CDK2 protein and a compound of the present disclosure.

In some embodiments, the present disclosure provides a method of inhibiting the activity of a cyclin-dependent kinase (CDK). In some embodiments, the method comprises contacting a compound of the present disclosure with a CDK. In some embodiments, the compound and the CDK are contacted in vivo. In some embodiments, the compound and the CDK are contacted in vitro. In some embodiments, the CDK is selected from CDK1, CDK2, CDK3, CDK4, CDK5, CDK6, CDK7, CDK8, CDK9, CDK10, CDK11, CDK12 and CDK13. In some embodiments, the CDK is CDK2. In some embodiments, the CDK is CDK3. In some embodiments, the CDK is CDK4. In some embodiments, the CDK is CDK6. In some embodiments, the method inhibits the activity of both CDK2 and CDK3. In some embodiments, the method inhibits the activity of CDK2 and one or both of CDK4 and CDK6.

In some embodiments, the compounds of the present disclosure inhibit the activity of one or more CDKs selected from CDK1, CDK2, CDK3, CDK4, CDK5, CDK6, CDK7, CDK8, CDK9, CDK10, CDK11, CDK12 and CDK13. In some embodiments, the compounds of the present disclosure inhibit CDK2. In some embodiments, the compounds of the present disclosure inhibit CDK3. In some embodiments, the compounds of the present disclosure inhibit CDK4. In some embodiments, the compounds of the present disclosure inhibit CDK5. In some embodiments, the compounds of the present disclosure inhibit CDK6. In some embodiments, the compounds of the present disclosure are CDK2/3 inhibitors. In some embodiments, the compounds of the present disclosure are CDK2/4/6 inhibitors.

In some embodiments, the present disclosure provides compounds that selectively inhibit CDK2 over other cyclin-dependent kinases (CDKs). In some embodiments, the compounds of the present disclosure selectively inhibit CDK2 over one or more other CDKs, selected from CDK1, CDK3, CDK4, CDK5, CDK6. CDK7. CDK8, CDK9, CDK10, CDK11, CDK12 and CDK13. In some embodiments, the compounds of the present disclosure selectively inhibit CDK2 over CDK4. In some embodiments, the compounds of the present disclosure selectively inhibit CDK2 over CDK6. In some embodiments, the compounds of the present disclosure selectively inhibit CDK2 over CDK4 and CDK6.

In some embodiments, the present disclosure provides compounds that selectively inhibit CDK2/cyclin E complexes over other CDK complexes.

### 4. General Methods of Providing the Present Compounds

The compounds of this disclosure may be prepared or isolated in general by synthetic and/or semi-synthetic methods known to those skilled in the art for analogous compounds and by methods described in detail in the Examples, herein.

In the Schemes below, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

Compounds of the present disclosure, including those of Formula I and the compounds of Table 7, can generally be prepared according the methods described below. Reagents and conditions can be modified and substituted using knowledge common to one of ordinary skill in the art, as needed, in order to arrive at the compounds of the present disclosure.

Amines which correspond to the side group R^{A} such as 3 in Scheme 1 may be prepared by amide coupling of acid 1 and optional further functionalization. Alternatively, instead of amide coupling, alkylation of the acid of 1 and optional further functionalization such as reduction or displacement via substitution reaction of the carbonyl oxygen may produce R² in compound 3. 3 may be coupled with spirocyclic compounds 5, 7, or 10 (see also Scheme 2) to provide compounds of the disclosure. Attachment of aryl groups directly bonded to spirocyclic cores in the present compounds may be achieved via Buchwald-Hartwig Cross-Coupling as shown in Schemes 1 and 2.

Spriocyclic precursors (i.e., compounds 17-19) to compounds, for example, 4 and 7, may be prepared according to Scheme 3 and subsequently functionalized at the amine using alkylation or amidation chemistries. To prepare the spirocycles, Horner-Wadsworth-Emmons reaction between 11 and 12 furnishes 13 which when reacted with tertiary amine 14 produces the spirocyclic core of the present compounds in compound 15. 15 may be further functionalized, deprotected and/or protected using orthogonal protecting group strategies as known in the art to protect or deprotect either of the spirocyclic amines or pendant carboxylic acid of compounds 16 or 17 to furnish the appropriate spirocycle for completing the compounds, for example, compounds 17-19.

Scheme 4 illustrates a more detailed synthesis of a specific compound 3 (compound 31, see Scheme 4).

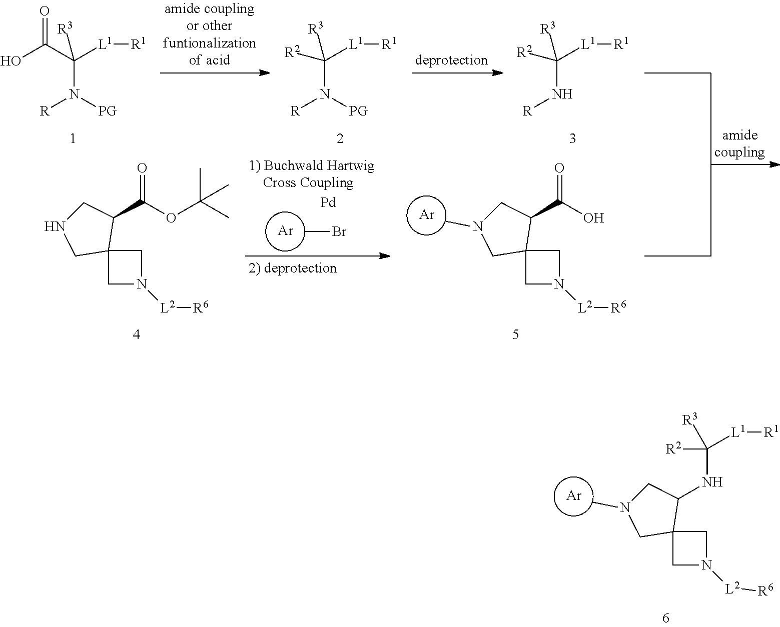

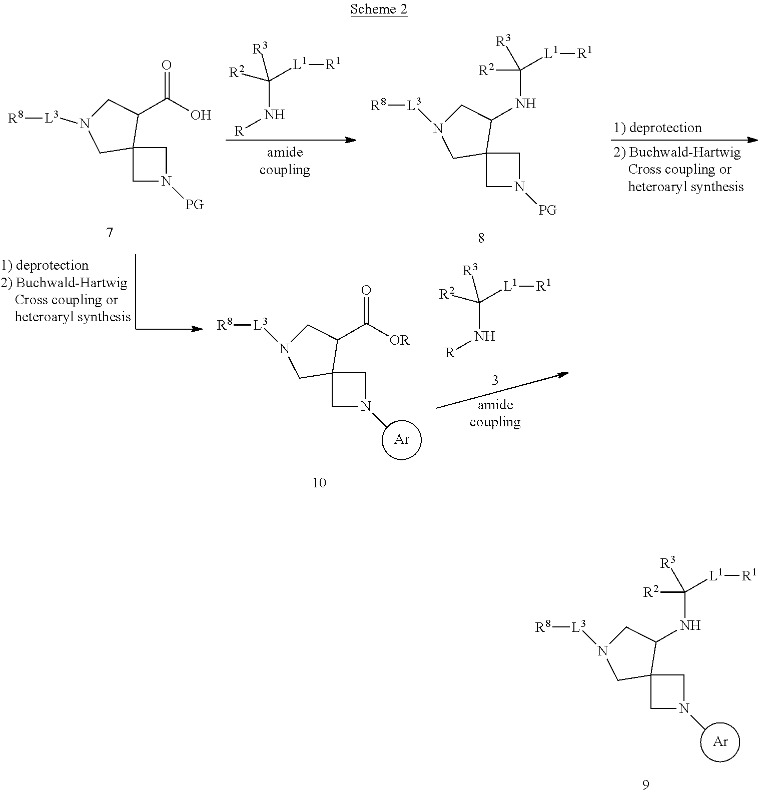

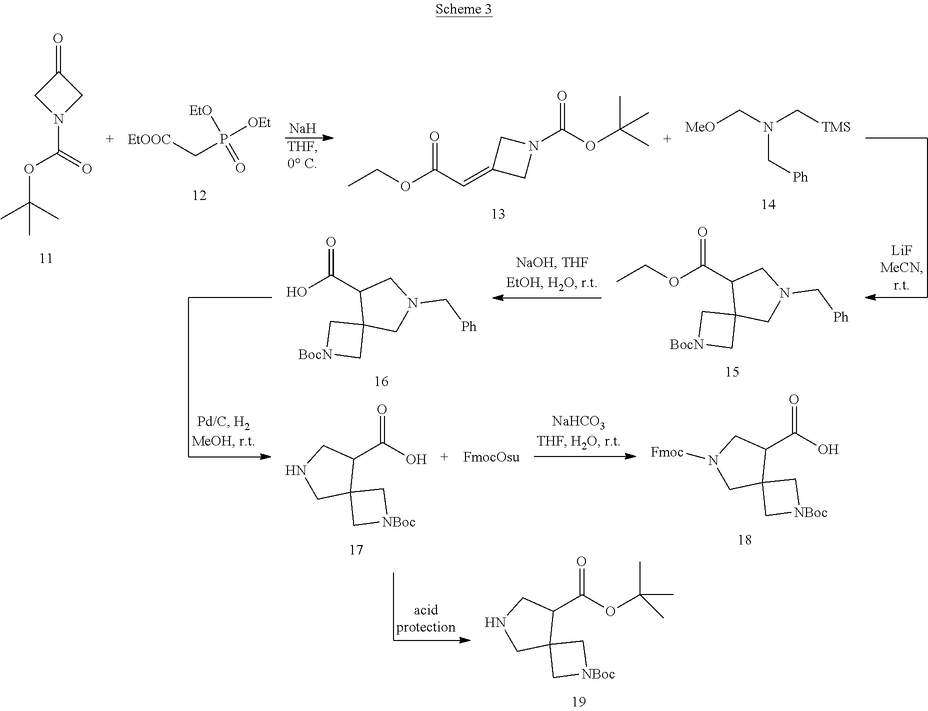

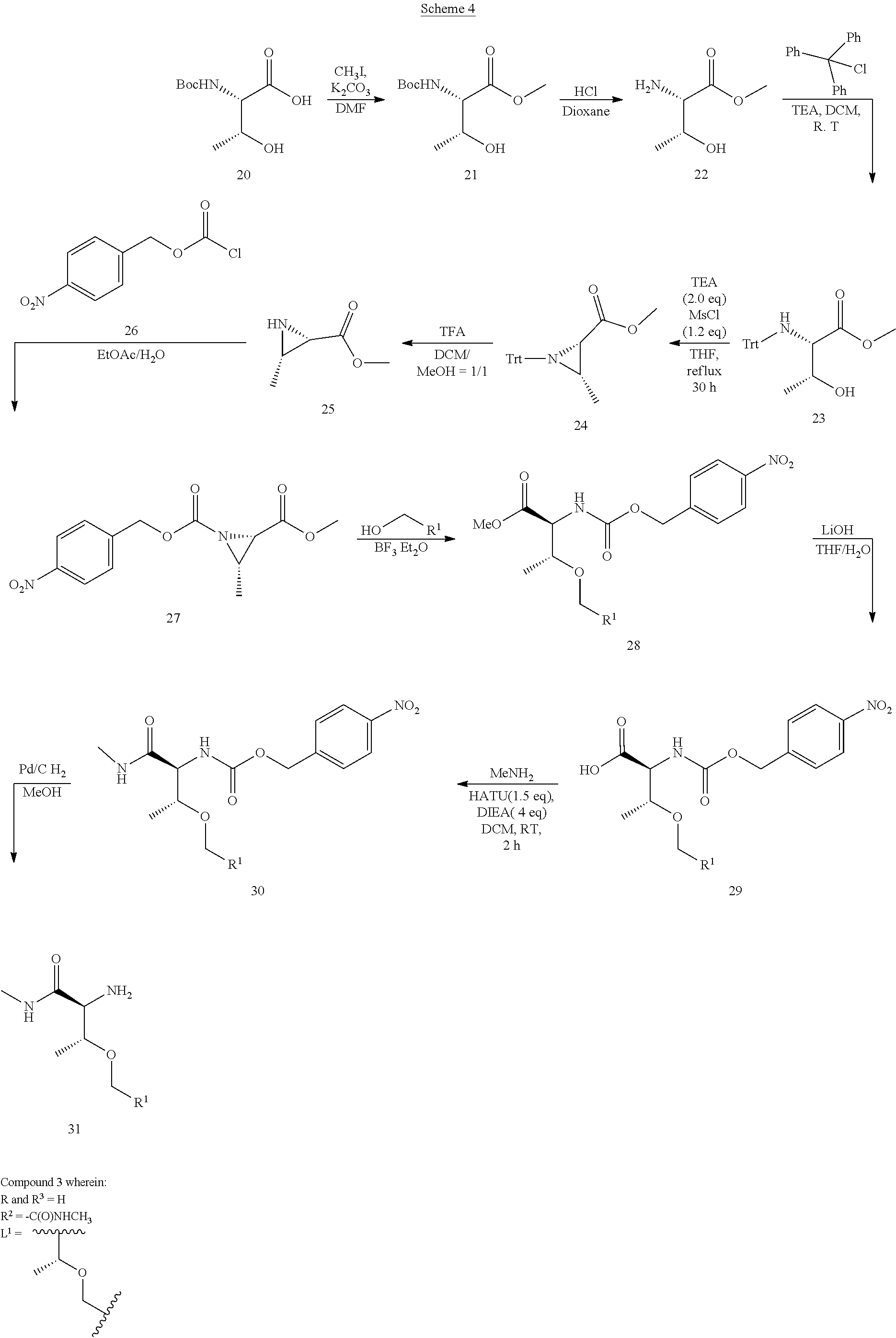

### 5. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably inhibit a CDK2 protein, or a mutant thereof, in a biological sample or in a patient. In certain embodiments, the amount of compound in compositions of this disclosure is such that it is effective to measurably inhibit a CDK2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered subcutaneously, orally, intraperitoneally or intravenously. In some embodiments, the compositions are administered orally. In some embodiments, the compositions are administered intraperitoneally. In some embodiments, the compositions are administered intravenously. In some embodiments, the compositions are administered subcutaneously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens. Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the modulation of the activity CDK2. In some embodiments, the compounds and compositions described herein are CDK2 inhibitors.

In some embodiments, the compounds and compositions of the present disclosure are useful for treating diseases and disorders associated with CDK2 activity, including, but not limited to cancers, myeloproliferative disorders, autoimmune disorders, inflammatory disorders, viral infections, fibrotic disorders, and neurodegenerative disorders.

In some embodiments, the disclosure provides a method of inhibiting the activity of a CDK2, the method comprising contacting a compound of the present disclosure, or a pharmaceutically acceptable salt thereof with the CDK2. In some embodiments, the contacting takes place in vitro. In some embodiments, the contacting takes place in vivo.

In some embodiments, the disclosure provides a method of treating, preventing or lessening the severity of a disease or disorder associated with CDK2 activity in a patient, including, but not limited to cancers, myeloproliferative disorders, autoimmune disorders, inflammatory disorders, fibrotic disorders, and neurodegenerative disorders, said method comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the treatment of a disease or disorder associated with CDK2 activity.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the manufacture of a medicament for treating a disease or disorder associated with CDK2 activity.

In some embodiments, the disease or disorder associated with CDK2 activity is a CDK2-mediated disease or disorder. In some embodiments, the disease or disorder associated with CDK2 activity is a disease or disorder caused by CDK2 over-activity.

In some embodiments, the disease or disorder associated with CDK2 activity is cancer.

In some embodiments, the cancer is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

In some embodiments, the cancer is characterized by amplification or overexpression of CCNE1 and/or CCNE2.

In some embodiments, the cancer is breast cancer. In some embodiments, the breast cancer is a breast cancer selected from ER-positive/HR-positive breast cancer, HER2-negative breast cancer, ER-positive/HR-positive breast cancer, HER2-positive breast cancer, triple negative breast cancer (TNBC), inflammatory breast cancer, endocrine resistant breast cancer, trastuzumab resistant breast cancer, breast cancer with primary or acquired resistance to CDK4/CDK6 inhibition, advanced breast cancer and metastatic breast cancer. In some embodiments the breast cancer is characterized by amplification or overexpression of CCNE1 and/or CCNE2.

In some embodiments, the cancer is ovarian cancer. In some embodiments, the ovarian cancer is high-grade serous ovarian cancer (HGSOC). In some embodiments the ovarian cancer is characterized by amplification or overexpression of CCNE1 and/or CCNE2.

In some embodiments, the cancer is bladder cancer. In some embodiments, the bladder cancer is characterized by amplification or overexpression of CCNE1 and/or CCNE2.

In some embodiments, the cancer is uterine cancer. In some embodiments, the uterine cancer is characterized by amplification or overexpression of CCNE1 and/or CCNE2.

In some embodiments, the cancer is prostate cancer. In some embodiments, the prostate cancer is characterized by amplification or overexpression of CCNE1 and/or CCNE2.

In some embodiments, the cancer is lung cancer. In some embodiments, the lung cancer is a lung cancer selected from non-small cell lung cancer, small cell lung cancer, squamous cell carcinoma, adenocarcinoma, and mesothelioma. In some embodiments, the lung cancer is characterized by amplification or overexpression of CCNE1 and/or CCNE2. In some embodiments, the lung cancer is CCNE1 amplified squamous cell carcinoma or CCNE1 amplified adenocarcinoma.

In some embodiments, the cancer is head and neck cancer. In some embodiments, the head and neck cancer is characterized by amplification or overexpression of CCNE1 and/or CCNE2.

In some embodiments, the cancer is colorectal cancer. In some embodiments, the colorectal cancer is characterized by amplification or overexpression of CCNE1 and/or CCNE2.

In some embodiments, the cancer is kidney cancer. In some embodiments, the kidney cancer is renal cell carcinoma (RCC). In some embodiments, the kidney cancer is characterized by amplification or overexpression of CCNE1 and/or CCNE2.

In some embodiments, the cancer is liver cancer. In some embodiments, the liver cancer is hepatocellular carcinoma (HCC). In some embodiments, the liver cancer is characterized by amplification or overexpression of CCNE1 and/or CCNE2.

In some embodiments, the cancer is pancreatic cancer. In some embodiments, the pancreatic cancer is characterized by amplification or overexpression of CCNE1 and/or CCNE2.

In some embodiments, the cancer is stomach cancer. In some embodiments, the stomach cancer is characterized by amplification or overexpression of CCNE1 and/or CCNE2.

In some embodiments, the cancer is melanoma. In some embodiments, the melanoma is characterized by amplification or overexpression of CCNE1 and/or CCNE2. CDK2 expression is regulated by essential melanocytic transcription factor MITF. It has been found that CDK2 depletion suppresses the growth of melanoma (Du et al., Cancer Cell. 2004 December; 6(6): 565-576)

In some embodiments, the cancer is thyroid cancer. In some embodiments, the thyroid cancer is characterized by amplification or overexpression of CCNE1 and/or CCNE2.

In some embodiments, the disease or disorder associated with CDK2 activity is a myeloproliferative disorder.

In some embodiments, the disease or disorder associated with CDK2 activity is a neurodegenerative disease or disorder. In some embodiments, the neurodegenerative disease or disorder is Alzheimer's disease (AD). It has been reported that neuronal cell death in subjects suffering from AD is preceded by cell cycle events. Inhibition of one or more CDKs can inhibit cell cycle events and therefore stave off neuronal cell death (Yang et al., J Neurosci. 2003 Apr. 1; 23(7): 2557-2563).

In some embodiments, the disease or disorder associated with CDK2 activity is a liver disease.

In some embodiments, the disease or disorder associated with CDK2 activity is liver fibrosis. It has been reported that CCNE1 knockout mice do not develop liver fibrosis upon exposure to pro-fibrotic toxin CCl₄, suggesting that liver fibrosis can be treated via administration of a CDK2 inhibitor (Nevzorova, et al., Hepatology. 2012 September; 56(3): 1140-1149).

In some embodiments, the disease or disorder associated with CDK2 activity is Cushing disease. Pituitary cyclin E/E2F1 signaling is a molecular mechanism underlying neuroendocrine regulation of the hypothalamic-pituitary-adrenal axis, and therefore provides a subcellular therapeutic target for CDK2 inhibitors of pituitary ACTH-dependent hypercortisolism, also known as Cushing disease (Liu, et al., J Clin Endocrinol Metab. 2015 July; 100(7): 2557-2564).

In some embodiments, the disease or disorder associated with CDK2 activity is a kidney disease.

In some embodiments, the disease or disorder associated with CDK2 activity is polycystic kidney disease. It has been reported that CDK2/CDK5 inhibitor roscovitine yields effective arrest of cystic kidney disease in mouse models of polycystic kidney disease (Bukanov, et al., Nature. 2006 Dec. 14; 444(7121): 949-52).

In some embodiments, the disease or disorder associated with CDK2 activity is an autoimmune disorder. CDK2 ablation has been shown to promote immune tolerance by supporting the function of regulatory T cells (Chunder et al., J Immunol. 2012 Dec. 15; 189(12): 5659-66).

In some embodiments, the disease or disorder associated with CDK2 activity is an inflammatory disorder. Cyclin E ablation has been shown to attenuate hepatitis in mice, while p27 knockout mice display exacerbation of renal inflammation (Ehedego et al., Oncogene. 2018 June; 37(25): 3329-3339: Ophascharoensuk et al., Nat Med. 1998 May; 4(5): 575-80). In some embodiments, the inflammatory disorder is hepatitis.

In some embodiments, the compounds and compositions of the present disclosure are useful as male contraceptives. Based on the finding that male CDK2 knockout mice are sterile, CDK2 inhibitors have been studied as possible male contraceptives (Faber, et al., Biol Reprod. 2020 August; 103(2): 357-367). In some embodiments, the present disclosure provides a method of reducing male fertility comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

In some embodiments, the compounds and compositions of the present disclosure are useful for treating diseases and disorders associated with CDK5 activity, including, but not limited to cancers, myeloproliferative disorders, autoimmune disorders, inflammatory disorders, viral infections, fibrotic disorders, and neurodegenerative disorders. In some embodiments, the compounds and compositions of the present disclosure are useful for treating neurodegenerative disorders associated with CDK5 activity.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents that the compounds of the present disclosure may also be combined with include, without limitation: endocrine therapeutic agents, chemotherapeutic agents and other CDK inhibitory compounds.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of an endocrine therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional CDK inhibitory compounds. In some embodiments, the one or more additional CDK inhibitory compounds are CDK4, or CDK4/CDK6 inhibitors. In some embodiments, the one or more additional CDK inhibitory compounds are CDK4, CDK6, CDK7 or CDK4/CDK6 inhibitors. In some embodiments, the one or more additional CDK inhibitory compounds are CDK4 inhibitors. In some embodiments, the one or more additional CDK inhibitory compounds are CDK6 inhibitors. In some embodiments, the one or more additional CDK inhibitory compounds are CDK7 inhibitors. In some embodiments, the one or more additional CDK inhibitory compounds are CDK4/CDK6 inhibitors.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of a chemotherapeutic agent. In some embodiments, the chemotherapeutic agent is a taxane. In some embodiments, the chemotherapeutic agent is a platinum agent. In some embodiments, the chemotherapeutic agent is trastuzumab.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In some embodiments, one or more other therapeutic agent and a compound or composition the present disclosure are administered as a multiple dosage regimen within greater than 24 hours a parts.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### EXAMPLES

As depicted in the Examples below, in certain exemplary embodiments, compounds are prepared according to the procedures provided herein. It will be appreciated that, although the methods depict the synthesis of certain compounds of the present disclosure, the methods, and other methods known to one of ordinary skill in the art, can be applied to all compounds and subclasses and species of each of these compounds, as described herein.

### Example 1: Synthesis of Compounds of the Disclosure

### Synthesis of (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(4-(trifluoromethyl)thiazol-2-yl)-2,6-diazaspiro[3.4]octane-8-carboxamide I-17

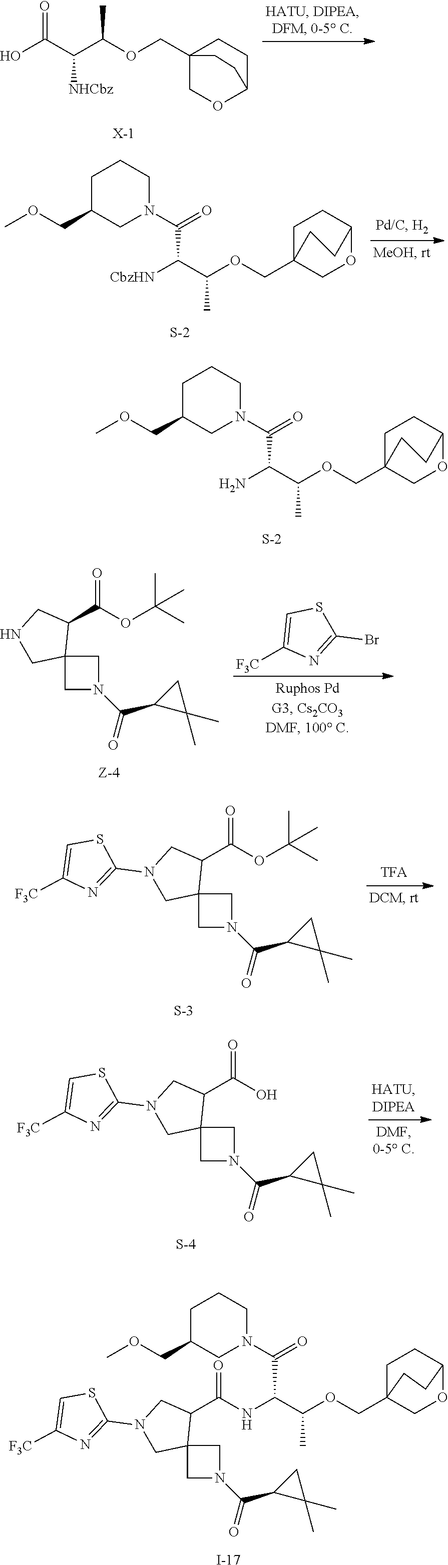

Step 1: Benzyl ((2S,3R)-3-(2-oxabicyclo[2.2.2]octan-4-ylmethoxy)-1-((S)-3-(methoxymethyl)piperidin-1-yl)-1-oxobutan-2-yl)carbamate (S-1): To a solution of (2S,3R)-3-(2-oxabicyclo[2.2.2]octan-4-ylmethoxy)-2-(((benzyloxy)carbonyl)amino)butanoic acid (X-1) (0.330 g, 0.87 mmol), (S)-3-(methoxymethyl)piperidine hydrochloride (0.159 g, 0.96 mmol), and N-ethyl-N-isopropylpropan-2-amine (0.339 g, 2.62 mmol) in N,N-dimethylformamide (3 mL) at 0-5° C. was added (2-(7-aza-1H-benzotriazole-1-yl)-1,1,3,3-tetramethyluronium hexafluorophosphate) (0.399 g, 1.05 mmol). The resulting mixture was stirred at 0-5° C. for 30 minutes. The reaction mixture was poured into water (20 mL) and extracted with ethyl acetate (10 mL×3). The combined organic layers were washed with brine (20 mL×2), dried over anhydrous sodium sulfate, and concentrated under reduced pressure to give a crude residue, which was purified by column chromatography using 50% ethyl acetate in hexane gradient to afford benzyl ((2S,3R)-3-(2-oxabicyclo[2.2.2]octan-4-ylmethoxy)-1-((S)-3-(methoxymethyl)piperidin-1-yl)-1-oxobutan-2-yl)carbamate (S-1) (0.350 g, 82%) as a colorless oil. MS: [MH]: 489.3.

Step 2: (2S,3R)-3-(2-oxabicyclo[2.2.2]octan-4-ylmethoxy)-2-amino-1-((S)-3-(methoxymethyl)piperidin-1-yl)butan-1-one (S-2): To a solution of benzyl ((2S,3R)-3-(2-oxabicyclo[2.2.2]octan-4-ylmethoxy)-1-((S)-3-(methoxymethyl)piperidin-1-yl)-1-oxobutan-2-yl)carbamate (S-1) (0.350 g, 0.72 mmol) in methanol (10 mL) was added palladium on carbon (10%, 0.100 g). The resulting mixture was stirred at room temperature under H₂ overnight. TLC showed the reaction was complete. Palladium on carbon was removed through filtration and washed with methanol (10 mL×2). The combined filtrates were concentrated under reduced pressure to afford (2S,3R)-3-(2-oxabicyclo[2.2.2]octan-4-ylmethoxy)-2-amino-1-((S)-3-(methoxymethyl)piperidin-1-yl)butan-1-one (S-2) (0.240 g, 94%) as a colorless oil. MS: [MH]+ 355.3.

Step 3: tert-butyl2-((S)-2,2-dimethylcyclopropanecarbonyl)-6-(4-(trifluoro methyl)thiazol-2-yl)-2,6-diazaspiro[3.4]octane-8-carboxylate (S-3): To a mixture of (S)-tert-butyl 2-((S)-2,2-dimethylcyclopropanecarbonyl)-2,6-diazaspiro[3.4]octane-8-carboxylate (Z-4) (0.100 g, 0.32 mmol), 2-bromo-4-(trifluoromethyl)thiazole (0.083 g, 0.36 mmol), and cesium carbonate (0.211 g, 0.65 mmol) in N,N-dimethylformamide (2.5 mL) was added RuPhos Pd G3 (0.054 g, 0.065 mmol) under nitrogen atmosphere. The resulting mixture was stirred at 100° C. for 3 hours. The reaction mixture was poured into water (20 mL) and extracted with ethyl acetate (5 mL×3). The combined organic layers were washed with brine (20 mL×2), dried over anhydrous sodium sulfate, and concentrated under reduced pressure to give a crude residue, which was purified by silica gel column chromatography using a 25% ethyl acetate in hexane gradient to afford (S)-tert-butyl 2-((S)-2,2-dimethylcyclopropanecarbonyl)-6-(4-(trifluoromethyl)thiazol-2-yl)-2,6-diazaspiro[3.4]octane-8-carboxylate (S-3) (0.094 g, 63%) as a yellow oil. MS: [MH]+ 460.4.

Step 4: 2-((S)-2,2-dimethylcyclopropanecarbonyl)-6-(4-(trifluoromethyl)thiazol-2-yl)-2,6-diazaspiro[3.4]octane-8-carboxylic acid (S-4): To a solution of (S)-tert-butyl 2-((S)-2,2-dimethylcyclopropanecarbonyl)-6-(4-(trifluoromethyl)thiazol-2-yl)-2,6-diazaspiro[3.4]octane-8-carboxylate (S-3) (60 mg, 0.13 mmol) in dichloromethane (3 mL) was added 2,2,2-trifluoroacetic acid (2 mL). The resulting mixture was stirred at room temperature for 3 hours. TLC showed the reaction was complete. The volatiles were evaporated under reduced pressure to afford crude (S)-2-((S)-2,2-dimethylcyclopropanecarbonyl)-6-(4-(trifluoromethyl)thiazol-2-yl)-2,6-diazaspiro[3.4]octane-8-carboxylic acid (S-4) as a yellow oil, which was used in the next step without further purification. MS: [MH]⁺ 404.3.

Step 5: N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(4-(trifluoromethyl)thiazol-2-yl)-2,6-diazaspiro[3.4]octane-8-carboxamide (1-17): To a solution of crude (S)-2-((S)-2,2-dimethylcyclopropanecarbonyl)-6-(4-(trifluoromethyl)thiazol-2-yl)-2,6-diazaspiro[3.4]octane-8-carboxylic acid (S-4) (0.13 mmol), (2S,3R)-3-(2-oxabicyclo[2.2.2]octan-4-ylmethoxy)-2-amino-1-((S)-3-(methoxymethyl)piperidin-1-yl)butan-1-one (S-2) (0.051 g, 0.14 mmol), and N-ethyl-N-isopropylpropan-2-amine (0.051 g, 0.39 mmol) in N,N-dimethylformamide (1 mL) at 0-5° C. was added (2-(7-Aza-1H-benzotriazole-1-yl)-1,1,3,3-tetramethyluronium hexafluorophosphate) (0.060 g, 0.16 mmol). The resulting mixture was stirred at 0-5° C. for 1 hour. The reaction mixture was poured into water (20 mL) and extracted with ethyl acetate (5 mL×3). The combined organic layers were washed with brine (20 mL×2), dried over anhydrous sodium sulfate, and concentrated under reduced pressure to give a crude residue, which was purified by preparative TLC using 5% methanol in dichloromethane gradient to afford a diastereomeric mixture of N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(4-(trifluoromethyl)thiazol-2-yl)-2,6-diazaspiro[3,4]octane-8-carboxamide I-17A (0.021 g, 21%) and I-17B (0.018 g, 19%) as a white solid. I-17A: ¹HNMR (400 MHz, CD₃OD): δ 7.37-7.11 (m, 1H), 7.22 (s, 1H), 4.96-4.90 (m, 1H), 4.42-4.13 (m, 4H), 4.07-4.03 (m, 1H), 3.98-3.64 (m, 10H), 3.54-3.45 (m, 1H), 3.27-3.12 (m, 3H), 3.06-3.02 (m, 1H), 2.93-2.86 (m, 1H), 2.73-2.62 (m, 1H), 2.04-1.94 (m, 2H), 1.85-1.62 (m, 7H), 1.53-1.34 (m, 6H), 1.19-1.14 (m, 9H), 1.07-1.04 (m, 1H), 0.81-0.77 (m, 1H): MS: [MH]⁺ 740.6. I-17B: ¹HNMR (400 MHz. CD₃OD): δ 7.36-7.11 (m, 1H), 7.21 (s, 1H), 4.96-4.86 (m, 1H), 4.53 (d, J=9.6 Hz, 1H), 4.41-4.23 (m, 2H), 4.18-3.63 (m, 13H), 3.50-3.44 (m, 1H), 3.28-3.11 (m, 4H), 3.01 (d, J=9.2 Hz, 1H), 2.75-2.60 (m, 1H), 2.02-1.91 (m, 2H), 1.87-1.77 (m, 2H), 1.72-1.60 (m, 5H), 1.51-1.41 (m, 4H), 1.38-1.31 (m, 2H), 1.18 (d, J=3.2 Hz, 3H), 1.13-1.10 (m, 6H), 1.06-1.02 (m, 1H), 0.80-0.76 (m, 1H); MS: [MH]⁺ 740.6.

### Synthesis of (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(1H-pyrazolo[3,4-b]pyridin-3-yl)-2,6-diazaspiro[3.4]octane-8-carboxamide (I-24)

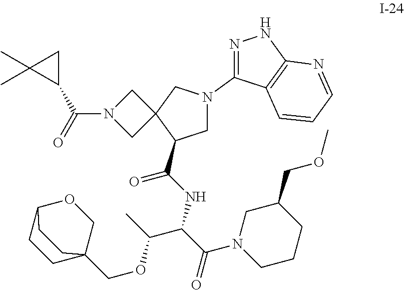 [SMILES: COC[C@H]1CCCN(C(=O)[C@@H](NC(=O)[C@@H]2CN(c3n[nH]c4ncccc34)CC23CN(C(=O)[C@H]2CC2(C)C)C3)[C@@H](C)OCC23CCC(CC2)OC3)C1]

I-24 was prepared (0.013 g, 35%) as a white solid using a procedure analogous to that used for I-17. ¹H NMR (400 MHz, CD₃OD): δ 8.38-8.31 (m, 2H), 7.06-7.03 (m, 1H), 4.97-4.89 (m, 1H), 4.56-3.84 (m, 11H), 3.69-3.63 (m, 4H), 3.48-3.41 (m, 1H), 3.30-3.29 (m, 2H) 3.27-3.13 (m, 5H), 3.03-2.85 (m, 2H), 2.76-2.60 (m, 1H), 1.91-1.39 (m, 13H), 1.19-1.18 (m, 3H), 1.15-1.11 (m, 6H), 1.07-1.06 (m, 1H), 0.81-0.77 (m, 1H). MS: [MH]⁺ 706.95.

### Synthesis of (S)-N-((3S,4R)-4-((2-oxabicyclo[2.2.2]octan-4-yl)methoxy)-2-hydroxy-2-methylpentan-3-yl)-6-(benzo[d]thiazol-7-yl)-2-((S)-2,2-dimethylcyclopropane-1-carbonyl)-2,6-diazaspiro[3.4]octane-8-carboxamide I-16

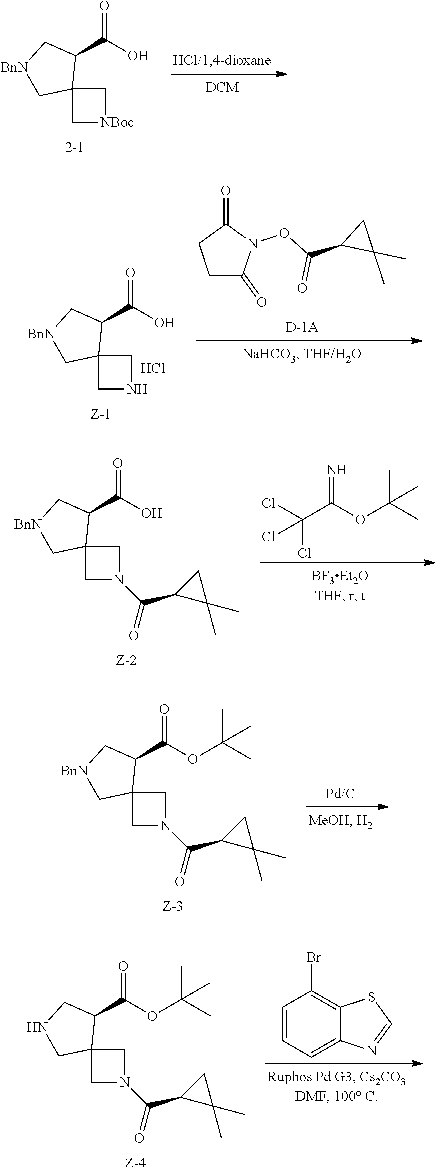

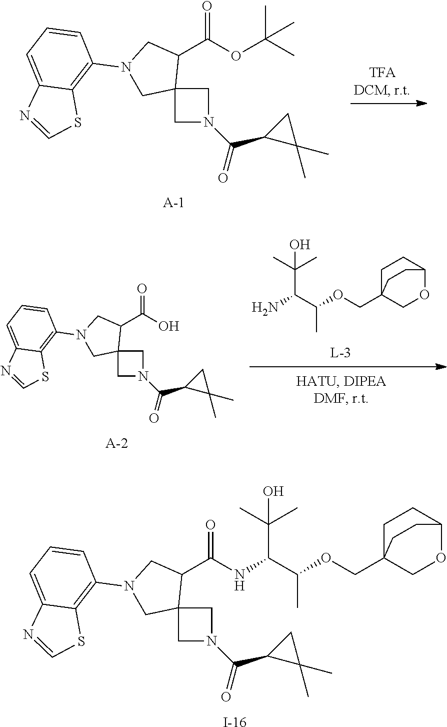

Step 1: (S)-6-benzyl-2,6-diazaspiro[3.4]octane-8-carboxylic acid hydrochloride (Z-1): A mixture of (S)-6-benzyl-2-(tert-butoxycarbonyl)-2,6-diazaspiro[3.4]octane-8-carboxylic acid 2-1 (0.500 g, 5.60 mmol) and hydrogen chloride 1,4-dioxane solution (4.0 N, 2 mL) in dichloromethane (5 mL) was stirred at room temperature under nitrogen atmosphere for 2 hours. TLC showed the reaction was complete. The mixture was concentrated under reduced pressure to afford (S)-6-benzyl-2,6-diazaspiro[3.4]octane-8-carboxylic acid hydrochloride (Z-1) (0.500 g, crude) as a white solid, which was used in the next step without further purification. MS: [MH]⁺ 247.0.

Step 2: (S)-6-benzyl-2-((S)-2,2-dimethylcyclopropane-1-carbonyl)-2,6-diazaspiro[3.4]octane-8-carboxylic acid (Z-2): To a stirred mixture of (S)-6-benzyl-2,6-diazaspiro[3.4]octane-8-carboxylic acid hydrochloride Z-1 (3.000 g, crude) in water (20 mL) was added sodium bicarbonate (3.640 g, 43.33 mmol) and a solution of 2,5-dioxopyrrolidin-1-yl (S)-2,2-dimethylcyclopropane-1-carboxylate (D-1A) (1.830 g, 8.67 mmol: prepared as described in J. Med. Chem. 1987, 30, 6, 1074-1090, the entire contents of which are incorporated herein by reference) in tetrahydrofuran (20 mL). The resulting mixture was stirred at room temperature for 2 hours. The reaction mixture was partitioned between ethyl acetate (20 mL) and water (20 mL). The aqueous layer was extracted with a mixture of dichloromethane and isopropyl alcohol (3/1, v/v, 40 mL×3). The combined extracts were dried over anhydrous sodium sulfate and concentrated to give a crude residue, which was purified by silica gel column chromatography using a 5% methanol in dichloromethane gradient to afford (S)-6-benzyl-2-((S)-2,2-dimethylcyclopropane-1-carbonyl)-2,6-diazaspiro[3.4]octane-8-carboxylic acid (Z-2) (2.100 g. 66% of 2 steps) as a colorless solid. MS: [MH]⁺ 343.4.

Step 3: (S)-6-benzyl-2-((S)-2,2-dimethylcyclopropane-1-carbonyl)-2,6-diazaspiro[3.4]octane-8-carboxylate (Z-3): A mixture of (S)-6-benzyl-2-((S)-2,2-dimethylcyclopropane-1-carbonyl)-2,6-diazaspiro[3.4]octane-8-carboxylic acid Z-2 (2.100 g, 6.14 mmol), tert-butyl 2,2,2-trichloroacetimidate (6.700 g, 30.73 mmol), and boron trifluoride etherate (4.300 g, 30.49 mmol) in tetrahydrofuran (10 mL) was stirred at room temperature under nitrogen atmosphere overnight. The reaction mixture was poured into water (20 ml) and extracted with a mixture of dichloromethane and isopropyl alcohol (3/1, v/v, 40 mL×3). The combined organic layers were dried over anhydrous sodium sulfate, and concentrated to give a crude residue, which was purified by silica gel column chromatography using a 5% methanol in dichloromethane gradient to afford tert-butyl (S)-6-benzyl-2-((S)-2,2-dimethylcyclopropane-1-carbonyl)-2,6-diazaspiro[3.4]octane-8-carboxylate (Z-3) (1.200 g, 49%) as a yellow oil. MS: [MH]⁺ 399.6.

Step 4: tert-Butyl (S)-2-((S)-2,2-dimethylcyclopropane-1-carbonyl)-2,6-diazaspiro[3.4]octane-8-carboxylate (Z-4): A mixture of tert-butyl (S)-6-benzyl-2-((S)-2,2-dimethylcyclopropane-1-carbonyl)-2,6-diazaspiro[3.4]octane-8-carboxylate Z-3 (1.200 g, 3.02 mmol) and Pd/C (0.240 g) in methanol (20 mL) was stirred at room temperature under hydrogen atmosphere for 2 hours. The Pd—C was filtered off, and the filtrate was concentrated to give a crude residue, which was purified by silica gel column chromatography using a 5% methanol in dichloromethane gradient to afford tert-butyl (S)-2-((S)-2,2-dimethylcyclopropane-1-carbonyl)-2,6-diazaspiro[3.4]octane-8-carboxylate (Z-4) (0.850 g, 86%) as a yellow solid. MS: [MH]⁺ 309.4.

Step 5: tert-Butyl 6-(benzo[d]thiazol-7-yl)-2-((S)-2,2-dimethylcyclopropane-1-carbonyl)-2,6-diazaspiro[3.4]octane-8-carboxylate (A-1): A mixture of tert-butyl (S)-2-((S)-2,2-dimethylcyclopropane-1-carbonyl)-2,6-diazaspiro[3.4]octane-8-carboxylate Z-4 (0.155 g, 0.50 mmol), 7-bromobenzo[d]thiazole (0.118 g, 0.55 mmol). Ruphos Pd G3 (0.084 g, 0.101 mmol), and cesium carbonate (0.327 g, 1.01 mmol) in N,N-dimethylformamide (5 mL) was stirred at 100° C. under nitrogen atmosphere overnight. The mixture was concentrated to give a crude residue, which was purified by silica gel column chromatography using a 30% ethyl acetate in hexane gradient to afford tert-butyl (S)-6-(benzo[d]thiazol-7-yl)-2-((S)-2,2-dimethylcyclopropane-1-carbonyl)-2,6-diazaspiro[3.4]octane-8-carboxylate (A-1) (0.190 g, 86%) as a yellow solid. MS: [MH]⁺ 442.4.

Step 6: 6-(Benzo[d]thiazol-7-yl)-2-((S)-2,2-dimethylcyclopropane-1-carbonyl)-2,6-diazaspiro[3.4]octane-8-carboxylic acid (A-2): A mixture of tert-butyl (S)-6-(benzo[d]thiazol-7-yl)-2-((S)-2,2-dimethylcyclopropane-1-carbonyl)-2,6-diazaspiro[3.4]octane-8-carboxylate A-1 (0.048 g, 0.11 mmol) and trifluoroacetic acid (1 mL) in dichloromethane (1 mL) was stirred at room temperature under nitrogen atmosphere for 2 hours. The mixture was concentrated under reduced pressure to afford crude (S)-6-(benzo[d]thiazol-7-yl)-2-((S)-2,2-dimethylcyclopropane-1-carbonyl)-2,6-diazaspiro[3.4]octane-8-carboxylic acid A-2 as a white solid, which was used in the next step without further purification. MS: [MH]⁺ 386.1.

Step 7: N-((3S,4R)-4-((2-oxabicyclo[2.2.2]octan-4-yl)methoxy)-2-hydroxy-2-methylpentan-3-yl)-6-(benzo[d]thiazol-7-yl)-2-((S)-2,2-dimethylcyclopropane-1-carbonyl)-2,6-diazaspiro[3.4]octane-8-carboxamide I-16: A mixture of (S)-6-(benzo[d]thiazol-7-yl)-2-((S)-2,2-dimethylcyclopropane-1-carbonyl)-2,6-diazaspiro[3.4]octane-8-carboxylic acid A-2 (0.042 g, 0.11 mmol), N,N-diisopropylethylamine (0.042 g, 0.33 mmol), (3S,4R)-4-((2-oxabicyclo[2.2.2]octan-4-yl)methoxy)-3-amino-2-methylpentan-2-ol (0.028 g, 0.11 mmol), and 2-(7-azabenzotriazol-1-yl)-N,N,N′,N-tetramethyluronium hexafluorophosphate (0.042 g, 0.13 mmol) in N,N-dimethylformamide (1 mL) was stirred at room temperature under nitrogen atmosphere for 2 hours. The mixture was partitioned between ethyl acetate (10 mL) and water (10 mL). The organic layer was collected, and the aqueous layer was extracted with ethyl acetate (10 mL×2). The combined organic lavers were washed with brine (10 mL), dried over anhydrous sodium sulfate, and concentrated to give a crude residue, which was purified by prep-HPLC to afford a diastereomeric mixture of N-((3S,4R)-4-((2-oxabicyclo[2.2.2]octan-4-yl)methoxy)-2-hydroxy-2-methylpentan-3-yl)-6-(benzo[d]thiazol-7-yl)-2-((S)-2,2-dimethylcyclopropane-1-carbonyl)-2,6-diazaspiro[3.4]octane-8-carboxamide I-16 (0.014 g, 21%) as a white solid. I-16A: ¹HNMR (400 MHz, CD₃OD): δ 9.19 (s, 1H), 7.55 (d, J=8.0 Hz, 1H), 7.45 (t, J=8.0 Hz, 1H), 6.79-6.69 (m, 1H), 4.59-4.24 (m, 3H), 4.15-3.85 (m, 7H), 3.66-3.48 (m, 4H), 3.21-3.10 (m, 1H), 2.99 (t, J=8.8 Hz, 1H), 1.87-1.85 (m, 2H), 1.54-1.41 (m, 6H), 1.36-1.31 (m, 2H), 1.25-1.16 (m, 10H), 1.25-1.08 (m, 4H), 0.82-0.79 (m, 1H): MS: [MH]⁺ 625.6. I-16B: ¹HNMR (400 MHz, CD₃OD): δ 9.17 (s, 1H), 7.53 (d, J=8.0 Hz, 1H), 7.43 (t, J=8.0 Hz, 1H), 6.77-6.74 (m, 1H), 4.61 (d, J=9.2 Hz, 1H), 4.38-4.22 (m, 2H), 4.06-3.81 (m, 7H), 3.76-3.72 (m, 3H), 3.56-3.53 (m, 1H), 3.21 (d, J=7.2 Hz, 1H), 3.01 (d, J=9.2 Hz, 1H), 1.99-1.97 (m, 2H), 1.68-1.62 (m, 4H), 1.52-1.41 (m, 3H), 1.32-1.28 (m, 5H), 1.20-1.16 (m, 8H), 1.05-1.01 (m, 4H), 1.36-1.31 (m, 2H), 1.25-1.16 (m, 10H). 1.25-1.08 (m, 4H), 0.83-0.79 (m, 1H): MS: [MH]⁺ 625.5.

The following compound was prepared in a manner analogous to the procedure described above for I-16: (S)-N-((2S,3R)-3-(2-oxabicyclo[2.2.2]octan-4-ylmethoxy)-1-(dimethylamino)-1-oxobutan-2-yl)-6-(benzo[d]thiazol-7-yl)-2-((S)-2,2-dimethylcyclopropanecarbonyl)-2,6-diazaspiro[3.4]octane-8-carboxamide I-22:

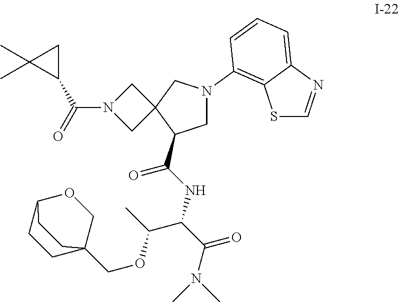 [SMILES: C[C@@H](OCC12CCC(CC1)OC2)[C@H](NC(=O)[C@@H]1CN(c2cccc3ncsc23)CC12CN(C(=O)[C@H]1CC1(C)C)C2)C(=O)N(C)C]

The procedure afforded I-22 (0.035 g, 37%) as a yellow solid. ¹HNMR (400 MHz, CD₃OD): δ 9.15 (s, 1H), 7.52-7.49 (m, 1H), 7.42-7.38 (m, 1H), 6.74-6.71 (m, 1H), 4.93-4.88 (m, 1H), 4.56-4.39 (m, 1H), 4.31-4.24 (m, 1H), 4.18-3.82 (m, 6H), 3.74-3.59 (m, 4H), 3.49-3.39 (m, 1H), 3.20-3.14 (m, 4H), 3.03-2.93 (m, 4H), 1.97-1.80 (m, 2H), 1.65-1.33 (m, 8H), 1.19-1.08 (m, 9H), 1.07-1.03 (m, 1H), 0.81-0.76 (m, 1H). MS: [MH]⁺ 638.30.

### Synthesis of (S)-N-((2S,3R)-3-(2-oxabicyclo[2.2.2]octan-4-ylmethoxy)-1-oxo-1-(2-oxa-7-azaspiro[3.5]nonan-7-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide I-19

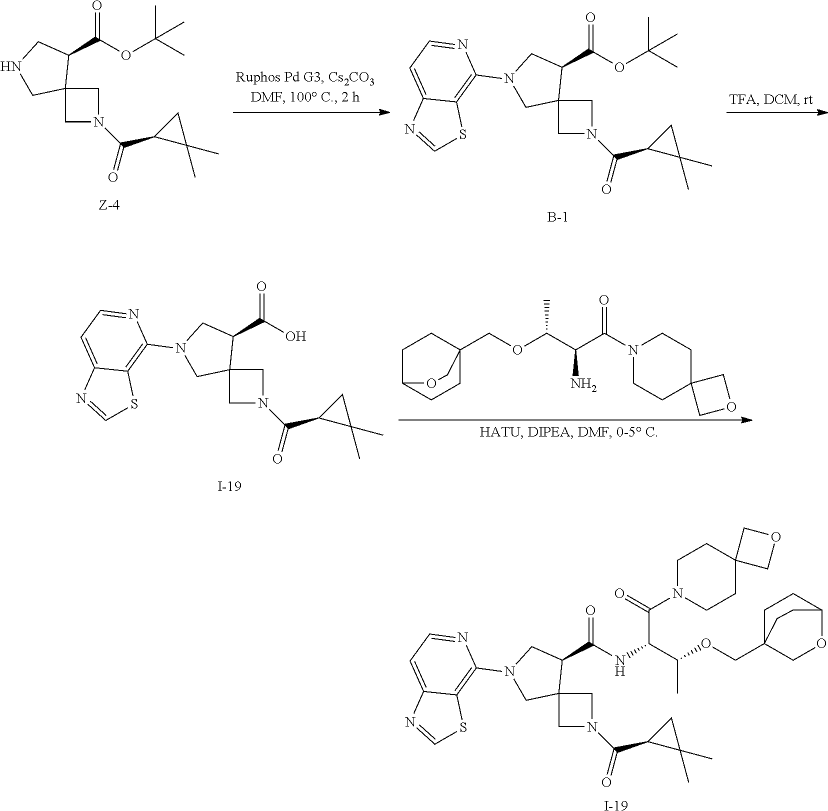

Step 1: (S)-tert-butyl 2-((S)-2,2-dimethylcyclopropanecarbonyl)-6-(thiazolo[5,4-c]pyridin-4-yl)-2,6-diazaspiro[3.4]octane-8-carboxylate (B-1): To a mixture of (S)-tert-butyl 2-((S)-2,2-dimethylcyclopropanecarbonyl)-2,6-diazaspiro[3.4]octane-8-carboxylate Z-4 (0.275 g, 0.89 mmol), 4-chlorothiazolo[5,4-c]pyridine (0.167 g, 0.98 mmol), and cesium carbonate (0.581 g, 1.78 mmol) in N,N-dimethylformamide (5 mL) was added RuPhos Pd G3 (0.149 g, 0.18 mmol) under nitrogen atmosphere. The resulting mixture was stirred at 100° C. under nitrogen atmosphere for 2 hours. The reaction mixture was poured into water (20 mL) and extracted with ethyl acetate (5 mL×3). The combined organic layers were washed with brine (20 mL×2), dried over anhydrous sodium sulfate, and concentrated under reduced pressure to give a crude residue, which was purified by silica gel column chromatography using a 50% ethyl acetate in hexane gradient to afford (S)-tert-butyl 2-((S)-2,2-dimethylcyclopropanecarbonyl)-6-(thiazolo[5,4-c]pyridin-4-yl)-2,6-diazaspiro[3.4]octane-8-carboxylate B-1 (0.057 g, 14%) as a yellow oil. MS: [MH]⁺ 443.4.

Step 2: (S)-2-((S)-2,2-dimethylcyclopropanecarbonyl)-6-(thiazolo[5,4-c]pyridin-4-yl)-2,6-diazaspiro[3.4]octane-8-carboxylic acid (B-2): To a solution of (S)-tert-butyl 2-((S)-2,2-dimethylcyclopropanecarbonyl)-6-(thiazolo[5,4-c]pyridin-4-yl)-2,6-diazaspiro[3.4]octane-8-carboxylate B-1 (0.053 g, 0.12 mmol) in dichloromethane (3 mL) was added 2,2,2-trifluoroacetic acid (2 mL). The reaction mixture was stirred at room temperature for 3 hours. The volatiles were evaporated under reduced pressure to afford crude (S)-2-((S)-2,2-dimethylcyclopropanecarbonyl)-6-(thiazolo[5,4-c]pyridin-4-yl)-2,6-diazaspiro[3.4]octane-8-carboxylic acid B-2 as a yellow oil, which was used in the next step without further purification. MS: [MH]⁺ 387.3.

Step 3: (S)-N-((2S,3R)-3-(2-oxabicyclo[2.2.2]octan-4-ylmethoxy)-1-oxo-1-(2-oxa-7-azaspiro[3.5]nonan-7-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide I-19: To a solution of crude (S)-2-((S)-2,2-dimethylcyclopropanecarbonyl)-6-(thiazolo[5,4-c]pyridin-4-yl)-2,6-diazaspiro[3.4]octane-8-carboxylic acid B-2 (0). 12 mmol), (2S,3R)-3-(2-oxabicyclo[2.2.2]octan-4-ylmethoxy)-2-amino-1-(2-oxa-7-azaspiro[3.5]nonan-7-yl)butan-1-one X-4 (0.046 g, 0.13 mmol), and N-ethyl-N-isopropylpropan-2-amine (0.046 g, 0.36 mmol) in N,N-dimethylformamide (1 mL) at 0-5° C. was added (2-(7-aza-1H-benzotriazole-1-yl)-1,1,3,3-tetramethyluronium hexafluorophosphate) (0.055 g, 0.14 mmol). The resulting mixture was stirred at 0-5° C. for 1 hour. The reaction mixture was poured into water (20 mL) and extracted with ethyl acetate (5 ml×3). The combined organic lavers were washed with brine (20 mL×2), dried over anhydrous sodium sulfate, and concentrated under reduced pressure to give a crude residue, which was purified by preparative TLC using a 5% methanol in dichloromethane gradient to afford (S)-N-((2S,3R)-3-(2-oxabicyclo[2.2.2]octan-4-ylmethoxy)-1-oxo-1-(2-oxa-7-azaspiro[3.5]nonan-7-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide I-19 (0.040 g, 52%) as a light-yellow solid. MS: [MH]⁺ 721.25. ¹HNMR (400 MHz, CD₃OD): δ 9.37 (s, 1H), 8.07-8.05 (m, 1H), 7.29 (d, J=6.0 Hz, 1H), 4.94-4.90 (m, 1H), 4.58-4.08 (m, 11H), 4.00-3.93 (m, 1H), 3.72-3.40 (m, 9H), 3.23-3.16 (m, 1H), 3.03-2.97 (m, 1H), 1.99-1.74 (m, 6H), 1.66-1.33 (m, 8H), 1.20-1.04 (m, 10H), 0.82-0.76 (m, 1H).

The following compound was prepared in a manner analogous to that described above for the synthesis of I-19:

### (S)-N-((2S,3R)-3-(2-oxabicyclo[2.2.2]octan-4-ylmethoxy)-1-(dimethylamino)-1-oxobutan-2-yl)-2-((S)-2,2-dimethylcyclopropanecarbonyl)-6-(thiazolo[5,4-c]pyridin-4-yl)-2,6-diazaspiro[3.4]octane-8-carboxamide I-23

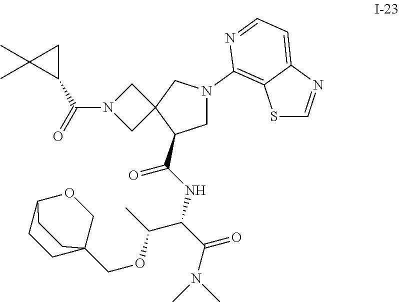 [SMILES: C[C@@H](OCC12CCC(CC1)OC2)[C@H](NC(=O)[C@@H]1CN(c2nccc3ncsc23)CC12CN(C(=O)[C@H]1CC1(C)C)C2)C(=O)N(C)C]

The target compound, I-23, was obtained (0.035 g, 50% yield) as a yellow solid. ¹HNMR (400 MHz, CD₃OD): δ 9.36 (s, 1H), 8.06 (d, J=5.6 Hz, 1H), 7.29 (d, J=5.6 Hz, 1H), 4.94-4.88 (m, 1H), 4.56-4.39 (m, 1H), 4.32-4.07 (m, 6H), 4.01-3.96 (m, 1H), 3.75-3.63 (m, 4H), 3.52-3.43 (m, 1H), 3.22-3.14 (m, 4H), 3.06-3.00 (m, 1H), 2.95-2.93 (m, 3H), 2.00-1.87 (m, 2H), 1.66-1.33 (m, 8H), 1.20-1.04 (m, 10H), 0.82-0.76 (m, 1H). MS: [MH]⁺ 639.70.

### Synthesis of (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-pyrazolo[4,3-c]pyridin-4-yl)-2,6-diazaspiro[3.4]octane-8-carboxamide I-9

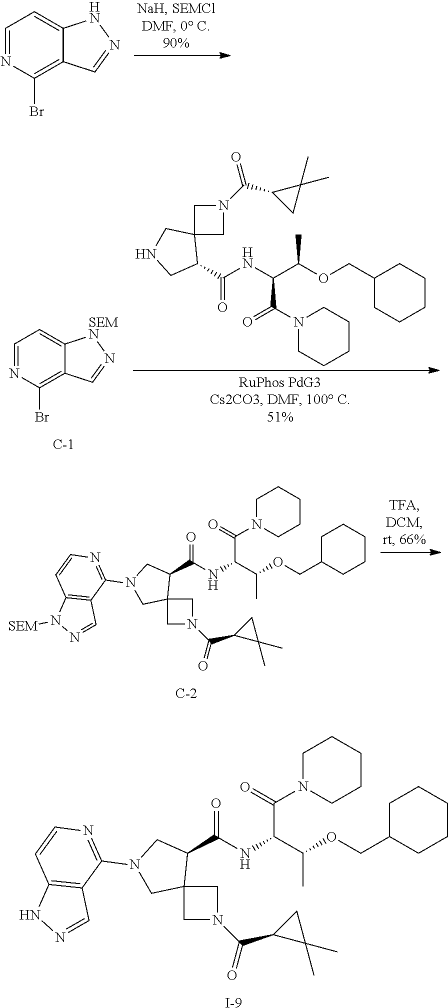

Step 1:4-Bromo-1-((2-(trimethylsilyl)ethoxy)methyl)-1H-pyrazolo[4,3-c]pyridine (C-1): To a solution of 4-bromo-1H-pyrazolo[4,3-c]pyridine (500 mg, 2.53 mmol) in N,N-dimethylformamide (4 mL) at 0-5° C. was added sodium hydride (60% in mineral oil, 202 mg, 5.05 mmol). The resulting mixture was stirred at room temperature for 30 minutes. 2-(Trimethylsilyl)ethoxymethyl chloride (463 mg, 2.78 mmol) was added to the mixture at 0-5° C., and the resulting mixture was stirred for an additional one hour. TLC showed the reaction was complete. The reaction was quenched with water and extracted with ethyl acetate. The combined organic phases were washed with brine, dried over anhydrous sodium sulfate, and concentrated under reduced pressure to give a crude residue, which was purified by column chromatography using 10% ethyl acetate/hexane gradient to afford 4-bromo-1-((2-(trimethylsilyl)ethoxy)methyl)-1H-pyrazolo[4,3-c]pyridine C-1 (585 mg, 90% yield) as a white solid. LCMS: m/z 327.55/329.75 [M+H]⁺. ¹H NMR (400 MHz, CDCl₃): δ 8.25 (s, 1H), 8.05 (d, J=6.4 Hz, 1H), 7.51 (d, J=6.4 Hz, 1H), 5.74 (s, 2H), 3.64-3.70 (m, 2H), 0.95-0.99 (m, 2H), −0.01 (s, 9H).

Step 2: (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1-((2-(trimethylsilyl)ethoxy)methyl)-1H-pyrazolo[4,3-c]pyridin-4-yl)-2,6-diazaspiro[3.4]octane-8-carboxamide (C-2): To a mixture of (S)-N-((2S,3R)-3-(cyclohexylmethoxy)-1-oxo-1-(piperidin-1-yl)butan-2-yl)-2-((S)-2,2-dimethylcyclopropanecarbonyl)-2,6-diaraspiro[3.4]octane-8-carboxamide C-7 (100 mg, 0.19 mmol: prepared as described below in reference to the synthesis of I-14), 4-bromo-1-((2-(trimethylsilyl)ethoxy)methyl)-1H-pyrazolo[4,3-c]pyridine C-1 (70 mg, 0.21 mmol), and cesium carbonate (125 mg, 0.38 mmol) in N,N-dimethylformamide (1 mL) was added RuPhos Pd G3 (33 mg, 0.038 mmol) under nitrogen atmosphere. The resulting mixture was stirred at 100° C. overnight. TLC showed the reaction was complete. The reaction mixture was diluted with ethyl acetate, washed with water and then brine, dried over anhydrous sodium sulfate, and concentrated under reduced pressure to give a crude residue, which was purified by preparative TLC using 3.3% ammonia solution in methanol/dichloromethane gradient to afford (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1-((2-(trimethylsilyl)ethoxy)methyl)-1H-pyrazolo[4,3-c]pyridin-4-yl)-2,6-diazaspiro[3.4]octane-8-carboxamide C-2 (75 mg, 51% yield) as a white solid. LCMS: m/z. 765.1 [M+H]⁺. ¹HNMR (400 MHz, CD₃OD): δ 8.40 (s, 1H), 7.89 (d, J=6.4 Hz, 1H), 7.00 (d, J=6.0 Hz, 1H), 5.75 (s, 2H), 5.00-5.03 (m, 1H), 4.07-4.53 (m, 8H), 3.78-3.85 (m, 1H), 3.52-3.67 (m, 7H), 3.40-3.44 (m, 1H), 3.24-3.30 (m, 1H), 1.47-1.83 (m, 14H), 1.22-1.31 (m, 11H), 1.11-1.14 (m, 1H), 0.91-1.04 (m, 5H), 0.85-0.89 (m, 1H), 0.00 (s, 9H).

Step 3: (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-pyrazolo[4,3-c]pyridin-4-yl)-2,6-diazaspiro[3.4]octane-8-carboxamide 1-9: To a solution of (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1-((2-(trimethylsilyl)ethoxy)methyl)-1H-pyrazolo[4,3-c]pyridin-4-yl)-2,6-diazaspiro[3.4]octane-8-carboxamide C-2 (72 mg, 0.09 mmol) in dichloromethane (1 mL) at 0-5° C. was added 2,2,2-trifluoroacetic acid (1 mL). The resulting mixture was stirred at room temperature for 2 hours. TLC showed the reaction was complete. After concentration, the reaction mixture was basified with saturated aqueous sodium bicarbonate solution to a pH of 8-9 and extracted with dichloromethane (10 mL×2). The combined organic phases were washed with brine, dried over anhydrous sodium sulfate, and concentrated under reduced pressure to give a crude residue, which was purified by preparative TLC using 10% methanol/dichloromethane gradient to afford (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-pyrazolo[4,3-c]pyridin-4-yl)-2,6-diazaspiro[3.4]octane-8-carboxamide I-19 (40 mg, 66% yield) as a white solid. LCMS: m/z 635.0 [M+H]⁺. ¹HNMR (400 MHz, CD₃OD): δ 8.36 (s, 1H), 7.72 (d, J=6.4 Hz, 1H), 6.84 (d, J=6.4 Hz, 1H), 4.93-4.96 (m, 1H), 4.01-4.47 (m, 8H), 3.73-3.78 (m, 1H), 3.51-3.58 (m, 5H), 3.33-3.38 (m, 1H), 3.17-3.23 (m, 1H), 1.44-1.76 (m, 13H), 1.56-1.24 (m, 11H), 1.05-1.08 (m, 1H), 0.89-0.97 (m, 3H), 0.78-0.83 (m, 1H).

### Synthesis of (S)-6-(benzo[d]thiazol-7-yl)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide I-7

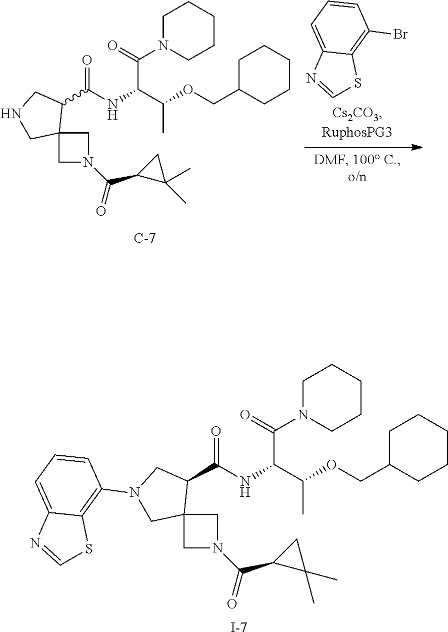

To a solution of N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide C-7 (50 mg, 0.10 mmol) in N,N-dimethylformamide (2 ml) was added 7-bromobenzo[d]thiazole (22.6 mg, 0.11 mmol), cesium carbonate (63 mg, 0.19 mmol), and RuPhos Pd G3 (16.2 mg, 0.02 mmol). The resulting mixture was stirred at 100° C. under nitrogen atmosphere overnight. TLC showed the reaction was complete. The reaction mixture was filtered and concentrated under reduced pressure to give a crude residue, which was purified by column chromatography using 5-8% methanol/dichloromethane gradient to afford (S)-6-(benzo[d]thiazol-7-yl)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide I-7 (26.4 mg, 42% yield) as a yellow solid. LCMS: m/z 650.4 [M+H]⁺. ¹HNMR (400 MHz, CD₃OD): δ 9.14 (s, 1H), 7.49 (d, J=7.6 Hz, 1H), 7.40 (1, J=8.0 Hz, 1H), 6.70-6.72 (m, 1H), 4.92-4.95 (m, 1H), 4.42 (d, J=8.8 Hz, 1H), 4.06-4.32 (m, 3H), 3.85-4.01 (m, 5H), 3.72-3.75 (m, 1H), 3.42-3.54 (m, 5H), 3.34-3.36 (m, 1H), 3.14-3.20 (m, 1H), 1.42-1.70 (m, 13H), 1.14-1.18 (m, 11H), 1.04-1.07 (m, 1H), 0.88-0.93 (m, 2H), 0.76-0.79 (m, 1H).

### Synthesis of (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indazol-7-yl)-2,6-diazaspiro[3.4]octane-8-carboxamide I-11

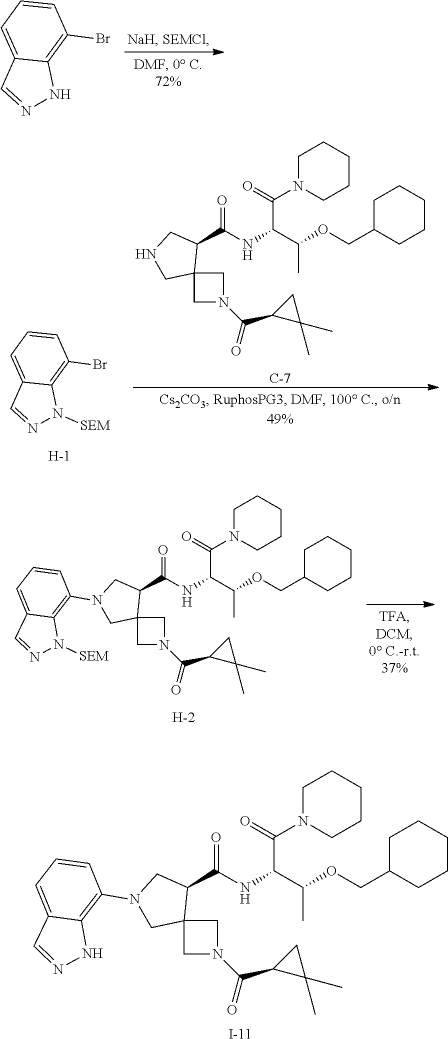

Step 1:7-bromo-1-((2-(trimethylsilyl)ethoxy)methyl)-1H-indazole (H-1): To a solution of 7-bromo-1H-indazole (500 mg, 2.54 mmol) in N,N-dimethylformamide (5 mL) was added sodium hydride at 0° C. The resulting mixture was stirred for 0.5 hour after which a solution of 2-(trimethylsilyl)ethoxymethyl chloride (465 mg, 2.79 mmol) in N,N-dimethylformamide (1 mL) was added dropwise at 0° C. The resulting mixture was stirred at 0° C. under nitrogen atmosphere for 1.5 hours. TLC showed the reaction was complete. The reaction was quenched with water (8 mL) and extracted with ethyl acetate (12 mL). The combined organic layer was washed with water (8 mL×2) and brine (8 mL), dried over anhydrous sodium sulfate, and concentrated to give a crude residue, which was purified by silica gel flash column chromatography using a 5% ethyl acetate/hexane gradient to afford 7-bromo-1-((2-(trimethylsilyl)ethoxy)methyl)-1H-indazole H-1 (600 mg, 72% yield) as a yellow oil. LCMS: m/z 328.7 [M+H]⁺. ¹H NMR (400 MHz, CDCl₃): δ 8.03 (s, 1H), 7.69 (d, J=4 Hz, 1H), 7.61 (d, J=4.0 Hz, 1H), 7.05 (t, J=8 Hz, 1H), 6.08 (s, 2H), 3.59 (t, J=8.0 Hz, 2H), 0.89 (t, J=8.0 Hz, 2H), −0.07 (s, 9H).

Step 2: (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1-((2-(trimethylsilyl)ethoxy)methyl)-1H-indazol-7-yl)-2,6-diazaspiro[3.4]octane-8-carboxamide (H-2): To a solution of (S)-N-((2S,3R)-3-(cyclohexylmethoxy)-1-oxo-1-(piperidin-1-yl)butan-2-yl)-2-((S)-2,2-dimethylcyclopropanecarbonyl)-2,6-diaraspiro[3.4]octane-8-carboxamide C-7 (100 mg, 0.19 mmol), 7-bromo-1-((2-(trimethylsilyl)ethoxy)methyl)-1H-indazole H-1 (82 mg, 0.25 mmol), and cesium carbonate (126 mg, 0.39 mmol) in N,N-dimethylformamide (2 mL) was added RuPhos-Pd-G3 (32 mg, 0.039 mmol) under nitrogen atmosphere. The resulting mixture was stirred at 100° C. overnight. TLC showed the reaction was complete. The reaction was quenched with water (5 mL) and extracted with ethyl acetate (10 mL). The combined organic layer was washed with water (5 mL×2) and brine (5 mL), dried over anhydrous sodium sulfate, and concentrated to give a crude residue, which was purified through silica gel flash column chromatography using an n-Hex/EtOAc/MeOH 100/100/5 gradient to afford (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1-((2-(trimethylsilyl)ethoxy)methyl)-1H-indazol-7-yl)-2,6-diazaspiro[3.4]octane-8-carboxamide H-2 (95 mg, 49% yield) as a yellow solid. LCMS: m/z 763.5[M+H]⁺. ¹H NMR (400 MHz, CDCl₃): δ 8.01 (s, 1H), 7.48 (d, J=4 Hz, 1H), 7.21 (d, J=3.8 Hz, 1H), 7.09-7.13 (m, 1H), 6.97-7.03 (m, 1H), 5.77-6.17 (m, 2H), 5.02 (d, J=3.8 Hz, 1H), 4.24 (s, 1H), 3.96-4.05 (m, 2H), 3.57-3.77 (m, 8H), 3.42-3.49 (m, 3H), 3.27-3.37 (m, 2H), 3.10-3.17 (m, 1H), 1.60-1.69 (m, 14H), 1.06-1.17 (m, 13H), 0.84-0.89 (m, 4H), 0.71-0.74 (m, 1H), −0.06 (d, J=6.8 Hz, 9H).

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indazol-7-yl)-2,6-diazaspiro[3.4]octane-8-carboxamide I-11: To a solution of (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1-((2-(trimethylsilyl)ethoxy)methyl)-1H-indazol-7-yl)-2,6-diazaspiro[3.4]octane-8-carboxamide H-2 (80 mg, 0.105 mmol) in dichloromethane (2 mL) was added trifluoroacetic acid (2 mL) at 0° C. The resulting mixture was stirred at room temperature for 7 hours. The reaction mixture was basified with saturated aqueous sodium bicarbonate solution (3 mL) and extracted with dichloromethane (8 mL×2). The combined organic layers were dried over anhydrous sodium sulfate and concentrated to give a crude residue, which was purified by prep-TLC using an n-hexane/EtOAc/MeOH 100/100/5 gradient to afford (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indazol-7-yl)-2,6-diazaspiro[3.4]octane-8-carboxamide I-11 (28 mg, 37% yield) as a green solid. LCMS: m/z 633.6 [M+H]⁺. ¹H NMR (400 MHz, CDCl₃) δ 8.70 (d, J=4.4 Hz, 1H), 8.44-8.46 (m, 1H), 8.06 (s, 1H), 7.41 (d, J=4.0 Hz, 1H), 7.07 (t, J=7.6 Hz, 1H), 6.78-6.83 (m, 1H), 4.97 (d, J=4.0 Hz, 1H), 4.25-4.42 (m, 2H), 4.11-4.19 (m, 2H), 3.96-4.04 (m, 2H), 3.80-3.85 (m, 1H), 3.67-3.70 (m, 2H), 3.50-3.60 (m, 3H), 3.35-3.45 (m, 2H), 3.09-3.16 (m, 2H), 2.94-3.02 (m, 1H), 1.57-1.66 (m, 7H), 1.20-1.34 (m, 6H), 1.08-1.15 (m, 9H), 0.86-0.94 (m, 3H), 0.71-0.74 (m, 1H), 0.50-0.64 (m, 2H).

### Synthesis of (S)-N-((2S,3R)-3-(cyclohexylmethoxy)-1-oxo-1-(piperidin-1-yl)butan-2-yl)-2-((S)-2,2-dimethylcyclopropanecarbonyl)-6-(thiazolo[4,5-c]pyridin-4-yl)-2,6-diazaspiro[3.4]octane-8-carboxamide I-10

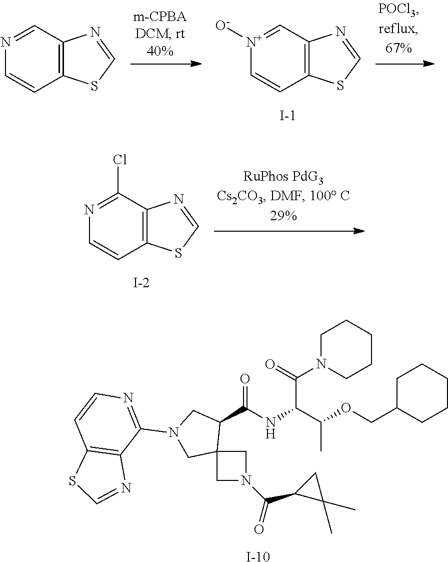

Step 1: Thiazolo[4,5-c]pyridine 5-oxide (I-1): To a solution of thiazolo[4,5-c]pyridine (900 mg, 6.61 mmol) in dichloromethane (10 mL) at 0-5° C. was added 3-cloroperoxybenzoic acid (2.88 g, 16.69 mmol) in portions. The resulting mixture was stirred at room temperature for 3 hours. TLC showed the reaction was complete. The reaction was quenched with aqueous potassium carbonate solution (1M) and extracted with dichloromethane. The combined organic phases were dried over anhydrous sodium sulfate and concentrated under reduced pressure to give a crude residue, which was purified in chromatography using 10% methanol/dichloromethane gradient to afford thiazolo[4,5-c]pyridine 5-oxide I-1 (400 mg, 40% yield) as a white solid. LCMS: m/z 152.8 [M+H]⁺. ¹H NMR (400 MHz, DMSO-d₆): δ 9.59 (s, 1H), 9.09 (s, 1H), 8.30-8.32 (m, 1H), 8.23 (d, J=6.8 Hz, 1H).

Step 2: 4-chlorothiazolo[4,5-c]pyridine (I-2): A mixture of thiazolo[4,5-c]pyridine 5-oxide I-1 (200 mg, 1.31 mmol) in phosphorus oxytrichloride (2 mL) was refluxed for 2 hours. TLC showed the reaction was complete. After concentration, the residue was taken up in water, neutralized with saturated aqueous sodium bicarbonate solution to a pH of 8, and extracted with ethyl acetate (20 mL×2). The combined organic phases were dried over anhydrous sodium sulfate and concentrated under reduced pressure to give a crude residue, which was purified by column chromatography using 10% ethyl acetate/hexane gradient to afford 4-chlorothiazolo[4,5-c]pyridine I-2 (150 mg, 67% yield) as a white solid. LCMS: m/z 170.7 [M+H]⁺. ¹HNMR (400 MHz, CDCl₃): δ 9.14 (s, 1H), 8.39 (d, J=5.2 Hz, 1H), 7.88 (d, J=5.6 Hz, 1H).

Step 3: (S)-N-((2S,3R)-3-(cyclohexylmethoxy)-1-oxo-1-(piperidin-1-yl)butan-2-yl)-2-((S)-2,2-dimethylcyclopropanecarbonyl)-6-(thiazolo[4,5-c]pyridin-4-yl)-2,6-diazaspiro[3.4]octane-8-carboxamide 1-10: To a mixture of (S)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide C-7 (50 mg, 0.097 mmol), 4-chlorothiazolo[4,5-c]pyridine I-2 (18 mg, 0.11 mmol), and cesium carbonate (63 mg, 0.19 mmol) in N,N-dimethylformamide (1 mL) was added RuPhos Pd G3 (16 mg, 0.019 mmol) under nitrogen atmosphere. The resulting mixture was stirred at 100° C. overnight. TLC showed the reaction was complete. The reaction mixture was diluted with ethyl acetate (20 mL), washed with water (15 mL) and then brine (15 mL), dried over anhydrous sodium sulfate, and concentrated under reduced pressure to give a crude residue, which was purified by preparative TLC using 3.3% ammonia solution in methanol/dichloromethane gradient to afford I-10 (22 mg, 29% yield) as a yellow solid. LCMS: m/z 651.7 [M+H]⁺. ¹HNMR (400 MHz, CD₃OD): δ 8.98 (s, 1H), 7.93 (d, J=5.6 Hz, 1H), 7.26 (d, J=5.6 Hz, 1H), 4.93-4.96 (m, 1H), 4.07-4.47 (m, 7H), 3.95-3.99 (m, 1H), 3.71-3.77 (m, 1H), 3.34-3.59 (m, 6H), 3.17-3.22 (m, 1H), 1.60-1.76 (m, 6H), 1.40-1.56 (m, 7H), 1.14-1.27 (m, 11H), 1.04-1.07 (m, 1H), 0.88-0.96 (m, 2H), 0.76-0.81 (m, 1H).

### Synthesis of 6-(benzo[d]thiazol-4-yl)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide I-8

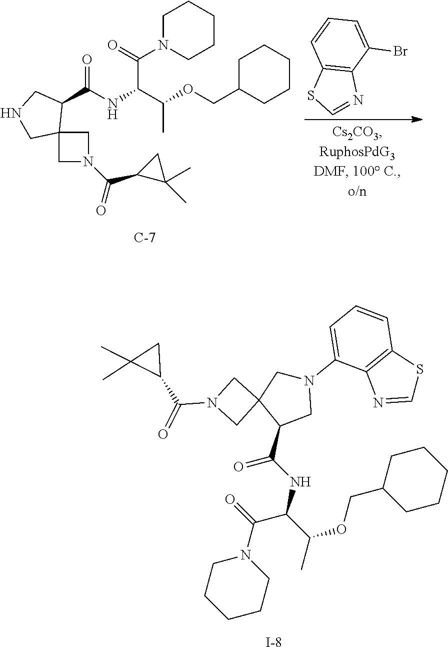

To stirred solution of N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide C-7 (70 mg, 0.14 mmol), 4-bromobenzo[d]thiazole (33 mg, 0.15 mmol), and Cs₂CO₃ (91 mg, 0.28 mmol) in DMF (2 mL) under nitrogen was added Ruphos Pd G3 (23.4 mg, 0.028 mmol). The resulting mixture was stirred at 100° C. under N₂ overnight. TLC showed the reaction was complete. The mixture was poured into saturated ammonium chloride solution (15 mL) and extracted with ethyl acetate (10 mL×2). The combined organic layers were washed with brine (15 mL), dried over anhydrous sodium sulfate, and concentrated under reduced pressure to give a crude residue, which was purified by prep-HPLC afford to 6-(benzo[d]thiazol-4-yl)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide (33 mg, 36% yield) as a white solid. LCMS: m/z 650.3 [M+H]⁺. ¹HNMR (400 MHz, CD₃OD): δ 8.89 (d, J=2.9 Hz. 1H), 7.34 (d, J=7.8 Hz, 1H), 7.27 (t, J=7.8 Hz, 1H), 6.65 (dd, J=7.8, 1.8 Hz, 1H), 4.91-4.94 (m, 1H), 4.42 (t, J=8.8 Hz, 1H), 3.95-4.32 (m, 8H), 3.69-3.75 (m, 1H), 3.48-3.53 (m, 4H), 3.33-3.42 (m, 2H), 3.13-3.20 (m, 1H), 1.41-1.70 (m, 13H), 1.15-1.29 (m, 12H), 1.04-1.07 (m, 1H), 0.88-0.94 (m, 2H), 0.76-0.81 (m, 1H).

### Synthesis of (S)-N-((2S,3R)-3-((2-oxabicyclo[2.2.2]octan-4-yl)methoxy)-1-(methylamino)-1-oxobutan-2-yl)-6-(thiazole-5-carbonyl)-2-(4-(trifluoromethyl)oxazol-2-yl)-2,6-diazaspiro[3.4]octane-8-carboxamide I-32

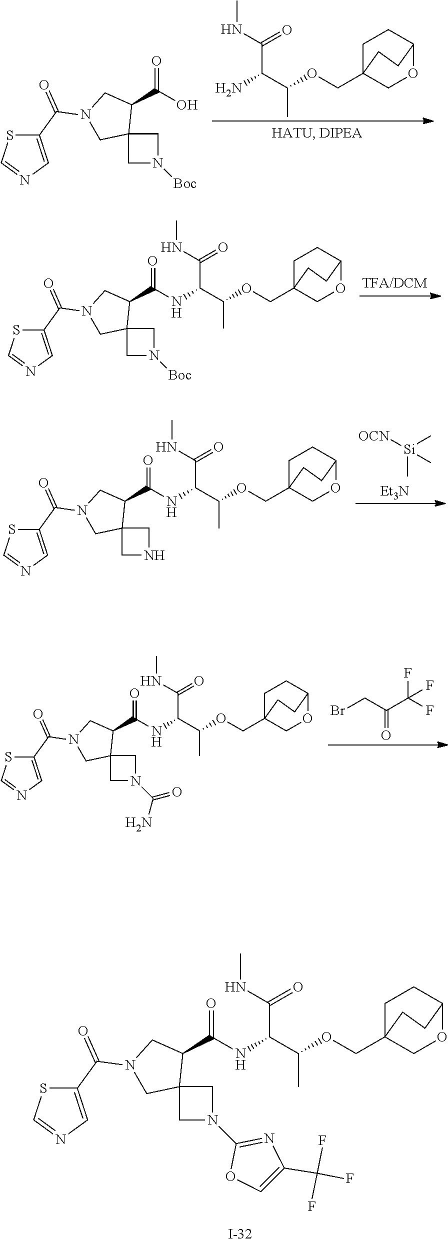

Step 1: Synthesis of tert-butyl (S)-8-(((2S,3R)-3-((2-oxabicyclo[2.2.2]octan-4-yl)methoxy)-1-(methylamino)-1-oxobutan-2-yl)carbamoyl)-6-(thiazole-5-carbonyl)-2,6-diazaspiro[3.4]octane-2-carboxylate: To a solution of (S)-2-(tert-butoxycarbonyl)-6-(thiazole-5-carbonyl)-2,6-diazaspiro[3.4]octane-8-carboxylic acid (120 mg, 0.33 mmol) in DCM (3 mL) was added HATU (126 mg, 0.33 mmol) and DIPEA (170 mg, 1.32 mmol). The mixture was stirred at room temperature for 30 minutes, and then (2S,3R)-3-((2-oxabicyclo[2.2.2]octan-4-yl)methoxy)-2-amino-N-methylbutanamide (prepared as described infra) (110 mg, 0.43 mmol) was added. The reaction was stirred an additional 2 hours. The mixture was diluted with water (20 mL) and extracted with DCM (50 mL×2). The combined organic layers were washed with brine, dried over Na₂SO₄, filtered, and concentrated. The residue was purified by prep-TLC (eluent: DCM/MeOH=10/1) to afford tert-butyl (S)-8-(((2S,3R)-3-((2-oxabicyclo[2.2.2]octan-4-yl)methoxy)-1-(methylamino)-1-oxobutan-2-yl)carbamoyl)-6-(thiazole-5-carbonyl)-2,6-diazaspiro[3.4]octane-2-carboxylate (75 mg, 38%) as a yellow oil. LCMS m/z=606.2 [M+H]⁺. ¹H NMR (400 MHz, DMSO-d₆): δ 9.24 (d, J=7.2 Hz, 1H), 8.35 (d, J=19.8 Hz, 1H), 7.80-7.68 (m, 1H), 7.51-7.44 (m, 1H), 4.42 (1, J=5.4 Hz, 1H), 4.24 (d, J=17.8 Hz, 2H), 3.68-3.61 (m, 4H), 3.57-3.53 (m, 4H), 3.05-3.03 (m, 3H), 2.91 (d, J=15.4 Hz, 3H), 2.60 (d, J=4.6 Hz, 2H), 1.83 (t, J=4.2 Hz, 2H), 1.55-1.54 (m, 2H), 1.50 (d, J=4.0 Hz, 2H), 1.36 (s, 9H), 1.24 (m, 2H), 1.01 (d, J=6.4 Hz, 3H).

Step 2: Synthesis of (S)-N-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-(methylamino)-1-oxobutan-2-yl)carbamoyl)-6-(thiazole-5-carbonyl)-2,6-diazaspiro[3.4]octane-2-carboxylate (70 mg, 0.11 mmol) in DCM (2 mL) was added TFA (1 mL). The reaction mixture was stirred at room temperature for 2 hours, after which the solvent was removed under vacuum to afford (S)-N-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-8-carboxamide (60 mg, 100%), which was used directly in the next step. LCMS m/z=506.2 [M+H]⁺.

Step 3: Synthesis of (S)-N8-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-2,8-dicarboxamide: To a solution of (S)-N-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-8-carboxamide (60 mg, 0.11 mmol) in DCM (2 mL) at 0° C. was added TEA (0.07 mL) and isocyanatotrimethylsilane (0.02 mL, 0.15 mmol). The mixture was stirred at room temperature for 2 hours after which the solvent was removed under vacuum to afford (S)-N8-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-2,8-dicarboxamide (65 mg, 100%), which was used directly in the next step. LCMS m/z=549.2 [M+H]⁺.

Step 4: Synthesis of (S)-N-((2S,3R)-3-((2-oxabicyclo[2.2.2]octan-4-yl)methoxy)-1-(methylamino)-1-oxobutan-2-yl)-6-(thiazole-5-carbonyl)-2-(4-(trifluoromethyl)oxazol-2-yl)-2,6-diazaspiro[3.4]octane-8-carboxamide I-32: To a solution of (S)-N8-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-2,8-dicarboxamide (65 mg, 0.11 mmol) in 2-methylpropan-2-ol (2 mL) was added 3-bromo-1,1,1-trifluoropropan-2-one (0.06 mL, 0.59 mmol). The mixture was heated at 90° C. for 5 hours after which the solvent was removed under reduced pressure. The residue obtained was purified by prep-HPLC to afford (S)-N-((2S,3R)-3-((2-oxabicyclo[2.2.2]octan-4-yl)methoxy)-1-(methylamino)-1-oxobutan-2-yl)-6-(thiazole-5-carbonyl)-2-(4-(trifluoromethyl)oxazol-2-yl)-2,6-diazaspiro[3.4]octane-8-carboxamide (3.5 mg, 5%). LCMS m/z=641.4 [M+H]⁺. ¹H NMR (400 MHz, Methanol-d₄): δ 9.16 (s, 1H), 8.37 (d, J=7.8 Hz, 1H), 7.94 (s, 1H), 4.42-4.19 (m, 5H), 4.16-4.08 (m, 2H), 3.98-3.84 (m, 2H), 3.81-3.45 (m, 5H), 3.22 (dd, J=9.2, 4.0 Hz, 1H), 2.98 (dd, J=9.4, 4.4 Hz, 1H), 2.75 (d, J=11.6 Hz, 3H), 1.95 (m, 2H), 1.74-1.58 (m, 4H), 1.46 (m, 2H), 1.07 (dd, J=9.2, 6.4 Hz, 3H).

### Synthesis of (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-29A and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethoxy)piperidin-1-yl)butan-2-yl)-6-(thiazolo[4,5-d]pyrimidin-7-yl)-2,6-diazaspiro[3.4]octane-8-carboxamide I-35

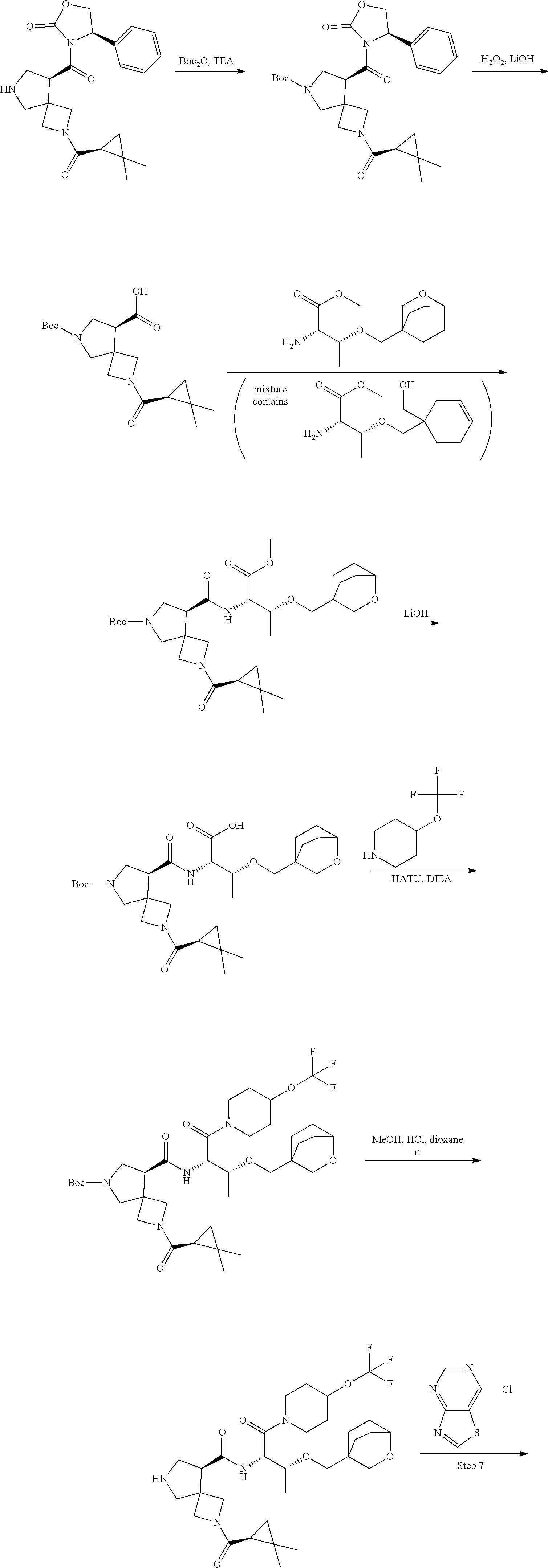

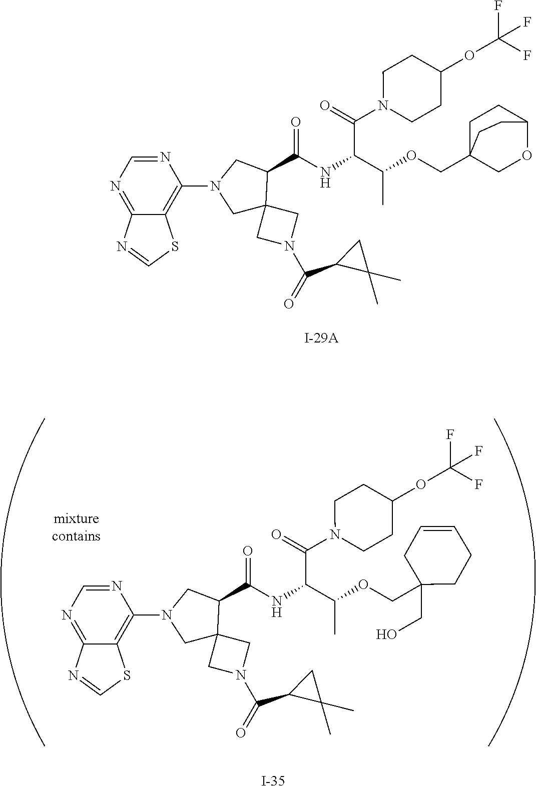

Step 1: tert-Butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate: To a solution of (R)-3-((S)-2-((S)-2,2-dimethylcyclopropane-1-carbonyl)-2,6-diazaspiro[3.4]octane-8-carbonyl)-4-phenyloxazolidin-2-one (200 mg, 0.5 mmol) in DCM (2 mL) was added TEA (101 mg, 1.0 mmol) and (Boc)₂O (164 mg, 0.75 mmol). The reaction mixture was stirred at room temperature for 1.5 hours and then diluted with water (20 mL) and extracted with DCM (30 mL×3). The combined organic lavers were washed with brine, dried over Na₂SO₄, filtered, and purified by column chromatography on silica gel (eluent: DCM/MeOH=100/1) to afford tert-butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate (95 mg, 38%) as a colorless oil. LCMS m/z=498.2 [M+H]⁺. ¹H NMR (400 MHz, DMSO-d₆): δ 7.41-7.23 (m, 5H), 5.49-5.43 (m, 1H), 4.81-4.72 (m, 1H). 4.33-4.12 (m, 3H), 4.06-3.30 (m, 7H), 1.41-1.33 (m, 9H), 1.28-1.20 (m, 2H), 1.13-1.04 (m, 5H), 0.89-0.83 (m, 1H), 0.70-0.64 (m, 1H).

Step 2: (S)-6-(tert-butoxycarbonyl)-2-((S)-2,2-dimethylcyclopropane-1-carbonyl)-2,6-diazaspiro[3.4]octane-8-carboxylic acid: To a solution of tert-butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate (90 mg, 0.18 mmol) in a mixture of THF (0.8 mL) and water (0.2 mL) at 0° C. was added a solution of lithium hydroxide monohydrate (11 mg, 0.45 mmol) in water (0.2 mL) and 30% H₂O₂ (12 mg, 0.36 mmol) in water (0.2 mL). The reaction mixture was stirred at 0° C.) for 2 hour and then diluted with water (10 mL) and extracted with EtOAc (30 mL). The aqueous layer was collected and acidified with HCl (1M) to a pH of ˜3 and extracted with EtOAc (50 mL×3). The combined organic layers were washed with brine, dried over Na₂SO₄, filtered, and concentrated to afford (S)-6-(tert-butoxycarbonyl)-2-((S)-2,2-dimethylcyclopropane-1-carbonyl)-2,6-diazaspiro[3.4]octane-8-carboxylic acid (60 mg, 95%) as a colorless oil, which was used directly in the next step. LCMS m/z=353.3 [M+H]⁺. ¹H NMR (400 MHz, DMSO-d₆): δ 3.55-3.41 (m, 9H), 1.42-1.37 (m, 10H), 1.13-1.03 (m, 6H), 0.89-0.83 (m, 1H), 0.70-0.63 (m, 1H).

Step 3: tert-Butyl (S)-8-(((2S,3R)-3-((2-oxabicyclo[2.2.2]octan-4-yl)methoxy)-1-methoxy-1-oxobutan-2-yl)carbamoyl)-2-((S)-2,2-dimethylcyclopropane-1-carbonyl)-2,6-diazaspiro[3.4]octane-6-carboxylate: To a solution of methyl O-((2-oxabicyclo[2.2.2]octan-4-yl)methyl)-L-threoninate (345 mg, 0.98 mmol; (prepared as described infra) in DCM (3 mL) was added HATU (403 mg, 1.06 mmol) and DIPEA (316 mg, 2.45 mmol). The mixture was stirred at room temperature for 30 minutes, and then (S)-6-(tert-butoxy carbonyl)-2-((S)-2,2-dimethylcyclopropane-1-carbonyl)-2,6-diazaspiro[3.4]octane-8-carboxylic acid (210 mg, 0.82 mmol) was added. The reaction was stirred at room temperature for an additional 1.5 hour and then diluted with water (20 mL) and extracted with EtOAc (30 mL×3). The combined organic layers were washed with brine, dried over Na₂SO₄, filtered, and concentrated. The residue obtained was purified by prep-TLC (eluent: DCM/MeOH=20/1) to afford tert-butyl (S)-8-(((2S,3R)-3-((2-oxabicyclo[2.2.2]octan-4-yl)methoxy)-1-methoxy-1-oxobutan-2-yl)carbamoyl)-2-((S)-2,2-dimethylcyclopropane-1-carbonyl)-2,6-diazaspiro[3.4]octane-6-carboxylate (370 mg, 77%) as a white solid. LCMS m/z=592.3 [M+H]⁺. ¹H NMR (400 MHz, DMSO-d₆): δ 8.39-8.27 (m, 1H), 5.62-5.50 (m, 1H), 4.55-4.43 (m, 1H), 4.35-3.98 (m, 5H), 3.94-3.78 (m, 3H), 3.72-3.61 (m, 6H), 3.22-3.09 (m, 3H), 1.43-1.37 (m, 15H), 1.14-1.10 (m, 3H), 1.08-0.99 (m, 9H), 0.87-0.83 (m, 2H), 0.71-0.63 (m, 1H).

Step 4: O-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine: To a solution of tert-butyl (S)-8-(((2S,3R)-3-((2-oxabicyclo[2.2.2]octan-4-yl)methoxy)-1-methoxy-1-oxobutan-2-yl)carbamoyl)-2-((S)-2,2-dimethylcyclopropane-1-carbonyl)-2,6-diazaspiro[3.4]octane-6-carboxylate (50 mg, 0.85 mmol) in a mixture of THF (0.4 mL), MeOH (0.1 mL), and water (0.1 mL) was added LiOH (6 mg, 0.25 mmol). The mixture was stirred at room temperature for 4 hours and then diluted with water (10 mL) and extracted with EtOAc (20 mL×3). The aqueous phase was acidified to pH of 3 with HCl (1M) and extracted with EtOAc (50 mL×3). The combined organic layers were washed with brine, dried over Na₂SO₄ and concentrated to afford O-((2-oxabicyclo[2.2.2]octan-4-yl)methyl)-N-((S)-6-(tert-butoxy carbonyl)-2-((S)-2,2-dimethylcyclopropane-1-carbonyl)-2,6-diazaspiro[3.4]octane-8-carbonyl)-L-threonine (20 mg, 42%) as a colorless oil. LCMS m/z=578.3 [M+H]⁺.

Step 5: tert-Butyl (S)-8-(((2S,3R)-3-((2-oxabicyclo[2.2.2]octan-4-yl)methoxy)-1-oxo-1-(4-(trifluoromethoxy)piperidin-1-yl)butan-2-yl)carbamoyl)-2-((S)-2,2-dimethylcyclopropane-1-carbonyl)-2,6-diazaspiro[3.4]octane-6-carboxylate: To a solution of O-((2-oxabicyclo[2.2.2]octan-4-yl)methyl)-N-((S)-6-(tert-butoxy carbonyl)-2-((S)-2,2-dimethylcyclopropane-1-carbonyl)-2,6-diazaspiro[3.4]octane-8-carbonyl)-L-threonine (153 mg, 0.27 mmol) in DCM (2 mL) was added HATU (111 mg, 0.29 mmol) and DIPEA (103 mg, 0.80 mmol). The mixture was stirred at room temperature for 30 minutes. 4-(Trifluoromethoxy)piperidine (45 mg, 0.27 mmol) was added, and the reaction stirred at room temperature for an additional 1.5 hours. The mixture was diluted with water (20 mL) and extracted with EtOAc (30 mL×3). The combined organic layers were washed with brine, dried over Na₂SO₄, filtered, and concentrated. The residue obtained was purified by RP-column to afford tert-butyl (S)-8-(((2S,3R)-3-((2-oxabicyclo[2.2.2]octan-4-yl)methoxy)-1-oxo-1-(4-(trifluoromethoxy)piperidin-1-yl)butan-2-yl)carbamoyl)-2-((S)-2,2-dimethylcyclopropane-1-carbonyl)-2,6-diazaspiro[3.4]octane-6-carboxylate (75 mg, 39%) as a colorless oil. LCMS m/z=729.2 [M+H]⁺. ¹H NMR (400 MHz, DMSO-d₆): δ 4.36-3.56 (m, 11H), 1.57-1.50 (m, 3H), 1.38 (s, 15H), 1.12-0.96 (m, 13H), 0.86-0.84 (m, 2H), 0.69-0.64 (m, 1H).

Step 6: (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-oxo-1-(4-(trifluoromethoxy)piperidin-1-yl)butan-2-yl)carbamoyl)-2-((S)-2,2-dimethylcyclopropane-1-carbonyl)-2,6-diazaspiro[3.4]octane-6-carboxylate (65 mg, 0.1 mmol) in MeOH (3 mL) was added a solution of HCl in dioxane (1M, 0.7 mL). The reaction mixture was stirred at room temperature for 1.5 hours, and then the solvent was removed under vacuum to afford (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-2,6-diazaspiro[3.4]octane-8-carboxamide (56 mg, 100%), which was used directly in the next step. LCMS m/z=629.3 [M+H]⁺.

Step 7: (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-29A and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethoxy)piperidin-1-yl)butan-2-yl)-6-(thiazolo[4,5-d]pyrimidin-7-yl)-2,6-diazaspiro[3.4]octane-8-carboxamide I-35: To a solution of (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-2,6-diazaspiro[3.4]octane-8-carboxamide (50) mg, 0.08 mmol) and 7-chlorothiazolo[4,5-d]pyrimidine (14 mg, 0.08 mmol) in CH₃CN (1 mL) was added Na₂CO₃ (25 mg, 0.2 mmol). The reaction mixture was heated at 70° C. for 2 hours and then concentrated under reduced pressure. The residue was purified by prep-HPLC to afford (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-29A (3.3 mg, 5%) as a yellow solid. Further elution provided (85)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethoxy)piperidin-1-yl)butan-2-yl)-6-(thiazolo[4,5-d]pyrimidin-7-yl)-2,6-diazaspiro[3.4]octane-8-carboxamide I-35 (3.7 mg, 6%) as a yellow solid. I-29A: LCMS m/z=764.51H NMR (400 MHz, Methanol-d₄) δ 9.54 (s, 1H), 8.50 (s, 1H), 4.98-4.93 (m, 1H), 4.62-4.56 (m, 1H), 4.46-3.92 (m, 10H), 3.78-3.64 (m, 5H), 3.63-3.52 (m, 2H), 3.49-3.41 (m, 1H), 3.07-2.96 (m, 1H), 2.04-1.90 (m, 4H), 1.83-1.72 (m, 2H), 1.70-1.59 (m, 4H), 1.55-1.39 (m, 4H), 1.19-1.14 (m, 8H), 1.08-1.03 (m, 1H), 0.83-0.77 (m, 1H), 1-35: LCMS m/z=764.4 ¹H NMR (400 MHz, Methanol-d₄) δ 9.54 (s, 1H), 8.50) (s, 1H), 5.68-5.55 (m, 2H), 4.95-4.89 (m, 1H), 4.62-4.54 (m, 1H), 4.45-3.95 (m, 9H), 3.80-3.71 (m, 2H), 3.64-3.53 (m, 2H), 3.49-3.41 (m, 3H), 3.27-3.21 (m, 1H), 2.12-1.88 (m, 5H), 1.87-1.38 (m, 9H), 1.20-1.13 (m, 8H), 1.08-1.04 (m, 1H), 0.83-0.76 (m, 1H).

### Synthesis of (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-30A and synthesis of (85)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazolo[4,5-d]pyrimidin-7-yl)-2,6-diazaspiro[3.4]octane-8-carboxamide I-36

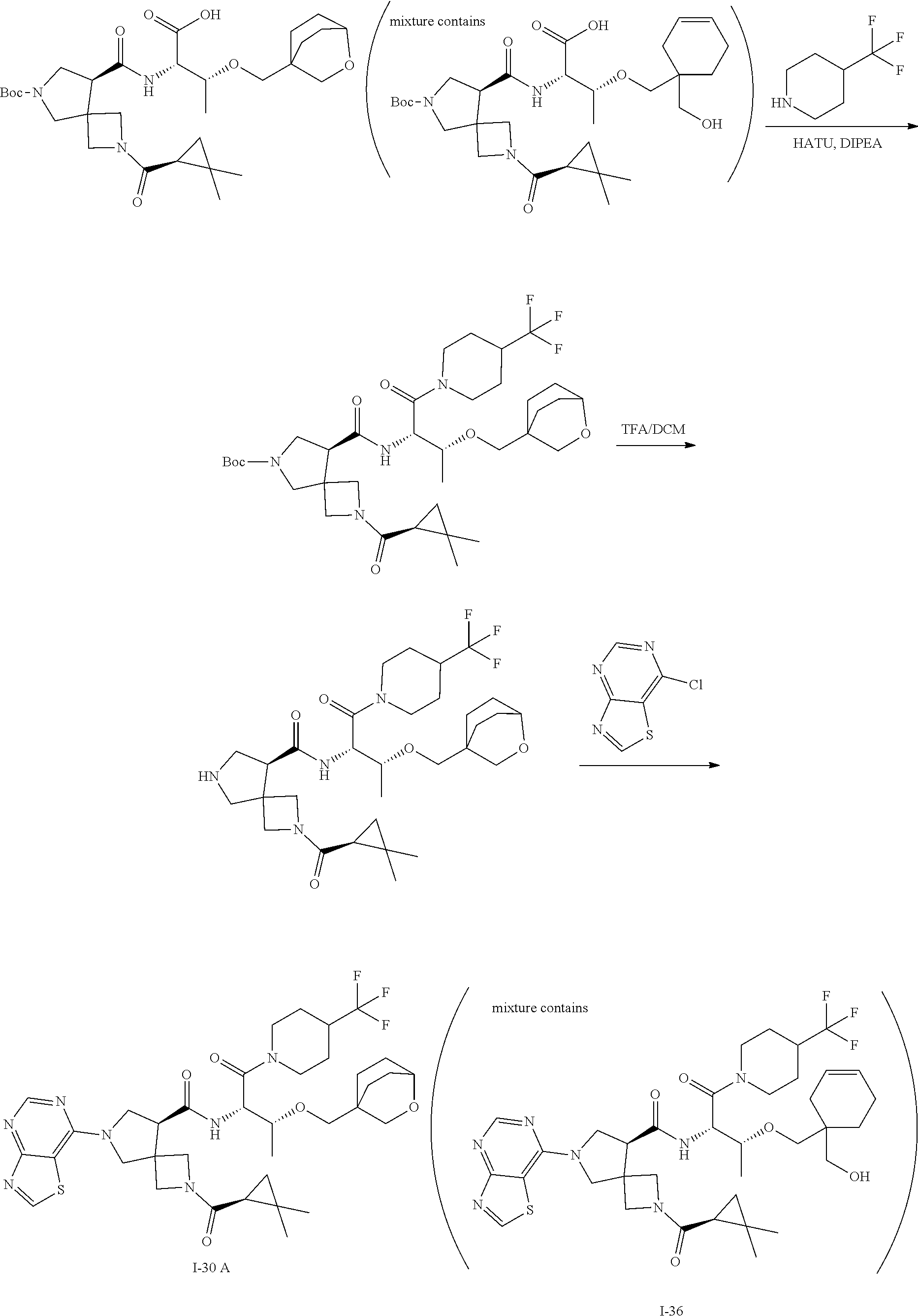

Step 1: tert-Butyl (S)-8-(((2S,3R)-3-((2-oxabicyclo[2.2.2]octan-4-yl)methoxy)-1-oxo-1-(4-(trifluoromethyl)piperidin-1-yl)butan-2-yl)carbamoyl)-2-((S)-2,2-dimethylcyclopropane-1-carbonyl)-2,6-diazaspiro[3.4]octane-6-carboxylate: To a solution of O-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine (see synthesis of I-29A, step 4, supra) (115 mg, 0.2 mmol) in DCM (3 mL) was added HATU (114 mg, 0.3 mmol) and DIPEA (76 mg, 0.6 mmol). The mixture was stirred at room temperature for 30) minutes, and then 4-(trifluoromethyl)piperidine (34 mg, 0.2 mmol) was added. The reaction mixture was stirred at room temperature for 2 hours and then diluted with water (20 mL) and extracted with DCM (50 mL×2). The combined organic layers were washed with brine, dried over Na₂SO₄, filtered, and concentrated. The residue was purified by prep-TLC (eluent: DCM/MeOH=10/1) to afford tert-butyl (S)-8-(((2S,3R)-3-((2-oxabicyclo[2.2.2]octan-4-yl)methoxy)-1-oxo-1-(4-(trifluoromethyl)piperidin-1-yl)butan-2-yl)carbamoyl)-2-((S)-2,2-dimethylcyclopropane-1-carbonyl)-2,6-diazaspiro[3.4]octane-6-carboxylate (95 mg, 67%) as a yellow solid. LCMS m/z=713.5 [M+H]⁺.

Step 2: (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-oxo-1-(4-(trifluoromethyl)piperidin-1-yl)butan-2-yl)carbamoyl)-2-((S)-2,2-dimethylcyclopropane-1-carbonyl)-2,6-diazaspiro[3.4]octane-6-carboxylate (85 mg, 0.12 mmol) in DCM (2 mL) was added TFA (1 mL). The reaction mixture was stirred at room temperature for 1 hour. Next, the solvent was removed under vacuum to afford ((S)-N-((2S,3R)-3-((2-oxabicyclo[2.2.2]octan-4-yl)methoxy)-1-oxo-1-(4-(trifluoromethyl) piperidin-1-yl)butan-2-yl)-2-((S)-2,2-dimethylcyclopropane-1-carbonyl)-2,6-diazaspiro[3.4]octane-8-carboxamide (70 mg, 96%), which was used directly in the next step. LCMS m/z=613.4 [M+H]⁺.

Step 3: Synthesis of (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-30A and synthesis of (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazolo[4,5-d]pyrimidin-7-yl)-2,6-diazaspiro[3.4]octane-8-carboxamide I-36: To a solution of (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-2,6-diazaspiro[3.4]octane-8-carboxamide (70) mg. 0.11 mmol) in MeCN (2 mL) was added Na₂CO₃ (36 mg, 0.33 mmol). The mixture was stirred at room temperature for 30 minutes, and then 7-chlorothiazolo[4,5-d]pyrimidine (19 mg, 0.22 mmol) was added. The reaction mixture was stirred at room temperature overnight and then filtered through Celite and concentrated. The residue was purified by prep-HPLC to afford (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-30A (6.3 mg, 7.4%) as a white solid. Further elution provided (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazolo[4,5-d]pyrimidin-7-yl)-2,6-diazaspiro[3.4]octane-8-carboxamide I-36 (6.4 mg, 7.4%) as a white solid. I-30A: LCMS m/z=748.4 [M+H]: ¹H NMR (400 MHz. Methanol-d₄): δ 9.54 (s, 1H), 8.50 (s, 1H), 5.00-4.92 (m, 2H), 4.65-4.54 (m, 1H), 4.44-4.38 (m, 1H), 4.37-4.31 (m, 1H), 4.31-4.26 (m, 1H), 4.23-4.12 (m, 3H), 4.12-4.05 (m, 1H), 4.05-3.97 (m, 1H), 3.76-3.67 (m, 3H), 3.59-3.50 (m, 1H), 3.26-3.15 (m, 2H), 3.03 (s, 1H), 2.74-2.62 (m, 1H), 2.49 (s, 1H), 2.05-1.81 (m, 4H), 1.73-1.58 (m, 4H), 1.56-1.36 (m, 5H), 1.35-1.28 (m, 1H), 1.22-1.10 (m, 9H), 1.08-1.03 (m, 1H), 0.84-0.76 (m, 1H). I-36: LCMS m/z=748.4[M+H]⁺: ¹H NMR (400 MHz. Methanol-d₄): δ 9.54 (s, 1H), 8.50 (s. 1H), 5.68-5.57 (m, 2H), 4.64-4.53 (m, 2H), 4.47-4.39 (m, 1H), 4.38-4.32 (m, 1H), 4.31-4.26 (m, 1H), 4.24-4.14 (m, 3H), 4.13-4.07 (m, 1H), 4.05-3.99 (m, 1H), 3.82-3.73 (m, 1H). 3.50-3.47 (m, 1H), 3.46-3.40 (m, 2H), 3.25-3.21 (m, 1H), 3.15-3.12 (m, 1H), 2.71-2.64 (m, 1H), 2.52-2.44 (m, 1H), 2.03-1.98 (m, 2H), 1.86-1.79 (m, 2H), 1.55-1.40 (m, 5H), 1.35-1.29 (m, 1H), 1.24-1.13 (m, 11H), 1.09-1.03 (m, 2H), 0.84-0.77 (m, 2H).

### Synthesis of (S)-N-((2S,3R)-3-((2-oxabicyclo[2.2.2]octan-4-yl)methoxy)-1-oxo-1-(7-oxa-2-azaspiro[3.5]nonan-2-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-25A and synthesis of (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(7-oxa-2-azaspiro[3.5]nonan-2-yl)butan-2-yl)-6-(thiazolo[4,5-d]pyrimidin-7-yl)-2,6-diazaspiro[3.4]octane-8-carboxamide I-37

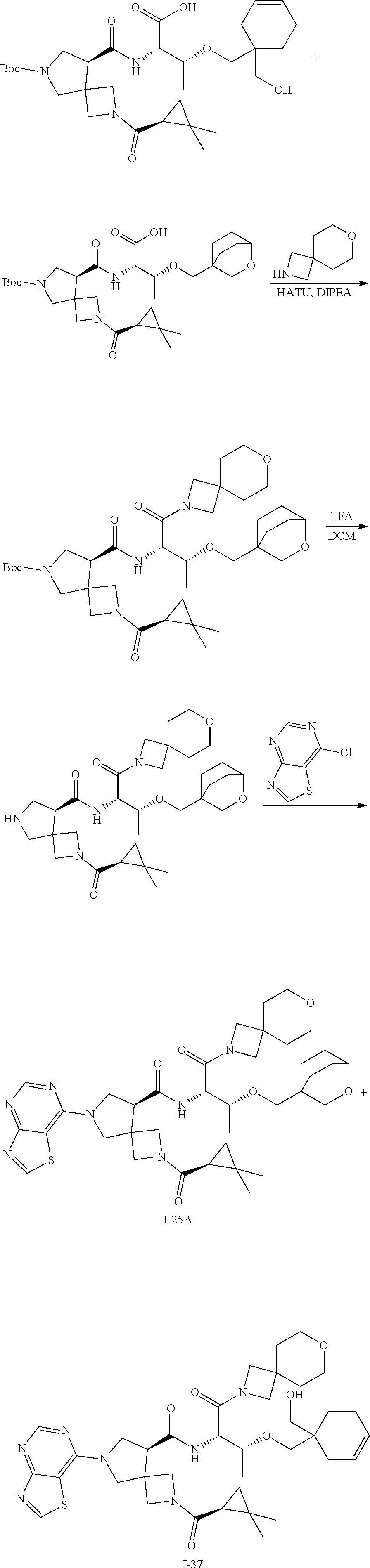

I-25A and I-37 were synthesized from a mixture of O-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine (see synthesis of I-29A, step 4, supra) and N-((S)-6-(tert-butoxycarbonyl)-2-((S)-2,2-dimethylcyclopropane-1-carbonyl)-2,6-diazaspiro[3.4]octane-8-carbonyl)-O-((1-(hydroxymethyl)cyclohex-3-en-1-yl)methyl)-L-threonine (see synthesis of I-29 and I-35, step 3, supra) according to the procedures outlined for I-30 and I-36 supra, using the appropriate commercially available reagents and/or intermediates described elsewhere. I-25A: LCMS m/z=722.4 [M+H]⁺; ¹H NMR (400 MHz, DMSO-d₆): δ 9.72-9.71 (m, 1H), 8.51-8.50 (m, 1H), 8.45-8.42 (m, 1H), 4.44-4.39 (m, 1H), 4.26-4.14 (m, 3H), 3.98-3.77 (m, 6H), 3.64-3.38 (m, 12H), 3.16-3.10 (m, 1H), 3.05-2.97 (m, 1H), 1.86-1.79 (m, 1H), 1.66-1.50 (m, 8H), 1.49-1.26 (m, 4H), 1.11-1.03 (m, 9H), 0.89-0.85 (m, 1H), 0.71-0.66 (m, 1H). I-37: LCMS m/z=722.3 [M+H]⁺; ¹H NMR (400 MHz, DMSO-d₆): δ 9.72-9.70 (m, 1H), 8.51-8.40 (m, 2H), 5.64-5.52 (m, 2H), 4.45-4.38 (m, 1H), 4.37-4.31 (m, 1H), 4.28-4.15 (m, 3H), 4.00-3.88 (m, 5H), 3.66-3.39 (m, 9H), 3.26-3.15 (m, 3H), 1.97-1.88 (m, 2H), 1.80-1.75 (m, 2H), 1.70-1.50 (m, 5H), 1.47-1.29 (m, 4H), 1.11-1.05 (m, 9H), 0.89-0.84 (m, 1H), 0.71-0.66 (m, 1H).

### Synthesis of (S)-N-((2S,3R)-3-((2-oxabicyclo[2.2.2]octan-4-yl)methoxy)-1-oxo-1-(2-oxa-6-azaspiro[3.5]nonan-6-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-26A and synthesis of (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(2-oxa-6-azaspiro[3.5]nonan-6-yl)butan-2-yl)-6-(thiazolo[4,5-d]pyrimidin-7-yl)-2,6-diazaspiro[3.4]octane-8-carboxamide I-38

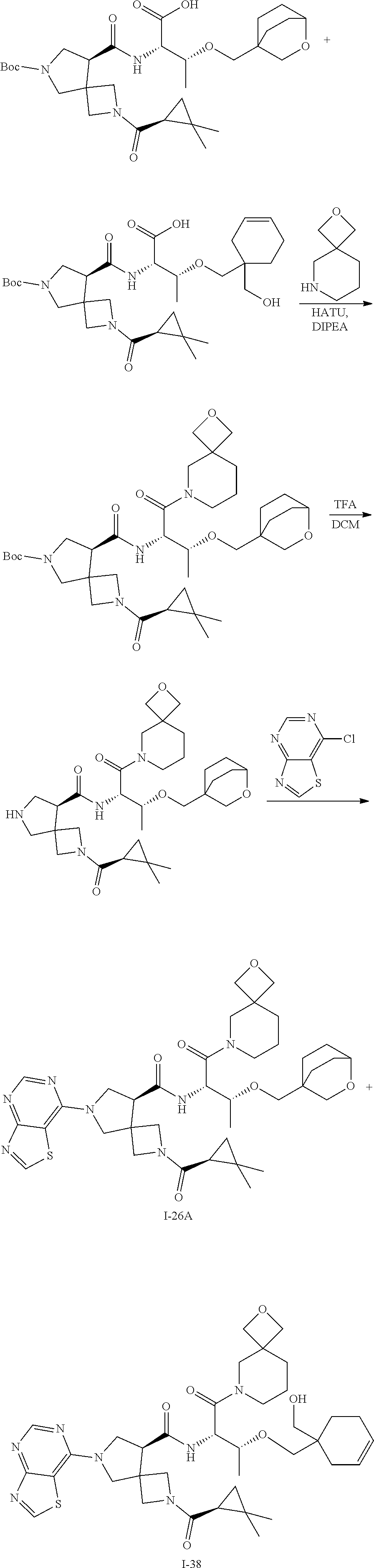

A mixture of (S)-N-((2S,3R)-3-((2-oxabicyclo[2.2.2]octan-4-yl)methoxy)-1-oxo-1-(2-oxa-6-azaspiro[3.5]nonan-6-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-26A and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(2-oxa-6-azaspiro[3.5]nonan-6-yl)butan-2-yl)-6-(thiazolo[4,5-d]pyrimidin-7-yl)-2,6-diazaspiro[3.4]octane-8-carboxamide I-38 was synthesized from a mixture of O-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine (see synthesis s of I-29A, step 4, supra) and N-((S)-6-(tert-butoxycarbonyl)-2-((S)-2,2-dimethylcyclopropane-1-carbonyl)-2,6-diazaspiro[3.4]octane-8-carbonyl)-O-((1-(hydroxymethyl)cyclohex-3-en-1-yl)methyl)-L-threonine (see synthesis of I-29 and I-35, step 3, supra) according to the procedures outlined for the synthesis of I-30A and I-36 supra using the appropriate commercially available reagents and/or intermediates described elsewhere. The ratio was about 5/6. LCMS m/z=722.4 [M+H]⁺. ¹H NMR (400 MHz, Chloroform-d): δ 9.27 (s, 1H), 8.65 (s, 1H), 7.14-6.86 (m, 1H), 5.71-5.53 (m, 1.14H), 5.17-4.83 (m, 1H), 4.43-4.03 (m, 14H), 3.69-3.13 (m, 10H), 2.08-1.90 (m, 4H), 1.60-1.44 (m, 5H), 1.22-1.11 (m, 12H), 0.78 (s, 1H).

### Synthesis of N-((2S,3R)-3-((2-oxabicyclo[2.2.2]octan-4-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27 and synthesis of N-((2S,3R)-3-((1-(hydroxymethyl)cyclohex-3-en-1-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-39

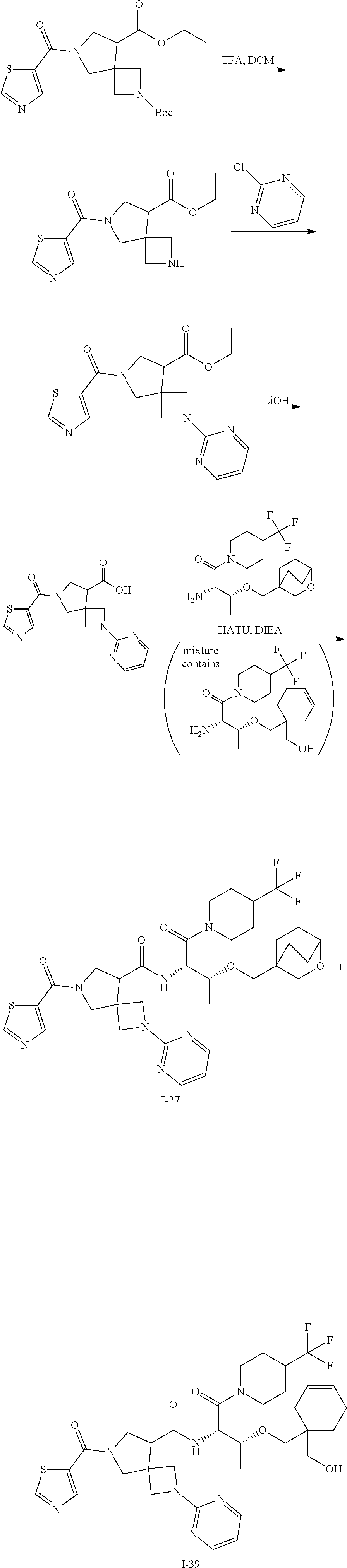

Step 1: Ethyl 6-(thiazole-5-carbonyl)-2,6-diazaspiro[3.4]octane-8-carboxylate: To a solution of 2-(tert-butyl)-8-ethyl-6-(thiazole-5-carbonyl)-2,6-diazaspiro[3.4]octane-2,8-dicarboxylate (400 mg, 1.0 mmol) in DCM (4 mL) was added TFA (2 mL). The reaction mixture was stirred at room temperature for 1.5 hours. The solvent was removed under vacuum to afford crude ethyl 6-(thiazole-5-carbonyl)-2,6-diazaspiro[3.4]octane-8-carboxylate (298 mg, 100%), which was used directly in the next step. LCMS m/z=296.1 [M+H]⁺.

Step 2: Ethyl 2-(pyrimidin-2-yl)-6-(thiazole-5-carbonyl)-2,6-diazaspiro[3.4]octane-8-carboxylate: To a solution of ethyl 6-(thiazole-5-carbonyl)-2,6-diazaspiro[3.4]octane-8-carboxylate (270 mg, 0.91 mmol) and 2-chloropyrimidine (104 mg, 0.91 mmol) in CH₃CN (3 mL) was added Na₂CO₃ (290 mg, 2.7 mmol). The reaction mixture was heated at 70° C. for 2 hours, and then the solvent was removed under reduced pressure. The residue was purified by column chromatography on silica gel (eluent: DCM/MeOH=40/1) to afford ethyl 2-(pyrimidin-2-yl)-6-(thiazole-5-carbonyl)-2,6-diazaspiro[3.4]octane-8-carboxylate (460) mg, 100%) as a colorless oil. LCMS m/z=374.1 [M+H]⁺. ¹H NMR (400 MHz. DMSO-d₆): δ 9.26 (s, 1H), 8.41-8.33 (m, 3H). 6.72-6.67 (m, 1H), 4.13-3.99 (m, 12H), 1.09-1.00 (m, 3H).

Step 3: 2-(Pyrimidin-2-yl)-6-(thiazole-5-carbonyl)-2,6-diazaspiro[3.4]octane-8-carboxylic acid: To a solution of ethyl 2-(pyrimidin-2-yl)-6-(thiazole-5-carbonyl)-2,6-diazaspiro[3.4]octane-8-carboxylate (936 mg, 2.5 mmol) in a mixture of THF (8 mL). MeOH (2 mL), and water (2 mL) was added LiOH·H₂O (315 mg, 7.5 mmol). The reaction mixture was stirred at room temperature for 2 hours and then diluted with water (50 mL) and extracted with EtOAc (80 mL). The aqueous layer was collected and acidified to a pH of 4 with HCl (1M) and extracted with EtOAc (100 mL×3). The combined organic layers were washed with brine, dried over Na₂SO₄, filtered, and concentrated to afford 2-(pyrimidin-2-yl)-6-(thiazole-5-carbonyl)-2,6-diazaspiro[3.4]octane-8-carboxylic acid (282 mg, 33%) as a yellow solid. LCMS m/z=346.1 [M+H]⁺. ¹H NMR (400 MHz. DMSO-d₆): δ 9.33-9.31 (m, 1H), 9.27-9.24 (m, 1H), 8.45-8.43 (m, 1H), 8.41-8.39 (m, 1H), 8.38-8.34 (m, 2H), 6.72-6.67 (m, 1H), 4.28-4.00 (m, 8H), 3.21-3.18 (m, 3H).

Step 4: Mixture of N-((2S,3R)-3-((2-oxabicyclo[2.2.2]octan-4-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27 and N-((2S,3R)-3-((1-(hydroxymethyl)cyclohex-3-en-1-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-39: To a solution of 2-(pyrimidin-2-yl)-6-(thiazole-5-carbonyl)-2,6-diazaspiro[3.4]octane-8-carboxylic acid (50 mg. 0.14 mmol) in DCM (1 mL) was added HATU (66 mg, 0.17 mmol) and DIPEA (56 mg, 0.43 mmol). The mixture was stirred at room temperature for 30 minutes, and then (2S,3R)-3-((2-oxabicyclo[2.2.2]octan-4-yl)methoxy)-2-amino-1-(4-(trifluoromethyl)piperidin-1-yl)butan-1-one (prepared as described infra) (55 mg, 0.14 mmol) was added. The reaction was stirred at room temperature for an additional 2 hours. The mixture was diluted with water (10) mL) and extracted with DCM (30 mL×3). The combined organic layers were washed with brine, dried over Na₂SO₁, filtered, and concentrated. The residue was purified by prep-TLC (eluent: DCM/MeOH=20/1) to afford a mixture of N-((2S,3R)-3-((2-oxabicyclo[2.2.2]octan-4-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27 and N-((2S,3R)-3-((1-(hydroxymethyl)cyclohex-3-en-1-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-39 in a ratio of about 1/1 (7.5 mg, 7%) as a white solid. I-27: LCMS m/z=248.1 [M+H]⁺; ¹H NMR (400 MHz, DMSO-d₆): δ 9.29-9.23 (m, 1H), 8.45-8.31 (m, 4H), 6.72-6.65 (m, 1H), 5.63-5.47 (m, 1H), 4.93-4.75 (m, 1H), 4.49-4.39 (m, 1H), 4.30-3.75 (m, 9H), 3.71-3.40 (m, 5H), 3.22-2.84 (m, 3H), 2.66-2.55 (m, 1H), 1.94-1.67 (m, 5H), 1.58-1.27 (m, 6H), 1.17-0.87 (m, 4H).

### Synthesis of N-((3R)-3-((2-oxabicyclo[2.2.2]octan-4-yl)methoxy)-1-(methylamino)-1-oxobutan-2-yl)-2-(benzo[d]oxazol-2-yl)-6-(thiazole-5-carbonyl)-2,6-diazaspiro[3.4]octane-8-carboxamide I-28

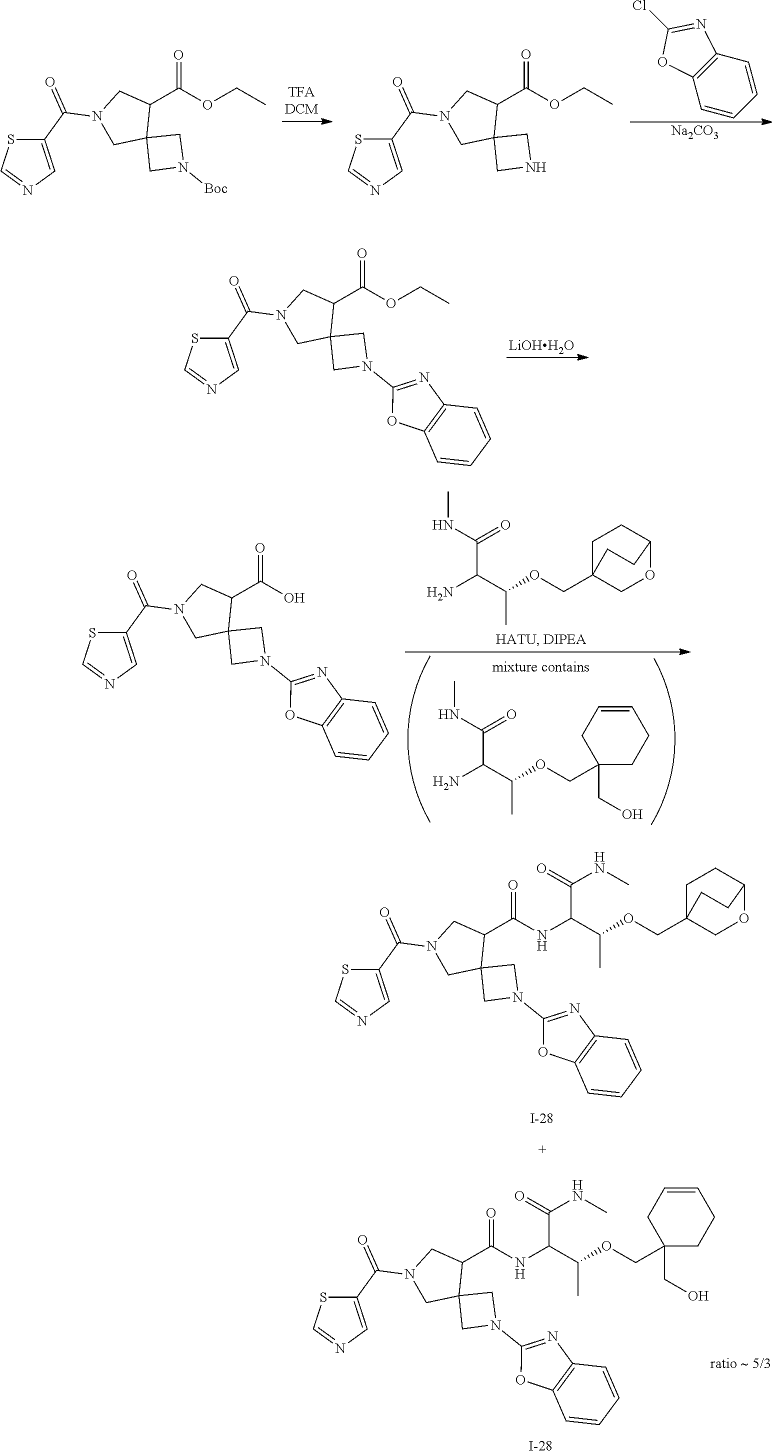

Step 1: Ethyl 6-(thiazole-5-carbonyl)-2,6-diazaspiro[3.4]octane-8-carboxylate: To a solution of 2-(tert-butyl) 8-ethyl 6-(thiazole-5-carbonyl)-2,6-diazaspiro[3.4]octane-2,8-dicarboxylate (350 mg, 0.89 mmol) in DCM (2 mL) was added TFA (1 mL). The reaction mixture was stirred at room temperature for 2 hours. The solvent was removed under vacuum to afford crude ethyl 6-(thiazole-5-carbonyl)-2,6-diazaspiro[3.4]octane-8-carboxylate (260 mg, 100%), which was used directly in the next step. LCMS m/z=296.1 [M+H]⁺.

Step 2: Ethyl 6-(thiazole-5-carbonyl)-2,6-diazaspiro[3.4]octane-8-carboxylate: To a solution of ethyl 6-(thiazole-5-carbonyl)-2,6-diazaspiro[3.4]octane-8-carboxylate (250 mg, 0.85 mmol) in MeCN (4 mL) was added Na₂CO₃ (270 mg, 2.54 mmol) and 2-chlorobenzo[d]oxazole (130 mg, 0.85 mmol). The reaction was heated at 70° C. for 2 hours and then filtered through Celite and concentrated to afford crude ethyl 6-(thiazole-5-carbonyl)-2,6-diazaspiro[3.4]octane-8-carboxylate (350 mg, 100%), which was used directly in the next step. LCMS m/z=413.1 [M+H]⁺. ¹H NMR (400 MHz, DMSO-d₆): δ 9.26 (d, J=2.4 Hz, 1H), 8.38 (d, J=10.4 Hz, 1H), 7.47-7.40 (m, 1H), 7.36-7.28 (m, 1H), 7.16 (t, J=7.6 Hz, 1H), 7.04 (t, J=7.8 Hz, 1H), 4.34 (dd, J=8.2, 5.4 Hz, 1H), 4.29-4.18 (m, 4H), 4.15-3.97 (m, 3H), 3.90 (d, J=4.8 Hz, 1H), 3.77 (d, J=6.6 Hz, 1H), 3.54 (dt, J=21.8, 6.6 Hz, 1H), 1.10 (dt, J=13.2, 7.2 Hz, 3H).

Step 3: 2-(Benzo[d]oxazol-2-yl)-6-(thiazole-5-carbonyl)-2,6-diazaspiro[3.4]octane-8-carboxylic acid: To a solution of ethyl 6-(thiazole-5-carbonyl)-2,6-diazaspiro[3.4]octane-8-carboxylate (300 mg, 0.73 mmol) in a mixture of THF (4 mL), water (1 mL), and EtOH (1 mL) was added LiOH (214 mg, 2.19 mmol). The reaction mixture was stirred at room temperature for 2 hours and then diluted with water (10 mL) and extracted with ether (15 mL). The aqueous layer was collected and acidified to pH of 2 with HCl (1M) and then extracted with EtOAc (50 mL×3). The combined organic layers were washed with brine, dried over Na₂SO₄, filtered, and concentrated to afford 2-(benzo[d]oxazol-2-yl)-6-(thiazole-5-carbonyl)-2,6-diazaspiro[3.4]octane-8-carboxylic acid (227 mg, 81%) as a yellow solid, which was used directly in the next step. LCMS m/z=385.1 [M+H]⁺. ¹H NMR (400 MHz, DMSO-d₆): δ 9.31-9.23 (m, 1H), 8.40 (t, J=10.8 Hz, 1H), 7.42 (dd, J=7.8, 3.4 Hz, 1H), 7.31 (dd, J=7.8, 4.2 Hz, 1H), 7.16 (td, J=7.6, 2.6 Hz, 1H), 7.05 (m, 1H), 4.37-4.19 (m, 4H), 3.90 (d, J=4.2 Hz, 1H), 3.75 (d, J=6.6 Hz, 1H), 3.48-3.39 (m, 3H).

Step 4: Mixture of N-((3R)-3-((2-oxabicyclo[2.2.2]octan-4-yl)methoxy)-1-(methylamino)-1-oxobutan-2-yl)-2-(benzo[d]oxazol-2-yl)-6-(thiazole-5-carbonyl)-2,6-diazaspiro[3.4]octane-8-carboxamide I-28: To a solution of 2-(benzo[d]oxazol-2-yl)-6-(thiazole-5-carbonyl)-2,6-diazaspiro[3.4]octane-8-carboxylic acid (50 mg, 0.13 mmol) in DCM (2 mL) was added HATU (50 mg, 0.13 mmol) and DIPEA (50 mg, 0.39 mmol). The reaction mixture was stirred at room temperature for 30 minutes, and then (3R)-3-((2-oxabicyclo[2.2.2]octan-4-yl)methoxy)-2-amino-N-methylbutanamide (47 mg, 0.18 mmol: prepared similarly to (2S,3R)-3-((2-oxabicyclo[2.2.2]octan-4-yl)methoxy)-2-amino-N-methylbutanamide as described infra) was added and stirring continued for an additional 2 hours. The mixture was diluted with water (30 mL) and extracted with DCM (30 mL×2). The combined organic layers were washed with brine, dried over Na₂SO₄, filtered, and concentrated. The mixture was purified by prep-HPLC to afford a mixture of N-((3R)-3-((2-oxabicyclo[2.2.2]octan-4-yl)methoxy)-1-(methylamino)-1-oxobutan-2-yl)-2-(benzo[d]oxazol-2-yl)-6-(thiazole-5-carbonyl)-2,6-diazaspiro[3.4]octane-8-carboxamide I-27 and 2-(benzo[d]oxazol-2-yl)-N-((3R)-3-((1-(hydroxymethyl)cyclohex-3-en-1-yl)methoxy)-1-(methylamino)-1-oxobutan-2-yl)-6-(thiazole-5-carbonyl)-2,6-diazaspiro[3.4]octane-8-carboxamide in a ratio of about 5/3 (12.7 mg, 16%) as a yellow solid. I-27 was purified via LCMS. m/z=623.4 [M+H]⁺. ¹H NMR (400 MHz, Methanol-d₄): δ 9.16 (s, 0.84H), 8.42-8.35 (m, 1H), 7.38-7.27 (m, 2H), 7.25-7.06 (m, 2H), 5.72-5.47 (m, 0.55H), 4.58-3.63 (m, 12H), 3.60-3.36 (m, 2H), 3.24-2.89 (m, 2H), 2.74 (d, J=11.4 Hz, 1H), 2.61 (d, J=23.4 Hz, 1H), 1.97 (d, J=17.2 Hz, 2H), 1.78 (s, 1H), 1.72-1.35 (m, 5H), 1.19-0.99 (m, 3H).

### Synthesis of Intermediate (2S,3R)-3-((2-oxabicyclo[2.2.2]octan-4-yl)methoxy)-2-amino-N-methylbutanamide

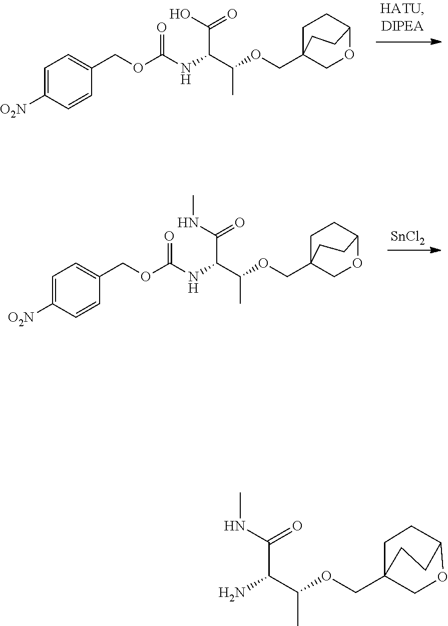

Step 1:4-Nitrobenzyl ((2S,3R)-3-((2-oxabicyclo[2.2.2]octan-4-yl)methoxy)-1-(methylamino)-1-oxobutan-2-yl)carbamate: To a solution of O-((2-oxabicyclo[2.2.2]octan-4-yl)methyl)-N-(((4-nitrobenzyl)oxy)carbonyl)-L-threonine (1.2 g, 2.84 mmol: prepared as described infra) in DCM (5 mL) was added HATU (0.98 g, 2.58 mmol) and DIPEA (1.33 g, 10.32 mmol). The mixture was stirred at room temperature for 30 minutes after which methanamine hydrochloride (175 mg, 2.58 mmol) was added. The reaction mixture was stirred for an additional 2 hours. The mixture was diluted with water (20 mL) and extracted with DCM (30 mL×2). The combined organic layers were washed with brine, dried over Na₂SO₄, filtered, and concentrated. The residue obtained was purified by column chromatography on silica gel (eluent: DCM/MeOH 70/1) to afford 4-nitrobenzyl ((2S,3R)-3-((2-oxabicyclo[2.2.2]octan-4-yl)methoxy)-1-(methylamino)-1-oxobutan-2-yl)carbamate (470 mg, 38%) as a yellow oil. LCMS m/z=436.1 [M+H]⁺. ¹H NMR (400 MHz, DMSO-d₆): δ 8.24 (d, J=8.4 Hz, 2H), 7.83 (d, J=5.0 Hz, 1H), 7.65 (d, J=8.4 Hz, 2H), 7.15 (d, J=9.1 Hz, 1H), 5.19 (d, J=3.0 Hz, 2H), 3.96 (dd, J=9.4, 4.8 Hz, 1H), 3.54-3.50 (m, 2H), 3.16-3.11 (m, 2H), 3.04 (d, J=5.2 Hz, 1H), 2.90 (d, J=9.0 Hz, 1H), 2.59 (d, J=4.6 Hz, 3H), 1.82 (m, 2H), 1.50 (m, 3H), 1.39-1.31 (m, 3H), 1.02 (t, J=6.8 Hz, 3H).

Step 2: (2S,3R)-3-((2-oxabicyclo[2.2.2]octan-4-yl)methoxy)-2-amino-N-methylbutanamide: To a solution of 4-nitrobenzyl ((2S,3R)-3-((2-oxabicyclo[2.2.2]octan-4-yl)methoxy)-1-(methylamino)-1-oxobutan-2-yl)carbamate (450 mg, 1.03 mmol) in ethanol (6 mL) was added tin (II) chloride (980 mg, 5.2 mmol). The mixture was stirred at room temperature overnight. The solvent was removed under reduced pressure, and the residue obtained was purified on an Al₂O₃ column (eluent: DCM/MeOH=50/1) to afford (2S,3R)-3-((2-oxabicyclo[2.2.2]octan-4-yl)methoxy)-2-amino-N-methylbutanamide (120 mg, 45%) as a yellow oil. LCMS m/z=257.2 [M+H]⁺. ¹H NMR (400 MHz, DMSO-d₆): δ 6.95 (d, J=8.2 Hz, 1H), 6.51 (d, J=8.4 Hz, 1H), 4.45 (t, J=5.4 Hz, 1H), 4.04-3.96 (m, 1H), 3.68-3.48 (m, 6H), 3.04 (d, J=5.2 Hz, 1H), 2.62 (d, J=4.6 Hz, 1H), 1.83 (m, 2H), 1.58-1.47 (m, 4H), 1.40-1.33 (m, 2H), 1.18 (d, J=6.8 Hz, 3H).

### Synthesis of (2S,3R)-3-((2-oxabicyclo[2.2.2]octan-4-yl)methoxy)-2-amino-1-(4-(trifluoromethyl)piperidin-1-yl)butan-1-one (Intermediate A) and Synthesis of (2S,3R)-2-amino-3-((1-(hydroxymethyl)cyclohex-3-en-1-yl)methoxy)-1-(4-(trifluoromethyl)piperidin-1-yl)butan-1-one (Intermediate B)

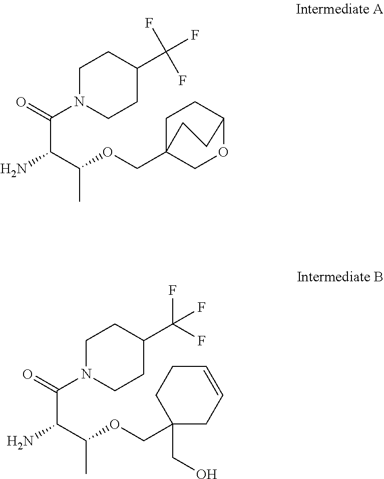 [SMILES: C[C@@H](OCC12CCC(CC1)OC2)[C@H](N)C(=O)N1CCC(C(F)(F)F)CC1 ; C[C@@H](OCC1(CO)CC=CCC1)[C@H](N)C(=O)N1CCC(C(F)(F)F)CC1]

A mixture of (2S,3R)-3-((2-oxabicyclo[2.2.2]octan-4-yl)methoxy)-2-amino-1-(4-(trifluoromethyl)piperidin-1-yl)butan-1-one (Intermediate A) and (2S,3R)-2-amino-3-((1-(hydroxymethyl)cyclohex-3-en-1-yl)methoxy)-1-(4-(trifluoromethyl)piperidin-1-yl)butan-1-one (Intermediate B) was synthesized from 2-methyl 1-(4-nitrobenzyl) (2S,3S)-3-methylaziridine-1,2-dicarboxylate according to the procedures outlined for (2S,3R)-3-((2-oxabicyclo[2.2.2]octan-4-yl)methoxy)-2-amino-N-methylbutanamide (prepared as described supra) using the appropriate commercially available reagents and/or intermediates described elsewhere. LCMS m/z=379.2 [M+H]⁺. ¹H NMR (400 MHz, DMSO-d₆): δ 5.63-5.50 (m, 1H), 3.68-3.43 (m, 5H), 3.27-2.91 (m, 4H), 2.03-1.95 (m, 1H), 1.94-1.70 (m, 6H), 1.69-1.47 (m, 4H), 1.46-1.32 (m, 4H).

### Synthesis of 2-methyl 1-(4-nitrobenzyl) (2S,3S)-3-methylaziridine-1,2-dicarboxylate

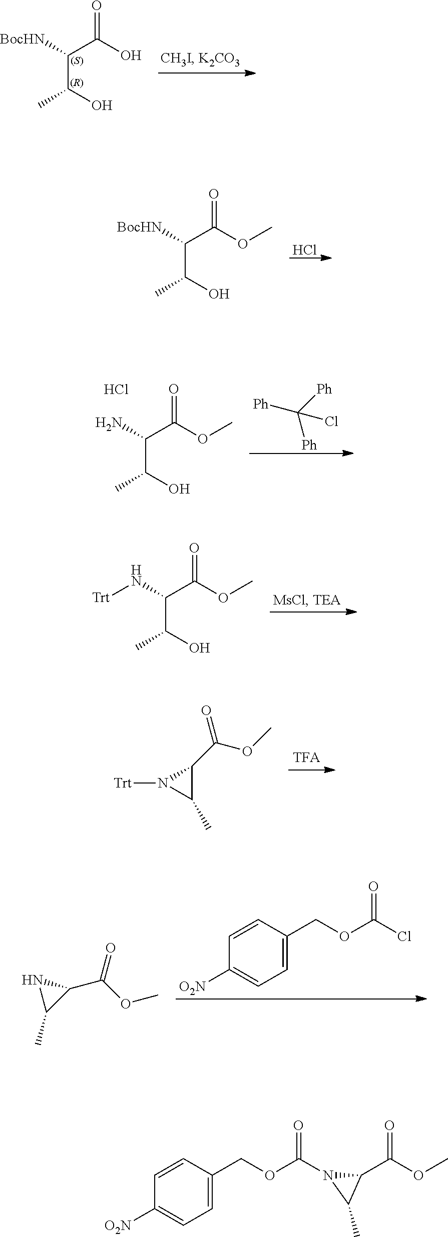

Step 1: Methyl (tert-butoxycarbonyl)-L-threoninate: To a solution of (tert-butoxycarbonyl)-L-threonine (25.0 g, 0.11 mol) in DMF (250 mL) was added K₂CO₃ (23.0 g, 0.16 mol) and CH₃I (19.4 g, 0.13 mol). The reaction mixture was stirred at room temperature for 4 hours and then diluted with water (300 mL). The reaction mixture was extracted with EtOAc (500 mL×2). The combined organic layers were washed with brine, dried over Na₂SO₄, filtered, and concentrated. The mixture was purified by column chromatography on silica gel (eluent: petroleum ether/EtOAc=100:1 to 10:1) to afford methyl (tert-butoxycarbonyl)-L-threoninate (20 g, 80%) as a yellow oil. LCMS m/z=256.2 [M+Na]⁺. ¹H NMR (400 MHz, DMSO-d₆): δ 6.53 (d, J=8.8 Hz, 1H), 4.78 (d, J=7.2 Hz, 1H), 4.01-3.96 (m, 1H), 3.62 (s, 3H), 1.38 (s, 9H), 1.07 (d, J=6.2 Hz, 3H).

Step 2: Methyl L-threoninate hydrochloride: A mixture of methyl (tert-butoxycarbonyl)-L-threoninate (20 g, 85.7 mmol) in a solution of HCl in 1,4-dioxane (4 M, 250 mL) was stirred at room temperature for 6 hours. The solvent was removed under vacuum to afford crude methyl L-threoninate hydrochloride (14.5 g, 100%), which was used directly in the next step. LCMS m/z=134.2 [M+H]⁺; ¹H NMR (400 MHz, DMSO-d₆) δ 8.52 (s, 3H), 4.14-4.07 (m, 1H), 3.90 (d, J=3.8 Hz, 1H), 3.73 (s, 3H), 3.55 (s, 1H), 1.20 (d, J=6.6 Hz, 3H).

Step 3: Methyl trityl-L-threoninate: To a solution of methyl L-threoninate hydrochloride (14.5 g, 85.5 mmol) in DCM (300 mL) was added TEA (45 g, 0.44 mol) and Trt-Cl (28.6 g, 102.6 mmol). The reaction mixture was stirred at room temperature overnight and then diluted with water (200 mL). The reaction mixture was extracted with DCM (300 mL×2). The combined organic layers were washed with brine, dried over Na₂SO₄, filtered, and concentrated. The mixture was purified by column chromatography on silica gel (eluent: petroleum ether/EtOAc=100:1 to 10:1) to afford methyl trityl-L-threoninate (24 g, 75%) as a white solid. ¹H NMR (400 MHz, DMSO-d₆): δ 7.41 (d, J=7.4 Hz, 7H), 7.28 (d, J=7.4 Hz, 7H), 7.23-7.16 (m, 3H), 5.05 (d, J=4.8 Hz, 1H), 3.98-3.87 (m, 1H), 3.23-3.16 (m, 4H). 3.02 (s, 3H), 2.68 (d, J=10.0 Hz, 1H), 1.08 (d, J=6.4 Hz, 3H).

Step 4: Methyl (2S,3S)-3-methyl-1-tritylaziridine-2-carboxylate: To a solution of methyl trityl-L-threoninate (12 g, 32 mmol) in THF (130 mL) was added TEA (6.5 g, 64 mmol) and MsCl (5.4 g, 38.4 mmol). The reaction mixture was heated at 80° C. for 30 hours and then cooled to room temperature. The reaction mixtures was diluted with water (100 mL) and extracted with EtOAc (250 mL×2). The combined organic layers were washed with brine, dried over Na₂SO₄, filtered, and concentrated. The mixture was purified by column chromatography on silica gel (eluent: petroleum ether/EtOAc=100:1 to 20:1) to afford methyl (2S,3S)-3-methyl-1-tritylaziridine-2-carboxylate (8 g, 70%) as a white solid. ¹H NMR (400 MHz, DMSO-d₆) δ 7.45-7.38 (m, 6H), 7.36-7.27 (m, 7H), 7.29-7.21 (m, 4H), 3.65 (s, 3H), 1.71 (d, J=6.6 Hz, 1H), 1.61-1.50 (m, 1H), 1.27 (d, J=5.4 Hz, 3H).

Steps 5 and 6: 2-Methyl 1-(4-nitrobenzyl) (2S,3S)-3-methylaziridine-1,2-dicarboxylate: To a solution of methyl (2S,3S)-3-methyl-1-tritylaziridine-2-carboxylate (2 g, 5.6 mmol) in a mixture of DCM (20 mL) and MeOH (1 mL) was added TFA (10 mL). The mixture was stirred at room temperature for 30 minutes and then diluted with water (20 mL). The reaction mixture was extracted with Et₂O (30 mL×2). The aqueous layer was made basic to pH˜9 with the addition of solid NaHCO₃(LCMS: m/z=116.15 [M+H]⁺). The aqueous layer was partitioned against EtOAc (20 mL), and 4-nitrobenzyl chloroformate (1.3 g, 5.6 mmol) was added. The mixture was stirred at room temperature overnight and then extracted with EtOAc (50 mL×3). The combined organic layers were washed with brine, dried over Na₂SO₄, filtered, and concentrated. The mixture was purified by column chromatography on silica gel (eluent: petroleum ether/EtOAc=100:1 to 20:1) to afford 2-methyl 1-(4-nitrobenzyl) (2S,3S)-3-methylaziridine-1,2-dicarboxylate (500 mg, 31%) as a white solid. LCMS: m/z=295.0 [M+H]⁺. ¹H NMR (400 MHz, DMSO-d₆): δ 8.28-8.21 (m, 2H), 7.68-7.61 (m, 2H), 5.24 (s, 2H), 3.70 (s, 3H), 3.42 (d, J=6.8 Hz, 1H), 3.08-2.97 (m, 1H), 1.21 (d, J=5.6 Hz, 3H).

### Synthesis of O-((2-oxabicyclo[2.2.2]octan-4-yl)methyl)-N-(((4-nitrobenzyl)oxy)carbonyl)-L-threonine

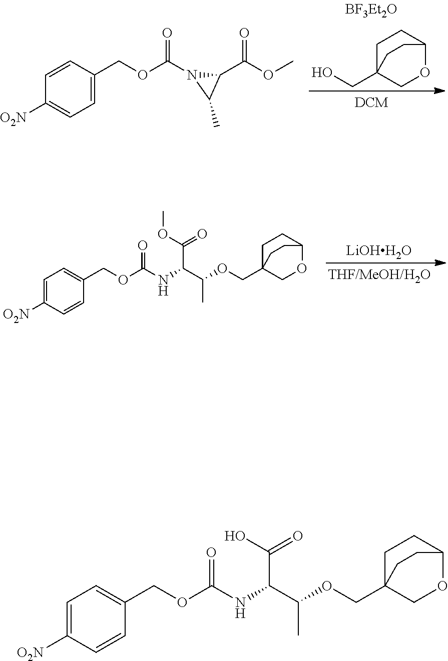

Step 1: Methyl O-((2-oxabicyclo[2.2.2]octan-4-yl)methyl)-N-(((4-nitrobenzyl)oxy)carbonyl)-L-threoninate: To a solution of 2-methyl 1-(4-nitrobenzyl) (2S,3S)-3-methylaziridine-1,2-dicarboxylate (500 mg, 1.7 mmol) in DCM (0.5 mL) were added (2-oxabicyclo[2.2.2]octan-4-yl)methanol (242 mg, 1.7 mmol) and BF₃·Et₂O (724 mg, 5.1 mmol). The mixture was stirred at room temperature for 2 hours. The mixture was concentrated under vacuum, and the residue was purified by silica gel column (eluent: petroleum ether/EtOAc=7:1 to 4:1) to afford methyl O-((2-oxabicyclo[2.2.2]octan-4-yl)methyl)-N-(((4-nitrobenzyl)oxy) carbonyl)-L-threoninate (100 mg, 13.4% yield) as a yellow oil. LCMS m/z=437.2 [M+H]⁺. 1H NMR (400 MHz, DMSO-d₆): δ 8.25 (d, J=8.8 Hz, 2H), 7.66-7.61 (m, 3H), 5.21 (s, 3H), 4.21 (dd, J₁=4 Hz, J₂=4 Hz, 1H), 3.83-3.76 (m, 1H), 3.65-3.60 (m, 4H), 3.55-3.35 (m, 4H), 1.81-1.45 (m, 6H), 1.39-1.29 (m, 2H), 1.09-1.05 (m, 3H).

Step 2: 0-((2-Oxabicyclo[2.2.2]octan-4-yl)methyl)-N-(((4-nitrobenzyl)oxy)carbonyl)-L-threonine: To a solution of methyl O-((2-oxabicyclo[2.2.2]octan-4-yl)methyl)-N-(((4-nitrobenzyl)oxy)carbonyl)-L-threoninate (2.1 g, 4.8 mmol) in THF (16 mL), MeOH (4 mL), and H₂O (4 mL) was added LiOH (505 mg, 12 mmol) at 0° C. The mixture was stirred at room temperature for 2 hours. The mixture was quenched with water and extracted with EtOAc (30 ml×2). The aqueous phase was adjusted to pH of 2-3 with 1M HCl and extracted with EtOAc (50 mL×3). The combined organic phases were washed with brine, dried over Na₂SO₄, and concentrated under vacuum to afford O-((2-oxabicyclo[2.2.2]octan-4-yl)methyl)-N-(((4-nitrobenzyl)oxy)carbonyl)-L-threonine (1.6 g, 80% yield) as a yellow oil. LCMS m/z=423.2 [M+H]⁺. ¹H NMR (400 MHz, DMSO-d₆): δ 12.7 (s, 1H), 8.24 (d, J=8.4 Hz, 2H), 7.65 (d, J=8.4 Hz, 2H), 7.36-7.32 (m, 1H), 5.21 (s, 2H), 4.10-4.06 (m, 1H), 3.86-3.79 (m, 1H), 3.64-3.61 (m, 1H), 3.56-3.53 (m, 2H), 3.47-3.37 (m, 2H), 1.82-1.66 (m, 2H), 1.55-1.52 (m, 4H), 1.39-1.34 (m, 2H), 1.09-1.04 (m, 3H).

### Synthesis of methyl O-((2-oxabicyclo[2.2.2]octan-4-yl)methyl)-L-threoninate

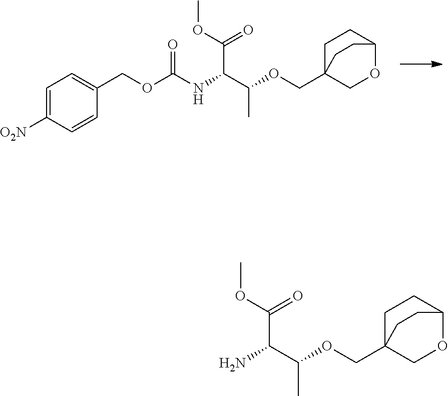 [SMILES: COC(=O)[C@@H](NC(=O)OCc1ccc([N+](=O)[O-])cc1)[C@@H](C)OCC12CCC(CC1)OC2 ; COC(=O)[C@@H](N)[C@@H](C)OCC12CCC(CC1)OC2]

To a solution of methyl O-((2-oxabicyclo[2.2.2]octan-4-yl)methyl)-N-(((4-nitrobenzyl)oxy)carbonyl)-L-threoninate (200 mg, 0.46 mmol) in EtOH (4 mL) was added anhydrous stannous chloride (435 mg, 2.29 mmol). The reaction mixture was heated at reflux for 2 hours and then filtered and concentrated. The residue was purified by prep-TLC (eluent: DCM/MeOH=10:1) to afford methyl O-((2-oxabicyclo[2.2.2]octan-4-yl)methyl)-L-threoninate (100 mg, 95%) as a yellow oil. LCMS m/z=258.2 [M+H]⁺.

### Synthesis of (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indol-4-yl)-2,6-diazaspiro[3.4]octane-8-carboxamide I-14

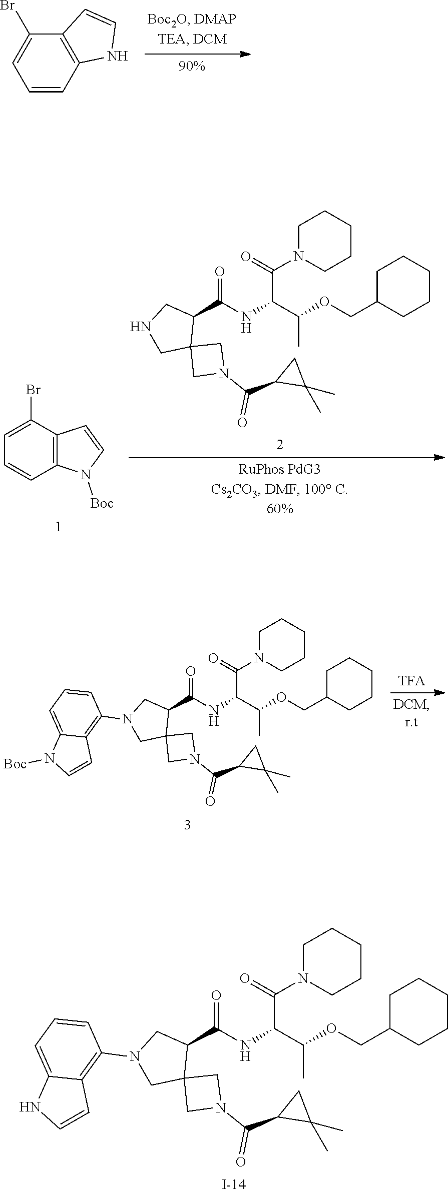

Step 1: tert-Butyl 4-bromo-1H-indole-1-carboxylate (1): To a solution of 4-bromo-1H-indole (1 g, 5.1 mmol), di-tert-butyl dicarbonate (1.67 g, 7.65 mmol), and triethylamine (1.03 g, 10.20 mmol) in dichloromethane (10 mL) was added 4-dimethylaminopyridine (125 mg, 1.02 mmol). The resulting mixture was stirred at room temperature under nitrogen atmosphere for 40 minutes. The reaction mixture was poured into water (8 mL) and extracted with dichloromethane (12 mL). The combined organic layers were washed with water (8 mL×2) and brine (8 mL), dried over anhydrous sodium sulfate, and concentrated to give a crude residue, which was purified by silica gel column chromatography using a 0.5% ethyl acetate in hexane gradient to afford tert-butyl 4-bromo-1H-indole-1-carboxylate 1 (1.35 g, 90%) as a colorless oil. ¹H NMR (400 MHz, DMSO-d₆): δ 8.07 (d, J=4.2 Hz, 1H), 7.79 (d, J=1.8 Hz, 1H), 7.47 (d, J=3.8 Hz, 1H), 7.27 (t, J=8.2 Hz, 1H), 6.66 (d, J=1.8 Hz, 1H), 1.63 (s, 9H).

Step 2: tert-Butyl 4-((S)-8-(((2S,3R)-3-(cyclohexylmethoxy)-1-oxo-1-(piperidin-1-yl) butan-2-yl)carbamoyl)-2-((S)-2,2-dimethylcyclopropane-1-carbonyl)-2,6-diazaspiro[3.4]octan-6-yl)-1H-indole-1-carboxylate (3): To a solution of (S)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide (2) (0.090 g, 0.17 mmol), tert-butyl 4-bromo-1H-indole-1-carboxylate (1) (0.067 g, 0.23 mmol), and cesium carbonate (0.113 g, 0.35 mmol) in N,N-dimethylformamide (1.5 mL) was added RuPhos-Pd-G3 (0.029 g, 0.035 mmol) under nitrogen atmosphere. The mixture was stirred at 100° C. overnight. The reaction mixture was poured into water (5 mL) and extracted with ethyl acetate (8 mL). The organic layer was washed with water (5 mL×2) and brine (5 mL), dried over anhydrous sodium sulfate, and concentrated to give a crude residue, which was purified by silica gel column chromatography using a 10%-50% ethyl acetate in dichloromethane gradient to afford tert-butyl 4-((S)-8-(((2S,3R)-3-(cyclohexylmethoxy)-1-oxo-1-(piperidin-1-yl)butan-2-yl)carbamoyl)-2-((S)-2,2-dimethylcyclopropane-1-carbonyl)-2,6-diazaspiro[3.4]octan-6-yl)-1H-indole-1-carboxylate (3) (0.077 g, 62% yield) as a green solid. MS: [MH]⁺ 732.5.

Step 3: (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indol-4-yl)-2,6-diazaspiro[3.4]octane-8-carboxamide (I-14): To a solution of tert-butyl 4-((S)-8-(((2S,3R)-3-(cyclohexylmethoxy)-1-oxo-1-(piperidin-1-yl)butan-2-yl)carbamoyl)-2-((S)-2,2-dimethylcyclopropane-1-carbonyl)-2,6-diazaspiro[3.4]octan-6-yl)-1H-indole-1-carboxylate (3) (0.072 g, 0.098 mmol) in dichloromethane (0.5 mL) was added trifluoroacetic acid (0.5 mL). The resulting mixture was stirred at room temperature for 2 hours. The reaction mixture was basified with saturated aqueous sodium bicarbonate solution (3 mL) and extracted with dichloromethane (5 mL×3). The combined organic layers were dried over anhydrous sodium sulfate and concentrated to give a crude residue, which was purified by prep-TLC using a 3% methanol in dichloromethane gradient to afford (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indol-4-yl)-2,6-diazaspiro[3.4]octane-8-carboxamide (I-14) (0.028 g, 37% yield) as a green solid. ¹H NMR (400 MHz, CD₃OD): δ 8.22-8.14 (m, 1H), 7.11 (s, 1H), 6.97 (t, J=8.0 Hz, 1H), 6.88 (d, J=4.0 Hz, 1H), 6.68 (s, 1H), 6.23 (d, J=3.8 Hz, 1H), 4.96-4.93 (m, 1H), 4.44-4.39 (m, 1H), 4.31-4.18 (m, 2H), 4.11-3.79 (m, 6H), 3.75-3.70 (m, 1H), 3.56 (s. 4H), 3.37 (s, 1H), 3.30 (d, J=3.8 Hz, 1H), 3.19-3.12 (m, 1H), 1.67-1.52 (m, 11H), 1.20-1.07 (m, 13H), 0.92-0.74-(m, 4H). MS: [MH]⁺ 632.6.

The following compounds were prepared in a manner analogous to the procedures described above for (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indol-4-yl)-2,6-diazaspiro[3.4]octane-8-carboxamide (I-14):

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indazol-4-yl)-2,6-diazaspiro[3.4]octane-8-carboxamide I-13 (0.025 g, 20% yield) as a green solid. ¹HNMR (400 MHz, CD₃OD): δ 8.21 (s. 1H), 7.19 (t, J=7.8 Hz, 1H), 6.81 (d, J=8.4 Hz, 1H), 6.11 (d, J=8.4 Hz, 1H), 4.97-4.93 (m, 1H), 4.43 (t, J=9.8 Hz, 1H), 4.35-4.19 (m, 2H), 4.15-3.99 (m, 3H), 3.97-3.87 (m, 3H), 3.76-3.70 (m, 1H), 3.61-3.38 (m, 6H), 3.20-3.14 (m, 1H), 1.74-1.64 (m, 6H), 1.58-1.42 (m, 7H), 1.19-1.15 (m, 11H), 1.07-1.04 (m, 1H), 0.92-0.85 (m, 2H), 0.81-0.77 (m, 1H). MS: [MH]⁺ 633.3.

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-pyrazolo[4,3-c]pyridin-4-yl)-2,6-diazaspiro[3.4]octane-8-carboxamide 1-9 (0.040 g, 66% yield) as a white solid. ¹HNMR (400 MHz, CD₃OD): δ 8.36 (s, 1H), 7.72 (d, J=6.4 Hz, 1H), 6.84 (d, J=6.4 Hz, 1H), 4.96-4.93 (m, 1H), 4.47-4.01 (m, 8H), 3.78-3.73 (m, 1H), 3.58-3.51 (m, 5H), 3.38-3.33 (m, 1H), 3.23-3.17 (m, 1H), 1.76-1.44 (m, 13H), 1.56-1.24 (m, 11H), 1.08-1.05 (m, 1H), 0.97-0.89 (m, 3H), 0.83-0.78 (m, 1H). MS: [MH]⁺ 635.0.

(S)-6-(benzo[d]thiazol-7-yl)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide I-7 (0.026 g, 42% yield) as a yellow solid. ¹HNMR (400 MHz, CD₃OD): δ 9.14 (s, 1H), 7.49 (d, J=7.6 Hz, 1H), 7.40 (t, J=8.0 Hz, 1H), 6.72-6.70 (m, 1H), 4.95-4.92 (m, 1H), 4.42 (d, J=8.8 Hz, 1H), 4.32-4.06 (m, 3H), 4.01-3.85 (m, 5H), 3.75-3.72 (m, 1H), 3.54-3.42 (m, 5H), 3.36-3.34 (m, 1H), 3.20-3.14 (m, 1H), 1.70-1.42 (m, 13H), 1.18-1.14 (m, 11H), 1.07-1.04 (m, 1H), 0.93-0.88 (m, 2H), 0.79-0.76 (m, 1H). MS: [MH]⁺ 650.4.

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide I-15 (0.040 g, 31% yield) as a yellow solid. ¹HNMR (400 MHz. CD₃OD): δ 9.36 (s, 1H), 8.05 (d, J=5.6 Hz, 1H), 7.28 (d, J=5.6 Hz, 1H), 4.96-4.93 (m, 1H), 4.45-4.40 (m, 1H). 4.34-4.16 (m, 3H), 4.15-4.07 (m, 3H), 4.01-3.96 (m, 1H), 3.70-3.77 (m, 1H), 3.43-3.56 (m, 5H), 3.32-3.36 (m, 1H), 3.17-3.22 (m, 1H), 1.75-1.46 (m, 13H), 1.26-1.21 (m, 2H), 1.18-1.14 (m, 10H), 1.07-1.04 (m, 1H), 0.96-0.90 (m, 2H), 0.81-0.76 (m, 1H). MS: [MH]⁺ 651.7.

(S)-N-((2S,3R)-3-(cyclohexylmethoxy)-1-oxo-1-(piperidin-1-yl)butan-2-yl)-2-((S)-2,2-dimethylcyclopropane-1-carbonyl)-6-(1H-pyrazolo[3,4-c]pyridin-7-yl)-2,6-diazaspiro[3.4]octane-8-carboxamide 2,2,2-trifluoroacetate I-12 (60 mg, 61% yield) as an off-white solid. ¹HNMR (400 MHz. CD₃OD): δ 8.37 (s, 1H), 7.23 (d, J=6.4 Hz, 1H), 7.14 (d, J=6.8 Hz, 1H), 4.99-4.93 (m, 1H), 4.89-4.82 (m, 1H), 4.75-3.92 (m, 7H), 3.82-3.44 (m, 6H). 3.40-3.34 (m, 1H), 3.25-3.17 (m, 1H), 1.81-1.31 (m, 13H), 1.31-1.13 (m, 11H), 1.11-1.04 (m, 1H), 0.99-0.88 (m, 2H), 0.85-0.78 (m, 1H). MS: [MH]⁺ 634.9.

6-(Benzo[d]thiazol-4-yl)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide I-8 (33 mg, 36% yield) as a white solid. ¹HNMR (400 MHz, CD₃OD): δ 8.89 (d, J=2.8 Hz, 1H), 7.34 (d, J=7.8 Hz, 1H), 7.27 (t, J=7.8 Hz, 1H), 6.65 (dd, J=7.8, 1.8 Hz, 1H). 4.94-4.91 (m, 1H), 4.42 (t, J=8.8 Hz, 1H), 4.32-3.95 (m, 8H), 3.75-3.69 (m, 1H), 3.53-3.48 (m, 4H), 3.42-3.33 (m, 2H), 3.20-3.13 (m, 1H), 1.70-1.41 (m, 13H), 1.29-1.15 (m, 12H), 1.07-1.04 (m, 1H), 0.94-0.88 (m, 2H), 0.81-0.76 (m, 1H). MS: [MH]⁺ 650.3.

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indazol-7-yl)-2,6-diazaspiro[3.4]octane-8-carboxamide I-11 (28 mg, 37% yield) as a green solid. ¹H NMR (400 MHz, CDCl₃) δ8.70 (d, J=4.4 Hz, 1H), 8.46-8.44 (m, 1H), 8.06 (s, 1H), 7.41 (d, J=4.0 Hz, 1H), 7.07 (t, J=7.6 Hz, 1H). 6.83-6.78 (m, 1H), 4.97 (d, J=4.0 Hz, 1H), 4.42-4.25 (m, 2H), 4.19-4.11 (m, 2H), 4.04-3.96 (m, 2H), 3.85-3.80) (m, 1H), 3.70-3.67 (m, 2H), 3.60-3.50) (m, 3H), 3.45-3.35 (m, 2H), 3.16-3.09 (m, 2H), 3.02-2.94 (m, 1H), 1.66-1.57 (m, 7H), 1.34-1.20 (m, 6H), 1.15-1.08 (m, 9H), 0.94-0.86 (m, 3H), 0.74-0.71 (m, 1H), 0.64-0.50 (m, 2H). MS: [MH]⁺ 633.6.

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(thiazolo[4,5-c]pyridin-4-yl)-2,6-diazaspiro[3.4]octane-8-carboxamide 1-10 (22 mg, 29% yield) as a yellow solid. ¹HNMR (400 MHz, CD₃OD): δ 8.98 (s. 1H), 7.93 (d, J=5.6 Hz, 1H), 7.26 (d, J=5.6 Hz, 1H), 4.96-4.93 (m, 1H), 4.07-4.47 (m, 7H), 3.95-3.99 (m, 1H), 3.71-3.77 (m, 1H), 3.34-3.59 (m, 6H), 3.17-3.22 (m, 1H), 1.60-1.76 (m, 6H), 1.40-1.56 (m, 7H), 1.14-1.27 (m, 11H), 1.04-1.07 (m, 1H), 0.88-0.96 (m, 2H), 0.76-0.81 (m, 1H). MS: [MH]⁺ 651.7.

### Synthesis of (S)-N-((3S,4R)-4-(2-oxabicyclo[2.2.2]octan-4-ylmethoxy)-2-hydroxy-2-methylpentan-3-yl)-2-(oxazol-2-yl)-6-(thiazole-5-carbonyl)-2,6-diazaspiro[3.4]octane-8-carboxamide 1-3

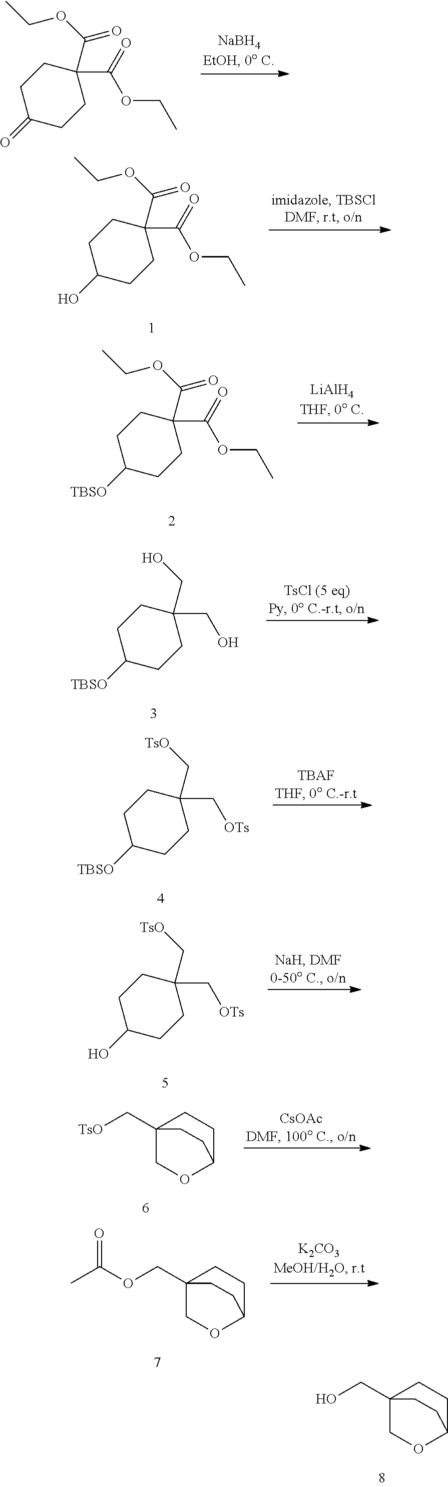

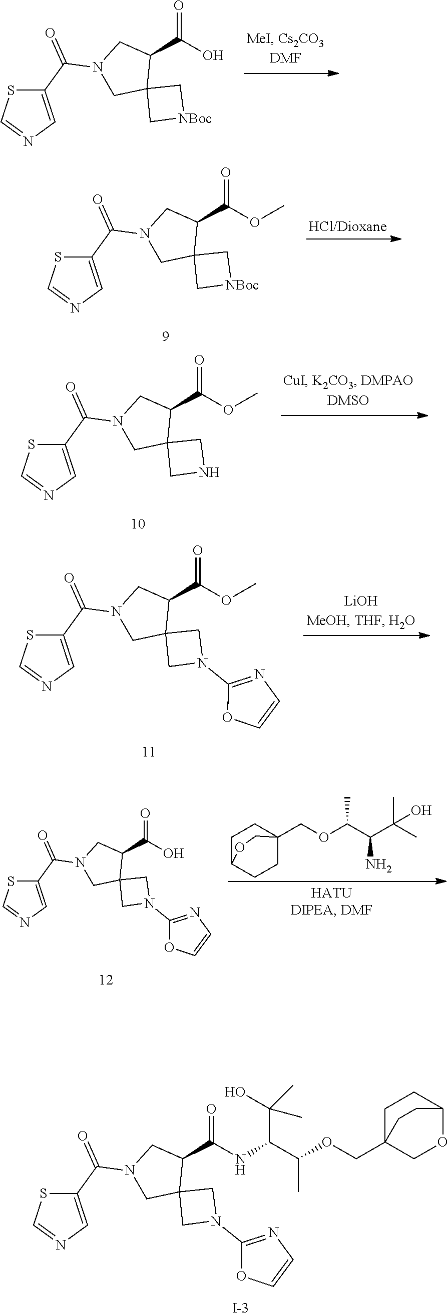

Step 1: Diethyl 4-hydroxycyclohexane-1,1-dicarboxylate (1): To a solution of diethyl 4-oxocyclohexane-1,1-dicarboxylate (10.000 g, 41.27 mmol) in ethanol (100 mL) was added sodium borohydride (1.561 g, 41.27 mmol) at 0° C. The resulting mixture was stirred at 0° C. under nitrogen atmosphere for 30 minutes. The reaction mixture was concentrated and then saturated aqueous ammonium chloride (80 mL) was added. The mixture was extracted with ethyl acetate (90 mL×2). The combined organic layers were washed with water (100 mL) and brine (100 mL), dried over anhydrous sodium sulfate, and concentrated to give a crude residue, which was purified by silica gel column chromatography using a 33% ethyl acetate in dichloromethane gradient to afford diethyl 4-hydroxycyclohexane-1,1-dicarboxylate 1 (10.080 g, 99%) as a colorless oil. MS: [MH]⁺ 245.3.

Step 2: Diethyl 4-((tert-butyldimethylsilyl)oxy)cyclohexane-1,1-dicarboxylate (2): To a solution of diethyl 4-hydroxycyclohexane-1,1-dicarboxylate 1 (10.080 g, 41.27 mmol) in N,N-dimethylformamide (80 mL) was added imidazole (5.620 g, 82.55 mmol) and tert-butyldimethylsilyl chloride (6.840 g, 45.40 mmol). The resulting mixture was stirred at room temperature under nitrogen atmosphere overnight. The reaction mixture was quenched with water (70 mL) and extracted with ethyl acetate (90 mL). The organic layer was washed with water (70 mL×2) and brine (70 mL), dried over anhydrous sodium sulfate, and concentrated to give a crude residue, which was purified by silica gel column chromatography using a 5% ethyl acetate in hexane gradient to afford diethyl 4-((tert-butyldimethylsilyl)oxy)cyclohexane-1,1-dicarboxylate 2 (13.620 g, 92%) as a colorless oil. MS: [MH]⁺ 318.2.

Step 3: (4-((Tert-butyldimethylsilyl)oxy)cyclohexane-1,1-diyl)dimethanol (3): To a mixture of LiAlH₄ (2.160 g, 56.986 mmol) in anhydrous tetrahydrofuran (100 mL) at 0° C. was added a solution of 4-((tert-butyldimethylsilyl)oxy)cyclohexane-1,1-dicarboxylate 2 (13.620 g, 37.99 mmol) in anhydrous tetrahydrofuran (40 mL) dropwise. The resulting mixture was stirred at 0° C.) under nitrogen atmosphere for 3 hours. The reaction mixture was quenched with water (2 mL), 15% sodium hydroxide solution (2 mL), and water (6 mL) at 0° C. The resulting mixture was stirred at room temperature for 30 minutes and filtered. The filtrate was concentrated and extracted with ethyl acetate (100 mL). The organic layer was washed with water (80 mL×2) and brine (80 mL), dried over anhydrous sodium sulfate, and concentrated to give a crude residue, which was purified by silica gel column chromatography using ethyl acetate gradient to afford (4-((tert-butyldimethylsilyl)oxy)cyclohexane-1,1-diyl)dimethanol 3 (7.310 g, 70%) as a white solid. MS: [MH]⁺ 275.2.

Step 4: (4-((tert-Butyldimethylsilyl)oxy)cyclohexane-1,1-diyl)bis(methylene) bis(4-methylbenzenesulfonate)(4): To a solution of (4-((tert-butyldimethylsilyl)oxy)cyclohexane-1,1-diyl)dimethanol 3 (7.310 g, 26.63 mmol) in pyridine (73 mL) at 0° C. was added tosyl chloride (25.390 g, 133.17 mmol). The resulting mixture was stirred at room temperature overnight. The reaction mixture was poured into water (40 mL) and extracted with ethyl acetate (70 mL). The organic layer was washed with water (50 mL×2) and brine (50 mL), dried over anhydrous sodium sulfate, and concentrated to give a crude residue, which was purified by silica gel column chromatography using a 33% ethyl acetate in dichloromethane gradient to afford (4-((tert-butyldimethylsilyl)oxy)cyclohexane-1,1-diyl)bis(methylene)bis(4-methylbenzenesulfonate) 4 (13.700 g, 88%) as a white solid. MS: [MH]⁺ 583.3.

Step 5: (4-Hydroxycyclohexane-1,1-diyl)bis(methylene)bis(4-methylbenzenesulfonate)(5): To a solution of (4-((tert-butyldimethylsilyl)oxy)cyclohexane-1,1-diyl)bis(methylene)bis(4-methylbenzenesulfonate) 4 (13.700 g, 23.506 mmol) in tetrahydrofuran (130 mL) at 0° C. was added tetrabutylammonium fluoride (71 mL) dropwise. The mixture was stirred at room temperature under nitrogen atmosphere overnight. The reaction mixture was concentrated. The residue was diluted with water (80 mL), and extracted with ethyl acetate (90 mL×2). The combined organic layers were washed with water (100 mL×2) and brine (100 mL), dried over anhydrous sodium sulfate, and concentrated to give a crude residue, which was purified by silica gel column chromatography using ethyl acetate gradient to afford (4-hydroxycyclohexane-1,1-diyl)bis(methylene)bis(4-methylbenzenesulfonate) 5 (11.000 g, 99%) as a yellow oil. MS: [MH]⁺ 469.1.

Step 6: 2-Oxabicyclo[2.2.2]octan-4-ylmethyl 4-methylbenzenesulfonate (6): To a solution of (4-hydroxycyclohexane-1,1-diyl)bis(methylene)bis(4-methylbenzenesulfonate) 5 (10.800 g, 23.05 mmol) in N,N-dimethylformamide (75 mL) at 0° C. was added sodium hydride (1.840 g, 46.10 mmol). The resulting mixture was stirred at 0° C. for one hour and then warmed to 50° C. and stirred overnight. The reaction mixture was quenched with water (70 mL) and extracted with ethyl acetate (70 mL). The organic layer was washed with water (70 mL×4) and brine (70 mL), dried over anhydrous sodium sulfate, and concentrated to give a crude residue, which was purified by silica gel column chromatography using a 25% ethyl acetate in hexane gradient to afford 2-oxabicyclo[2.2.2]octan-4-ylmethyl 4-methylbenzenesulfonate 6 (5.030 g, 73%) as a white solid. MS: [MH]⁺ 297.0.

Step 7: 2-Oxabicyclo[2.2.2]octan-4-ylmethyl acetate (7): To a solution of 2-oxabicyclo[2.2.2]octan-4-ylmethyl 4-methylbenzenesulfonate 6 (14.900 g, 50.273 mmol) in N,N-dimethylformamide (100 mL) was added cesium acetate (24.120 g, 125.683 mmol), and the resulting mixture was stirred at 100° C. under nitrogen atmosphere overnight. The reaction mixture was quenched with water (80 mL) and extracted with ethyl acetate (100 mL×3). The combined organic layers were washed with brine (300 mL), dried over anhydrous sodium sulfate, and concentrated to give a crude residue, which was purified by silica gel column chromatography using a 10% ethyl acetate in hexane gradient to afford 2-oxabicyclo[2.2.2]octan-4-ylmethyl acetate 7 (8.500 g, 91%) as a colorless oil. MS: [MH]⁺ 185.0.

Step 8: 2-Oxabicyclo[2.2.2]octan-4-ylmethanol (8): To a solution of 2-oxabicyclo[2.2.2]octan-4-ylmethyl acetate 7 (8.500 g, 46.138 mmol) in methanol (84 mL) and water (28 mL) was added potassium carbonate (31.880 g, 230.690 mmol). The resulting mixture was stirred at room temperature under nitrogen atmosphere for 3 hours. The mixture was concentrated. The residue was diluted with water (80 mL) and extracted with dichloromethane (90 mL×2). The combined organic layers were washed with water (100 mL) and brine (100 mL), dried over anhydrous sodium sulfate, and concentrated to give a crude residue, which was purified by silica gel column chromatography using a 33% ethyl acetate in hexane gradient to afford 2-oxabicyclo[2.2.2]octan-4-ylmethanol 8 (5.430 g, 83%) as a colorless oil. ¹H NMR (400 MHz, CDCl₃): δ 3.80-3.79 (m, 1H), 3.73 (t, J=1.4 Hz, 2H), 3.30 (s, 2H), 2.06-2.00 (m, 2H). 1.66-1.65 (m, 1H), 1.62 (s, 2H), 1.59 (m, 1H), 1.51-1.48 (m, 2H). MS: [MH]⁺ 143.0.

Step 9:2-(Tert-butyl) 8-methyl(S)-6-(thiazole-5-carbonyl)-2,6-diazaspiro[3.4]octane-2,8-dicarboxylate (9): To a solution of (S)-2-(tert-butoxy carbonyl)-6-(thiazole-5-carbonyl)-2,6-diazaspiro[3.4]octane-8-carboxylic acid 8 (0.500 g, 1.36 mmol) and cesium carbonate (0.666 g, 2.04 mmol) in N,N-dimethylformamide (8 mL) at room temperature was added iodomethane (0.398 g, 2.80 mmol). The resulting mixture was stirred at room temperature for 0.5 hour. The reaction mixture was poured into water (15 mL) and extracted with ethyl acetate (15 mL×3). The combined organic layers were washed with brine (10 mL×2), dried over anhydrous sodium sulfate, and concentrated under reduced pressure to give a crude residue, which was purified by silica gel column chromatography using a 30% ethyl acetate in hexane gradient to afford 2-(tert-butyl) 8-methyl(S)-6-(thiazole-5-carbonyl)-2,6-diazaspiro[3.4]octane-2,8-dicarboxylate 9 (0.699 g, 90%) as a white solid. MS: [MH]⁺ 382.2.

Step 10: Methyl(S)-6-(thiazole-5-carbonyl)-2,6-diazaspiro[3.4]octane-8-carboxylate (10): To a solution of 2-(tert-butyl) 8-methyl(S)-6-(thiazole-5-carbonyl)-2,6-diazaspiro[3.4]octane-2,8-dicarboxylate 9 (0.699 g, 1.83 mmol) in dichloromethane (20 mL) was added hydrogen chloride in dioxane (4.0 M, 10 mL). The resulting mixture was stirred at room temperature for 1 hour. The reaction mixture was concentrated under reduced pressure to give crude product as a residue. The residue was taken up in water (1 mL), basified with saturated aqueous sodium carbonate solution to pH of 8-9, and extracted with dichloromethane (10 mL×2). The combined organic layers were dried over anhydrous sodium sulfate and concentrated under reduced pressure to give a crude residue, which was purified by silica gel column chromatography using a 8% methanol in dichloromethane gradient to afford methyl(S)-6-(thiazole-5-carbonyl)-2,6-diazaspiro[3.4]octane-8-carboxylate 10 (0.437 g, 85%) as a white solid. MS: [MH]⁺ 282.1.

Step 11: Methyl(S)-2-(oxazol-2-yl)-6-(thiazole-5-carbonyl)-2,6-diazaspiro[3.4]octane-8-carboxylate (11): A solution of (S)-methyl 6-(thiazole-5-carbonyl)-2,6-diazaspiro[3.4]octane-8-carboxylate 10 (0.333 g, 1.18 mmol), CuI (0.045 g, 0.23 mmol), DMPAO (0.092 g, 0.474 mmol), and K₂CO₃ (0.327 g, 2.37 mmol) in DMSO (3 mL) was heated at 100° C. under N₂ overnight. The reaction mixture was cooled to room temperature and then poured into water (10 mL) and extracted with ethyl acetate (5 mL×3). The combined organic layers were washed with brine (20 mL×2), dried over anhydrous sodium sulfate, and concentrated under reduced pressure to give a crude residue, which was purified by silica gel column chromatography using a 50% ethyl acetate in hexane gradient to afford methyl(S)-2-(oxazol-2-yl)-6-(thiazole-5-carbonyl)-2,6-diazaspiro[3.4]octane-8-carboxylate 11 (0.131 g, 32%) as a white solid. MS: [MH]⁺ 349.1.

Step 12: (S)-2-(oxazol-2-yl)-6-(thiazole-5-carbonyl)-2,6-diazaspiro[3.4]octane-8-carboxylic acid (12): To as solution of methyl(S)-2-(oxazol-2-yl)-6-(thiazole-5-carbonyl)-2,6-diazaspiro[3.4]octane-8-carboxylate 11 (0.131 g, 0.37 mmol) in tetrahydrofuran (2 mL)-water (0.5 mL)-methanol (0.5 mL) was added lithium hydroxide monohydrate (0.031 g, 0.74 mmol). The mixture was stirred at room temperature for 1 hour. The reaction mixture solution was acidified with diluted hydrochloric acid (3N) to pH of 3-4 and extracted with dichloromethane (15 mL×2). The combined organic layers were washed with brine (5 mL), dried over sodium sulfate, and concentrated under reduced pressure to afford (S)-2-(oxazol-2-yl)-6-(thiazole-5-carbonyl)-2,6-diazaspiro[3.4]octane-8-carboxylic acid 12 (0.111 g, 89%) as a white solid. MS: [MH]⁺ 335.0.

Step 13: (S)-N-((3S,4R)-4-((2-oxabicyclo[2.2.2]octan-4-yl)methoxy)-2-hydroxy-2-methylpentan-3-yl)-2-(oxazol-2-yl)-6-(thiazole-5-carbonyl)-2,6-diazaspiro[3.4]octane-8-carboxamide I-3A and I-3B: To a solution of (S)-2-(oxazol-2-yl)-6-(thiazole-5-carbonyl)-2,6-diazaspiro[3.4]octane-8-carboxylic acid 12 (0.075 g, 0.21 mmol), (3S,4R)-4-((2-oxabicyclo[2.2.2]octan-4-yl)methoxy)-3-amino-2-methylpentan-2-ol (0.048 mg, 0.21 mmol), and N-ethyl-N-isopropylpropan-2-amine (0.0741 g, 0.6 mmol) in N,N-dimethylformamide (5 mL) at 0-5° C. was added 2-(7-aza-1H-benzotriazole-1-yl)-1,1,3,3-tetramethyluronium hexafluorophosphate (0.110 g, 0.3 mmol). The resulting mixture was stirred at room temperature for 0.5 hour. The reaction mixture was poured into water (20 mL) and extracted with ethyl acetate (5 mL×3). The combined organic layers were washed with brine (20 mL×2), dried over anhydrous sodium sulfate, and concentrated under reduced pressure to give a crude residue, which was purified by preparative TLC using a 10% methanol in dichloromethane gradient to afford (S)-N-((3S,4R)-4-((2-oxabicyclo[2.2.2]octan-4-yl)methoxy)-2-hydroxy-2-methylpentan-3-yl)-2-(oxazol-2-yl)-6-(thiazole-5-carbonyl)-2,6-diazaspiro[3.4]octane-8-carboxamide I-3A (0.020 g, 17.5% yield) and I-3B (0.016 g, 14% yield) as a white solid. I-3A: ¹HNMR (400 MHz, CD₃OD): δ 9.06 (s, 1H), 8.28 (d, J=20.0 Hz, 1H), 7.31 (d, J=4.4 Hz, 1H), 6.72 (d, J=4.4 Hz, 1H), 4.28-4.20 (m, 1H), 4.16-4.00 (m, 4H), 3.94-3.77 (m, 3H), 3.71-3.54 (m, 4H), 3.44-3.42 (m, 1H), 3.17-3.06 (m, 2H), 2.93-2.87 (m, 1H), 1.94-1.88 (m, 2H), 1.61-1.40 (m, 6H), 1.13-1.02 (m, 6H), 0.97-0.86 (m, 3H). MS: [MH]⁺ 574.6. I-3B: ¹HNMR (400 MHz, CD₃OD): δ 9.06 (s, 1H), 8.28 (d, J=13.2 Hz, 1H), 7.55-7.63 (m, 1H), 7.32 (d, J=6.0 Hz, 1H). 6.72 (d, J=4.8 Hz, 1H), 4.26-4.19 (m, 1H), 4.12-3.97 (m, 5H), 3.94-3.77 (m, 3H), 3.86-3.64 (m, 7H), 3.46-3.44 (m, 1H), 3.13-3.09 (m, 1H), 2.91-2.87 (m, 1H), 1.89-1.85 (m, 2H), 1.62-1.44 (m, 6H), 1.14-1.03 (m, 6H), 0.91-0.86 (m, 3H). MS: [MH]⁺ 574.6.

The following compounds were prepared in a manner analogous to the procedures described above for (S)-N-((3S,4R)-4-((2-oxabicyclo[2.2.2]octan-4-yl)methoxy)-2-hydroxy-2-methylpentan-3-yl)-2-(oxazol-2-yl)-6-(thiazole-5-carbonyl)-2,6-diazaspiro[3.4]octane-8-carboxamide (1-3A):

(S)-N-((3S,4R)-4-(2-oxabicyclo[2.2.2]octan-4-ylmethoxy)-2-hydroxy-2-methylpentan-3-yl)-2-(1H-pyrazol-3-yl)-6-(thiazole-5-carbonyl)-2,6-diazaspiro[3.4]octane-8-carboxamide I-31 (0.009 g, 18% yield) as a white solid. ¹HNMR (400 MHz, CD₃OD): δ 9.14 (d, J=7.8 Hz, 1H), 8.35 (d, J=12.0 Hz, 1H), 7.20-7.19 (m, 1H), 5.36-5.26 (m, 1H), 4.20-4.02 (m, 4H), 3.94-3.73 (m, 6H), 3.68 (s, 2H), 3.49-3.36 (m, 2H), 3.20 (d, J=9.2 Hz, 2H), 3.00-2.97 (m, 1H), 2.02-1.94 (m, 2H), 1.68-1.61 (m, 4H), 1.58-1.44 (m, 3H), 1.29-1.08 (m, 10H). MS: [MH]⁺ 573.6.

(S)-N-((3S,4R)-4-((4,4-difluorocyclohexyl)methoxy)-2-hydroxy-2-methylpentan-3-yl)-2-(1H-pyrazol-3-yl)-6-(thiazole-5-carbonyl)-2,6-diazaspiro[3.4]octane-8-carboxamide I-6 (0.005 g, 5% yield) as a colorless oil. ¹HNMR (400 MHz, CD₃OD): δ 9.17 (d, J=4.2 Hz, 1H), 8.31-8.33 (m, 1H), 7.83 (q, J=2.4 Hz, 1H), 6.36-6.38 (m, 1H), 4.29-4.35 (m, 4H), 4.00-4.13 (m, 3H), 3.91-3.94 (m, 2H), 3.71-3.84 (3H), 3.40-3.57 (m, 3H), 2.00-2.03 (m, 2H), 1.80-1.86 (m, 4H), 1.67-1.73 (m, 2H), 1.23-1.36 (m, 9H). MS: [MH]⁺ 581.3.

(S)-N-((3S,4R)-4-((4,4-difluorocyclohexyl)methoxy)-2-hydroxy-2-methylpentan-3-yl)-2-(oxazol-2-yl)-6-(thiazole-5-carbonyl)-2,6-diazaspiro[3.4]octane-8-carboxamide I-4 (0.050 g, 48% yield) as white solid. ¹HNMR (400 MHz, DMSO-d₆): δ 9.25 (s, 1H), 8.41-8.35 (m, 1H), 7.58-7.56 (dd, J=8 Hz, 1H), 6.83-6.82 (t, J=4 Hz, 1H), 4.24-3.82 (m, 9H), 3.69-3.61 (m, 2H), 3.55-3.50 (m, 1H), 3.28-3.27 (m, 1H), 3.17-3.08 (m, 1H), 2.00-1.97 (m, 2H), 1.85-1.62 (m, 5H), 1.18-1.14 (m, 2H), 1.07-1.02 (m, 6H), 0.97-0.85 (m, 3H). MS: [MH]⁺ 582.6.

### Synthesis of (S)-N-((3S,4R)-4-((2-oxabicyclo[2.2.2]octan-4-yl)methoxy)-2-hydroxy-2-methylpentan-3-yl)-6-(benzo[d]thiazol-7-yl)-2-((S)-2,2-dimethylcyclopropane-1-carbonyl)-2,6-diazaspiro[3.4]octane-8-carboxamide I-16 A and I-16B

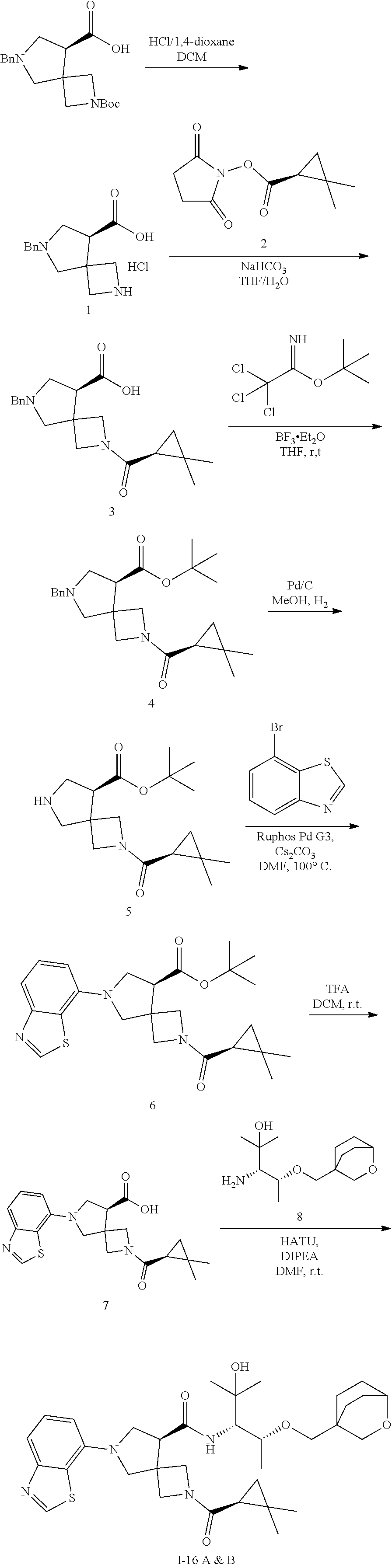

Step 1: (S)-6-Benzyl-2,6-diazaspiro[3.4]octane-8-carboxylic acid hydrochloride (1): A mixture of (S)-6-benzyl-2-(tert-butoxycarbonyl)-2,6-diazaspiro[3.4]octane-8-carboxylic acid (0.500 g, 5.60 mmol) and hydrogen chloride (4.0 N in 1,4-dioxane, 2 mL) in dichloromethane (5 mL) was stirred at room temperature under nitrogen atmosphere for 2 hours. TLC showed the reaction was complete. The reaction mixture was concentrated under reduced pressure to afford (S)-6-benzyl-2,6-diazaspiro[3.4]octane-8-carboxylic acid hydrochloride 1 (0.500 g, crude) as a white solid, which was used in next step without feather purification. MS: [MH]⁺ 247.0

Step 2: (S)-6-Benzyl-2-((S)-2,2-dimethylcyclopropane-1-carbonyl)-2,6-diazaspiro[3.4]octane-8-carboxylic acid (3): A mixture of (S)-6-benzyl-2,6-diazaspiro[3.4]octane-8-carboxylic acid hydrochloride 1 (3.000 g, crude) in water (20 mL) was stirred at room temperature. Sodium bicarbonate (3.640 g, 43.33 mmol) and a solution of 2,5-dioxopyrrolidin-1-yl(S)-2,2-dimethylcyclopropane-1-carboxylate 2 (1.830 g, 8.67 mmol) in tetrahydrofuran (20 mL) were then added. The resulting mixture was stirred at room temperature for 2 hours. The reaction mixture was partitioned between ethyl acetate (20 mL) and water (20 mL). The organic layer was collected, and the aqueous layer was extracted three times with a mixture of isopropyl alcohol in dichloromethane (1/3, 40 mL). The combined extracts were dried over anhydrous sodium sulfate and concentrated to give a crude residue, which was purified by silica gel column chromatography using a using a 5% methanol in dichloromethane gradient to afford (S)-6-benzyl-2-((S)-2,2-dimethylcyclopropane-1-carbonyl)-2,6-diazaspiro[3.4]octane-8-carboxylic acid 3 (2.100 g, 66% over 2 steps) as a colorless solid. MS: [MH]⁺ 343.4.

Step 3: (S)-6-Benzyl-2-((S)-2,2-dimethylcyclopropane-1-carbonyl)-2,6-diazaspiro[3.4]octane-8-carboxylate (4): A mixture of (S)-6-benzyl-2-((S)-2,2-dimethylcyclopropane-1-carbonyl)-2,6-diazaspiro[3.4]octane-8-carboxylic acid 3 (2.100 g, 6.14 mmol), tert-butyl 2,2,2-trichloroacetimidate (6.700 g, 30.73 mmol), and boron trifluoride etherate (4.300 g, 30.49 mmol) in tetrahydrofuran (10 mL) was stirred at room temperature under nitrogen atmosphere overnight. The reaction mixture was poured into water (20 ml) and extracted with a mixture of isopropyl alcohol in dichloromethane (1/3, 40 mL×3). The combined organic layers were dried over anhydrous sodium sulfate and concentrated to give a crude residue, which was purified by silica gel column chromatography using a 5% methanol in dichloromethane gradient to afford tert-butyl (S)-6-benzyl-2-((S)-2,2-dimethylcyclopropane-1-carbonyl)-2,6-diazaspiro[3.4]octane-8-carboxylate 4 (1.200 g, 49%) as a yellow oil. MS: [MH]⁺ 399.6.

Step 4: tert-Butyl (S)-2-((S)-2,2-dimethylcyclopropane-1-carbonyl)-2,6-diazaspiro[3.4]octane-8-carboxylate (5): A mixture of tert-butyl (S)-6-benzyl-2-((S)-2,2-dimethylcyclopropane-1-carbonyl)-2,6-diazaspiro[3.4]octane-8-carboxylate 4 (1.200 g, 3.02 mmol) and Pd/C (10%, 0.240 g) in methanol (20 mL) was stirred at room temperature under hydrogen atmosphere for 2 hours. The mixture was filtered, and the filtrate was concentrated to give a crude residue, which was purified by silica gel column chromatography using a 5% methanol in dichloromethane gradient to afford tert-butyl (S)-2-((S)-2,2-dimethylcyclopropane-1-carbonyl)-2,6-diazaspiro[3.4]octane-8-carboxylate 5 (0.850 g, 86%) as a yellow solid. MS: [MH]⁺ 309.4.

Step 5: tert-Butyl (S)-6-(benzo[d]thiazol-7-yl)-2-((S)-2,2-dimethylcyclopropane-1-carbonyl)-2,6-diazaspiro[3.4]octane-8-carboxylate (6): A mixture of tert-butyl (S)-2-((S)-2,2-dimethylcyclopropane-1-carbonyl)-2,6-diazaspiro[3.4]octane-8-carboxylate 5 (0.155 g, 0.50 mmol), 7-bromobenzo[d]thiazole (0.118 g, 0.55 mmol), Ruphos Pd G3 (0.084 g, 0.101 mmol), and cesium carbonate (0.327 g, 1.01 mmol) in N,N-dimethylformamide (5 mL) was stirred at 100° C. under nitrogen atmosphere overnight. The mixture was concentrated to give a crude residue, which was purified by silica gel column chromatography using a 30% ethyl acetate in hexane gradient to afford tert-butyl (S)-6-(benzo[d]thiazol-7-yl)-2-((S)-2,2-dimethylcyclopropane-1-carbonyl)-2,6-diazaspiro[3.4]octane-8-carboxylate 6 (0.190 g, 86%) as a yellow solid. MS: [MH]⁺ 442.4.

Step 6: (S)-6-(Benzo[d]thiazol-7-yl)-2-((S)-2,2-dimethylcyclopropane-1-carbonyl)-2,6-diazaspiro[3.4]octane-8-carboxylic acid (7): A mixture of tert-butyl (S)-6-(benzo[d]thiazol-7-yl)-2-((S)-2,2-dimethylcyclopropane-1-carbonyl)-2,6-diazaspiro[3.4]octane-8-carboxylate 6 (0.048 g, 0.11 mmol) and trifluoroacetic acid (1 mL) in dichloromethane (1 mL) was stirred at room temperature under nitrogen atmosphere for 2 hours. The mixture was concentrated under reduced pressure to afford (S)-6-(benzo[d]thiazol-7-yl)-2-((S)-2,2-dimethylcyclopropane-1-carbonyl)-2,6-diazaspiro[3.4]octane-8-carboxylic acid 7 (crude) as a white solid, which was used in the next step without feather purification. MS: [MH]⁺ 386.1.

Step 7: (S)-N-((3S,4R)-4-((2-oxabicyclo[2.2.2]octan-4-yl)methoxy)-2-hydroxy-2-methylpentan-3-yl)-6-(benzo[d]thiazol-7-yl)-2-((S)-2,2-dimethylcyclopropane-1-carbonyl)-2,6-diazaspiro[3.4]octane-8-carboxamide (I-16A & I-16B): A mixture of (S)-6-(benzo[d]thiazol-7-yl)-2-((S)-2,2-dimethylcyclopropane-1-carbonyl)-2,6-diazaspiro[3.4]octane-8-carboxylic acid 7 (0.042 g, 0.11 mmol), N,N-diisopropylethylamine (0.042 g, 0.33 mmol), (3S,4R)-4-((2-oxabicyclo[2.2.2]octan-4-yl)methoxy)-3-amino-2-methylpentan-2-ol (0.028 g, 0.11 mmol), and 2-(7-azabenzotriazol-1-yl)-N,N,N′,N′-tetramethyluronium hexafluorophosphate (0.042 g, 0.13 mmol) in N,N-dimethylformamide (1 mL) was stirred at room temperature under nitrogen atmosphere for 2 hours. The mixture was partitioned between ethyl acetate (10 mL) and water (10 mL). The organic layer was collected, and the aqueous layer was extracted with ethyl acetate (10) mL×2). The combined organic layers were washed with brine (10 mL), dried over anhydrous sodium sulfate, and concentrated to give a crude residue, which was purified by prep-HPLC to afford (S)-N-((3S,4R)-4-((2-oxabicyclo[2.2.2]octan-4-yl)methoxy)-2-hydroxy-2-methylpentan-3-yl)-6-(benzo[d]thiazol-7-yl)-2-((S)-2,2-dimethylcyclopropane-1-carbonyl)-2,6-diazaspiro[3.4]octane-8-carboxamide I-16A (0.013 g, 20% yield) and I-16B (0.014 g, 21% yield) as a white solid. I-16A: ¹HNMR (400 MHz, CD₃OD): δ 9.19 (s, 1H), 7.55 (d, J=8.0 Hz, 1H), 7.45 (t, J=8.0 Hz, 1H), 6.79-6.69 (m, 1H), 4.59-4.24 (m, 3H), 4.15-3.85 (m, 7H), 3.66-3.48 (m, 4H), 3.21-3.10 (m, 1H), 2.99 (t, J=8.8 Hz, 1H), 1.87-1.85 (m, 2H), 1.54-1.41 (m, 6H), 1.36-1.31 (m, 2H), 1.25-1.16 (m, 10H), 1.25-1.08 (m, 4H), 0.82-0.79 (m, 1H). MS: [MH]⁺ 625.6. I-16B: ¹HNMR (400 MHz, CD₃OD): δ 9.17 (s, 1H), 7.53 (d, J=8.0 Hz, 1H), 7.43 (t, J=8.0 Hz, 1H), 6.77-6.74 (m, 1H), 4.61 (d, J=9.2 Hz, 1H), 4.38-4.22 (m, 2H), 4.06-3.81 (m, 7H), 3.76-3.72 (m, 3H), 3.56-3.53 (m, 1H), 3.21 (d, J=7.2 Hz, 1H), 3.01 (d, J=9.2 Hz, 1H), 1.99-1.97 (m, 2H), 1.68-1.62 (m, 4H), 1.52-1.41 (m, 3H), 1.32-1.28 (m, 5H), 1.20-1.16 (m, 8H), 1.05-1.01 (m, 4H), 1.36-1.31 (m, 2H), 1.25-1.16 (m, 10H), 1.25-1.08 (m, 4H), 0.83-0.79 (m, 1H). MS: [MH]⁺ 625.5.

The following compound was prepared in a manner analogous to the procedures described above for (S)-N-((3S,4R)-4-((2-oxabicyclo[2.2.2]octan-4-yl)methoxy)-2-hydroxy-2-methylpentan-3-yl)-6-(benzo[d]thiazol-7-yl)-2-((S)-2,2-dimethylcyclopropane-1-carbonyl)-2,6-diazaspiro[3.4]octane-8-carboxamide (I-16):

(S)-N-((2S,3R)-3-(2-oxabicyclo[2.2.2]octan-4-ylmethoxy)-1-(dimethylamino)-1-oxobutan-2-yl)-6-(benzo[d]thiazol-7-yl)-2-((S)-2,2-dimethylcyclopropanecarbonyl)-2,6-diazaspiro[3.4]octane-8-carboxamide I-22 (0.035 g, 37%) as a yellow solid. ¹HNMR (400 MHz, CD₃OD): δ 9.15 (s, 1H), 7.52-7.49 (m, 1H), 7.42-7.38 (m, 1H), 6.74-6.71 (m, 1H), 4.93-4.88 (m, 1H), 4.56-4.39 (m, 1H), 4.31-4.24 (m, 1H), 4.18-3.82 (m, 6H), 3.74-3.59 (m, 4H), 3.49-3.39 (m, 1H), 3.20-3.14 (m, 4H), 3.03-2.93 (m, 4H), 1.97-1.80 (m, 2H), 1.65-1.33 (m, 8H), 1.19-1.08 (m, 9H), 1.07-1.03 (m, 1H), 0.81-0.76 (m, 1H). MS: [MH]⁺ 638.30.

### Synthesis of (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(4-(trifluoromethyl)thiazol-2-yl)-2,6-diazaspiro[3.4]octane-8-carboxamide I-17

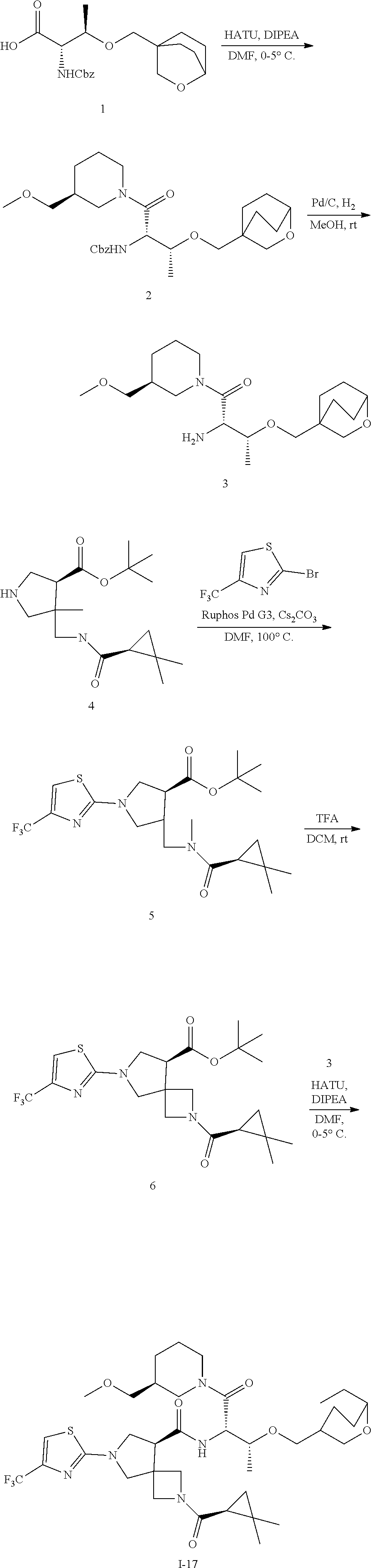

Step 1: Benzyl ((2S,3R)-3-(2-oxabicyclo[2.2.2]octan-4-ylmethoxy)-1-((S)-3-(methoxymethyl)piperidin-1-yl)-1-oxobutan-2-yl)carbamate (2): To a solution of (2S,3R)-3-(2-oxabicyclo[2.2.2]octan-4-ylmethoxy)-2-(((benzyloxy)carbonyl)amino)butanoic acid 1 (0.330 g, 0.87 mmol), (S)-3-(methoxymethyl)piperidine hydrochloride (0.159 g, 0.96 mmol), and N-ethyl-N-isopropylpropan-2-amine (0.339 g, 2.62 mmol) in N,N-dimethylformamide (3 mL) at 0-5° C. was added (2-(7-aza-1H-benzotriazole-1-yl)-1,1,3,3-tetramethyluronium hexafluorophosphate) (0.399 g, 1.05 mmol). The resulting mixture was stirred at 0-5° C. for 30 minutes. The reaction mixture was poured into water (20 mL) and extracted with ethyl acetate (10 mL×3). The combined organic layers were washed with brine (20 mL×2), dried over anhydrous sodium sulfate, and concentrated under reduced pressure to give a crude residue, which was purified by column chromatography using 50% ethyl acetate in hexane gradient to afford benzyl ((2S,3R)-3-(2-oxabicyclo[2.2.2]octan-4-ylmethoxy)-1-((S)-3-(methoxymethyl)piperidin-1-yl)-1-oxobutan-2-yl)carbamate 2 (0.350 g, 82%) as a colorless oil. MS: [MH]⁺ 489.3.

Step 2: (2S,3R)-3-(2-Oxabicyclo[2.2.2]octan-4-ylmethoxy)-2-amino-1-((S)-3-(methoxymethyl)piperidin-1-yl)butan-1-one (3): To a solution of benzyl ((2S,3R)-3-(2-oxabicyclo[2.2.2]octan-4-ylmethoxy)-1-((S)-3-(methoxymethyl)piperidin-1-yl)-1-oxobutan-2-yl)carbamate 2 (0.350 g, 0.72 mmol) in methanol (10 mL) was added palladium on carbon (10%, 0.100 g). The resulting mixture was stirred at room temperature under H₂ overnight. TLC showed the reaction was complete. Palladium on carbon was removed through filtration and washed with methanol (10 mL×2). The combined filtrates were concentrated under reduced pressure to afford (2S,3R)-3-(2-oxabicyclo[2.2.2]octan-4-ylmethoxy)-2-amino-1-((S)-3-(methoxymethyl)piperidin-1-yl)butan-1-one 3 (0.240 g, 94%) as a colorless oil. MS: [MH]⁺ 355.3.

Step 3: (S)-tert-Butyl 2-((S)-2,2-dimethylcyclopropanecarbonyl)-6-(4-(trifluoromethyl)thiazol-2-yl)-2,6-diazaspiro[3.4]octane-8-carboxylate (5): To a mixture of (S)-tert-butyl 2-((S)-2,2-dimethylcyclopropanecarbonyl)-2,6-diazaspiro[3.4]octane-8-carboxylate 4 (0.100 g, 0.32 mmol), 2-bromo-4-(trifluoromethyl)thiazole (0.083 g, 0.36 mmol), and cesium carbonate (0.211 g, 0.65 mmol) in N,N-dimethylformamide (2.5 mL) was added RuPhos Pd G3 (0.054 g, 0.065 mmol) under nitrogen atmosphere. The resulting mixture was stirred at 100° C. for 3 hours. The reaction mixture was poured into water (20 mL) and extracted with ethyl acetate (5 mL×3). The combined organic layers were washed with brine (20 mL×2), dried over anhydrous sodium sulfate, and concentrated under reduced pressure to give a crude residue, which was purified by silica gel column chromatography using a 25% ethyl acetate in hexane gradient to afford (S)-tert-butyl 2-((S)-2,2-dimethylcyclopropanecarbonyl)-6-(4-(trifluoromethyl)thiazol-2-yl)-2,6-diazaspiro[3.4]octane-8-carboxylate 5 (0.094 g, 63%) as a yellow oil. MS: [MH]⁺ 460.4.

Step 4: (S)-2-((S)-2,2-Dimethylcyclopropanecarbonyl)-6-(4-(trifluoromethyl)thiazol-2-yl)-2,6-diazaspiro[3.4]octane-8-carboxylic acid (6): To a solution of (S)-tert-butyl 2-((S)-2,2-dimethylcyclopropanecarbonyl)-6-(4-(trifluoromethyl)thiazol-2-yl)-2,6-diazaspiro[3.4]octane-8-carboxylate 5 (60 mg, 0.13 mmol) in dichloromethane (3 mL) was added 2,2,2-trifluoroacetic acid (2 mL). The resulting mixture was stirred at room temperature for 3 hours. TLC showed the reaction was complete. The volatiles were evaporated under reduced pressure to afford crude (S)-2-((S)-2,2-dimethylcyclopropanecarbonyl)-6-(4-(trifluoromethyl)thiazol-2-yl)-2,6-diazaspiro[3.4]octane-8-carboxylic acid 6 as a yellow oil, which was used in the next step without further purification. MS: [MH]⁺ 404.3.

Step 5: (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(4-(trifluoromethyl)thiazol-2-yl)-2,6-diazaspiro[3.4]octane-8-carboxamide (I-17A &I-17B): To a solution of crude (S)-2-((S)-2,2-dimethylcyclopropanecarbonyl)-6-(4-(trifluoromethyl)thiazol-2-yl)-2,6-diazaspiro[3.4]octane-8-carboxylic acid 6 (0.13 mmol), (2S,3R)-3-(2-oxabicyclo[2.2.2]octan-4-ylmethoxy)-2-amino-1-((S)-3-(methoxymethyl)piperidin-1-yl)butan-1-one 3 (0.051 g, 0.14 mmol), and N-ethyl-N-isopropylpropan-2-amine (0.051 g, 0.39 mmol) in N,N-dimethylformamide (1 mL) at 0-5° C. was added (2-(7-aza-1H-benzotriazole-1-yl)-1,1,3,3-tetramethyluronium hexafluorophosphate) (0.060 g, 0.16 mmol). The resulting mixture was stirred at 0-5° C. for an hour. The reaction mixture was poured into water (20 mL) and extracted with ethyl acetate (5 mL×3). The combined organic layers were washed with brine (20 mL×2), dried over anhydrous sodium sulfate, and concentrated under reduced pressure to give a crude residue, which was purified by preparative TLC using 5% methanol in dichloromethane gradient to afford (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(4-(trifluoromethyl)thiazol-2-yl)-2,6-diazaspiro[3.4]octane-8-carboxamide I-17A (0.021 g, 21%) and I-17B (0.018 g, 19%) as a white solid I-17A: ¹HNMR (400 MHz, CD₃OD): δ 7.37-7.11 (m, 1H), 7.22 (s, 1H), 4.96-4.90 (m, 1H), 4.42-4.13 (m, 4H), 4.07-4.03 (m, 1H), 3.98-3.64 (m, 10H), 3.54-3.45 (m, 1H), 3.27-3.12 (m, 3H), 3.06-3.02 (m, 1H), 2.93-2.86 (m, 1H), 2.73-2.62 (m, 1H), 2.04-1.94 (m, 2H), 1.85-1.62 (m, 7H), 1.53-1.34 (m, 6H), 1.19-1.14 (m, 9H), 1.07-1.04 (m, 1H), 0.81-0.77 (m, 1H). MS: [MH]⁺ 740.6. I-17B: ¹HNMR (400 MHz, CD₃OD): δ 7.36-7.11 (m, 1H), 7.21 (s, 1H), 4.96-4.86 (m, 1H), 4.53 (d, J=9.6 Hz, 1H), 4.41-4.23 (m, 2H), 4.18-3.63 (m, 13H), 3.50-3.44 (m, 1H), 3.28-3.11 (m, 4H), 3.01 (d, J=9.2 Hz, 1H), 2.75-2.60 (m, 1H), 2.02-1.91 (m, 2H), 1.87-1.77 (m, 2H), 1.72-1.60 (m, 5H), 1.51-1.41 (m, 4H), 1.38-1.31 (m, 2H), 1.18 (d, J=3.2 Hz, 3H), 1.13-1.10 (m, 6H), 1.06-1.02 (m, 1H), 0.80-0.76 (m, 1H). MS: [MH]⁺ 740.6.

The following compounds were prepared in a manner analogous to the procedures described above for (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(4-(trifluoromethyl)thiazol-2-yl)-2,6-diazaspiro[3.4]octane-8-carboxamide (1-17):

(S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(1H-pyrazolo[3,4-b]177yridine-3-yl)-2,6-diazaspiro[3.4]octane-8-carboxamide I-24 (0.013 g, 35%) as a white solid. ¹H NMR (400 MHz, CD₃OD): δ 8.38-8.31 (m, 2H), 7.06-7.03 (m, 1H), 4.97-4.89 (m, 1H), 4.56-3.84 (m, 11H), 3.69-3.63 (m, 4H), 3.48-3.41 (m, 1H), 3.30-3.29 (m, 2H) 3.27-3.13 (m, 5H), 3.03-2.85 (m, 2H), 2.76-2.60 (m, 1H), 1.91-1.39 (m, 13H), 1.19-1.18 (m, 3H), 1.15-1.11 (m, 6H), 1.07-1.06 (m, 1H), 0.81-0.77 (m, 1H). MS: [MH]⁺ 706.95.

### Synthesis of (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(5-(trifluoromethyl)benzo[d]thiazol-7-yl)-2,6-diazaspiro[3.4]octane-8-carboxamide I-21

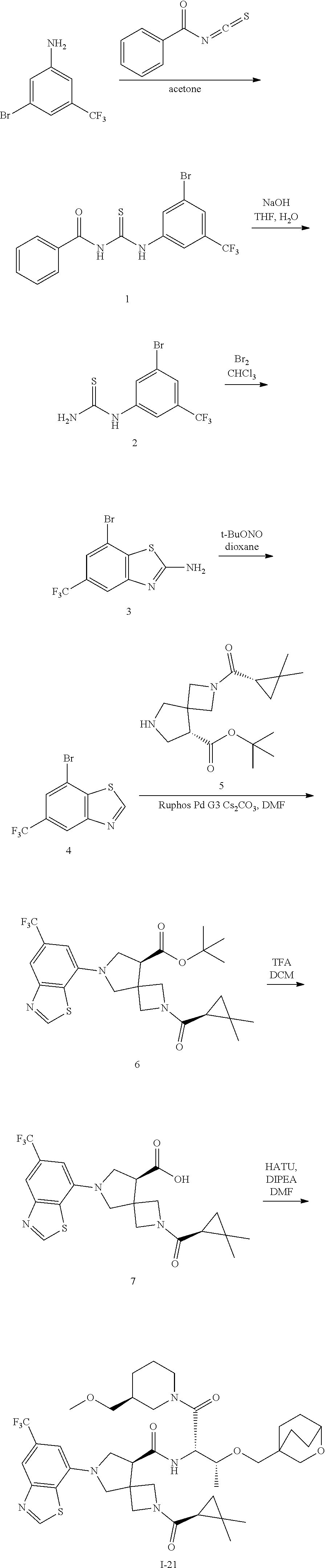

Step 1: N-((3-Bromo-5-(trifluoromethyl)phenyl)carbamothioyl)benzamide (1): To a solution of 3-bromo-5-(trifluoromethyl) aniline (5.500 g, 22.91 mmol) in acetone (100 mL) with stirring was added benzoyl isothiocyanate (4.114 g, 25.21 mmol) at room temperature. The resulting mixture was stirred for 1 hour. The volatiles were removed under reduced pressure to give a crude residue, which was triturated with hexane (50 mL). The resulting solid precipitate was collected through filtration and dried under vacuum to afford N-((3-bromo-5-(trifluoromethyl)phenyl)carbamothioyl)benzamide 1 (8.700 g, 94%) as a yellow solid. MS: [MH]⁺ 402.90.

Step 2: 1-(3-Bromo-5-(trifluoromethyl)phenyl)thiourea 2: A mixture of N-((3-bromo-5-(trifluoromethyl)phenyl)carbamothioyl)benzamide 1 (7.800 g, 19.34 mmol) and sodium hydroxide (3.900 g, 96.72 mmol) in THF (150 mL) and water (10 mL) was stirred at 85° C. for 5 hours. The reaction mixture was cooled down to room temperature and poured into water (20 mL). The mixture was extracted with ethyl acetate (5 mL×3). The combined organic layers were washed with brine (20 mL×2), dried over anhydrous sodium sulfate, and concentrated to ⅕ of the volume under reduced pressure. The solid was collected through filtration and dried in vacuo to afford 1-(3-bromo-5-(trifluoromethyl)phenyl)thiourea 2 (4.200 g, 73%) as a white solid. MS: [MH]⁺ 298.80; 300.65.

Step 3: 7-Bromo-5-(trifluoromethyl)benzo[d]thiazol-2-amine (3): To a solution of 1-(3-bromo-5-(trifluoromethyl)phenyl)thiourea 2 (1.800 g, 6.02 mmol) in chloroform (50 mL) at −60° C. was added a solution of bromine (1.400 g, 9.03 mmol) in chloroform (15 mL). The resulting reaction mixture was stirred at room temperature for 15 minutes, and then the temperature was raised to 70° C. and the mixture was stirred for an additional 1 hour. After cooling to room temperature, the reaction mixture was poured into saturated aqueous ammonium hydroxide solution (20 mL) and water (60 mL), and extracted with ethyl acetate (50 mL×3). The combined organic layers were washed with brine (50 mL×2), dried over anhydrous sodium sulfate, and concentrated under reduced pressure to give a crude residue, which was purified by silica gel column chromatography using a 10-30% ethyl acetate in hexane gradient to afford 7-bromo-5-(trifluoromethyl)benzo[d]thiazol-2-amine 3 (0.200 g, 11%) as a red solid. MS: [MH]⁺ 296.70; 298.55.

Step 4: 7-Bromo-5-(trifluoromethyl)benzo[d]thiazole (4): To a suspension of 7-bromo-5-(trifluoromethyl)benzo[d]thiazol-2-amine (3) (0.200 g, 0.71 mmol) in dioxane (10 mL) under nitrogen, was added tert-butyl nitrite (0.146 g, 1.41 mmol). The resulting mixture was heated at 85° C. for 1.5 hour. The reaction mixture was cooled to room temperature and concentrated under reduced pressure to give a crude residue, which was purified by silica gel column chromatography using a 2.5% ethyl acetate in hexane gradient to afford 7-bromo-5-(trifluoromethyl)benzo[d]thiazole 4 (0.138 g, 69%) as a white solid. MS: [MH]⁺ 281.70; 283.65.

Step 5: (S)-tert-butyl 2-((S)-2,2-dimethylcyclopropanecarbonyl)-6-(5-(trifluoromethyl)benzo[d]thiazol-7-yl)-2,6-diazaspiro[3.4]octane-8-carboxylate (6): To a mixture of (S)-tert-butyl 2-((S)-2,2-dimethylcyclopropanecarbonyl)-2,6-diazaspiro[3.4]octane-8-carboxylate 5 (0.135 g, 0.44 mmol), 7-bromo-5-(trifluoromethyl)benzo[d]thiazole 4 (0.135 g, 0.44 mmol), and cesium carbonate (0.285 g, 0.88 mmol) in N,N-dimethylformamide (4 mL) was added RuPhos Pd G3 (0.073 g, 0.09 mmol) under nitrogen atmosphere. The resulting mixture was stirred at 100° C. for 4 hours. The reaction mixture was cooled to room temperature, poured into water (20 mL), and extracted with ethyl acetate (15 mL×3). The combined organic layers were washed with brine (20 mL×2), dried over anhydrous sodium sulfate, and concentrated under reduced pressure to give a crude residue, which was purified by preparative TLC using a 10% dichloromethane in methanol to gradient afford (S)-tert-butyl 2-((S)-2,2-dimethylcyclopropanecarbonyl)-6-(5-(trifluoromethyl)benzo[d]thiazol-7-yl)-2,6-diazaspiro[3.4]octane-8-carboxylate 6 (0.060 g, 40%) as a yellow solid. MS. [MH]⁺ 510.15.

Step 6: (S)-2-((S)-2,2-dimethylcyclopropanecarbonyl)-6-(5-(trifluoromethyl)benzo[d]thiazol-7-yl)-2,6-diazaspiro[3.4]octane-8-carboxylic acid (7): To a solution of (S)-tert-butyl 2-((S)-2,2-dimethylcyclopropanecarbonyl)-6-(5-(trifluoromethyl)benzo[d]thiazol-7-yl)-2,6-diazaspiro[3.4]octane-8-carboxylate 6 (0.056 g, 0.11 mmol) in dichloromethane (2 mL) was added 2,2,2-trifluoroacetic acid (2 mL). The resulting mixture was stirred at room temperature for 3 hours. The volatiles were evaporated under reduced pressure to afford crude (S)-2-((S)-2,2-dimethylcyclopropanecarbonyl)-6-(5-(trifluoromethyl)benzo[d]thiazol-7-yl)-2,6-diazaspiro[3.4]octane-8-carboxylic acid 7 as a yellow oil, which was used in the next step without further purification. MS: [MH]⁺ 402.30.

Step 7: (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(5-(trifluoromethyl)benzo[d]thiazol-7-yl)-2,6-diazaspiro[3.4]octane-8-carboxamide (I-21): To a solution of crude (S)-2-((S)-2,2-dimethylcyclopropanecarbonyl)-6-(5-(trifluoromethyl)benzo[d]thiazol-7-yl)-2,6-diazaspiro[3.4]octane-8-carboxylic acid 7 (0.11 mmol), (2S,3R)-3-(2-oxabicyclo[2.2.2]octan-4-ylmethoxy)-2-amino-1-((S)-3-(methoxymethyl)piperidin-1-yl)butan-1-one (0.043 g, 0.12 mmol), and N-ethyl-N-isopropylpropan-2-amine (0.1 mL, 0.33 mmol) in N,N-dimethylformamide (1 mL) at 0-5° C. was added (2-(7-aza-1H-benzotriazole-1-yl)-1,1,3,3-tetramethyluronium hexafluorophosphate) (0.050 g. 0.13 mmol). The resulting mixture was stirred at 0-5° C. for 1 hour. The reaction mixture was poured into water (10 mL) and extracted with ethyl acetate (5 mL×3). The combined organic layers were washed with brine (20 mL×2), dried over anhydrous sodium sulfate, and concentrated under reduced pressure to give a crude residue, which was purified by preparative TLC using a 10% dichloromethane in methanol gradient to afford (S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(5-(trifluoromethyl)benzo[d]thiazol-7-yl)-2,6-diazaspiro[3.4]octane-8-carboxamide I-21 (0.054 g, 52%) as a red solid. ¹HNMR (400 MHz, CD₃OD): δ 9.15 (s, 1H), 7.33 (s, 1H), 7.08 (s, 1H), 4.87-7.81 (m, 1H), 4.47-4.45 (m, 2H), 4.31-4.07 (m, 3H), 3.99-3.54 (m, 11H), 3.41-3.35 (m, 1H), 3.21-3.03 (m, 6H), 2.95-2.76 (m, 2H), 2.63-2.53 (m, 1H), 1.83-1.70 (m, 3H), 1.52-1.49 (m, 5H), 1.37-1.30 (m, 4H), 1.09-1.01 (m, 9H), 0.98-0.93 (m, 1H), 0.72-0.66 (m, 1H). MS. [MH]⁺ 790.35.

The following compounds were prepared in a manner analogous to the procedures described above for (S)-N-((2S,3R)-3-(2-oxabicyclo[2.2.2]octan-4-ylmethoxy)-1-((S)-3-(methoxy methyl)piperidin-1-yl)-1-oxobutan-2-yl)-2-((S)-2,2-dimethylcyclopropanecarbonyl)-6-(5-(trifluoromethyl)benzo[d]thiazol-7-yl)-2,6-diazaspiro[3.4]octane-8-carboxamide (I-21):

(S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(5-hydroxybenzo[d]thiazol-7-yl)-2,6-diazaspiro[3.4]octane-8-carboxamide I-20 (0.068 g, 43%) as a red solid. ¹HNMR (400 MHz, CD₃OD): δ 9.06 (s, 1H), 6.90-6.89 (m, 1H), 6.25-6.23 (m, 1H), 4.96-4.89 (m, 1H), 4.56-3.60 (m, 15H), 3.44-3.89 (m, 1H), 3.29-3.13 (m, 5H), 3.02-2.62 (m, 3H), 1.93-1.42 (m, 13H), 1.18-1.05 (m, 10H), 0.82-0.76 (m, 1H). MS: [MH]⁺ 738.6.

(S)-N-((2S,3R)-3-(2-oxabicyclo[2.2.2]octan-4-ylmethoxy)-1-((S)-3-(methoxymethyl)piperidin-1-yl)-1-oxobutan-2-yl)-2-((S)-2,2-dimethylcyclopropanecarbonyl)-6-(5-fluorobenzo[d]thiazol-7-yl)-2,6-diazaspiro[3.4]octane-8-carboxamide I-18A (28 mg, yield 25%) and I-18B (29 mg, yield 26%) as an orange solid. I-18A: ¹H NMR (400 MHz, CD₃OD): δ 9.19 (s, 1H), 8.15-8.07 (m, 1H), 7.14-7.11 (m, 1H), 6.45 (d, J=6.0 Hz, 1H), 4.97-4.91 (m, 1H), 4.43-3.82 (m, 11H), 3.71-3.64 (m, 4H), 3.49-3.42 (m, 1H), 3.29-3.26 (m, 3H), 3.21 (d, J=3.2 Hz, 2H), 3.18-3.12 (m, 1H), 3.01 (t, J=10.2 Hz, 1H), 2.92-2.84 (m, 1H), 2.70-2.60 (m, 1H), 1.95-1.92 (m, 2H), 1.82-1.69 (m, 2H), 1.64-1.55 (m, 5H), 1.51-1.39 (m, 4H), 1.18 (s, 3H), 1.14-1.11 (m, 6H), 1.07-1.04 (m, 1H), 0.81-0.77 (m, 1H). MS: [MH]⁺ 740.4. I-18B: ¹H NMR (400 MHz, CD₃OD): δ 9.20-9.19 (m, 1H), 7.14-7.11 (m, 1H), 6.49-6.44 (m, 1H), 4.97-4.88 (m, 1H), 4.56 (d, J=4.8 Hz, 1H), 4.35-4.28 (m, 2H), 4.20-3.86 (m, 8H), 3.68-3.62 (m, 4H), 3.44-3.42 (m, 1H), 3.28-3.15 (m, 6H), 3.00-2.97 (m, 2H), 2.77-2.56 (m, 1H), 1.91-1.81 (m, 4H), 1.72-1.67 (m, 1H), 1.60-1.37 (m, 8H), 1.19 (s, 3H), 1.14-1.03 (m, 7H), 0.79-0.76 (m, 1H), 0.81-0.77 (m, 1H). MS: [MH]⁺ 740.2.

N-((2S,3R)-3-(2-oxabicyclo[2.2.2]octan-4-ylmethoxy)-1-oxo-1-(4-(pyridin-4-yl) piperidin-1-yl)butan-2-yl)-2-((S)-2,2-dimethylcyclopropanecarbonyl)-6-(5-fluorobenzo[d]thiazol-7-yl)-2,6-diazaspiro[3.4]octane-8-carboxamide I-33 (0.039 g, 40%) as a white solid. 1H NMR (400 MHz. CD₃OD): δ9.19-9.18 (m, 1H), 8.44-8.36 (m, 2H), 7.34-7.12 (m, 3H), 6.47 (d, J=5.8 Hz, 1H), 4.99-4.94 (m, 1H), 4.67-3.90 (m, 10H), 3.74-3.64 (m, 4H), 3.51-3.43 (m, 1H), 3.35 (s, 1H), 3.24-2.78 (m, 5H), 2.00-1.87 (m, 4H), 1.65-1.36 (m, 9H), 1.18-1.12 (m, 9H), 1.07-1.02 (m, 1H), 0.80-0.75 (m, 1H). MS: [MH]⁺ 773.75.

### Synthesis of (S)-N-((2S,3R)-3-(2-oxabicyclo[2.2.2]octan-4-ylmethoxy)-1-oxo-1-(2-oxa-7-azaspiro[3.5]nonan-7-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide I-19

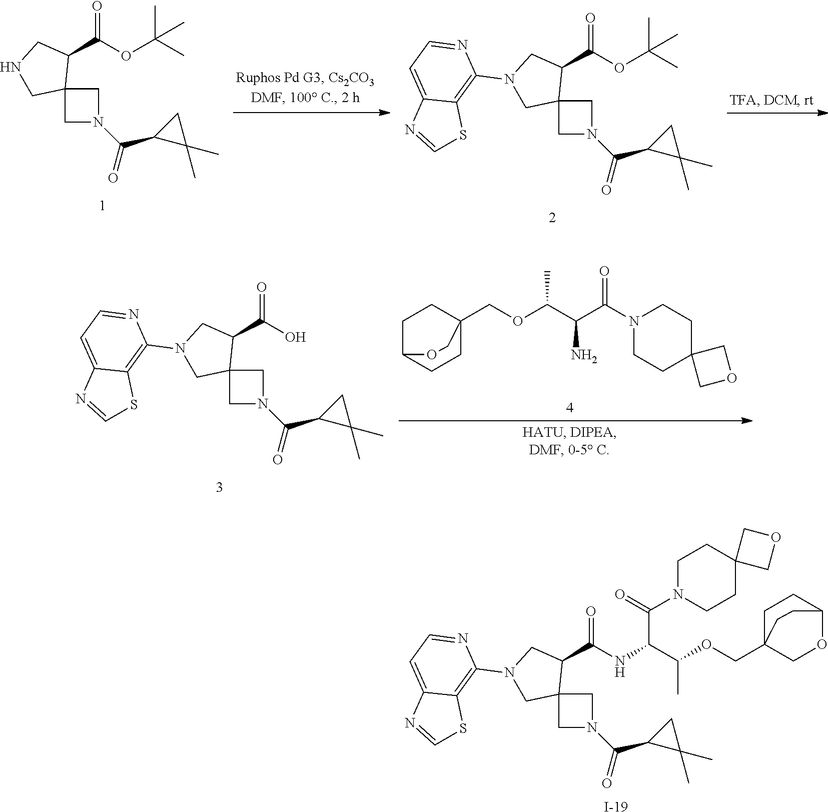

Step 1: (S)-tert-butyl 2-((S)-2,2-dimethylcyclopropanecarbonyl)-6-(thiazolo[5,4-c]pyridin-4-yl)-2,6-diazaspiro[3.4]octane-8-carboxylate (2): To a mixture of (S)-tert-butyl 2-((S)-2,2-dimethylcyclopropanecarbonyl)-2,6-diazaspiro[3.4]octane-8-carboxylate 1 (0.275 g, 0.89 mmol), 4-chlorothiazolo[5,4-c]pyridine (0.167 g, 0.98 mmol), and cesium carbonate (0.581 g, 1.78 mmol) in N,N-dimethylformamide (5 mL) was added RuPhos Pd G3 (0.149 g, 0.18 mmol) under nitrogen atmosphere. The resulting mixture was stirred at 100° C. under nitrogen atmosphere for 2 hours. The reaction mixture was cooled to room temperature. Next, the reaction mixture was poured into water (20 mL) and extracted with ethyl acetate (5 mL×3). The combined organic layers were washed with brine (20 mL×2), dried over anhydrous sodium sulfate, and concentrated under reduced pressure to give a crude residue, which was purified by silica gel column chromatography using a 50% ethyl acetate in hexane gradient to afford (S)-tert-butyl 2-((S)-2,2-dimethylcyclopropanecarbonyl)-6-(thiazolo[5,4-c]pyridin-4-yl)-2,6-diazaspiro[3.4]octane-8-carboxylate 2 (0.057 g, 14%) as a yellow oil. MS: [MH]⁺ 443.4.

Step 2: (S)-2-((S)-2,2-dimethylcyclopropanecarbonyl)-6-(thiazolo[5,4-c]pyridin-4-yl)-2,6-diazaspiro[3.4]octane-8-carboxylic acid (3): To a solution of (S)-tert-butyl 2-((S)-2,2-dimethylcyclopropanecarbonyl)-6-(thiazolo[5,4-c]pyridin-4-yl)-2,6-diazaspiro[3.4]octane-8-carboxylate 2 (0.053 g, 0.12 mmol) in dichloromethane (3 mL) was added 2,2,2-trifluoroacetic acid (2 mL). The reaction mixture was stirred at room temperature for 3 hours. The volatiles were evaporated under reduced pressure to afford crude (S)-2-((S)-2,2-dimethylcyclopropanecarbonyl)-6-(thiazolo[5,4-c]pyridin-4-yl)-2,6-diazaspiro[3.4]octane-8-carboxylic acid 3 as a yellow oil, which was used in next step without further purification. MS: [MH]⁺ 387.3.

Step 3: (S)-N-((2S,3R)-3-(2-oxabicyclo[2.2.2]octan-4-ylmethoxy)-1-oxo-1-(2-oxa-7-azaspiro[3.5]nonan-7-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide (I-19): To a solution of crude (S)-2-((S)-2,2-dimethylcyclopropanecarbonyl)-6-(thiazolo[5,4-c]pyridin-4-yl)-2,6-diazaspiro[3.4]octane-8-carboxylic acid 3 (0.12 mmol), (2S,3R)-3-(2-oxabicyclo[2.2.2]octan-4-ylmethoxy)-2-amino-1-(2-oxa-7-azaspiro[3.5]nonan-7-yl)butan-1-one 4 (0.046 g, 0.13 mmol), and N-ethyl-N-isopropylpropan-2-amine (0.046 g, 0.36 mmol) in N,N-dimethylformamide (1 mL) at 0-5° C. was added (2-(7-aza-1H-benzotriazole-1-yl)-1,1,3,3-tetramethyluronium hexafluorophosphate) (0.055 g, 0.14 mmol). The resulting mixture was stirred at 0-5° C. for 1 hour. The reaction mixture was poured into water (20 mL) and extracted with ethyl acetate (5 ml×3). The combined organic layers were washed with brine (20 mL×2), dried over anhydrous sodium sulfate, and concentrated under reduced pressure to give a crude residue, which was purified by preparative TLC using a 5% methanol in dichloromethane gradient to afford (S)-N-((2S,3R)-3-(2-oxabicyclo[2.2.2]octan-4-ylmethoxy)-1-oxo-1-(2-oxa-7-azaspiro[3.5]nonan-7-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide I-19 (0.040 g, 52%) as a light-yellow solid. ¹HNMR (400 MHz, CD₃OD): δ 9.37 (s, 1H), 8.07-8.05 (m, 1H), 7.29 (d, J=6.0 Hz, 1H), 4.94-4.90 (m, 1H), 4.58-4.08 (m, 11H), 4.00-3.93 (m, 1H), 3.72-3.40 (m, 9H), 3.23-3.16 (m, 1H), 3.03-2.97 (m, 1H), 1.99-1.74 (m, 6H), 1.66-1.33 (m, 8H), 1.20-1.04 (m, 10H), 0.82-0.76 (m, 1H). MS: [MH]⁺ 721.25.

The following compounds were prepared in a manner analogous to the procedures described above for (S)-N-((2S,3R)-3-(2-oxabicyclo[2.2.2]octan-4-ylmethoxy)-1-oxo-1-(2-oxa-7-azaspiro[3.5]nonan-7-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide (I-19):

(S)-N-((2S,3R)-3-(2-oxabicyclo[2.2.2]octan-4-ylmethoxy)-1-(dimethylamino)-1-oxobutan-2-yl)-2-((S)-2,2-dimethylcyclopropanecarbonyl)-6-(thiazolo[5,4-c]pyridin-4-yl)-2,6-diazaspiro[3.4]octane-8-carboxamide I-23 (0.035 g, 50% yield) as a yellow solid. ¹HNMR (400 MHz, CD₃OD): δ 9.36 (s, 1H), 8.06 (d, J=5.6 Hz, 1H), 7.29 (d, J=5.6 Hz, 1H), 4.94-4.88 (m, 1H), 4.56-4.39 (m, 1H), 4.32-4.07 (m, 6H), 4.01-3.96 (m, 1H), 3.75-3.63 (m, 4H), 3.52-3.43 (m, 1H), 3.22-3.14 (m, 4H), 3.06-3.00 (m, 1H), 2.95-2.93 (m, 3H), 2.00-1.87 (m, 2H), 1.66-1.33 (m, 8H), 1.20-1.04 (m, 10H), 0.82-0.76 (m, 1H). MS: [MH]⁺ 639.70.

Benzyl 2-((S)-2,2-dimethylcyclopropanecarbonyl)-6-(5-fluorobenzo[d]thiazol-7-yl)-N-((2S,3R)-1-oxo-1-(4-(pyridin-4-yl)piperidin-1-yl)-3-((1-(trifluoromethyl)cyclopropyl) methoxy)butan-2-yl)-2,6-diazaspiro[3.4]octane-8-carboxamide I-34 (0.026 g, 25%) as a white solid. ¹HNMR (400 MHz. CD₃OD): δ 9.18-9.16 (m, 1H), 8.44-8.34 (m, 2H), 7.34-7.11 (m, 3H), 6.47-6.43 (s, 1H), 5.04-4.99 (m, 1H), 4.70-4.57 (m, 1H), 4.44-4.18 (m, 3H), 4.08-3.81 (m, 6H), 3.75-3.44 (m, 3H), 3.27-3.18 (m, 1H), 2.95-2.71 (m, 2H), 2.01-1.41 (m, 5H), 1.21-1.12 (m, 9H), 1.07-1.05 (m, 1H), 0.96-0.78 (m, 5H). MS: [MH]⁺ 771.65.

### Synthesis of (S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-48

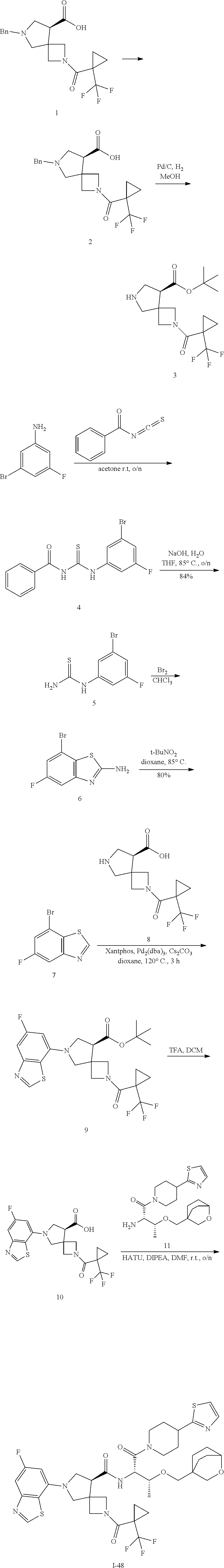

Step 1: (S)-tert-butyl 6-benzyl-2-(1-(trifluoromethyl)cyclopropanecarbonyl)-2,6-diazaspiro[3.4]octane-8-carboxylate (2): To a solution of (S)-6-benz: 1-2-(1-(trifluoromethyl)cyclopropanecarbonyl)-2,6-diazaspiro[3.4]octane-8-carboxylic acid 1 (5.600 g. 14.65 mmol) in DCM (73 mL) was added 2-tert-butyl-1,3-diisopropylisourea (8.800 g, 43.94 mmol). The mixture was stirred at room temperature for 30 minutes. An additional 1.5 eq of 2-tert-butyl-1,3-diisopropylisourea was added, and the resulting mixture was stirred at room temperature under nitrogen atmosphere overnight. The reaction mixture was filtered through a pad of Celite. and the filtrate was concentrated to give a crude residue, which was purified by silica gel flash column chromatography using a 20% ethyl acetate in hexane gradient to afford (S)-tert-butyl 6-benzyl-2-(1-(trifluoromethyl)cyclopropanecarbonyl)-2,6-diazaspiro[3.4]octane-8-carboxylate 2 (3.600 g, 56% yield) as a white solid. MS: [MH]⁺ 439.85.

Step 2: (S)-tert-butyl 2-(1-(trifluoromethyl)cyclopropanecarbonyl)-2,6-diazaspiro[3.4]octane-8-carboxylate (3): A mixture of (S)-tert-butyl 6-benzyl-2-(1-(trifluoromethyl)cyclopropanecarbonyl)-2,6-diazaspiro[3.4]octane-8-carboxylate 2 (3.600 g, 8.20 mmol) and Pd/C (10%, 0.400 g) in methanol (50 mL) was stirred at room temperature under hydrogen atmosphere overnight. The reaction mixture was filtered, and the filtrate was concentrated to give a crude residue, which was purified by silica gel flash column chromatography using a 10% methanol in dichloromethane gradient to afford (S)-tert-butyl 2-(1-(trifluoromethyl)cyclopropanecarbonyl)-2,6-diazaspiro[3.4]octane-8-carboxylate 3 (2.600 g, 94% yield) as a white solid. ¹HNMR (400 MHz, CDCl₃): δ 4.40-3.93 (m, 4H), 3.24-3.19 (m, 3H), 3.13 (d, J=10.8 Hz, 1H), 2.89 (t, J=6.8 Hz, 1H), 1.47 (s, 9H), 1.22 (s, 4H).

Step 3: N-((3-bromo-5-fluorophenyl)carbamothioyl)benzamide (4): To a solution of 3-bromo-5-fluoroaniline (4.000 g, 21.05 mmol) in acetone (80 mL) was added benzoyl isothiocyanate (3.780 g, 23.16 mmol), and the resulting mixture was stirred at room temperature under nitrogen atmosphere overnight. The reaction mixture was concentrated to give a crude residue, which was triturated with hexanes and dried to afford N-((3-bromo-5-fluorophenyl)carbamothioyl)benzamide 4 (7.05 g, yield 94%) as a yellow solid. MS: [MH]⁺ 353.1.

Step 4: 1-(3-Bromo-5-fluorophenyl)thiourea (5): To a solution of N-((3-bromo-5-fluorophenyl)carbamothioyl)benzamide 4 (6.050 g, 17.13 mmol) in tetrahydrofuran (90 mL) was added aqueous sodium hydroxide solution (3.430 g, 85.64 mmol, in 6 mL of water). The mixture was stirred at 85° C. under nitrogen atmosphere overnight. The reaction mixture was cooled to room temperature, poured into water (100 mL), and extracted with ethyl acetate (100 mL×3). The combined organic layers were washed with brine (300 mL), dried over anhydrous sodium sulfate, and concentrated to give a crude residue, which was purified by silica gel flash column chromatography using a 33% ethyl acetate in hexane gradient to afford 1-(3-bromo-5-fluorophenyl)thiourea 5 (3.60 g, yield 84%) as a white solid. MS: [MH]⁺ 249.0

Step 5: 7-Bromo-5-fluorobenzo[d]thiazol-2-amine (6): To a solution of 1-(3-bromo-5-fluorophenyl)thiourea 5 (0.700 g, 2.81 mmol) in chloroform (20 mL) at −60° C. was added a solution of bromine (0.449 g, 2.81 mmol) in chloroform (5 mL). The resulting mixture was stirred at room temperature for 15 minutes, and then the temperature was raised to 70° C. The mixture was stirred for 1 hour. The reaction mixture was cooled to room temperature, basified to pH 9 with saturated aqueous ammonium hydroxide solution, and extracted with ethyl acetate (15 mL×3). The combined organic layers were washed with brine (30 mL), dried over anhydrous sodium sulfate, and concentrated to give a crude residue, which was purified by silica gel flash column chromatography using a 20% ethyl acetate in hexane gradient to afford 7-bromo-5-fluorobenzo[d]thiazol-2-amine 6 (0.500 g. yield 72%) as a yellow solid. MS: [MH]⁺ 247.3

Step 6: 7-Bromo-5-fluorobenzo[d]thiazole (7): To a solution of 7-bromo-5-fluorobenzo[d]thiazol-2-amine 6 (0.500 g, 2.02 mmol) in 1,4-dioxane (20 mL) was added tert-butyl nitrite (0.422 g, 4.09 mmol). The resulting mixture was stirred at 85° C. under nitrogen atmosphere for 1 hour. The reaction mixture was concentrated to give a crude residue, which was purified by silica gel flash column chromatography using a 2.5% ethyl acetate in hexane gradient to afford 7-bromo-5-fluorobenzo[d]thiazole 7 (0.375 g, yield 80%) as a yellow solid. MS: [MH]⁺ 232.7

Step 7: tert-Butyl (S)-6-(5-fluorobenzo[d]thiazol-7-yl)-2-(1-(trifluoromethyl)cyclopropane-1-carbonyl)-2,6-diazaspiro[3.4]octane-8-carboxylate (9): To a solution of 7-bromo-5-fluorobenzo[d]thiazole 7 (0.050 g, 0.22 mmol), tert-butyl (S)-2-(1-(trifluoromethyl)cyclopropane-1-carbonyl)-2,6-diazaspiro[3.4]octane-8-carboxylate 8 (0.075 g, 0.22 mmol), 9,9-dimethyl-4,5-bisdiphenyl phosphinoxanthene (0.050 g, 0.096 mmol), and cesium carbonate (0.105 g, 0.32 mmol) in dioxane (2.5 mL) was added tris(dibenzylideneacetone) dipalladium (0)) (39.5 mg, 0.043 mmol) under nitrogen atmosphere. The mixture was stirred at 120° C. for 3 hours. The reaction mixture was quenched with water (10 mL), and extracted with ethyl acetate (15 mL). The organic layer was washed with brine (5 mL), dried over anhydrous sodium sulfate, and concentrated to give a crude residue, which was purified by silica gel column chromatography using a 17% ethyl acetate in hexane gradient to afford tert-butyl (S)-6-(5-fluorobenzo[d]thiazol-7-yl)-2-(1-(trifluoromethyl)cyclopropane-1-carbonyl)-2,6-diazaspiro[3.4]octane-8-carboxylate 9 (0.062 g, yield 50%) as a yellow solid. MS: [MH]⁺ 500.80.

Step 8: (S)-6-(5-fluorobenzo[d]thiazol-7-yl)-2-(1-(trifluoromethyl)cyclopropane-1-carbonyl)-2,6-diazaspiro[3.4]octane-8-carboxylic acid (10): To a solution of tert-butyl (S)-6-(5-fluorobenzo[d]thiazol-7-yl)-2-(1-(trifluoromethyl)cyclopropane-1-carbonyl)-2,6-diazaspiro[3.4]octane-8-carboxylate 9 (0.100 g, 0.20 mmol) in dichloromethane (3 mL) was added 2,2,2-trifluoroacetic acid (1 mL). The mixture was stirred at 40° C. for 16 hours. The reaction mixture was concentrated under reduced pressure to afford (S)-6-(5-fluorobenzo[d]thiazol-7-yl)-2-(1-(trifluoromethyl)cyclopropane-1-carbonyl)-2,6-diazaspiro[3.4]octane-8-carboxylic acid 10 (0.083 g, yield 93%) as a yellow solid, which was used in the next step without further purification. MS: [MH]⁺ 444.2

Step 9: (S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-48: To a solution of (S)-6-(5-fluorobenzo[d]thiazol-7-yl)-2-(1-(trifluoromethyl)cyclopropane-1-carbonyl)-2,6-diazaspiro[3.4]octane-8-carboxylic acid 10 (0.083 g, 0.19 mmol). (2S,3R)-3-((2-oxabicyclo[2.2.2]octan-4-yl)methoxy)-2-amino-1-(4-(thiazol-2-yl)piperidin-1-yl)butan-1-one TFA salt 11 (0.093 g, 0.19 mmol), and N-ethyl-N-isopropylpropan-2-amine (0.193 g, 1.50 mmol) in N,N-dimethylformamide (0.5 mL) at 0-5° C. was added HATU (2-(7-aza-1H-benzotriazole-1-yl)-1,1,3,3-tetramethyluronium hexafluorophosphate) (0.107 g, 0.28 mmol). The resulting mixture was stirred at room temperature for 16 hours. The reaction mixture was poured into water (10 mL) and extracted with ethyl acetate (5 mL×2). The combined organic layers were washed with water (10 mL) and brine (10 mL), dried over anhydrous sodium sulfate, and concentrated under reduced pressure to give a crude residue, which was purified by prep-TLC using a 5% methanol in dichloromethane gradient to afford (S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-48 (0.065 g, 40%) as a yellow solid. ¹HNMR (400 MHz, CD₃OD): δ 9.20-9.17 (m, 1H), 7.71-7.64 (m, 1H), 7.48-7.38 (m, 1H), 7.14-7.12 (m, 1H), 6.48-6.43 (m, 1H), 4.97-4.95 (m, 1H), 4.60-4.39 (m, 3H), 4.20-3.64 (m, 12H), 3.47-3.35 (m, 2H), 3.26-3.21 (m, 1H), 3.03-3.00 (m, 1H), 2.90-2.81 (m, 1H), 2.19-2.08 (m, 2H), 1.93 (s, 2H), 1.65-1.46 (m, 8H), 1.24 (s, 3H), 1.18-1.13 (m, 4H) MS: [MH]⁺ 819.8.

The following 6 compounds were prepared in a manner analogous to the procedures described above for (S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide (I-48):

(S)-N-((2S,3R)-3-(2-oxabicyclo[2.2.2]octan-4-ylmethoxy)-1-oxo-1-(4-(thiazol-2-yl)piperidin-1-yl)butan-2-yl)-6-(7-fluoroquinoxalin-5-yl)-2-(1-(trifluoromethyl)cyclopropanecarbonyl)-2,6-diazaspiro[3.4]octane-8-carboxamide I-44 (0.030 g, 40% yield) as a yellow solid. ¹HNMR (400 MHz, CD₃OD): δ 8.70-8.63 (m, 2H), 8.28-8.10 (m, 1H), 7.72-7.62 (m, 1H), 7.49-7.38 (m, 1H), 6.97-6.92 (m, 1H), 6.65-6.59 (m, 1H), 4.97-4.93 (m, 1H), 4.66-4.10 (m, 10H), 3.70-3.60 (m, 4H), 3.45-3.31 (m, 2H), 3.24-3.20 (m, 1H), 3.08-2.82 (m, 2H), 2.24-2.06 (m, 2H), 1.95-1.79 (m, 3H), 1.61-1.11 (m, 15H). MS: [MH]⁺ 814.90.

(S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(5-fluoro-1H-benzo[d]imidazol-7-yl)-2-(1-(trifluoromethyl)cyclopropane-1-carbonyl)-2,6-diazaspiro[3.4]octane-8-carboxamide I-41 (0.058 g, 61%) as an off white solid. ¹HNMR (400 MHz, CD₃OD): δ 8.28-8.12 (m, 1H), 7.96-7.93 (m, 1H), 7.71-7.65 (m, 1H), 7.48-7.40 (m, 1H), 6.57 (d, J=8.0 Hz, 1H), 6.12 (t, J=13.2 Hz, 1H). 4.95-4.93 (m, 1H), 4.61-3.88 (m, 11H), 3.71-3.62 (m, 4H), 3.43-3.35 (m, 2H), 3.22-3.16 (m, 1H), 3.04-2.99 (m, 1H), 2.91-2.83 (m, 1H), 2.23-2.07 (m, 2H), 2.03-1.79 (m, 3H), 1.65-1.35 (m, 8H), 1.24-1.20 (m, 3H), 1.16-1.10 (m, 3H). MS: [MH]⁺ 802.80.

(S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(2-hydroxyquinoxalin-5-yl)-2-(1-(trifluoromethyl)cyclopropane-1-carbonyl)-2,6-diazaspiro[3.4]octane-8-carboxamide I-45 (0.022 g, yield 47%) as a yellow solid. ¹H NMR (400 MHz. CD₃OD) δ 7.99-7.94 (m, 1H), 7.71-7.65 (m, 1H), 7.49-7.29 (m, 2H), 6.62-6.52 (m, 2H), 4.59-4.50 (m, 1H), 4.35-3.97 (m, 8H), 3.70-3.61 (m, 5H), 3.38-3.35 (m, 2H), 3.22-3.16 (m, 1H), 3.04-2.97 (m, 1H), 2.91-2.85 (m, 1H), 2.23-2.07 (m, 2H), 1.94-1.85 (m, 2H), 1.63-1.53 (m, 6H), 1.35 (d, J=6.4 Hz, 3H), 1.30-1.29 (m, 2H), 1.17-1.10 (m, 4H). MS: [MH]⁺ 812.85.

(S)-N-((2S,3R)-3-((2-oxabicyclo[2.2.2]octan-4-yl)methoxy)-1-oxo-1-(6-azaspiro[2.5]octan-6-yl)butan-2-yl)-6-(5-fluorobenzo[d]thiazol-7-yl)-2-(1-(trifluoromethyl)cyclopropane 1-carbonyl)-2,6-diazaspiro[3.4]octane-8-carboxamide I-43 (0.024 g. yield 39%) as a white solid. ¹HNMR (400 MHz. CD₃OD): δ 9.19 (s, 1H), 8.30-8.13 (m, 1H), 7.14-7.11 (m, 1H), 6.49-6.44 (m, 1H), 4.96-4.92 (m, 1H), 4.60-4.38 (m, 2H), 4.23-3.95 (m, 4H), 3.90-3.88 (m, 2H), 3.71-3.63 (m, 7H), 3.56-3.44 (m, 2H), 3.24-3.19 (m, 1H), 3.02-3.97 (m, 1H), 1.98-1.87 (m, 2H), 1.64-1.53 (m, 4H), 1.48-1.34 (m, 5H), 1.25 (s. 5H), 1.14-1.11 (m, 3H), 0.40-0.18 (m, 4H). LCMS: [MH]⁺ 762.70.

(S)-N-((2S,3R)-3-(2-oxabicyclo[2.2.2]octan-4-ylmethoxy)-1-oxo-1-(2-oxa-7-azaspiro[3.5]nonan-7-yl)butan-2-yl)-6-(5-fluorobenzo[d]thiazol-7-yl)-2-(1-(trifluoromethyl)cyclopropanecarbonyl)-2,6-diazaspiro[3.4]octane-8-carboxamide I-46 (0.057 g, 54%) as a white solid. ¹HNMR (400 MHz. CD₃OD): δ 9.19 (s, 1H), 7.15-7.11 (m, 1H), 6.48-6.43 (m, 1H). 4.91 (d, J=4.4 Hz, 1H), 4.49-4.25 (m, 6H), 4.11-3.96 (m, 3H), 3.89-3.87 (m, 2H), 3.71-3.58 (m, 6H), 3.46-3.41 (m, 2H), 3.22-3.18 (m, 1H), 3.00-2.96 (m, 1H), 2.01-1.79 (m, 6H), 1.64-1.53 (m, 4H), 1.47-1.29 (m, 5H), 1.23-1.19 (m, 1H), 1.13-1.09 (m, 3H). MS: [MH]⁺ 778.65.

(S)-N-((2S,3R)-3-((2-oxabicyclo[2.2.2]octan-4-yl)methoxy)-1-oxo-1-(4-(trifluoromethyl)piperidin-1-yl)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-42 (0.039 g. yield 59%) as a white solid. ¹HNMR (400 MHz. CD₃OD): δ 9.20-9.19 (m, 1H), 8.31-8.13 (m, 1H), 7.14-7.11 (m, 1H), 6.47-6.44 (m, 1H), 4.97-4.90 (m, 1H), 4.61-4.32 (m, 3H), 4.24-4.10 (m, 2H), 4.05-3.94 (m, 2H), 3.89-3.87 (m, 2H), 3.70-3.59 (m, 4H), 3.48-3.43 (m, 1H), 3.25-3.12 (m, 2H), 3.01-2.97 (m, 1H), 2.69-2.47 (m, 2H), 2.01-1.85 (m, 4H), 1.64-1.50 (m, 4H), 1.48-1.35 (m, 4H), 1.26-1.24 (s. 4H), 1.17-1.09 (m, 3H). LCMS: [MH]⁺ 804.75.

### Synthesis of 2-(4-((2R,3R)-3-(2-oxabicyclo[2.2.2]octan-4-ylmethoxy)-2-((S)-6-(5-fluorobenzo[d]thiazol-7-yl)-2-(1-(trifluoromethyl)cyclopropanecarbonyl)-2,6-diazaspiro[3.4]octane-8-carboxamido)butoxy)cyclohexyl)benzoic acid I-53

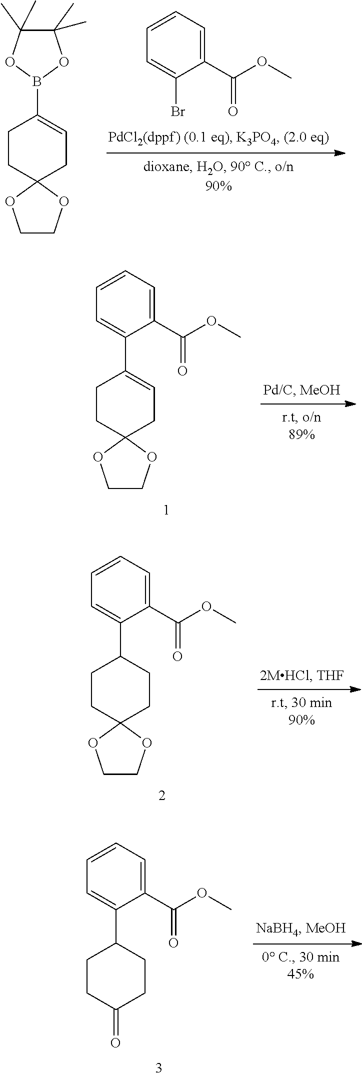

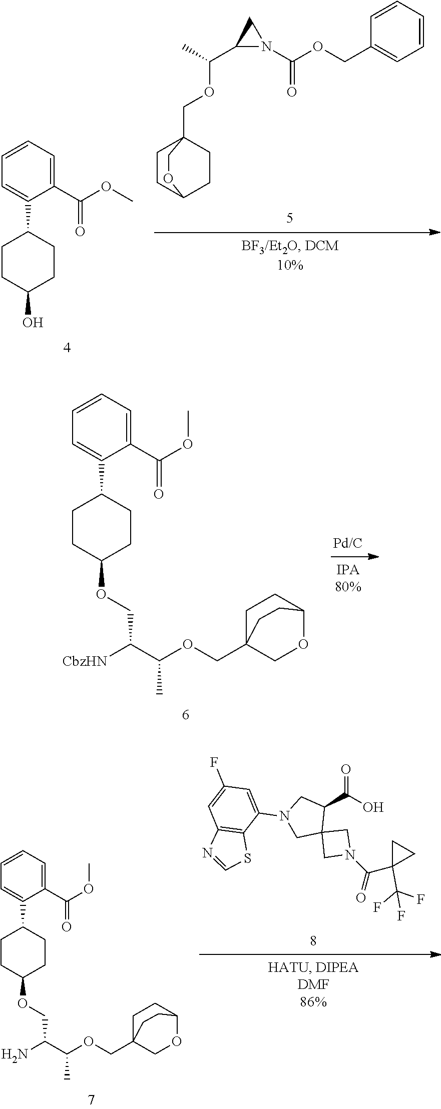

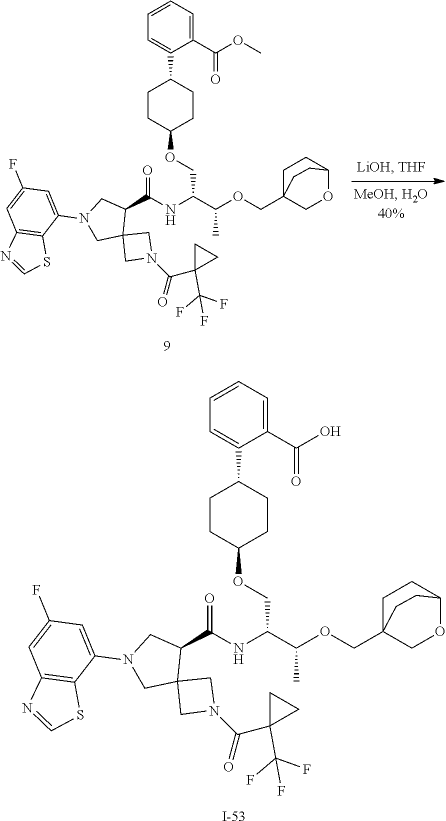

Step 1: Methyl 2-(1,4-dioxaspiro[4.5]dec-7-en-8-yl)benzoate (1): To a mixture of methyl 2-bromobenzoate (1.305 g, 6.10 mmol), 4,4,5,5-tetramethyl-2-(1,4-dioxaspiro[4.5]dec-7-en-8-yl)-1,3,2-dioxaborolane (1.947 g, 7.316 mmol), and sodium carbonate (3.231 g, 30.485 mmol) in 1,4-dioxane (30 mL)-water (15 mL) was added tetrakis(triphenylphosphine) palladium (0.352 g, 0.305 mmol) under N₂ atmosphere, and the resulting mixture was stirred at 90° C. under nitrogen atmosphere for 6 hours. The reaction mixture was poured into water (10 mL) and extracted with ethyl acetate (30 mL×2). The combined organic layers were washed with brine (30 mL), dried over anhydrous sodium sulfate, and concentrated to give a crude residue, which was purified by silica gel column chromatography using a 12.5% ethyl acetate in hexane gradient to afford methyl 2-(1,4-dioxaspiro[4.5]dec-7-en-8-yl)benzoate 1 (1.510 g, 90%) as a yellow oil. MS: [MH]⁺ 275.20.

Step 2: Methyl 2-(1,4-dioxaspiro[4.5]decan-8-yl)benzoate (2): To a solution of methyl 4-(1,4-dioxaspiro[4.5]dec-7-en-8-yl)benzoate 1 (1.510 g, 5.49 mmol) in ethyl acetate (70 mL) was added palladium (10% on carbon, 0.650 g), and the mixture was stirred at 40° C. under hydrogen overnight. The reaction mixture was filtered, and the filtrate was concentrated to give a crude residue, which was purified by silica gel column chromatography using a hexane-ethyl acetate-dichloromethane (3:1:1, v/v/v) gradient to afford methyl 2-(1,4-dioxaspiro[4.5]decan-8-yl)benzoate 2 (1.353 g, 89% yield) as a colorless oil. MS: [MH]⁺ 277.10.

Step 3: Methyl 2-(4-oxocyclohexyl)benzoate (3): A solution of methyl 2-(1,4-dioxaspiro[4.5]decan-8-yl)benzoate (2) (1.353 g, 4.89 mmol) and pyridinium p-toluenesulfonate (1.272 g, 5.06 mmol) in a mixture of acetone (12 mL) and water (12 mL) was refluxed overnight. The reaction mixture was concentrated in vacuo. The residue was taken up in ethyl acetate (25 mL), washed with brine (30 mL), dried over anhydrous sodium sulfate, and concentrated to give methyl 2-(4-oxocyclohexyl)benzoate 3 (1.166 g, 90%) as a white solid, which was used in the next step without further purification. MS: [MH]⁺ 233.30.

Step 4: Methyl 2-(trans-4-hydroxycyclohexyl)benzoate (4): To a solution of methyl 4-(4-oxocyclohexyl)benzoate 3 (1.025 g, 4.40 mmol) in methanol (15 mL) was added sodium borohydride (0.193 g, 16.55 mmol) at (° C.) The resulting mixture was stirred at room temperature for 1.5 hours. The reaction mixture was poured into water (10 mL) and extracted with ethyl acetate (20 mL×3). The combined organic layers were washed with brine (30 mL), dried over anhydrous sodium sulfate, and concentrated to give a crude residue, which was purified by silica gel flash column chromatography using a hexane-ethyl acetate-dichloromethane (1:1:1, v/v/v) gradient to give methyl 2-(trans-4-hydroxycyclohexyl)benzoate 4 (0.465 g, 45%) as a white solid. MS: [MH]⁺ 235.20.

Step 5: Methyl 2-(4-((2R,3R)-3-(2-oxabicyclo[2.2.2]octan-4-ylmethoxy)-2-(((benzyloxy)carbonyl)amino)butoxy)cyclohexyl)benzoate (6): To a solution of (R)-benzyl 2-((R)-1-(2-oxabicyclo[2.2.2]octan-4-ylmethoxy)ethyl) aziridine-1-carboxylate 5 (0.670 g, 1.94 mmol) and trans-methyl 2-(4-hydroxycyclohexyl)benzoate 4 (0.465 g, 1.98 mmol) in chloroform (30 mL) at 0° C.) was added boron trifluoride etherate (0.193 g, 1.36 mmol) slowly, and the resulting mixture was stirred at 0-5° C. under nitrogen atmosphere for one hour. The reaction mixture was concentrated to give a crude residue, which was purified through silica gel column chromatography using a hexane-ethyl acetate-dichloromethane (3:1:1, v/v/v) gradient to afford methyl 2-(4-((2R,3R)-3-(2-oxabicyclo[2.2.2]octan-4-ylmethoxy)-2-(((benzyloxy)carbonyl)amino)butoxy)cyclohexyl)benzoate 6 (0.116 g, 10% yield) as a colorless oil. MS: [MH]⁺ 580.65

Step 6: Trans-methyl 2-(4-((2R,3R)-3-(2-oxabicyclo[2.2.2]octan-4-ylmethoxy)-2-aminobutoxy)cyclohexyl)benzoate (7): To a solution of methyl 2-(4-((2R,3R)-3-(2-oxabicyclo[2.2.2]octan-4-ylmethoxy)-2-(((benzyloxy)carbonyl)amino)butoxy)cyclohexyl)benzoate 6 (0.116 g, 0.19 mmol) in iPrOH (15 mL) was added Pd/C (10%, 0.020 g), and the mixture was stirred at room temperature under hydrogen overnight. The reaction mixture was filtered, and the filtrate was concentrated to afford trans-methyl 2-(4-((2R,3R)-3-(2-oxabicyclo[2.2.2]octan-4-ylmethoxy)-2-aminobutoxy)cyclohexyl)benzoate 7 (0.068 g, 80% yield) as a white solid, which was used in the next step without further purification. MS: [MH]⁺ 447.00.

Step 7: Methyl 2-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoate (9): To a solution of trans-methyl 2-(4-((2R,3R)-3-(2-oxabicyclo[2.2.2]octan-4-ylmethoxy)-2-aminobutoxy)cyclohexyl)benzoate 7 (0.064 g, 0.14 mmol), (S)-6-(5-fluorobenzo[d]thiazol-7-yl)-2-(1-(trifluoromethyl)cyclopropane-1-carbonyl)-2,6-diazaspiro[3.4]octane-8-carboxylic acid 8 (0.080 g, 0.15 mmol), and N-ethyl-N-isopropylpropan-2-amine (0.056 g, 0.43 mmol) in N,N-dimethylformamide (2 mL) at 0-5° C. was added (2-(7-aza-1H-benzotriazole-1-yl)-1,1,3,3-tetramethyluronium hexafluorophosphate) (0.066 g, 0.17 mmol). The resulting mixture was stirred at room temperature under nitrogen for one hour. The reaction mixture was poured into aqueous ammonium chloride solution (10 mL) and extracted with ethyl acetate (10 mL×2). The combined organic layer was washed with water (8 mL×3) and brine (8 mL), dried over anhydrous sodium sulfate, and concentrated under reduced pressure to give a crude residue which was purified by prep-TLC using a 3.3% methanol in dichloromethane gradient to afford methyl 2-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoate (9) (0.100 g, 86% yield) as a white solid. MS: [MH]⁺ 871.7.

Step 8: 2-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoic acid (I-53): To a solution of methyl 2-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoate (9) (0.100 g, 0.11 mmol) in a mixture of tetrahydrofuran (4 mL)-methanol (2 mL)-water (1 mL) was added lithium hydroxide monohydrate (0.053 g, 1.30 mmol). The resulting mixture was stirred at 40° C. for 3 hours. The reaction mixture was concentrated. The residue was acidified to pH of 6 with diluted hydrochloric acid (1 N), and extracted with dichloromethane (6 mL×3). The combined organic layers were dried over anhydrous sodium sulfate and concentrated under reduced pressure to give a crude residue, which was purified by prep-TLC using a 5% methanol in dichloromethane gradient to afford 2-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoic acid I-53 (0.040 g, yield 40%) as a white solid. ¹H NMR (400 MHz, CD₃OD): δ 9.22 (s, 1H), 8.04-7.88 (m, 1H), 7.68-7.60 (m, 1H), 7.45-7.34 (m, 2H), 7.25-7.13 (m, 2H), 6.52-6.41 (m, 1H), 4.70-4.28 (m, 3H), 4.15-3.82 (m, 6H), 3.78-3.69 (m, 3H), 3.66-3.47 (m, 4H), 3.43-3.37 (m, 2H), 3.27-3.19 (m, 1H), 3.02-2.94 (m, 1H), 2.25-1.87 (m, 6H), 1.73-1.46 (m, 8H), 1.39-1.29 (m, 6H), 1.20-1.10 (m, 3H). MS: [MH]⁺ 857.35.

### Synthesis of 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexane-1-carboxylic acid I-56

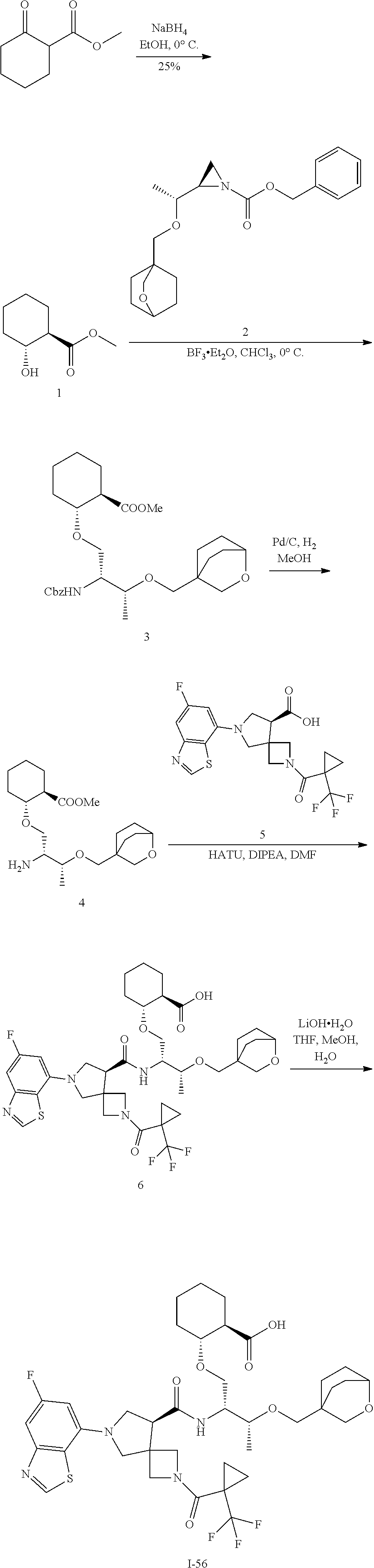

Step 1: Methyl trans-2-hydroxycyclohexane-1-carboxylate (1): To a stirred solution of methyl 2-oxocyclohexane-1-carboxylate (4.000 g, 30.10 mmol) in ethanol (80 mL) at 0° C. under nitrogen atmosphere was added sodium borohydride (0.292 g, 0.01 mmol). The resulting mixture was stirred for 2 hours. The reaction mixture was quenched with water (20 mL) and extracted with ethyl acetate (20 mL). The combined organic layers were washed with brine (10 mL), dried over anhydrous sodium sulfate, and concentrated to give a crude residue, which was purified by silica gel flash column chromatography using a 50% ethyl acetate in hexane gradient to afford methyl trans-2-hydroxycyclohexane-1-carboxylate 1 (1.000 g, yield 25%) as a colorless oil. ¹HNMR (400 MHz, CDCl₃): δ 3.79-3.73 (m, 1H), 3.72 (s, 3H), 2.89 (s, 1H), 2.30-2.23 (m, 2H), 2.04-2.01 (m, 2H), 1.79-1.69 (m, 2H), 1.42-1.18 (m, 4H).

Step 2: Methyl trans-2-((2R,3R)-3-((2-oxabicyclo[2.2.2]octan-4-yl)methoxy)-2-(((benzyloxy)carbonyl)amino)butoxy)cyclohexane-1-carboxylate (3): To a solution of methyl trans-2-hydroxycyclohexane-1-carboxylate 1 (0.300 g, 1.89 mmol) and benzyl (R)-2-((R)-1-((2-oxabicyclo[2.2.2]octan-4-yl)methoxy)ethyl) aziridine-1-carboxylate 2 (0.655 g, 1.89 mmol) in chloroform (10 mL) was added boron trifluoride diethyl etherate (0.189 g, 1.32 mmol) at 0° C. The resulting mixture was stirred at 0° C.) for 2 hours. The reaction mixture was concentrated under reduced pressure to give a crude, which was purified by silica gel flash column chromatography using a 10% ethyl acetate in hexane gradient to afford methyl trans-2-((2R,3R)-3-((2-oxabicyclo[2.2.2]octan-4-yl)methoxy)-2-(((benzyloxy)carbonyl)amino)butoxy)cyclohexane-1-carboxylate 3 (0.530 g, crude) as a colorless oil. MS: [MH]⁺ 504.25.

Step 3: Methyl trans-2-((2R,3R)-3-((2-oxabicyclo[2.2.2]octan-4-yl)methoxy)-2-aminobutoxy)cyclohexane-1-carboxylate (4): A mixture of methyl trans-2-((2R,3R)-3-((2-oxabicyclo[2.2.2]octan-4-yl)methoxy)-2-(((benzyloxy)carbonyl)amino)butoxy)cyclohexane-1-carboxylate 3 (0.530 g, crude) and Pd/C (10%, 0.016 g) in methanol (10 mL) was stirred at room temperature under hydrogen for 2 hours. Palladium on carbon was removed through filtration and washed with methanol (10 mL×2). The combined filtrate was concentrated under reduced pressure to give a residue, which was purified by silica gel flash column chromatography using a 5% methanol in dichloromethane gradient to afford methyl trans-2-((2R,3R)-3-((2-oxabicyclo[2.2.2]octan-4-yl)methoxy)-2-aminobutoxy)cyclohexane-1-carboxylate 4 (0.090 g. crude) as a colorless oil, which was used in the next step without further purification. MS: [MH]⁺ 370.25

Step 4: Methyl 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexane-1-carboxylate (6): A mixture of methyl trans-2-((2R,3R)-3-((2-oxabicyclo[2.2.2]octan-4-yl)methoxy)-2-aminobutoxy)cyclohexane-1-carboxylate 4 (0.090 g. crude). DIPEA (0.063 g, 0.49 mmol). (S)-6-(5-fluorobenzo[d]thiazol-7-yl)-2-(1-(trifluoromethyl)cyclopropane-1-carbonyl)-2,6-diazaspiro[3.4]octane-8-carboxylic acid 5 (0.109 g, 0.25 mmol), and N 2-(7-azabenzotriazol-1-yl)-N,N,N′,N′-tetramethyluronium hexafluorophosphate (HATU) (0.112 g, 0.29 mmol) in DMF (5 mL) was stirred at room temperature for 2 hours. The reaction mixture was poured into water (20 mL) and extracted with ethyl acetate (10 mL). The organic layer was washed with brine (10 mL), dried over anhydrous sodium sulfate, and concentrated to give a crude residue, which was purified by silica gel flash column chromatography using a 5% methanol in dichloromethane gradient to afford methyl 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexane-1-carboxylate 6 (0.083 g, 6% of yield over 3 steps) as an orange oil. MS: [MH]⁺ 795.70.

Step 5: 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexane-1-carboxylic acid (1-56): A solution of methyl 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexane-1-carboxylate 6 (0.080 g, 0.10 mmol) and lithium hydroxide monohydrate (0.014 g, 0.34 mmol) in a mixture of tetrahydrofuran (4 mL), methanol (2 mL), and water (2 mL) was stirred at room temperature for 4 hours. The reaction mixture was concentrated. The residue was diluted with water (10 mL) and extracted with dichloromethane (10) mL). The aqueous layer was acidified to pH of 3-4 with hydrochloric acid (2.0 N) and extracted with dichloromethane (15 mL×3). The combined organic layers were dried over anhydrous sodium sulfate and concentrated to give a crude, which was purified by silica gel column chromatography using a 5% methanol in dichloromethane gradient to afford 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy) cyclohexane-1-carboxylic acid I-56 (0.038 g, yield 48%) as a gray solid. ¹HNMR (400 MHz, CD₃OD): δ 9.19 (s, 1H), 7.12 (d, J=9.2 Hz, 1H), 6.46 (d, J=11.2 Hz, 1H), 4.42-4.27 (m, 2H), 4.09-3.86 (m, 7H), 3.72-3.66 (m, 5H), 3.48-3.39 (m, 4H), 3.22-3.11 (m, 2H), 3.03-2.91 (m, 1H), 2.29-2.12 (m, 2H), 2.02-1.84 (m, 5H), 1.66-1.50 (m, 7H), 1.50-1.40 (m, 3H), 1.08-0.99 (m, 5H). MS: [MH]⁺ 781.30.

### Synthesis of (S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(5-fluorobenzo[d]oxazol-7-yl)-2-(1-(trifluoromethyl)cyclopropane-1-carbonyl)-2,6-diazaspiro[3.4]octane-8-carboxamide I-52

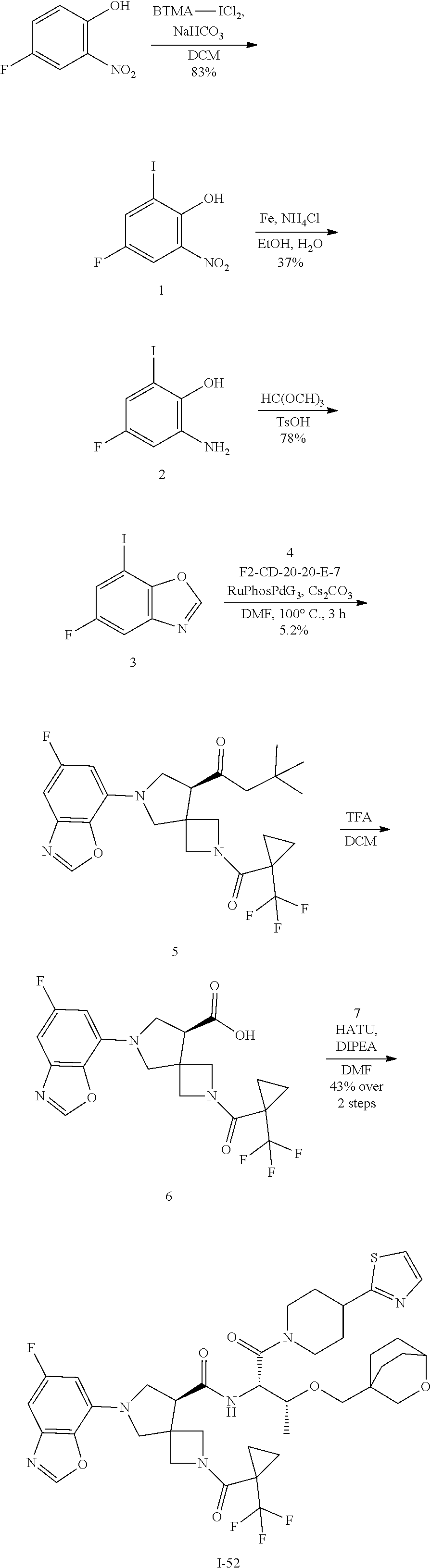

Step 1:4-Fluoro-2-iodo-6-nitrophenol (1): To a solution of 4-fluoro-2-nitrophenol (1.000 g, 6.36 mmol) in dichloromethane (15 mL) was added BTMA-ICI2 (2.4 g, 7.002 mmol) and sodium bicarbonate (3.7 g, 44.558 mmol). The resulting mixture was stirred at room temperature overnight. The solid was removed through filtration. The filtrate was acidified to pH of 3 with citric acid and extracted with ethyl acetate (20 mL×2). The combined organic layer was collected, washed with water (20 mL), dried over anhydrous sodium sulfate, and concentrated under reduced pressure to give a crude residue, which was purified by silica gel chromatography using a 2% ethyl acetate in hexane gradient to afford 4-fluoro-2-iodo-6-nitrophenol 1 (1.500 g, yield 83%) as a yellow oil. ¹HNMR (400 MHz, CDCl₃): δ 11.11 (s, 1H), 7.90-7.86 (m, 2H).

Step 2: 2-Amino-4-fluoro-6-iodophenol (2): A mixture of 4-fluoro-2-iodo-6-nitrophenol 1 (3.300 g, 11.66 mmol), iron powder (3.26 g, 58.30 mmol), and ammonium chloride (3.100 g, 58.30 mmol) in ethanol-water (30 mL-10 mL) was stirred at 80° C. for 2 hours. The solid was removed through filtration. The filtrate was concentrated under reduced pressure to give a crude residue, which was purified by silica gel chromatography using 12% ethyl acetate in hexane gradient to afford 2-amino-4-fluoro-6-iodophenol 2 (1.100 g, yield 37%) as a brown solid. MS: [MH]⁺ 253.70.

Step 3: 5-Fluoro-7-iodobenzo[d]oxazole (3): A mixture of 2-amino-4-fluoro-6-iodophenol 2 (0.150 g, 0.59 mmol) and p-toluenesulfonic acid (0.023 g, 0.12 mmol) in trimethyl orthoformate (2 mL) was stirred at 105° C. for 1.5 hour. The reaction mixture was poured into water (20 mL), extracted with ethyl acetate (10 mL), washed with water (10 mL×3) and brine (10 mL), dried over anhydrous sodium sulfate, and concentrated under reduced pressure to give a crude residue, which was purified by silica gel flash column chromatography using a 10% ethyl acetate in hexane gradient to afford 5-fluoro-7-iodobenzo[d]oxazole 3 (0.250 g, yield 78%) as a pink solid. MS: [MH]⁺ 263.60.

Step 4: tert-Butyl (S)-6-(5-fluorobenzo[d]oxazol-7-yl)-2-(1-(trifluoromethyl)cyclopropane-1-carbonyl)-2,6-diazaspiro[3.4]octane-8-carboxylate (5): A mixture of 5-fluoro-7-iodobenzo[d]oxazole 3 (0.400 g, 1.52 mmol), tert-butyl (S)-2-(1-(trifluoromethyl)cyclopropane-1-carbonyl)-2,6-diazaspiro[3.4]octane-8-carboxylate (0.441 g 1.27 mmol), RuPhosPdG; (0.212 g, 0.25 mmol), and cesium carbonate (0.834 g, 2.53 mmol) in N,N-dimethylformamide (4 mL) was stirred at 100° C. for 3.5 hours under N₂. The mixture was concentrated to give a crude residue, which was purified by silica gel column chromatography using a 10-30% ethyl acetate in hexane gradient to afford tert-butyl (S)-6-(5-fluorobenzo[d]oxazol-7-yl)-2-(1-(trifluoromethyl)cyclopropane-1-carbonyl)-2,6-diazaspiro[3.4]octane-8-carboxylate 5 (0.032 g, yield 5.2%) as a brown oil. MS: [MH]⁺ 484.30.

Step 5: (S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(5-fluorobenzo[d]oxazol-7-yl)-2-(1-(trifluoromethyl)cyclopropane-1-carbonyl)-2,6-diazaspiro[3.4]octane-8-carboxamide 1-52: To a solution of tert-butyl (S)-6-(5-fluorobenzo[d]oxazol-7-yl)-2-(1-(trifluoromethyl)cyclopropane-1-carbonyl)-2,6-diazaspiro[3.4]octane-8-carboxylate 5 (0.032 g, 0.066 mmol) in dichloromethane (1 mL) was added trifluoroacetate (1 mL). The mixture was stirred at 40° C. for 1.5 hour. The reaction mixture was concentrated under reduced pressure to afford crude (S)-6-(5-fluorobenzo[d]oxazol-7-yl)-2-(1-(trifluoromethyl)-cyclopropane-1-carbonyl)-2,6-diazaspiro[3.4]octane-8-carboxylic acid 6 as a brown oil, which was taken up in N,N-dimethylformamide (2 mL). Next, compound 7 (0.021 g, 0.053 mmol), N-ethyl-N-isopropylpropan-2-amine (0.014 mg, 0.105 mmol), and (2-(7-aza-1H-benzotriazole-1-yl)-1,1,3,3-tetramethyluronium hexafluorophosphate) (0.017 g, 0.046 mmol) were added under N₂. The resulting mixture was stirred at room temperature for 2 hours. The reaction mixture was poured into water (15 mL) and extracted with ethyl acetate (10 mL×2). The organic layer was collected, washed with water (10 mL×3) and brine (10 mL), dried over anhydrous sodium sulfate, and concentrated under reduced pressure to give a crude residue, which was purified by prep-TLC using a 7% methanol in dichloromethane gradient to afford (S)-N-((2S,3R)-3-((2-oxabicyclo[2.2.2]octan-4-yl)methoxy)-1-oxo-1-(4-(thiazol-2-yl)piperidin-1-yl)butan-2-yl)-6-(5-fluorobenzo[d]oxazol-7-yl)-2-(1-(trifluoromethyl)cyclopropane-1-carbonyl)-2,6-diazaspiro[3.4]octane-8-carboxamide I-52 (0.012 g, yield 43%) as a white solid. ¹HNMR (400 MHz, CD₃OD): δ 8.41-8.39 (m, 1H), 7.71-7.65 (m, 1H), 7.48-7.40 (m, 1H), 6.73-6.71 (m, 1H), 6.37-6.34 (m, 1H), 4.95 (s, 1H), 4.60-4.52 (m, 2H), 4.36-4.15 (m, 3H), 4.03-3.88 (m, 5H), 3.71-3.66 (m, 4H), 3.45-3.44 (m, 1H), 3.38-3.44 (m, 1H), 3.26-3.20 (m, 1H), 3.05-3.01 (m, 1H), 2.90-2.85 (m, 1H), 2.19-2.16 (m, 2H), 1.95-1.79 (m, 3H), 1.62-1.59 (m, 6H), 1.49-1.40 (m, 3H), 1.34-1.31 (m, 2H), 1.18-1.11 (m, 4H), 0.91-0.83 (m, 2H). LCMS: [MH]⁺ 803.75.

### Synthesis of (S)-N-((2S,3R)-3-((2-oxabicyclo[2.2.2]octan-4-yl)methoxy)-1-((4-methoxyphenyl) sulfonamido)-1-oxobutan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-50

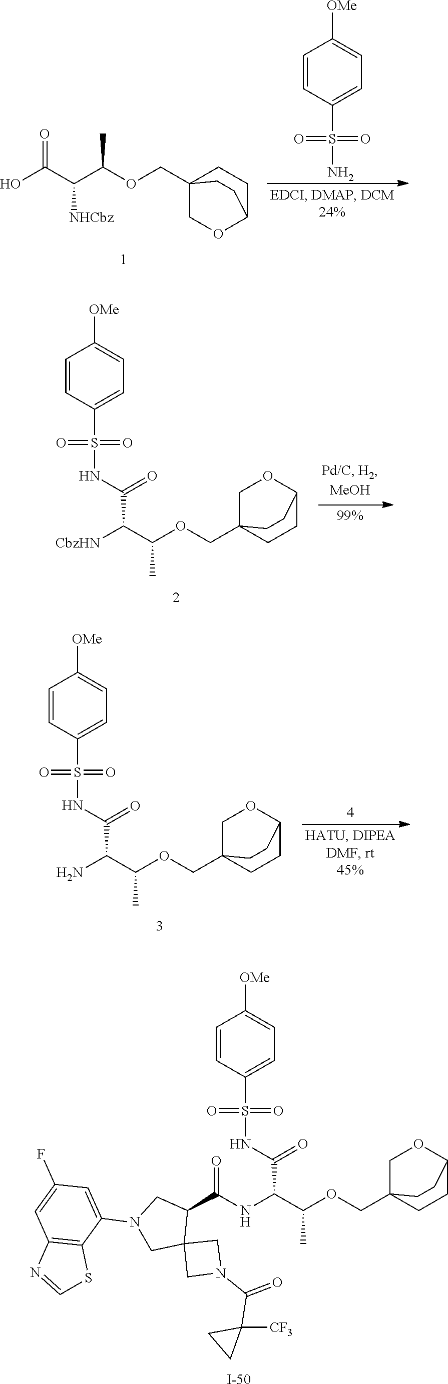

Step 1: Benzyl ((2S,3R)-3-((2-oxabicyclo[2.2.2]octan-4-yl)methoxy)-1-((4-methoxyphenyl) sulfonamido)-1-oxobutan-2-yl)carbamate (2): To a solution of O-((2-oxabicyclo[2.2.2]octan-4-yl)methyl)-N-((benzyloxy)carbonyl)-L-threonine 1 (0.390 g, 1.03 mmol) in dichloromethane (5 mL) was added 4-methoxybenzenesulfonamide (0.193 g, 1.03 mmol), 1-(3-dimethylaminopropyl)-3-ethylcarbodiimide Hydrochloride (EDCl) (0.197 g, 1.03 mmol), and N,N-dimethylpyridin-4-amine (0.025 g, 0.21 mmol). The resulting mixture was stirred at room temperature for 2 hours. The reaction mixture was poured into water (10 mL) and extracted with dichloromethane (25 mL). The organic layer was collected, washed with water (10 mL×2) and brine (15 mL), dried over anhydrous sodium sulfate, and concentrated under reduced pressure to give a crude residue, which was purified by silica gel flash chromatography using a 25-50% ethyl acetate in hexane gradient to afford benzyl ((2S,3R)-3-((2-oxabicyclo[2.2.2]octan-4-yl)methoxy)-1-((4-methoxyphenyl) sulfonamido)-1-oxobutan-2-yl)carbamate 2 (0.140 g, 24% yield) as a white solid. MS: [MH]⁺ 547.20.

Step 2: (2S,3R)-3-((2-Oxabicyclo[2.2.2]octan-4-yl)methoxy)-2-amino-N-((4-methoxyphenyl) sulfonyl)butanamide (3): A mixture of benzyl ((2S,3R)-3-((2-oxabicyclo[2.2.2]octan-4-yl)methoxy)-1-((4-methoxyphenyl) sulfonamido)-1-oxobutan-2-yl)carbamate 2 (0.140 g) and palladium on carbon (10%, 0.100 g) in isopropanol (10 mL) was stirred at room temperature under hydrogen atmosphere (hydrogen balloon) for 4 hours. Palladium on carbon was removed through filtration and washed with methanol (10 mL×2). The combined filtrate was concentrated under reduced pressure to afford (2S,3R)-3-((2-oxabicyclo[2.2.2]octan-4-yl)methoxy)-2-amino-N-((4-methoxyphenyl) sulfonyl)butanamide 3 (0.105 g, 99% yield) as gray solid. MS: [MH]⁺ 413.20.

Step 3: (S)-N-((2S,3R)-3-((2-Oxabicyclo[2.2.2]octan-4-yl)methoxy)-1-((4-methoxyphenyl) sulfonamido)-1-oxobutan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-50: To a solution of (2S,3R)-3-((2-oxabicyclo[2.2.2]octan-4-yl)methoxy)-2-amino-N-((4-methoxyphenyl) sulfonyl)butanamide 3 (0.098 g 0.24 mmol), (S)-6-(5-fluorobenzo[d]thiazol-7-yl)-2-(1-(trifluoromethyl)cyclopropane-1-carbonyl)-2,6-diazaspiro[3.4]octane-8-carboxylic acid 4 (0.105 g, 0.24 mmol) and N-ethyl-N-isopropylpropan-2-amine (0.093 g, 0.72 mmol) in N,N-dimethylformamide (3 mL) at 0-5° C. was added (2-(7-aza-1H-benzotriazole-1-yl)-1,1,3,3-tetramethyluronium hexafluorophosphate) (0.137 g, 0.36 mmol). The resulting mixture was stirred at room temperature under nitrogen for an hour. The reaction mixture was poured into saturated ammonium chloride (4 mL) and extracted with ethyl acetate (15 mL). The organic layer was collected and washed with water (8 mL×3) and brine (8 mL), dried over anhydrous sodium sulfate, and concentrated under reduced pressure to give a crude residue, which was purified by prep-TLC using a 10% methanol in dichloromethane gradient to afford (S)-N-((2S,3R)-3-((2-oxabicyclo[2.2.2]octan-4-yl)methoxy)-1-((4-methoxyphenyl) sulfonamido)-1-oxobutan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-50 (0.090 g, 45% yield) as a white solid. ¹H NMR (400 MHz, CD₃OD): δ 9.21-9.16 (m, 1H), 7.94 (d, J=8.8 Hz, 2H), 7.16-6.98 (m, 3H), 6.48-6.39 (m, 1H), 4.64-4.26 (m, 3H), 4.20-3.44 (m, 15H), 3.03 (d, J=8.8 Hz, 1H), 2.72 (d, J=9.2 Hz, 1H), 1.96-1.79 (m, 2H), 1.60-1.23 (m, 9H), 1.12-1.02 (m, 3H). MS: [MH]⁺ 838.25.

The following compounds were prepared in a manner analogous to the procedures described above for (S)-N-((2S,3R)-3-((2-oxabicyclo[2.2.2]octan-4-yl)methoxy)-1-((4-methoxyphenyl) sulfonamido)-1-oxobutan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-50:

(S)-N-((2S,3R)-3-(2-oxabicyclo[2.2.2]octan-4-ylmethoxy)-1-(2,4-difluorophenylsulfonamido)-1-oxobutan-2-yl)-6-(5-fluorobenzo[d]thiazol-7-yl)-2-(1-(trifluoromethyl)cyclopropanecarbonyl)-2,6-diazaspiro[3.4]octane-8-carboxamide I-51 (0.038 g, 50% yield) as a white solid. ¹HNMR (400 MHz, CD₃OD): δ 9.22 (s, 1H), 8.02-8.00 (m, 1H), 7.15 (d, J=8.8 Hz, 1H), 7.07-7.00 (m, 2H), 6.49 (d, J=12.0 Hz, 1H), 4.70-4.61 (m, 1H), 4.40-4.26 (m, 2H), 4.16-3.87 (m, 7H), 3.67-3.47 (m, 4H), 3.00-2.95 (m, 2H), 1.86 (s, 2H), 1.63-1.48 (m, 4H), 1.32 (s, 4H), 1.22 (s, 2H), 1.08-1.04 (m, 3H). MS: [MH]⁺ 844.1.

### Synthesis of 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)benzoic acid I-49

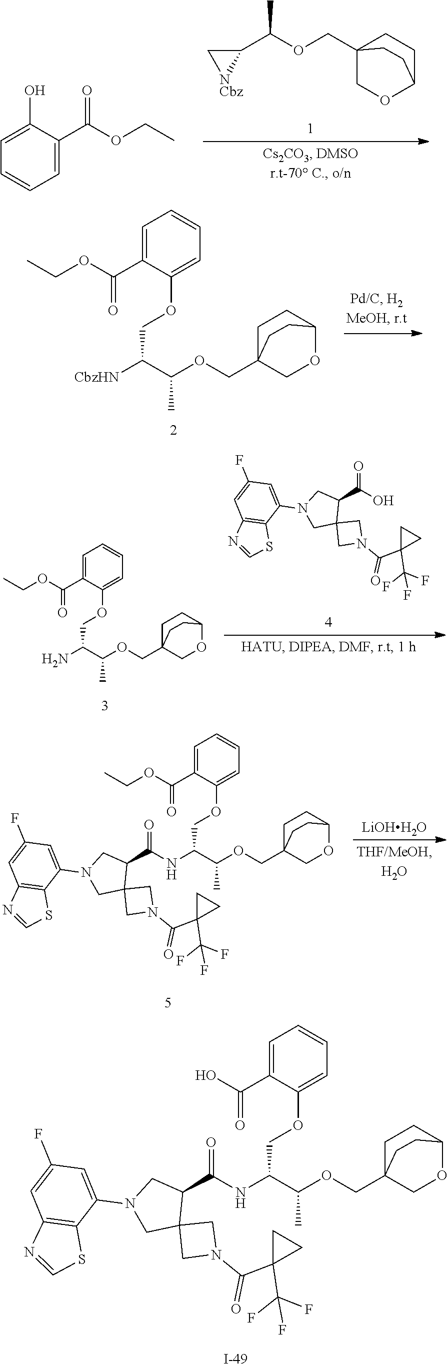

Step 1: Ethyl 2-((2R,3R)-3-((2-oxabicyclo[2.2.2]octan-4-yl)methoxy)-2-(((benzyloxy)carbonyl)amino)butoxy)benzoate (2): To a solution of ethyl 2-hydroxybenzoate (0.24 g, 1.45 mmol) in dimethyl sulfoxide (10 mL) was added benzyl (R)-2-((R)-1-((2-oxabicyclo[2.2.2]octan-4-yl)methoxy)ethyl) aziridine-1-carboxylate 1 (0.50 g, 1.45 mmol) and cesium carbonate (0.47 g, 1.15 mmol). The resulting mixture was stirred at 70° C. under nitrogen atmosphere for 16 hours. The reaction mixture was poured into water (20 mL), extracted with ethyl acetate (60 mL), washed with water (20 mL×2) and brine (10 mL), dried over anhydrous sodium sulfate, and concentrated under reduced pressure to give a crude residue, which was purified by silica gel column chromatography using a 17% ethyl acetate in hexane gradient to afford 2-((2R,3R)-3-((2-oxabicyclo[2.2.2]octan-4-yl)methoxy)-2-(((benzyloxy) carbonyl)amino)butoxy)benzoate 2 (0.162 g, 17%) as a yellow oil. MS: [MH]⁺ 512.30.

Step 2: Ethyl 2-((2R,3R)-3-((2-oxabicyclo[2.2.2]octan-4-yl)methoxy)-2-aminobutoxy)benzoate 3: A mixture of 2-((2R,3R)-3-((2-oxabicyclo[2.2.2]octan-4-yl)methoxy)-2-(((benzyloxy)carbonyl)amino)butoxy)benzoate 2 (0.140 g, 0.27 mmol) and Pd/C (10%, 0.015 g) in methanol (15 mL) was stirred at room temperature under hydrogen atmosphere for 16 hours. The reaction mixture was filtered, and the filtrate was concentrated to give a crude residue, which was purified by silica gel column chromatography using a 10% methanol in dichloromethane gradient to afford 2-((2R,3R)-3-((2-oxabicyclo[2.2.2]octan-4-yl)methoxy)-2-aminobutoxy)benzoate 3 (0.090 g, 76%) as a colorless oil. MS: [MH]⁺ 378.50.

Step 3: Ethyl 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)benzoate (5): To a solution of 2-((2R,3R)-3-((2-oxabicyclo[2.2.2]octan-4-yl)methoxy)-2-aminobutoxy)benzoate 3 (0.065 g, 0.17 mmol), (S)-6-(5-fluorobenzo[d]thiazol-7-yl)-2-(1-(trifluoromethyl)cyclopropane-1-carbonyl)-2,6-diazaspiro[3.4]octane-8-carboxylic acid 4 (0.076 g, 0.17 mmol), and N-ethyl-N-isopropylpropan-2-amine (0.067 g, 0.52 mmol) in N,N-dimethylformamide (1 mL) at 0-5° C. was added HATU (2-(7-aza-1H-benzotriazole-1-yl)-1,1,3,3-tetramethyluronium hexafluorophosphate) (0.098 g, 0.26 mmol). The resulting mixture was stirred at room temperature for one hour. The reaction mixture was poured into water (15 mL) and extracted with ethyl acetate (10 mL×2). The combined organic layer was washed with water (10) mL×3) and brine (10) mL), dried over anhydrous sodium sulfate, and concentrated under reduced pressure to give a crude residue, which was purified by silica gel column chromatography using a 7% methanol in dichloromethane gradient to afford 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)benzoate 5 (0.070 g, 47%) as a colorless oil. MS: [MH]⁺ 802.90.

Step 4: 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)benzoic acid I-49: To a solution of 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)benzoate 5 (0.070 g, 0.087 mmol) in a mixture of water (0.3 mL), methanol (0.3 mL), and tetrahydrofuran (0.9 mL) was added lithium hydroxide monohydrate (0.007 g, 0.174 mmol). The resulting mixture was stirred at room temperature for 3 hours. The reaction mixture was concentrated. The residue was diluted with water (10 mL) and extracted with dichloromethane (10 mL). The aqueous layer was acidified to pH of 3-4 with hydrochloric acid (2.0 N), and extracted with dichloromethane (15 mL×3). The combined organic layers were dried over anhydrous sodium sulfate and concentrated to give a residue, which was purified by silica gel column chromatography using a 5% methanol in dichloromethane gradient to afford 2-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)benzoic acid I-49 (0.030 g, 44% yield) as a white solid. ¹HNMR (400 MHz, CDCl₃): δ 8.95 (d, J=6.8 Hz, 1H), 8.03-7.98 (m, 1H), 7.55-7.51 (m, 1H), 7.30-7.27 (m, 1H), 7.10 (q, J=7.6 Hz, 1H), 7.04-7.00 (m, 1H), 6.38-6.30 (m, 1H), 4.42-4.10 (m, 7H), 3.92-3.77 (m, 4H), 3.68 (d, J=11.2 Hz, 3H), 3.25-3.19 (m, 2H), 3.03-2.97 (m, 1H), 2.05-1.95 (m, 4H), 1.63-1.54 (m, 5H), 1.45-1.38 (m, 2H), 1.25 (d, J=6.8 Hz, 4H), 1.15-1.12 (m, 3H). MS: [MH]⁺ 775.55.

### Synthesis of (S)-N-((2R,3R)-3-((2-oxabicyclo[2.2.2]octan-4-yl)methoxy)-1-((4-(trifluoromethyl)cyclohexyl)oxy)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-39

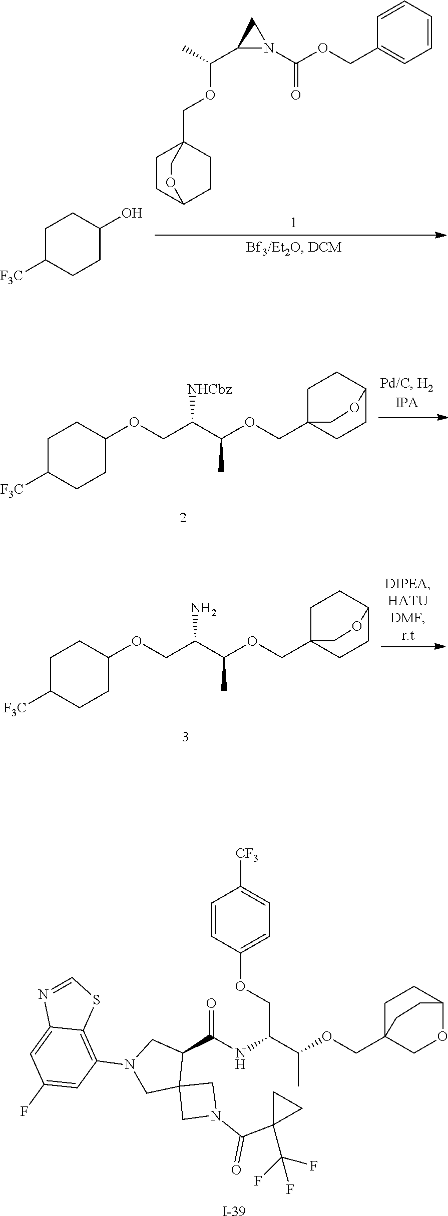

Step 1: Benzyl ((2S,3S)-3-((2-oxabicyclo[2.2.2]octan-4-yl)methoxy)-1-((4-(trifluoromethyl)cyclohexyl)oxy)butan-2-yl)carbamate (2): To a stirred solution of benzyl (R)-2-((R)-1-((2-oxabicyclo[2.2.2]octan-4-yl)methoxy)ethyl) aziridine-1-carboxylate 1 (0.500 g, 1.45 mmol) and 4-(trifluoromethyl)cyclohexan-1-ol (0.500 g, 2.97 mmol) in dichloromethane (3 mL) at −20° C. was added boron trifluoride-diethyl ether complex (0.062 g, 0.43 mmol). The resulting mixture was stirred at room temperature for 10 minutes. The reaction mixture was then concentrated under reduced pressure to give a crude residue, which was purified by silica gel flash chromatography using a 30% ethyl acetate in hexane gradient to afford benzyl ((2S,3S)-3-((2-oxabicyclo[2.2.2]octan-4-yl)methoxy)-1-((4-(trifluoromethyl)cyclohexyl)oxy)butan-2-yl)carbamate 2 (0.400 g, 50% purify) as a colorless oil. MS: [MH]⁺ 514.35.

Step 2: (2S,3S)-3-((2-oxabicyclo[2.2.2]octan-4-yl)methoxy)-1-((4-(trifluoromethyl)cyclohexyl)oxy)butan-2-amine (3): To a solution of benzyl ((2S,3S)-3-((2-oxabicyclo[2.2.2]octan-4-yl)methoxy)-1-((4-(trifluoromethyl)cyclohexyl)oxy)butan-2-yl)carbamate 2 (0.300 g, crude) in propan-2-ol (30 mL) was added palladium on carbon (10%, 0.06 g). The resulting mixture was stirred at room temperature under hydrogen atmosphere (hydrogen balloon) for 3 hours. Palladium on carbon was removed through filtration and washed with methanol (10 mL×2). The combined filtrate was concentrated under reduced pressure to afford (2S,3S)-3-((2-oxabicyclo[2.2.2]octan-4-yl)methoxy)-1-((4-(trifluoromethyl)cyclohexyl)oxy)butan-2-amine 3 (0.200 g, crude) as a colorless oil. MS: [MH]⁺ 380.15.

Step 3: (S)-N-((2R,3R)-3-((2-oxabicyclo[2.2.2]octan-4-yl)methoxy)-1-((4-(trifluoromethyl)cyclohexyl)oxy)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-39: To a solution of (2S,3S)-3-((2-oxabicyclo[2.2.2]octan-4-yl)methoxy)-1-((4-(trifluoromethyl)cyclohexyl)oxy)butan-2-amine 3 (0.100 g. crude), (S)-6-(5-fluorobenzo[d]thiazol-7-yl)-2-(1-(trifluoromethyl)cyclopropane-1-carbonyl)-2,6-diazaspiro[3.4]octane-8-carboxylic acid (0.051 g, 0.11 mmol), and N-ethyl-N-isopropylpropan-2-amine (0.072 g, 0.55 mmol) in N,N-dimethylformamide (2 mL) was added 2-(7-aza-1H-benzotriazole-1-yl)-1,1,3,3-tetramethyluronium hexafluorophosphate (0.058 g, 0.15 mmol) at 0-5° C. The resulting mixture was stirred at room temperature for 0.5 hour. The reaction mixture was poured into water (30 mL) and extracted with ethyl acetate (10 mL×3). The combined organic layers were washed with brine (20 mL×2), dried over anhydrous sodium sulfate, and concentrated under reduced pressure to give a crude residue, which was purified by preparative TLC using a 8% dichloromethane in methanol gradient to afford (S)-N-((2R,3R)-3-((2-oxabicyclo[2.2.2]octan-4-yl)methoxy)-1-((4-(trifluoromethyl)cyclohexyl)oxy)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-39 (0.036 g, 41% yield) as an off-white solid. ¹HNMR (400 MHz, CD₃OD): δ 9.20 (s, 1H), 7.14-7.12 (m, 1H), 6.47-6.44 (m, 1H), 4.65-4.57 (m, 1H), 4.44-4.36 (m, 1H), 4.14-3.82 (m, 7H), 3.73-3.70 (m, 3H), 3.59-3.48 (m, 3H), 3.40-3.37 (m, 1H), 3.24-3.18 (m, 2H), 2.99-2.94 (m, 1H), 2.13-1.81 (m, 8H), 1.72-1.56 (m, 5H), 1.52-1.45 (m, 2H), 1.36-1.23 (m, 4H), 1.14-1.03 (m, 5H). MS: [MH]⁺ 805.20.

### Synthesis of (8S)-N-((2R,3R)-3-(2-oxabicyclo[2.2.2]octan-4-ylmethoxy)-1-((4,4-dimethyl-6-oxotetrahydro-2H-pyran-2-yl)methoxy)butan-2-yl)-6-(5-fluorobenzo[d]thiazol-7-yl)-2-(1-(trifluoromethyl)cyclopropanecarbonyl)-2,6-diazaspiro[3.4]octane-8-carboxamide I-54

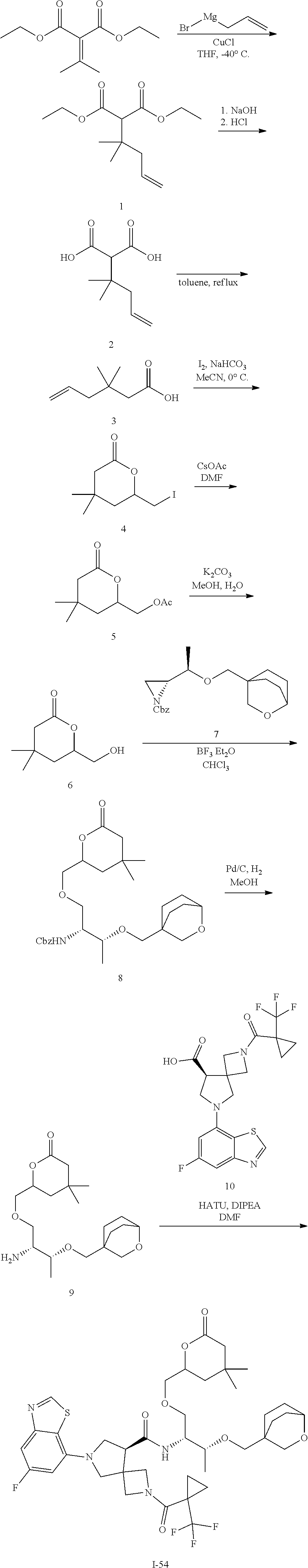

Step 1: Diethyl 2-(2-methylpent-4-en-2-yl) malonate (1): To a solution of CuCl (3.300 g, 37.71 mmol) in anhydrous Et₂O (320 mL) at −40° C. was added allyl magnesium bromide (1.0 M solution in Et₂O, 26.2 mL, 26.22 mmol). The mixture was stirred at −40° C. for 1 hour. Next, diethyl 2-(propan-2-ylidene) malonate (13.500 g, 67.42 mmol) was added. The resulting mixture was warmed to room temperature and stirred for 1 hour. The reaction mixture was quenched with saturated aqueous NH₄Cl solution, and extracted with ethyl acetate (100 mL×3). The combined organic layer was washed with brine, dried over anhydrous sodium sulfate, and concentrated under reduced pressure to give a crude residue, which was purified by column chromatography using 2% ethyl acetate in hexane gradient to afford diethyl 2-(2-methylpent-4-en-2-yl) malonate 1 (14.400 g, 88%) as a yellow oil. ¹HNMR (400 MHz, CDCl₃): δ 5.85-5.78 (m, 1H), 5.11-5.03 (m, 2H), 4.21-4.15 (m, 4H), 3.30 (s, 1H), 2.23 (d, J=7.6 Hz, 2H), 1.27 (t, J=7.2 Hz, 6H), 1.12 (s, 6H).

Step 2: 2-(2-Methylpent-4-en-2-yl) malonic acid (2): To a solution of diethyl 2-(2-methylpent-4-en-2-yl) malonate 1 (18.700 g, 77.17 mmol) in methanol (187 mL) was added aqueous NaOH solution (6N 187 mL). The mixture was stirred at 50° C. overnight. The reaction mixture was cooled to room temperature, quenched with hydrochloric acid (6.0 M), and extracted with dichloromethane (100 mL×3). The combined organic layers were washed with brine, dried over anhydrous sodium sulfate, and concentrated under reduced pressure to afford 2-(2-methylpent-4-en-2-yl) malonic acid 2 (14.200 g, 98%) as a yellow oil. ¹HNMR (400 MHz. CDCl₃): δ 8.91 (br, 2H), 5.87-5.77 (m, 1H), 5.15-5.08 (m, 2H), 3.42 (s, 1H), 2.26 (d, J=7.6 Hz, 2H), 1.16 (s, 6H).

Step 3: 3,3-Dimethylhex-5-enoic acid (3): A solution of 2-(2-methylpent-4-en-2-yl) malonic acid 2 (12.200 g, 65.49 mmol) in xylene (240 mL) was stirred at 130° C. overnight. The reaction mixture was cooled to room temperature, quenched with aqueous NaOH solution (6.0 M), and extracted with Et₂O (80 mL×3). The aqueous layer was acidified with hydrochloric acid (6.0 M) to a pH of below 3 and extracted with dichloromethane (50 mL×3). The combined organic layer was washed with brine, dried over anhydrous sodium sulfate, and concentrated under reduced pressure to afford 3,3-dimethylhex-5-enoic acid 3 (9.400 g, 87%) as a yellow oil. ¹HNMR (400 MHz, CDCl₃): δ 5.88-5.77 (m, 1H), 5.10-5.03 (m, 2H), 2.24 (s, 2H), 2.11 (d, J=7.6 Hz, 2H), 1.04 (s, 6H).

Step 4: 6-(Iodomethyl)-4,4-dimethyltetrahydro-H2-pyran-2-one (4): To a solution of 3,3-dimethylhex-5-enoic acid 3 (8.500 g, 59.77 mmol) in anhydrous MeCN (120 mL) at ambient temperature was added anhydrous NaHCO₃ (25.000 g, 298.87 mmol). The stirred suspension was cooled to 0° C. Next iodine (75.000 g, 298.87 mmol) was added with vigorous stirring. The reaction mixture was stirred at 0° C. for 4 hours. The reaction mixture was warmed to room temperature, quenched with saturated aqueous Na₂S₂O₃ solution, and extracted with ethyl acetate (50 mL×3). The combined organic layers were washed with brine, dried over anhydrous sodium sulfate, and concentrated under reduced pressure to give a crude residue, which was purified by column chromatography using 10-20% ethyl acetate in hexane gradient to afford 6-(iodomethyl)-4,4-dimethyltetrahydro-2H-pyran-2-one 4 (15.500 g, 97%) as a yellow oil. ¹HNMR (400 MHz, CDCl₃): δ 4.37-4.31 (m, 1H), 3.39-3.32 (m, 2H), 2.41-2.36 (m, 1H), 2.27-2.22 (m, 1H), 1.96-1.91 (m, 1H), 1.55-1.49 (m, 1H), 1.11 (d, J=5.6 Hz, 6H).

Step 5: (4,4-Dimethyl-6-oxotetrahydro-2H-pyran-2-yl)methyl acetate (5): To a solution of 6-(iodomethyl)-4,4-dimethyltetrahydro-2H-pyran-2-one 4 (10.000 g, 37.30 mmol) in DMF (70 mL) was added cesium acetate (17.900 g, 93.25 mmol). The mixture was stirred at 100° C. overnight. TLC showed the reaction was complete. The reaction mixture was warmed to room temperature, quenched with saturated aqueous NH₄Cl solution, and extracted with ethyl acetate (50 mL×3). The combined organic layers were washed with brine, dried over anhydrous sodium sulfate, and concentrated under reduced pressure to give a crude residue, which was purified by column chromatography using 10% ethyl acetate in hexane gradient to afford (4,4-dimethyl-6-oxotetrahydro-2H-pyran-2-yl)methyl acetate 5 (1.700 g, 23%) as a yellow oil. ¹HNMR (400 MHz, CDCl₃): δ 4.64-4.60 (m, 1H), 4.27-4.23 (m, 1H), 4.17-4.13 (m, 1H), 2.43-2.38 (m, 1H), 2.27-2.23 (m, 1H), 2.11 (s, 3H), 1.70-1.65 (m, 1H), 1.59-1.52 (m, 1H), 1.11 (d, J=6.8 Hz, 6H).

Step 6: 6-(Hydroxymethyl)-4,4-dimethyltetrahydro-2H-pyran-2-one (6): To a solution of (4,4-dimethyl-6-oxotetrahydro-2H-pyran-2-yl)methyl acetate 5 (1.77 g, 8.84 mmol) in methanol (24 mL) and water (8 mL) was added K₂CO₃ (3.000 g, 22.10 mmol). The mixture was stirred at room temperature for 4 hours. The reaction mixture was acidified with hydrochloric acid (2N) to a pH of 3-4 and extracted with dichloromethane (40) mL×2). The combined organic phases were washed with brine (20 mL×2), dried over anhydrous sodium sulfate, and concentrated under reduced pressure to give a crude residue, which was purified by column chromatography using a 30-50% ethyl acetate in hexane gradient to afford 6-(hydroxymethyl)-4,4-dimethyltetrahydro-2H-pyran-2-one 6 (1.000 g, 64%) as a colorless oil. ¹HNMR (400 MHz, CDCl₃): δ 4.52-4.48 (m, 1H), 3.83-3.79 (m, 1H), 3.67-3.62 (m, 1H), 3.03 (br, 1H), 2.41-2.37 (m, 1H), 2.27-2.22 (m, 1H), 1.69-1.59 (m, 2H), 1.10 (d, J=8.4 Hz, 6H).

Step 7: Benzyl ((2R,3R)-3-(2-oxabicyclo[2.2.2]octan-4-ylmethoxy)-1-((4,4-dimethyl-6-oxotetrahydro-2H-pyran-2-yl)methoxy)butan-2-yl)carbamate 8: To a solution of 6-(hydroxymethyl)-4,4-dimethyltetrahydro-2H-pyran-2-one 6 (0.229 g, 1.45 mmol) and (R)-benzyl 2-((R)-1-(2-oxabicyclo[2.2.2]octan-4-ylmethoxy)ethyl) aziridine-1-carboxylate 7 (0.500 g, 1.45 mmol) in chloroform (10 ml) at 0° C. was added boron trifluoride diethyl etherate (0.144 g, 1.01 mmol). The mixture was stirred at 0° C. for 1.5 hours. The reaction mixture was concentrated under reduced pressure to give a crude residue, which was purified by column chromatography using a 20-30% ethyl acetate in hexane gradient to afford benzyl ((2R,3R)-3-(2-oxabicyclo[2.2.2]octan-4-ylmethoxy)-1-((4,4-dimethyl-6-oxotetrahydro-2H-pyran-2-yl)methoxy)butan-2-yl)carbamate 8 (0.150 g, 21%) as a yellow oil. MS: [MH]⁺ 504.30.

Step 8: 6-(((2R,3R)-3-(2-oxabicyclo[2.2.2]octan-4-ylmethoxy)-2-aminobutoxy)methyl)-4,4-dimethyltetrahydro-2H-pyran-2-one 9: A mixture of benzyl ((2R,3R)-3-(2-oxabicyclo[2.2.2]octan-4-ylmethoxy)-1-((4,4-dimethyl-6-oxotetrahydro-2H-pyran-2-yl)methoxy)butan-2-yl)carbamate 8 (0.150 g, 0.30 mmol) and palladium on carbon (10%. 0.020 g) in methanol (20 mL) was stirred at room temperature under hydrogen atmosphere (hydrogen balloon) for 3 hours. Palladium on carbon was removed through filtration and washed with ethanol (10 mL×2). The combined filtrates were concentrated under reduced pressure to afford 6-(((2R,3R)-3-(2-oxabicyclo[2.2.2]octan-4-ylmethoxy)-2-aminobutoxy)methyl)-4,4-dimethyltetrahydro-2H-pyran-2-one 9 (0.100 g, 91%) as a colorless oil MS: [MH]⁺ 370.45.

Step 9: (8S)-N-((2R,3R)-3-(2-oxabicyclo[2.2.2]octan-4-ylmethoxy)-1-((4,4-dimethyl-6-oxotetrahydro-2H-pyran-2-yl)methoxy)butan-2-yl)-6-(5-fluorobenzo[d]thiazol-7-yl)-2-(1-(trifluoromethyl)cyclopropanecarbonyl)-2,6-diazaspiro[3.4]octane-8-carboxamide I-54: To a solution of 6-(((2R,3R)-3-(2-oxabicyclo[2.2.2]octan-4-ylmethoxy)-2-aminobutoxy)methyl)-4,4-dimethyltetrahydro-2H-pyran-2-one 9 (0.090 g, 0.27 mmol) in N,N-dimethylformamide (5 mL) was added(S)-6-(5-fluorobenzo[d]thiazol-7-yl)-2-(1-(trifluoromethyl)cyclopropanecarbonyl)-2,6-diazaspiro[3.4]octane-8-carboxylic acid 10 (0.100 g. 0.27 mmol), N-ethyl-N-isopropylpropan-2-amine (0.105 g, 0). 81 mmol), and (2-(7-aza-1H-benzotriazole-1-yl)-1,1,3,3-tetramethyluronium hexafluorophosphate) (0.123 g, 0.32 mmol). The resulting mixture was stirred at room temperature for 1 hour. The reaction mixture was then poured into water (10 mL) and extracted with ethyl acetate (20 mL×2). The combined organic phases were washed with brine (10 mL×2), dried over anhydrous sodium sulfate, and concentrated under reduced pressure to give a crude residue, which was purified by column chromatography using a 5% methanol in dichloromethane gradient to afford (8S)-N-((2R,3R)-3-(2-oxabicyclo[2.2.2]octan-4-ylmethoxy)-1-((4,4-dimethyl-6-oxotetrahydro-2H-pyran-2-yl)methoxy)butan-2-yl)-6-(5-fluorobenzo[d]thiazol-7-yl)-2-(1-(trifluoromethyl)cyclopropanecarbonyl)-2,6-diazaspiro[3.4]octane-8-carboxamide I-54 (0.030 g. 15%) as a white solid. ¹HNMR (400 MHz, CD₃OD): δ 9.22 (s, 1H), 7.15 (d, J=8.8 Hz, 1H), 6.48 (d, J=12.0 Hz, 1H), 4.67-4.40 (m, 4H), 4.14-3.91 (m, 7H), 3.75-3.57 (m, 9H), 3.45-3.42 (m, 1H), 3.26-3.21 (m, 1H), 3.02-2.97 (m, 1H), 2.46-2.20 (m, 2H), 2.06-1.96 (m, 2H), 1.66-1.52 (m, 9H), 1.13-1.01 (m, 10H). MS: [MH]⁺ 795.30.

### Synthesis of (S)-6-(5-fluorobenzo[d]thiazol-7-yl)-N-((2S,3R)-3-((1-(hydroxymethyl)cyclohexyl)methoxy)-1-oxo-1-(4-(thiazol-2-yl)piperidin-1-yl)butan-2-yl)-2-(1-(trifluoromethyl)cyclopropanecarbonyl)-2,6-diazaspiro[3.4]octane-8-carboxamide I-40

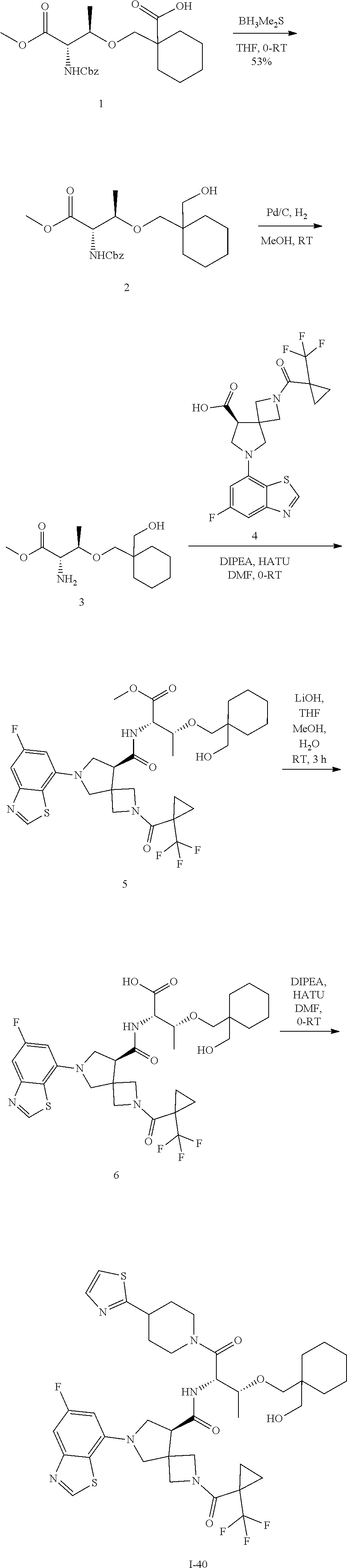

Step 1: (2S,3R)-methyl 2-(((benzyloxy)carbonyl)amino)-3-((1-(hydroxymethyl)cyclohexyl)methoxy)butanoate (2): To a solution of 1-((((2R,3S)-3-(((benzyloxy)carbonyl)amino)-4-methoxy-4-oxobutan-2-yl)oxy)methyl)cyclohexanecarboxylic acid 1 (0.407 g, 1.00 mmol) in anhydrous tetrahydrofuran (8 mL) at 0-5° C. under nitrogen atmosphere was added borane-methyl sulfide complex (1.0 mL, 2.0 mmol). The mixture was stirred at room temperature under nitrogen atmosphere for 15 hours. The resulting mixture was poured into ice water (20 mL), extracted with ethyl acetate (50 mL), washed with water (20 mL×2) and brine (20 mL), dried over anhydrous sodium sulfate, and concentrated under reduced pressure to give a crude, which was purified by column chromatography using a 15% ethyl acetate in hexane gradient to afford (2S,3R)-methyl 2-(((benzyloxy)carbonyl)amino)-3-((1-(hydroxymethyl)cyclohexyl)methoxy)butanoate 2 (0.210 g, 53%) as a colorless oil. MS: [MH]⁺ 394.25.

Step 2: (2S,3R)-methyl 2-amino-3-((1-(hydroxymethyl)cyclohexyl) methoxy)butanoate (3): A mixture of (2S,3R)-methyl 2-(((benzyloxy)carbonyl)amino)-3-((1-(hydroxymethyl)cyclohexyl)methoxy)butanoate 2 (0.205 g, 0.52 mmol) and palladium on carbon (10%, 0.030 g) in methanol (15 mL) was stirred at room temperature under hydrogen atmosphere (hydrogen balloon) for 15 hours. Palladium on carbon was removed through filtration and washed with methanol (20 ml×2). The combined filtrate was concentrated under reduced pressure to give (2S,3R)-methyl 2-amino-3-((1-(hydroxymethyl)cyclohexyl)methoxy)butanoate 3 (0.118 g, 87%) as a colorless oil, which was used in the next step without further purification.

Step 3: (2S,3R)-methyl 2-((S)-6-(5-fluorobenzo[d]thiazol-7-yl)-2-(1-(trifluoromethyl)cyclopropanecarbonyl)-2,6-diazaspiro[3.4]octane-8-carboxamido)-3-((1-(hydroxymethyl)cyclohexyl)methoxy)butanoate (5): To a mixture of (2S,3R)-methyl 2-amino-3-((1-(hydroxymethyl)cyclohexyl)methoxy)butanoate 3 (0.118 g, 0.45 mmol), (S)-6-(5-fluorobenzo[d]thiazol-7-yl)-2-(1-(trifluoromethyl)cyclopropanecarbonyl)-2,6-diazaspiro[3.4]octane-8-carboxylic acid 4 (0.201 g, 0.45 mmol), and N-ethyl-N-isopropylpropan-2-amine (0.174 g, 1.35 mmol) in N,N-dimethylformamide (1.5 mL) at 0-5° C. was added HATU (2-(7-aza-1H-benzotriazole-1-yl)-1,1,3,3-tetramethyluronium hexafluorophosphate) (0.256 g, 0.67 mmol). The resulting mixture was stirred at room temperature for 2 hours. The reaction mixture was poured into water (15 mL) and extracted with ethyl acetate (10 mL×2). The combined organic layers were washed with water (15 mL) and brine (15 mL), dried over anhydrous sodium sulfate, and concentrated under reduced pressure to give a crude residue, which was purified by silica gel column chromatography using a 50% ethyl acetate in hexane gradient to afford (2S,3R)-methyl 2-((S)-6-(5-fluorobenzo[d]thiazol-7-yl)-2-(1-(trifluoromethyl)cyclopropanecarbonyl)-2,6-diazaspiro[3.4]octane-8-carboxamido)-3-((1-(hydroxymethyl)cyclohexyl)methoxy)butanoate 5 (0.136 g, 44%) as a light-yellow solid. MS: [MH]⁺ 685.50.

Step 4: (2S,3R)-2-((S)-6-(5-fluorobenzo[d]thiazol-7-yl)-2-(1-(trifluoromethyl)cyclopropanecarbonyl)-2,6-diazaspiro[3.4]octane-8-carboxamido)-3-((1-(hydroxymethyl)cyclohexyl)methoxy)butanoic acid (6): A solution of (2S,3R)-methyl 2-((S)-6-(5-fluorobenzo[d]thiazol-7-yl)-2-(1-(trifluoromethyl)cyclopropanecarbonyl)-2,6-diazaspiro[3.4]octane-8-carboxamido)-3-((1-(hydroxymethyl)cyclohexyl)methoxy)butanoate 5 (0.136 g, 0.23 mmol) and lithium hydroxide monohydrate (0.019 g, 0.46 mmol) in a mixture of tetrahydrofuran, methanol, and water (1.5 mL/0.5 mL/0.5 mL) was stirred at room temperature for 2 hours. The reaction mixture was adjusted to pH of 5-6 with hydrochloric acid (2.0) M) at 0-5° C. and extracted with ethyl acetate (10 mL×2). The combined organic layers were washed with water (10 mL) and brine (10 mL), dried over anhydrous sodium sulfate, and concentrated under reduced pressure to give a crude residue, which was purified by silica gel column chromatography using a 5% methanol in dichloromethane gradient to afford (2S,3R)-2-((S)-6-(5-fluorobenzo[d]thiazol-7-yl)-2-(1-(trifluoromethyl)cyclopropanecarbonyl)-2,6-diazaspiro[3.4]octane-8-carboxamido)-3-((1-(hydroxymethyl)cyclohexyl)methoxy)butanoic acid 6 (0.076 g, 49%) as a light-yellow solid. MS: [MH]⁺ 671.15.

Step 5: (S)-6-(5-fluorobenzo[d]thiazol-7-yl)-N-((2S,3R)-3-((1-(hydroxymethyl)cyclohexyl)methoxy)-1-oxo-1-(4-(thiazol-2-yl)piperidin-1-yl)butan-2-yl)-2-(1-(trifluoromethyl)cyclopropanecarbonyl)-2,6-diazaspiro[3.4]octane-8-carboxamide (I-40): To a mixture of (2S,3R)-2-((S)-6-(5-fluorobenzo[d]thiazol-7-yl)-2-(1-(trifluoromethyl)cyclopropanecarbonyl)-2,6-diazaspiro[3.4]octane-8-carboxamido)-3-((1-(hydroxymethyl)cyclohexyl)methoxy)butanoic acid 6 (0.075 g, 0.11 mmol). 4-(1,3-thiazol-2-yl)piperidine dihydrochloride (0.029 g, 0.12 mmol), and N-ethyl-N-isopropylpropan-2-amine (0.071 g, 0.55 mmol) in N,N-dimethylformamide (1.0 mL) at 0-5° C. was added HATU (2-(7-aza-1H-benzotriazole-1-yl)-1,1,3,3-tetramethyluronium hexafluorophosphate) (0.061 g, 0.16 mmol). The resulting mixture was stirred at room temperature for 2 hours. The reaction mixture was poured into water (5 mL) and extracted with ethyl acetate (5 mL×3). The combined organic layers were washed with water (10 mL) and brine (10 mL), dried over anhydrous sodium sulfate, and concentrated under reduced pressure to give a crude residue, which was purified by silica gel column chromatography using a 6% methanol in dichloromethane gradient to afford (S)-6-(5-fluorobenzo[d]thiazol-7-yl)-N-((2S,3R)-3-((1-(hydroxymethyl)cyclohexyl)methoxy)-1-oxo-1-(4-(thiazol-2-yl)piperidin-1-yl)butan-2-yl)-2-(1-(trifluoromethyl)cyclopropanecarbonyl)-2,6-diazaspiro[3.4]octane-8-carboxamide 1-40 (0.036 g, 40% yield) as a white solid. ¹HNMR (400 MHz, CD₃OD): δ 9.18 (d, J=7.2 Hz, 1H), 8.28-8.23 (m, 1H), 7.71-7.63 (m, 1H), 7.48-7.38 (m, 1H), 7.12 (t, J=6.8 Hz, 1H), 6.47-6.43 (m, 1H), 4.96-4.91 (m, 1H), 4.65-4.34 (m, 3H), 4.23-3.78 (m, 8H), 3.49-3.35 (m, 4H), 3.27-3.23 (m, 1H), 2.91-2.81 (m, 1H), 2.22-2.08 (m, 2H), 1.87-1.60 (m, 2H), 1.42-1.18 (m, 19H). MS: [MH]⁺ 821.35.

### Synthesis of (S)-N-((2S,3R)-3-((2-oxabicyclo[2.2.2]octan-4-yl)methoxy)-1-(methylamino)-1-oxobutan-2-yl)-6-(thiazole-5-carbonyl)-2-(4-(trifluoromethyl)oxazol-2-yl)-2,6-diazaspiro[3.4]octane-8-carboxamide I-32

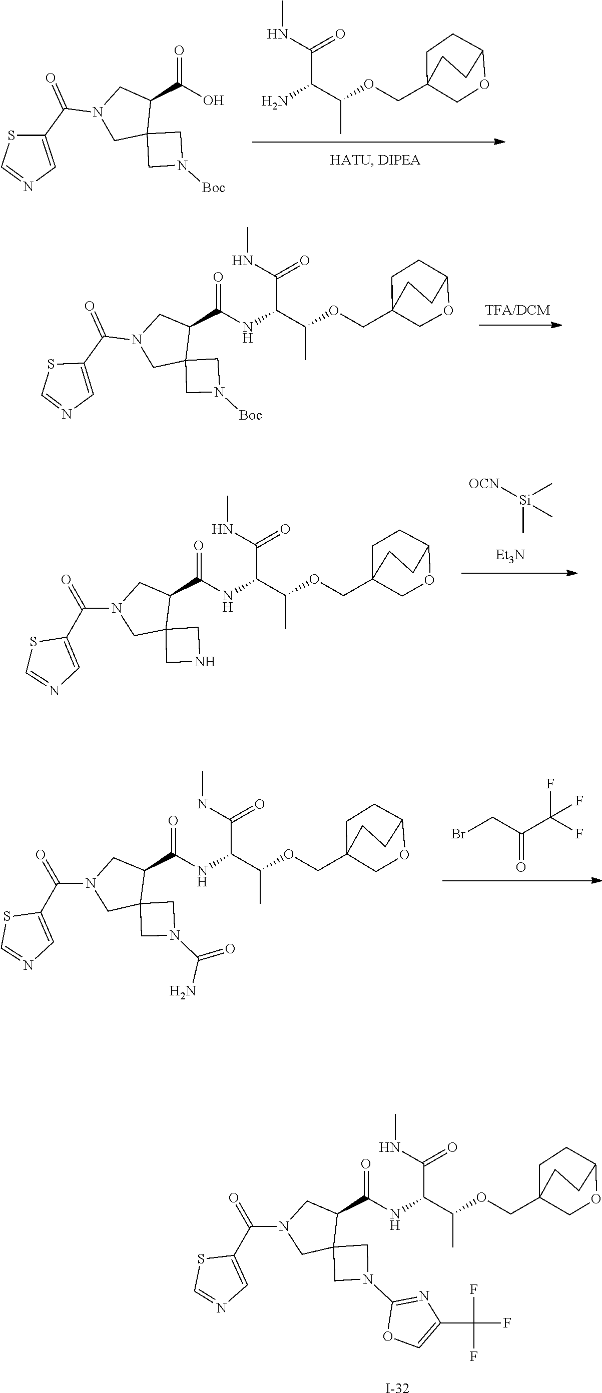

Step 1: tert-Butyl (S)-8-(((2S,3R)-3-((2-oxabicyclo[2.2.2]octan-4-yl)methoxy)-1-(methylamino)-1-oxobutan-2-yl)carbamoyl)-6-(thiazole-5-carbonyl)-2,6-diazaspiro[3.4]octane-2-carboxylate: To a solution of (S)-2-(tert-butoxycarbonyl)-6-(thiazole-5-carbonyl)-2,6-diazaspiro[3.4]octane-8-carboxylic acid (120 mg, 0.33 mmol) in DCM (3 mL) was added HATU (126 mg, 0.33 mmol) and DIPEA (170 mg, 1.32 mmol). The mixture was stirred at room temperature for 30 minutes. Next, (2S,3R)-3-((2-oxabicyclo[2.2.2]octan-4-yl)methoxy)-2-amino-N-methylbutanamide (110 mg, 0.43 mmol) was added, and the reaction stirred an additional 2 hours. The mixture was diluted with water (20 mL) and extracted with DCM (50 mL×2). The combined organic layers were washed with brine, dried over Na₂SO₄, filtered, and concentrated. The residue was purified by prep-TLC (eluent: DCM/MeOH=10/1) to afford tert-butyl (S)-8-(((2S,3R)-3-((2-oxabicyclo[2.2.2]octan-4-yl)methoxy)-1-(methylamino)-1-oxobutan-2-yl)carbamoyl)-6-(thiazole-5-carbonyl)-2,6-diazaspiro[3.4]octane-2-carboxylate (75 mg, 38%) as a yellow oil. LCMS m/z=606.2 [M+H]⁺. ¹H NMR (400 MHz, DMSO-d₆): δ 9.24 (d, J=7.2 Hz, 1H), 8.35 (d, J=19.8 Hz, 1H), 7.80-7.68 (m, 1H), 7.51-7.44 (m, 1H), 4.42 (t, J=5.4 Hz, 1H), 4.24 (d, J=17.8 Hz, 2H), 3.68-3.61 (m, 4H), 3.57-3.53 (m, 4H), 3.05-3.03 (m, 3H), 2.91 (d, J=15.4 Hz, 3H), 2.60 (d, J=4.6 Hz, 2H), 1.83 (t, J=4.2 Hz, 2H), 1.55-1.54 (m, 2H), 1.50 (d, J=4.0 Hz, 2H), 1.36 (s, 9H), 1.24 (m, 2H), 1.01 (d, J=6.4 Hz, 3H).

Step 2: (S)-N-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-(methylamino)-1-oxobutan-2-yl)carbamoyl)-6-(thiazole-5-carbonyl)-2,6-diazaspiro[3.4]octane-2-carboxylate (70 mg, 0.11 mmol) in DCM (2 mL) was added TFA (1 mL). The reaction mixture was stirred at room temperature for 2 hours, and then the solvent was removed under vacuum to afford (S)-N-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-8-carboxamide (60 mg, 100%), which was used directly in the next step. LCMS m/z=506.2 [M+H]⁺.

Step 3: (S)-N8-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-2,8-dicarboxamide: To a solution of (S)-N-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-8-carboxamide (60 mg, 0.11 mmol) in DCM (2 mL) at 0° C. was added TEA (0.07 mL) and isocyanatotrimethylsilane (0.02 mL, 0.15 mmol). The mixture was stirred at room temperature for 2 hours, and then the solvent was removed under vacuum to afford (S)-N8-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-2,8-dicarboxamide (65 mg, 100%), which was used directly in the next step. LCMS m/z=549.2 [M+H]⁺.

Step 4: (S)-N-((2S,3R)-3-((2-oxabicyclo[2.2.2]octan-4-yl)methoxy)-1-(methylamino)-1-oxobutan-2-yl)-6-(thiazole-5-carbonyl)-2-(4-(trifluoromethyl)oxazol-2-yl)-2,6-diazaspiro[3.4]octane-8-carboxamide I-32: To a solution of (S)-N8-((2S,3R)-3-((2-oxabicyclo[2.2.2]octan-4-yl)methoxy)-1-(methylamino)-1-oxobutan-2-yl)-6-(thiazole-5-carbonyl)-2,6-diazaspiro[3.4]octane-2,8-dicarboxamide (65 mg, 0.11 mmol) in 2-methylpropan-2-ol (2 mL) was added 3-bromo-1,1,1-trifluoropropan-2-one (0.06 mL, 0.59 mmol). The mixture was heated at 90° C. for 5 hours, and then the solvent was removed under reduced pressure. The residue obtained was purified by prep-HPLC to afford (S)-N-((2S,3R)-3-((2-oxabicyclo[2.2.2]octan-4-yl)methoxy)-1-(methylamino)-1-oxobutan-2-yl)-6-(thiazole-5-carbonyl)-2-(4-(trifluoromethyl)oxazol-2-yl)-2,6-diazaspiro[3.4]octane-8-carboxamide I-32 (3.5 mg, 5%). LCMS m/z=641.4 [M+H]⁺. ¹H NMR (400 MHz, Methanol-d₄): δ 9.16 (s, 1H), 8.37 (d, J=7.8 Hz, 1H), 7.94 (s, 1H), 4.42-4.19 (m, 5H), 4.16-4.08 (m, 2H), 3.98-3.84 (m, 2H), 3.81-3.45 (m, 5H), 3.22 (dd, J=9.2, 4.0 Hz, 1H), 2.98 (dd, J=9.4, 4.4 Hz, 1H), 2.75 (d, J=11.6 Hz, 3H), 1.95 (m, 2H), 1.74-1.58 (m, 4H), 1.46 (m, 2H), 1.07 (dd, J=9.2, 6.4 Hz, 3H).

### Synthesis of a mixture of (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-29 and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethoxy)piperidin-1-yl)butan-2-yl)-6-(thiazolo[4,5-d]pyrimidin-7-yl)-2,6-diazaspiro[3.4]octane-8-carboxamide I-35

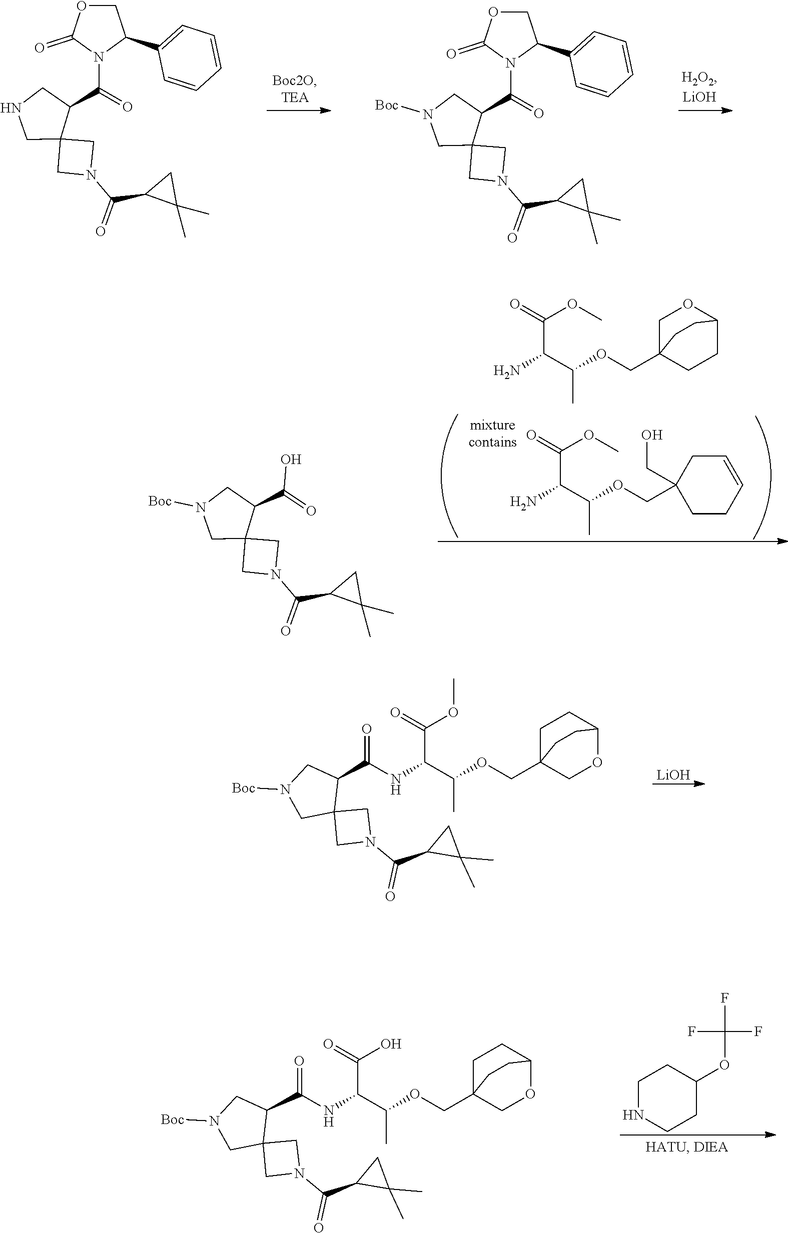

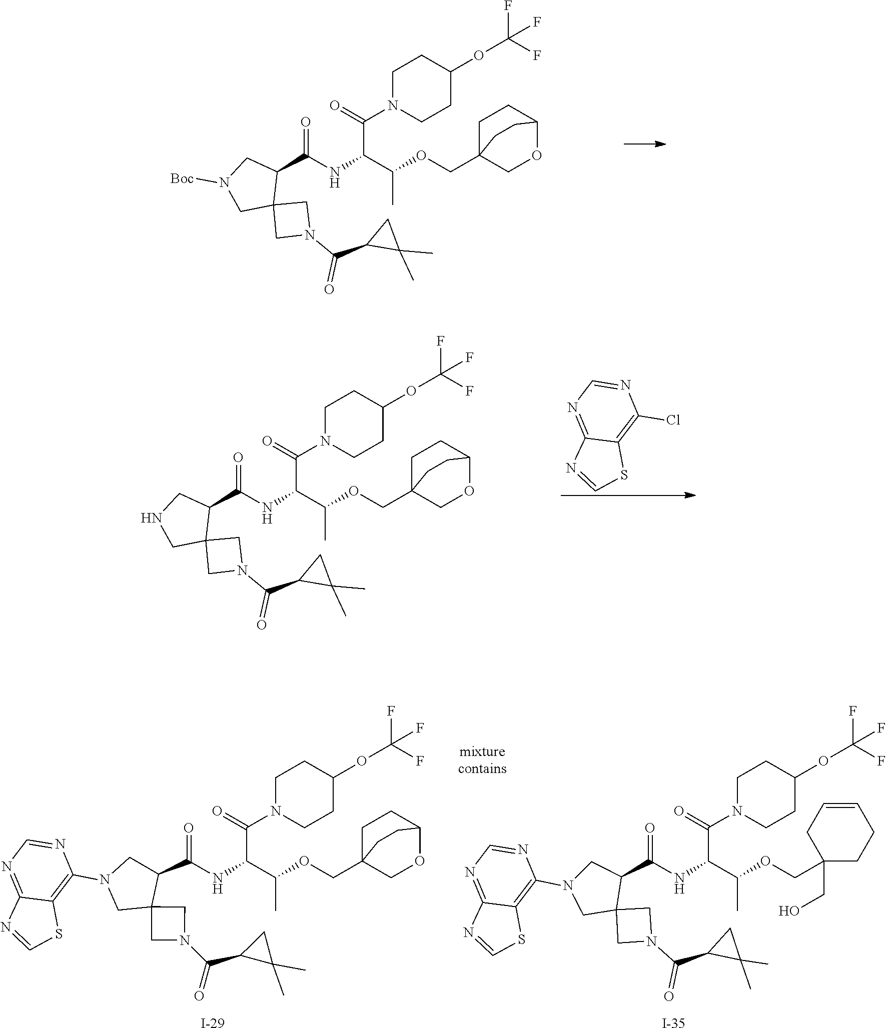

Step 1: tert-butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate: To a solution of (R)-3-((S)-2-((S)-2,2-dimethylcyclopropane-1-carbonyl)-2,6-diazaspiro[3.4]octane-8-carbonyl)-4-phenyloxazolidin-2-one (200 mg, 0.5 mmol) in DCM (2 mL) was added TEA (101 mg, 1.0 mmol) and (Boc)₂O (164 mg, 0.75 mmol). The reaction mixture was stirred at room temperature for 1.5 hour and then diluted with water (20 mL) and extracted with DCM (30 mL×3). The combined organic lavers were washed with brine, dried over Na₂SO₄, filtered, and purified by column chromatography on silica gel (eluent: DCM/MeOH=100/1) to afford tert-butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate (95 mg, 38%) as a colorless oil. LCMS m/z=498.2 [M+H]⁺. ¹H NMR (400 MHz, DMSO-d₆): δ 7.41-7.23 (m, 5H), 5.49-5.43 (m, 1H), 4.81-4.72 (m, 1H), 4.33-4.12 (m, 3H), 4.06-3.30 (m, 7H), 1.41-1.33 (m, 9H), 1.28-1.20 (m, 2H), 1.13-1.04 (m, 5H), 0.89-0.83 (m, 1H), 0.70-0.64 (m, 1H).

Step 2: (S)-6-(tert-butoxycarbonyl)-2-((S)-2,2-dimethylcyclopropane-1-carbonyl)-2,6-diazaspiro[3.4]octane-8-carboxylic acid: To a solution of tert-butyl (S)-2-((S)-2,2-dimethylcyclopropane-1-carbonyl)-8-((R)-2-oxo-4-phenyloxazolidine-3-carbonyl)-2,6-diazaspiro[3.4]octane-6-carboxylate (90 mg, 0.18 mmol) in a mixture of THF (0.8 mL) and water (0.2 mL) at 0° C. was added a solution of lithium hydroxide monohydrate (11 mg, 0.45 mmol) in water (0.2 mL) and 30% H₂O₂ (12 mg, 0.36 mmol) in water (0.2 mL). The reaction was stirred at 0° C. for 2 hours and then diluted with water (10 mL) and extracted with EtOAc (30 mL). The aqueous layer was collected and acidified with HCl (1M) to a pH of ˜3 and extracted with EtOAc (50 mL×3). The combined organic layers were washed with brine, dried over Na₂SO₄, filtered, and concentrated to afford (S)-6-(tert-butoxycarbonyl)-2-((S)-2,2-dimethylcyclopropane-1-carbonyl)-2,6-diazaspiro[3.4]octane-8-carboxylic acid (60 mg, 95%) as a colorless oil, which was used directly in the next step. LCMS m/z=353.3 [M+H]⁺. ¹H NMR (400 MHz, DMSO-d₆): δ 3.55-3.41 (m, 9H), 1.42-1.37 (m, 10H), 1.13-1.03 (m, 6H), 0.89-0.83 (m, 1H), 0.70-0.63 (m, 1H).

Step 3: tert-Butyl (S)-8-(((2S,3R)-3-((2-oxabicyclo[2.2.2]octan-4-yl)methoxy)-1-methoxy-1-oxobutan-2-yl)carbamoyl)-2-((S)-2,2-dimethylcyclopropane-1-carbonyl)-2,6-diazaspiro[3.4]octane-6-carboxylate: To a solution of methyl O-((2-oxabicyclo[2.2.2]octan-4-yl)methyl)-L-threoninate (345 mg, 0.98 mmol) in DCM (3 mL) was added HATU (403 mg, 1.06 mmol) and DIPEA (316 mg, 2.45 mmol). The mixture was stirred at room temperature for 30 minutes and then (S)-6-(tert-butoxy carbonyl)-2-((S)-2,2-dimethylcyclopropane-1-carbonyl)-2,6-diazaspiro[3.4]octane-8-carboxylic acid (210 mg, 0.82 mmol) was added. The reaction was stirred at room temperature for an additional 1.5 hours and then diluted with water (20 mL) and extracted with EtOAc (30 mL×3). The combined organic layers were washed with brine, dried over Na₂SO₄, filtered, and concentrated. The residue obtained was purified by prep-TLC (eluent: DCM/MeOH=20/1) to afford tert-butyl (S)-8-(((2S,3R)-3-((2-oxabicyclo[2.2.2]octan-4-yl)methoxy)-1-methoxy-1-oxobutan-2-yl)carbamoyl)-2-((S)-2,2-dimethylcyclopropane-1-carbonyl)-2,6-diazaspiro[3.4]octane-6-carboxylate (370) mg, 77%) as a white solid. LCMS m/z=592.3 [M+H]⁺. ¹H NMR (400 MHz, DMSO-d₆): δ 8.39-8.27 (m, 1H), 5.62-5.50 (m, 1H), 4.55-4.43 (m, 1H), 4.35-3.98 (m, 5H), 3.94-3.78 (m, 3H), 3.72-3.61 (m, 6H), 3.22-3.09 (m, 3H), 1.43-1.37 (m, 15H), 1.14-1.10 (m, 3H), 1.08-0.99 (m, 9H), 0.87-0.83 (m, 2H), 0.71-0.63 (m, 1H).

Step 4: 0-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine: To a solution of tert-butyl (S)-8-(((2S,3R)-3-((2-oxabicyclo[2.2.2]octan-4-yl)methoxy)-1-methoxy-1-oxobutan-2-yl)carbamoyl)-2-((S)-2,2-dimethylcyclopropane-1-carbonyl)-2,6-diazaspiro[3.4]octane-6-carboxylate (50 mg, 0.85 mmol) in a mixture of THF (0.4 mL), MeOH (0.1 mL), and water (0.1 mL) was added LiOH (6 mg, 0.25 mmol). The mixture was stirred at room temperature for 4 hours and then diluted with water (10 mL) and extracted with EtOAc (20 mL×3). The aqueous phase was acidified to a pH of 3 with HCl (1M) and extracted with EtOAc (50 mL×3). The combined organic layers were washed with brine, dried over Na₂SO₄, and concentrated to afford O-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine (20 mg, 42%) as a colorless oil. LCMS m/z=578.3 [M+H]⁺.

Step 5: tert-butyl (S)-8-(((2S,3R)-3-((2-oxabicyclo[2.2.2]octan-4-yl)methoxy)-1-oxo-1-(4-(trifluoromethoxy)piperidin-1-yl)butan-2-yl)carbamoyl)-2-((S)-2,2-dimethylcyclopropane-1-carbonyl)-2,6-diazaspiro[3.4]octane-6-carboxylate: To a solution of O-((2-oxabicyclo[2.2.2]octan-4-yl)methyl)-N-((S)-6-(tert-butoxy carbonyl)-2-((S)-2,2-dimethylcyclopropane-1-carbonyl)-2,6-diazaspiro[3.4]octane-8-carbonyl)-L-threonine (153 mg. 0.27 mmol) in DCM (2 mL) was added HATU (111 mg, 0.29 mmol) and DIPEA (103 mg, 0.80 mmol). The mixture was stirred at room temperature for 30 minutes. 4-(Trifluoromethoxy)piperidine (45 mg, 0.27 mmol) was added, and the reaction was stirred at room temperature for an additional 1.5 hours. The mixture was diluted with water (20 mL) and extracted with EtOAc (30 mL×3). The combined organic layers were washed with brine, dried over Na₂SO₄, filtered, and concentrated. The residue obtained was purified by RP-column to afford tert-butyl (S)-8-(((2S,3R)-3-((2-oxabicyclo[2.2.2]octan-4-yl)methoxy)-1-oxo-1-(4-(trifluoromethoxy)piperidin-1-yl)butan-2-yl)carbamoyl)-2-((S)-2,2-dimethylcyclopropane-1-carbonyl)-2,6-diazaspiro[3.4]octane-6-carboxylate (75 mg, 39%) as a colorless oil. LCMS m/z=729.2 [M+H]⁺. ¹H NMR (400 MHz. DMSO-d₆): δ 4.36-3.56 (m, 11H), 1.57-1.50 (m, 3H), 1.38 (s. 15H), 1.12-0.96 (m, 13H), 0.86-0.84 (m, 2H), 0.69-0.64 (m, 1H).

Step 6: (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-oxo-1-(4-(trifluoromethoxy)piperidin-1-yl)butan-2-yl)carbamoyl)-2-((S)-2,2-dimethylcyclopropane-1-carbonyl)-2,6-diazaspiro[3.4]octane-6-carboxylate (65 mg, 0.1 mmol) in MeOH (3 mL) was added a solution of HCl in dioxane (1M, 0.7 mL). The reaction mixture was stirred at room temperature for 1.5 hours and then the solvent was removed under vacuum to afford (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-2,6-diazaspiro[3.4]octane-8-carboxamide (56 mg, 100%), which was used directly in the next step. LCMS m/z=629.3 [M+H]⁺.

Step 7: (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide (1-29) and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethoxy)piperidin-1-yl)butan-2-yl)-6-(thiazolo[4,5-d]pyrimidin-7-yl)-2,6-diazaspiro[3.4]octane-8-carboxamide (1-35): To a solution of (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-2,6-diazaspiro[3.4]octane-8-carboxamide (50 mg, 0.08 mmol) and 7-chlorothiazolo[4,5-d]pyrimidine (14 mg, 0.08 mmol) in CH₃CN (1 mL) was added Na₂CO₃ (25 mg, 0.2 mmol). The reaction mixture was heated at 70° C. for 2 hours and then concentrated under reduced pressure. The residue was purified by prep-HPLC to afford (S)-N-((2S,3R)-3-((2-oxabicyclo[2.2.2]octan-4-yl)methoxy)-1-oxo-1-(4-(trifluoromethoxy)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-29 (3.3 mg, 5%) as a yellow solid. Further elution provided (85)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethoxy)piperidin-1-yl)butan-2-yl)-6-(thiazolo[4,5-d]pyrimidin-7-yl)-2,6-diazaspiro[3.4]octane-8-carboxamide 1-35 (3.7 mg, 6%) as a yellow solid. 1-29: LCMS m/z=764.5; ¹H NMR (400 MHz, Methanol-d₄): δ 9.54 (s, 1H), 8.50 (s, 1H), 4.98-4.93 (m, 1H), 4.62-4.56 (m, 1H), 4.46-3.92 (m, 10H), 3.78-3.52 (m, 2H), 3.49-3.41 (m, 1H), 3.07-2.96 (m, 1H), 2.04-1.90 (m, 4H), 1.83-1.72 (m, 2H), 1.70-1.59 (m, 4H), 1.55-1.39 (m, 4H), 1.19-1.14 (m, 8H), 1.08-1.03 (m, 1H), 0.83-0.77 (m, 1H). I-35: LCMS m/z=764.4; ¹H NMR (400 MHz. Methanol-d₄): δ 9.54 (s, 1H), 8.50 (s, 1H), 5.68-5.55 (m, 2H), 4.95-4.89 (m, 1H), 4.62-4.54 (m, 1H), 4.45-3.95 (m, 9H), 3.80-3.71 (m, 2H), 3.64-3.53 (m, 2H), 3.49-3.41 (m, 3H), 3.27-3.21 (m, 1H), 2.12-1.88 (m, 5H), 1.87-1.38 (m, 9H), 1.20-1.13 (m, 8H), 1.08-1.04 (m, 1H), 0.83-0.76 (m, 1H).

### Synthesis of a Mixture of (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazolo[4,5-d]pyrimidin-7-yl)-2,6-diazaspiro[3.4]octane-8-carboxamide I-36

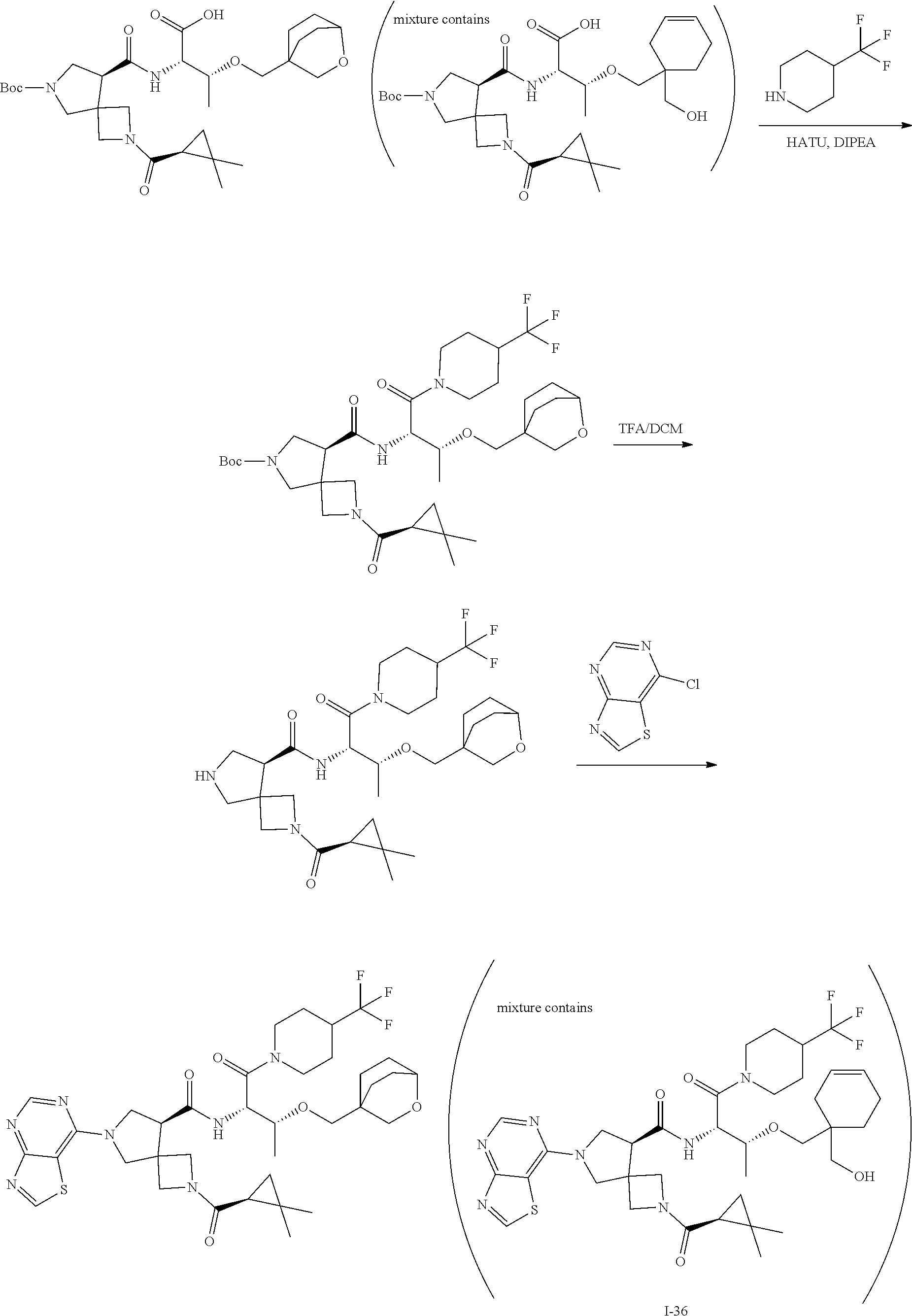

Step 1: tert-butyl (S)-8-(((2S,3R)-3-((2-oxabicyclo[2.2.2]octan-4-yl)methoxy)-1-oxo-1-(4-(trifluoromethyl)piperidin-1-yl)butan-2-yl)carbamoyl)-2-((S)-2,2-dimethylcyclopropane-1-carbonyl)-2,6-diazaspiro[3.4]octane-6-carboxylate: To a solution of 0-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine (115 mg, 0.2 mmol) in DCM (3 mL) was added HATU (114 mg, 0.3 mmol) and DIPEA (76 mg, 0.6 mmol). The mixture was stirred at room temperature for 30 minutes and then 4-(trifluoromethyl)piperidine (34 mg, 0.2 mmol) was added. The reaction mixture was stirred at room temperature for 2 hours and then diluted with water (20 mL) and extracted with DCM (50 mL×2). The combined organic layers were washed with brine, dried over Na₂SO₄, filtered, and concentrated. The residue was purified by prep-TLC (eluent: DCM/MeOH=10/1) to afford tert-butyl (S)-8-(((2S,3R)-3-((2-oxabicyclo[2.2.2]octan-4-yl)methoxy)-1-oxo-1-(4-(trifluoromethyl)piperidin-1-yl)butan-2-yl)carbamoyl)-2-((S)-2,2-dimethylcyclopropane-1-carbonyl)-2,6-diazaspiro[3.4]octane-6-carboxylate (95 mg, 67%) as a yellow solid. LCMS m/z=713.5 [M+H]⁺.

Step 2: (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-oxo-1-(4-(trifluoromethyl)piperidin-1-yl)butan-2-yl)carbamoyl)-2-((S)-2,2-dimethylcyclopropane-1-carbonyl)-2,6-diazaspiro[3.4]octane-6-carboxylate (85 mg, 0.12 mmol) in DCM (2 mL) was added TFA (1 mL). The reaction mixture was stirred at room temperature for 1 hour and then the solvent was removed under vacuum to afford ((S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-2,6-diazaspiro[3.4]octane-8-carboxamide (70) mg, 96%), which was used directly in the next step. LCMS m/z=613.4 [M+H]⁺.

Step 3: Mixture of (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazolo[4,5-d]pyrimidin-7-yl)-2,6-diazaspiro[3.4]octane-8-carboxamide (I-36): To a solution of (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-2,6-diazaspiro[3.4]octane-8-carboxamide (70) mg, 0.11 mmol) in MeCN (2 mL) was added Na₂CO₃ (36 mg, 0.33 mmol). The mixture was stirred at room temperature for 30 minutes and then 7-chlorothiazolo[4,5-d]pyrimidine (19 mg, 0.22 mmol) was added. The reaction mixture was stirred at room temperature overnight and then filtered through Celite and concentrated. The residue was purified by prep-HPLC to afford (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide (6.3 mg. 7.4%) as a white solid. Further elution provided (85)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazolo[4,5-d]pyrimidin-7-yl)-2,6-diazaspiro[3.4]octane-8-carboxamide I-36 (6.4 mg, 7.4%) as a white solid. First eluting isomer: LCMS m/z=748.4 [M+H]⁺: ¹H NMR (400 MHz, Methanol-d₄): δ 9.54 (s, 1H), 8.50 (s, 1H), 5.00-4.92 (m, 2H), 4.65-4.54 (m, 1H), 4.44-4.38 (m, 1H), 4.37-4.31 (m, 1H), 4.31-4.26 (m, 1H), 4.23-4.12 (m, 3H), 4.12-4.05 (m, 1H), 4.05-3.97 (m, 1H), 3.76-3.67 (m, 3H), 3.59-3.50 (m, 1H), 3.26-3.15 (m, 2H), 3.03 (s, 1H), 2.74-2.62 (m, 1H), 2.49 (s, 1H), 2.05-1.81 (m, 4H), 1.73-1.58 (m, 4H), 1.56-1.36 (m, 5H), 1.35-1.28 (m, 1H), 1.22-1.10 (m, 9H), 1.08-1.03 (m, 1H), 0.84-0.76 (m, 1H). I-36: LCMS m/z=748.4 [M+H]⁺: ¹H NMR (400 MHz, Methanol-d₄): δ 9.54 (s, 1H), 8.50 (s, 1H), 5.68-5.57 (m, 2H), 4.64-4.53 (m, 2H), 4.47-4.39 (m, 1H), 4.38-4.32 (m, 1H), 4.31-4.26 (m, 1H), 4.24-4.14 (m, 3H), 4.13-4.07 (m, 1H), 4.05-3.99 (m, 1H), 3.82-3.73 (m, 1H), 3.50-3.47 (m, 1H), 3.46-3.40 (m, 2H), 3.25-3.21 (m, 1H), 3.15-3.12 (m, 1H), 2.71-2.64 (m, 1H), 2.52-2.44 (m, 1H), 2.03-1.98 (m, 2H), 1.86-1.79 (m, 2H), 1.55-1.40 (m, 5H), 1.35-1.29 (m, 1H), 1.24-1.13 (m, 11H), 1.09-1.03 (m, 2H), 0.84-0.77 (m, 2H).

### Synthesis of a Mixture of (S)-N-((2S,3R)-3-((2-oxabicyclo[2.2.2]octan-4-yl)methoxy)-1-oxo-1-(7-oxa-2-azaspiro[3.5]nonan-2-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide 1-25 and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(7-oxa-2-azaspiro[3.5]nonan-2-yl)butan-2-yl)-6-(thiazolo[4,5-d]pyrimidin-7-yl)-2,6-diazaspiro[3.4]octane-8-carboxamide I-37

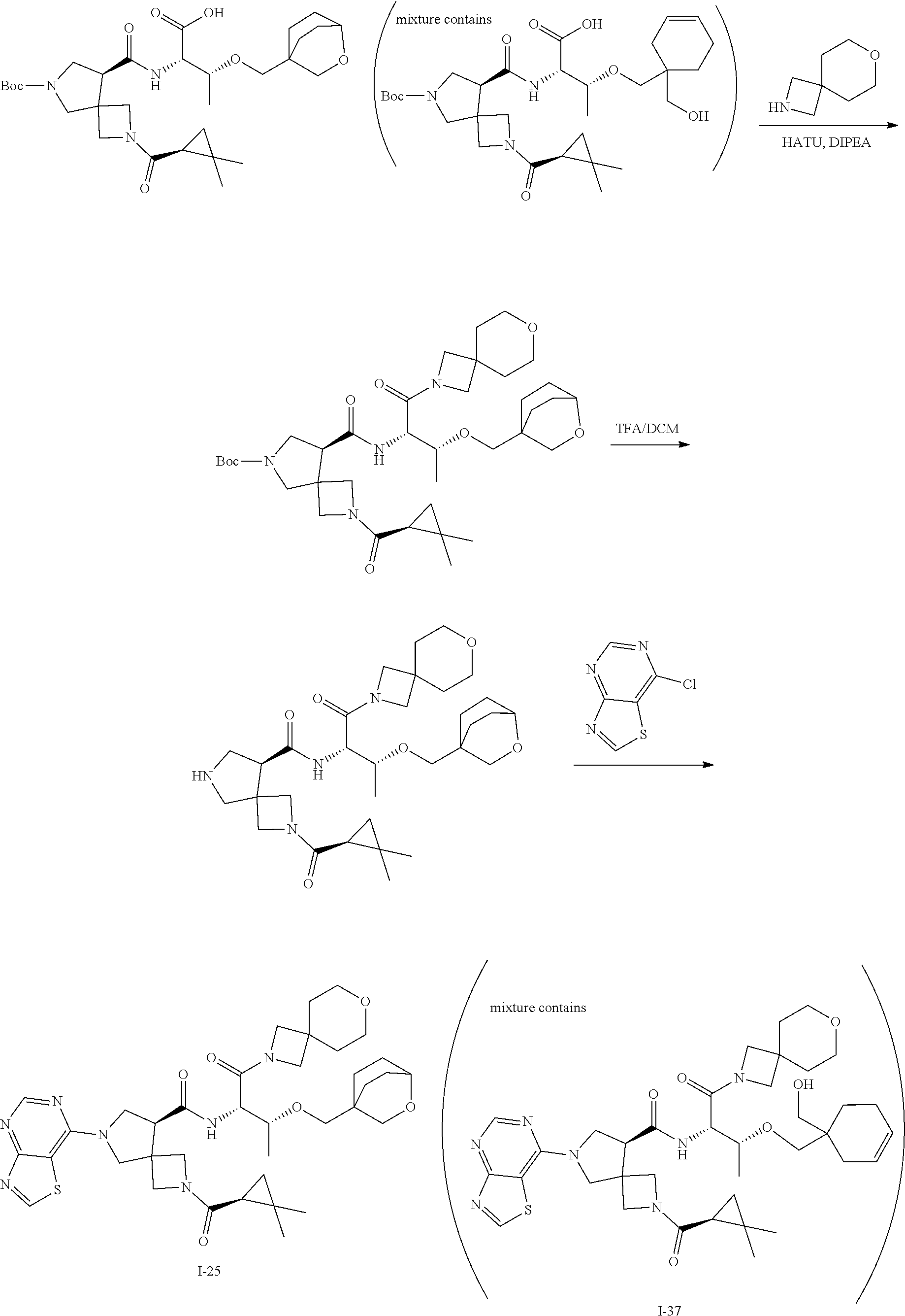

(S)-N-((2S,3R)-3-((2-oxabicyclo[2.2.2]octan-4-yl)methoxy)-1-oxo-1-(7-oxa-2-azaspiro[3.5]nonan-2-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-25 and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(7-oxa-2-azaspiro[3.5]nonan-2-yl)butan-2-yl)-6-(thiazolo[4,5-d]pyrimidin-7-yl)-2,6-diazaspiro[3.4]octane-8-carboxamide I-37 were synthesized from a mixture of O-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine and N-((S)-6-(tert-butoxycarbonyl)-2-((S)-2,2-dimethylcyclopropane-1-carbonyl)-2,6-diazaspiro[3.4]octane-8-carbonyl)-O-((1-(hydroxymethyl)cyclohex-3-en-1-yl)methyl)-L-threonine according to the procedures outlined above for the synthesis of (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide and I-36 using the appropriate commercially available reagents and/or intermediates described elsewhere. I-25: LCMS m/z=722.4 [M+H]⁺; ¹H NMR (400 MHz, DMSO-d₆): δ 9.72-9.71 (m, 1H), 8.51-8.50 (m, 1H), 8.45-8.42 (m, 1H), 4.44-4.39 (m, 1H), 4.26-4.14 (m, 3H), 3.98-3.77 (m, 6H), 3.64-3.38 (m, 12H), 3.16-3.10 (m, 1H), 3.05-2.97 (m, 1H), 1.86-1.79 (m, 1H), 1.66-1.50 (m, 8H), 1.49-1.26 (m, 4H), 1.11-1.03 (m, 9H), 0.89-0.85 (m, 1H), 0.71-0.66 (m, 1H). I-37: LCMS m/z=722.3 [M+H]⁺; ¹H NMR (400 MHz, DMSO-d₆): δ 9.72-9.70 (m, 1H), 8.51-8.40 (m, 2H), 5.64-5.52 (m, 2H), 4.45-4.38 (m, 1H), 4.37-4.31 (m, 1H), 4.28-4.15 (m, 3H), 4.00-3.88 (m, 5H), 3.66-3.39 (m, 9H), 3.26-3.15 (m, 3H), 1.97-1.88 (m, 2H), 1.80-1.75 (m, 2H), 1.70-1.50 (m, 5H), 1.47-1.29 (m, 4H), 1.11-1.05 (m, 9H), 0.89-0.84 (m, 1H), 0.71-0.66 (m, 1H).

### Synthesis of a mixture of (S)-N-((2S,3R)-3-((2-oxabicyclo[2.2.2]octan-4-yl)methoxy)-1-oxo-1-(2-oxa-6-azaspiro[3.5]nonan-6-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-26A and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(2-oxa-6-azaspiro[3.5]nonan-6-yl)butan-2-yl)-6-(thiazolo[4,5-d]pyrimidin-7-yl)-2,6-diazaspiro[3.4]octane-8-carboxamide I-26B

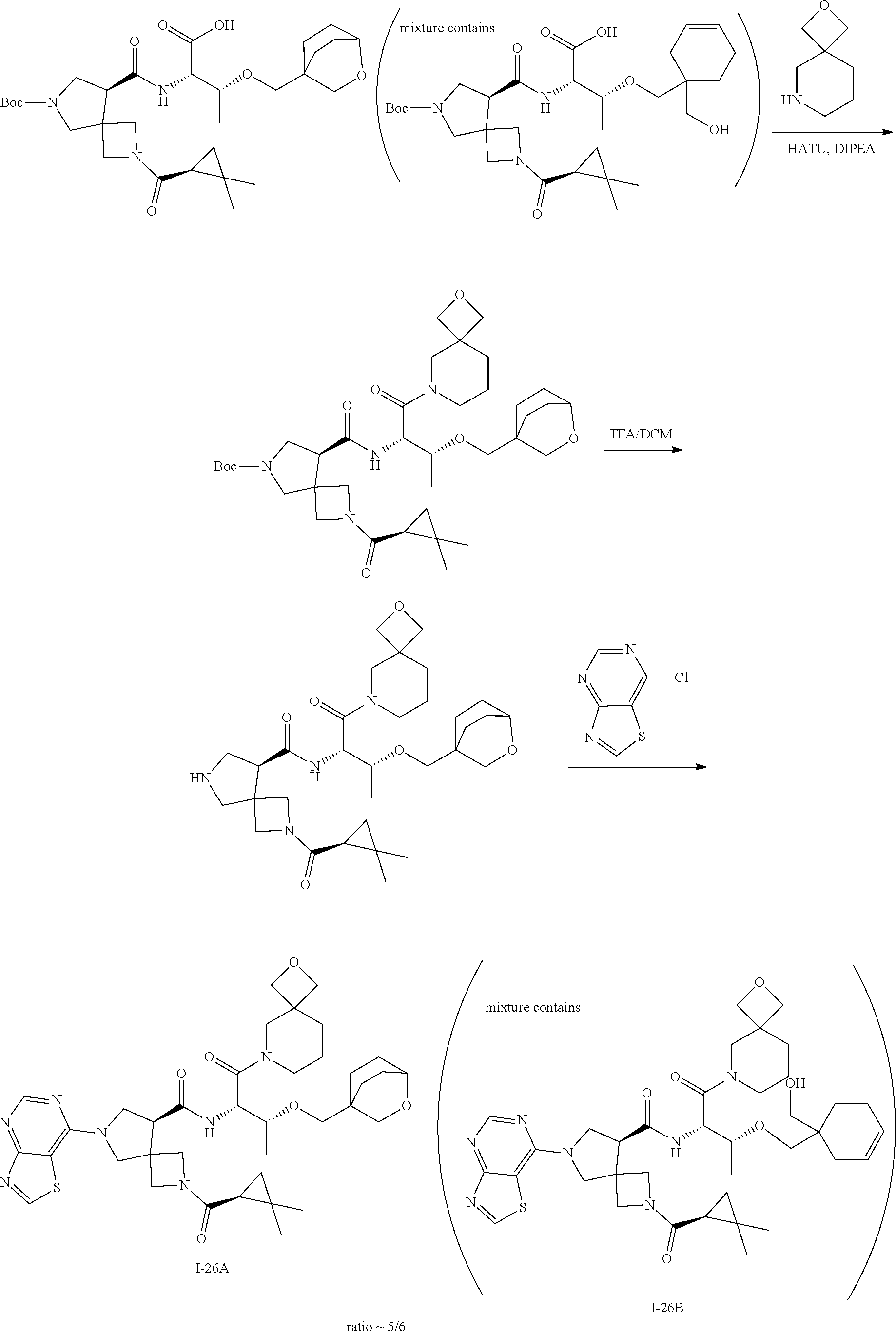

A mixture of (S)-N-((2S,3R)-3-((2-oxabicyclo[2.2.2]octan-4-yl)methoxy)-1-oxo-1-(2-oxa-6-azaspiro[3.5]nonan-6-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide I-26A and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(2-oxa-6-azaspiro[3.5]nonan-6-yl)butan-2-yl)-6-(thiazolo[4,5-d]pyrimidin-7-yl)-2,6-diazaspiro[3.4]octane-8-carboxamide I-26B was synthesized from a mixture of O-((2-oxabicyclo[2.2.2]octan-4-yl)methyl)-N-((S)-6-(tert-butoxycarbonyl)-2-((S)-2,2-dimethylcyclopropane-1-carbonyl)-2,6-diazaspiro[3.4]octane-8-carbonyl)-L-threonine and N-((S)-6-(tert-butoxycarbonyl)-2-((S)-2,2-dimethylcyclopropane-1-carbonyl)-2,6-diazaspiro[3.4]octane-8-carbonyl)-O-((1-(hydroxymethyl)cyclohex-3-en-1-yl)methyl)-L-threonine according to the procedures outlined above for the synthesis of (S)-N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazolo[4,5-d]pyrimidin-7-yl)-2,6-diazaspiro[3.4]octane-8-carboxamide and I-36 using the appropriate commercially available reagents and/or intermediates described elsewhere. The ratio of I-26A to I-26B was around 5/6. LCMS m/z=722.4 [M+H]⁺; ¹H NMR (400 MHz, Chloroform-d) δ 9.27 (s, 1H), 8.65 (s, 1H), 7.14-6.86 (m, 1H), 5.71-5.53 (m, 1.14H), 5.17-4.83 (m, 1H), 4.43-4.03 (m, 14H), 3.69-3.13 (m, 10H), 2.08-1.90 (m, 4H), 1.60-1.44 (m, 5H), 1.22-1.11 (m, 12H), 0.78 (s, 1H).

### Synthesis of a Mixture of N-((2S,3R)-3-((2-oxabicyclo[2.2.2]octan-4-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27A and N-((2S,3R)-3-((1-(hydroxymethyl)cyclohex-3-en-1-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27B

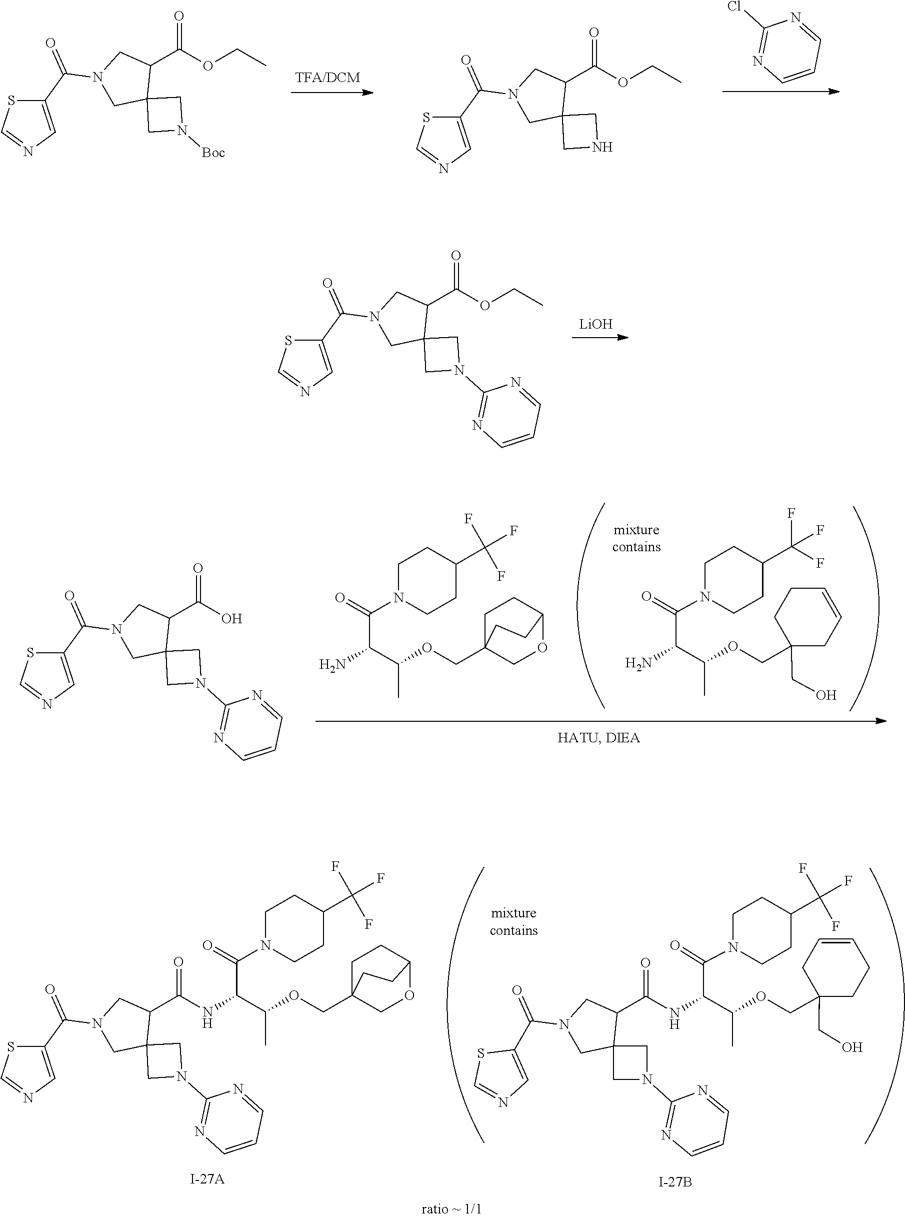

Step 1: Ethyl 6-(thiazole-5-carbonyl)-2,6-diazaspiro[3.4]octane-8-carboxylate: To a solution of 2-(tert-butyl) 8-ethyl 6-(thiazole-5-carbonyl)-2,6-diazaspiro[3.4]octane-2,8-dicarboxylate (400 mg, 1.0 mmol) in DCM (4 mL) was added TFA (2 mL). The reaction mixture was stirred at room temperature for 1.5 hour. The solvent was removed under vacuum to afford crude ethyl 6-(thiazole-5-carbonyl)-2,6-diazaspiro[3.4]octane-8-carboxylate (298 mg, 100%), which was used directly in the next step. LCMS m/z=296.1 [M+H]⁺.

Step 2: Ethyl 2-(pyrimidin-2-yl)-6-(thiazole-5-carbonyl)-2,6-diazaspiro[3.4]octane-8-carboxylate: To a solution of ethyl 6-(thiazole-5-carbonyl)-2,6-diazaspiro[3.4]octane-8-carboxylate (270) mg, 0.91 mmol) and 2-chloropyrimidine (104 mg, 0.91 mmol) in CH₃CN (3 mL) was added Na₂CO₃ (290) mg, 2.7 mmol). The reaction mixture was heated at 70° C. for 2 hours, and then the solvent was removed under reduced pressure. The residue was purified by column chromatography on silica gel (eluent: DCM/MeOH=40/1) to afford ethyl 2-(pyrimidin-2-yl)-6-(thiazole-5-carbonyl)-2,6-diazaspiro[3.4]octane-8-carboxylate (460 mg, 100%) as a colorless oil. LCMS m/z=374.1 [M+H]⁺. ¹H NMR (400 MHz, DMSO-d₆): δ 9.26 (s, 1H), 8.41-8.33 (m, 3H). 6.72-6.67 (m, 1H), 4.13-3.99 (m, 12H), 1.09-1.00 (m, 3H).

Step 3: 2-(Pyrimidin-2-yl)-6-(thiazole-5-carbonyl)-2,6-diazaspiro[3.4]octane-8-carboxylic acid: To a solution of ethyl 2-(pyrimidin-2-yl)-6-(thiazole-5-carbonyl)-2,6-diazaspiro[3.4]octane-8-carboxylate (936 mg, 2.5 mmol) in a mixture of THF (8 mL). MeOH (2 mL), and water (2 mL) was added LiOH·H₂O (315 mg, 7.5 mmol). The reaction mixture was stirred at room temperature for 2 hours and then diluted with water (50 mL) and extracted with EtOAc (80 mL). The aqueous layer was collected and acidified to pH of 4 with HCl (1M) and extracted with EtOAc (100 mL×3). The combined organic layers were washed with brine, dried over Na₂SO₄, filtered, and concentrated to afford 2-(pyrimidin-2-yl)-6-(thiazole-5-carbonyl)-2,6-diazaspiro[3.4]octane-8-carboxylic acid (282 mg, 33%) as a yellow solid. LCMS m/z=346.1 [M+H]⁺. ¹H NMR (400 MHz, DMSO-d₆): δ 9.33-9.31 (m, 1H), 9.27-9.24 (m, 1H), 8.45-8.43 (m, 1H), 8.41-8.39 (m, 1H), 8.38-8.34 (m, 2H), 6.72-6.67 (m, 1H), 4.28-4.00 (m, 8H), 3.21-3.18 (m, 3H).

Step 4: Mixture of N-((2S,3R)-3-((2-oxabicyclo[2.2.2]octan-4-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27A and N-((2S,3R)-3-((1-(hydroxymethyl)cyclohex-3-en-1-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27B: To a solution of 2-(pyrimidin-2-yl)-6-(thiazole-5-carbonyl)-2,6-diazaspiro[3.4]octane-8-carboxylic acid (50 mg, 0.14 mmol) in DCM (1 mL) was added HATU (66 mg, 0.17 mmol) and DIPEA (56 mg, 0.43 mmol). The mixture was stirred at room temperature for 30 minutes Next, (2S,3R)-3-((2-oxabicyclo[2.2.2]octan-4-yl)methoxy)-2-amino-1-(4-(trifluoromethyl)piperidin-1-yl)butan-1-one (55 mg, 0.14 mmol) was added, and the reaction was stirred at room temperature for another 2 hours. The mixture was diluted with water (10 mL) and extracted with DCM (30 mL×3). The combined organic layers were washed with brine, dried over Na₂SO₄, filtered, and concentrated. The residue was purified by prep-TLC (eluent: DCM/MeOH=20/1) to afford a mixture of N-((2S,3R)-3-((2-oxabicyclo[2.2.2]octan-4-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27A and N-((2S,3R)-3-((1-(hydroxymethyl)cyclohex-3-en-1-yl)methoxy)-1-oxo-1-(4-(trifluoromethyl)piperidin-1-yl)butan-2-yl)-2-(pyrimidin-2-yl)-6-(thiazole-5-carbonyl)-2,6-diazaspiro[3.4]octane-8-carboxamide I-27B in a ratio of about 1/1 (7.5 mg, 7%) as a white solid. LCMS m/z=248.1 [M+H]⁺. ¹H NMR (400 MHz, DMSO-d₆): δ 9.29-9.23 (m, 1H), 8.45-8.31 (m, 4H), 6.72-6.65 (m, 1H), 5.63-5.47 (m, 1H), 4.93-4.75 (m, 1H), 4.49-4.39 (m, 1H), 4.30-3.75 (m, 9H), 3.71-3.40 (m, 5H), 3.22-2.84 (m, 3H), 2.66-2.55 (m, 1H), 1.94-1.67 (m, 5H), 1.58-1.27 (m, 6H), 1.17-0.87 (m, 4H).

### Synthesis of a Mixture of N-((3R)-3-((2-oxabicyclo[2.2.2]octan-4-yl)methoxy)-1-(methylamino)-1-oxobutan-2-yl)-2-(benzo[d]oxazol-2-yl)-6-(thiazole-5-carbonyl)-2,6-diazaspiro[3.4]octane-8-carboxamide I-28A and 2-(benzo[d]oxazol-2-yl)-N-((3R)-3-((1-(hydroxymethyl)cyclohex-3-en-1-yl)methoxy)-1-(methylamino)-1-oxobutan-2-yl)-6-(thiazole-5-carbonyl)-2,6-diazaspiro[3.4]octane-8-carboxamide I-28B

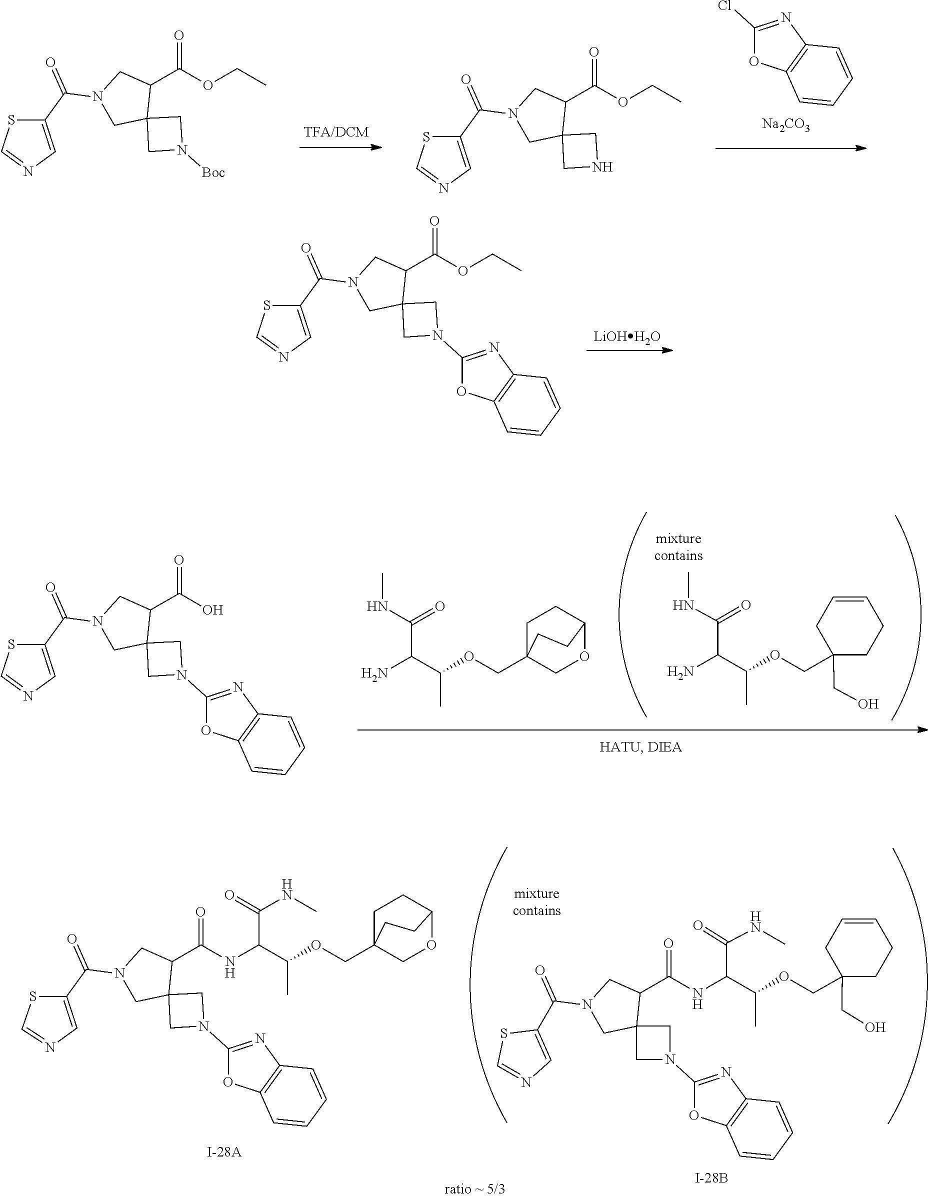

Step 1: Ethyl 6-(thiazole-5-carbonyl)-2,6-diazaspiro[3.4]octane-8-carboxylate: To a solution of 2-(tert-butyl) 8-ethyl 6-(thiazole-5-carbonyl)-2,6-diazaspiro[3.4]octane-2,8-dicarboxylate (350 mg, 0.89 mmol) in DCM (2 mL) was added TFA (1 mL). The reaction mixture was stirred at room temperature for 2 hours. The solvent was removed under vacuum to afford crude ethyl 6-(thiazole-5-carbonyl)-2,6-diazaspiro[3.4]octane-8-carboxylate (260 mg, 100%), which was used directly in the next step. LCMS m/z=296.1 [M+H]⁺.

Step 2: Ethyl 6-(thiazole-5-carbonyl)-2,6-diazaspiro[3.4]octane-8-carboxylate: To a solution of ethyl 6-(thiazole-5-carbonyl)-2,6-diazaspiro[3.4]octane-8-carboxylate (250 mg, 0.85 mmol) in MeCN (4 mL) was added Na₂CO₃ (270 mg, 2.54 mmol) and 2-chlorobenzo[d]oxazole (130 mg, 0.85 mmol). The reaction was heated at 70° C. for 2 hours. Next, the reaction mixture was filtered through Celite and concentrated to afford crude ethyl 6-(thiazole-5-carbonyl)-2,6-diazaspiro[3.4]octane-8-carboxylate (350 mg, 100%), which was used directly in the next step. LCMS m/z=413.1 [M+H]⁺. ¹H NMR (400 MHz, DMSO-d₆): δ 9.26 (d, J=2.4 Hz, 1H), 8.38 (d, J=10.4 Hz, 1H), 7.47-7.40 (m, 1H), 7.36-7.28 (m, 1H), 7.16 (t, J=7.6 Hz, 1H), 7.04 (t, J=7.8 Hz, 1H), 4.34 (dd, J=8.2, 5.4 Hz, 1H), 4.29-4.18 (m, 4H), 4.15-3.97 (m, 3H), 3.90 (d, J=4.8 Hz, 1H), 3.77 (d, J=6.6 Hz, 1H), 3.54 (dt. J=21.8, 6.6 Hz, 1H), 1.10 (dt. J=13.2, 7.2 Hz. 3H).

Step 3: 2-(Benzo[d]oxazol-2-yl)-6-(thiazole-5-carbonyl)-2,6-diazaspiro[3.4]octane-8-carboxylic acid: To a solution of ethyl 6-(thiazole-5-carbonyl)-2,6-diazaspiro[3.4]octane-8-carboxylate (300 mg, 0.73 mmol) in a mixture of THF (4 mL), water (1 mL), and EtOH (1 mL) was added LiOH (214 mg, 2.19 mmol). The reaction mixture was stirred at room temperature for 2 hours and then diluted with water (10 mL) and extracted with ether (15 mL). The aqueous layer was collected, acidified to a pH of 2 with 1M HCl, and extracted with EtOAc (50 mL×3). The combined organic layers were washed with brine, dried over Na₂SO₄, filtered, and concentrated to afford 2-(benzo[d]oxazol-2-yl)-6-(thiazole-5-carbonyl)-2,6-diazaspiro[3.4]octane-8-carboxylic acid (227 mg, 81%) as a yellow solid, which was used directly in the next step. LCMS m/z=385.1 [M+H]⁺. ¹H NMR (400 MHz, DMSO-d₆): δ 9.31-9.23 (m, 1H), 8.40 (t, J=10.8 Hz, 1H), 7.42 (dd, J=7.8, 3.4 Hz, 1H), 7.31 (dd, J=7.8, 4.2 Hz, 1H), 7.16 (td, J=7.6, 2.6 Hz, 1H), 7.05 (m, 1H). 4.37-4.19 (m, 4H), 3.90 (d, J=4.2 Hz, 1H), 3.75 (d, J=6.6 Hz, 1H), 3.48-3.39 (m, 3H).

Step 4: Mixture of N-((3R)-3-((2-oxabicyclo[2.2.2]octan-4-yl)methoxy)-1-(methylamino)-1-oxobutan-2-yl)-2-(benzo[d]oxazol-2-yl)-6-(thiazole-5-carbonyl)-2,6-diazaspiro[3.4]octane-8-carboxamide (1-28) and 2-(benzo[d]oxazol-2-yl)-N-((3R)-3-((1-(hydroxymethyl)cyclohex-3-en-1-yl)methoxy)-1-(methylamino)-1-oxobutan-2-yl)-6-(thiazole-5-carbonyl)-2,6-diazaspiro[3.4]octane-8-carboxamide (I-28B): To a solution of 2-(benzo[d]oxazol-2-yl)-6-(thiazole-5-carbonyl)-2,6-diazaspiro[3.4]octane-8-carboxylic acid (50) mg, 0.13 mmol) in DCM (2 mL) was added HATU (50 mg, 0.13 mmol) and DIPEA (50 mg, 0.39 mmol). The reaction was stirred at room temperature for 30 minutes. Next, (3R)-3-((2-oxabicyclo[2.2.2]octan-4-yl)methoxy)-2-amino-N-methylbutanamide (47 mg, 0.18 mmol) was added, and stirring was continued for another 2 hours. The mixture was diluted with water (30 mL) and extracted with DCM (30) mL×2). The combined organic layers were washed with brine, dried over Na₂SO₄, filtered, and concentrated. The mixture was purified by prep-HPLC to afford a mixture of N-((3R)-3-((2-oxabicyclo[2.2.2]octan-4-yl)methoxy)-1-(methylamino)-1-oxobutan-2-yl)-2-(benzo[d]oxazol-2-yl)-6-(thiazole-5-carbonyl)-2,6-diazaspiro[3.4]octane-8-carboxamide I-28A and 2-(benzo[d]oxazol-2-yl)-N-((3R)-3-((1-(hydroxymethyl)cyclohex-3-en-1-yl)methoxy)-1-(methylamino)-1-oxobutan-2-yl)-6-(thiazole-5-carbonyl)-2,6-diazaspiro[3.4]octane-8-carboxamide I-28B in a ratio of about 5/3 (12.7 mg, 16%) as a yellow solid. LCMS m/z=623.4 [M+H]⁺. ¹H NMR (400 MHz, Methanol-d₄): δ 9.16 (s, 0.84H), 8.42-8.35 (m, 1H), 7.38-7.27 (m, 2H), 7.25-7.06 (m, 2H), 5.72-5.47 (m, 0.55H), 4.58-3.63 (m, 12H), 3.60-3.36 (m, 2H), 3.24-2.89 (m, 2H), 2.74 (d, J=11.4 Hz, 1H), 2.61 (d, J=23.4 Hz, 1H), 1.97 (d, J=17.2 Hz, 2H), 1.78 (s, 1H), 1.72-1.35 (m, 5H), 1.19-0.99 (m, 3H).

### Building Block (2S,3R)-3-((2-oxabicyclo[2.2.2]octan-4-yl)methoxy)-2-amino-N-methylbutanamide

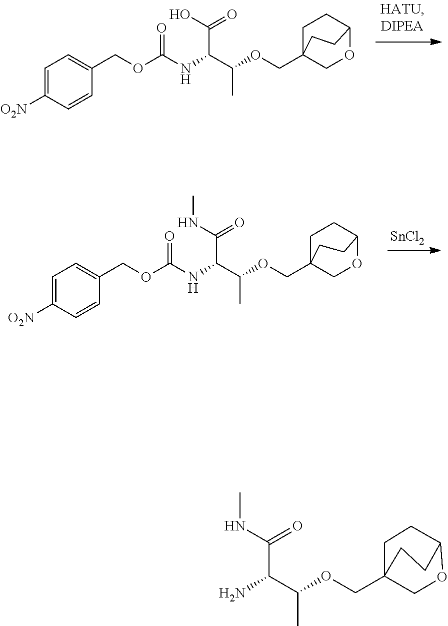

Step 1:4-Nitrobenzyl ((2S,3R)-3-((2-oxabicyclo[2.2.2]octan-4-yl)methoxy)-1-(methylamino)-1-oxobutan-2-yl)carbamate: To a solution of O-((2-oxabicyclo[2.2.2]octan-4-yl)methyl)-N-(((4-nitrobenzyl)oxy)carbonyl)-L-threonine (1.2 g, 2.84 mmol) in DCM (5 mL) was added HATU (0.98 g, 2.58 mmol) and DIPEA (1.33 g, 10.32 mmol). The mixture was stirred at room temperature for 30 minutes. Next, methanamine hydrochloride (175 mg, 2.58 mmol) was added, and the reaction was stirred for an additional 2 hours. The mixture was diluted with water (20 mL) and extracted with DCM (30 mL×2). The combined organic layers were washed with brine, dried over Na₂SO₄, filtered, and concentrated. The residue obtained was purified by column chromatography on silica gel (eluent: DCM/MeOH=70/1) to afford 4-nitrobenzyl ((2S,3R)-3-((2-oxabicyclo[2.2.2]octan-4-yl)methoxy)-1-(methylamino)-1-oxobutan-2-yl)carbamate (470 mg, 38%) as a yellow oil. LCMS m/z=436.1 [M+H]⁺. ¹H NMR (400 MHz, DMSO-d₆): δ 8.24 (d, J=8.4 Hz, 2H), 7.83 (d, J=5.0 Hz, 1H), 7.65 (d, J=8.4 Hz, 2H), 7.15 (d, J=9.1 Hz, 1H), 5.19 (d, J=3.0 Hz, 2H), 3.96 (dd, J=9.4, 4.8 Hz, 1H), 3.54-3.50 (m, 2H), 3.16-3.11 (m, 2H), 3.04 (d, J=5.2 Hz, 1H), 2.90 (d, J=9.0 Hz, 1H), 2.59 (d, J=4.6 Hz, 3H), 1.82 (m, 2H), 1.50 (m, 3H), 1.39-1.31 (m, 3H), 1.02 (t, J=6.8 Hz, 3H).

Step 2: (2S,3R)-3-((2-Oxabicyclo[2.2.2]octan-4-yl)methoxy)-2-amino-N-methylbutanamide: To a solution of 4-nitrobenzyl ((2S,3R)-3-((2-oxabicyclo[2.2.2]octan-4-yl)methoxy)-1-(methylamino)-1-oxobutan-2-yl)carbamate (450 mg, 1.03 mmol) in ethanol (6 mL) was added tin (II) chloride (980 mg, 5.2 mmol). The mixture was stirred at room temperature overnight. Next, the solvent was removed under reduced pressure, and the residue obtained was purified on an Al₂O₃ column (eluent: DCM/MeOH=50/1) to afford (2S,3R)-3-((2-oxabicyclo[2.2.2]octan-4-yl)methoxy)-2-amino-N-methylbutanamide (120 mg, 45%) as a yellow oil. LCMS m/z=257.2 [M+H]⁺ 1H NMR (400 MHz, DMSO-d₆): δ 6.95 (d, J=8.2 Hz, 1H), 6.51 (d, J=8.4 Hz, 1H), 4.45 (t, J=5.4 Hz, 1H), 4.04-3.96 (m, 1H), 3.68-3.48 (m, 6H), 3.04 (d, J=5.2 Hz, 1H), 2.62 (d, J=4.6 Hz, 1H), 1.83 (m, 2H), 1.58-1.47 (m, 4H), 1.40-1.33 (m, 2H), 1.18 (d, J=6.8 Hz, 3H).

### Synthesis of a Mixture of (2S,3R)-3-((2-oxabicyclo[2.2.2]octan-4-yl)methoxy)-2-amino-1-(4-(trifluoromethyl)piperidin-1-yl)butan-1-one and (2S,3R)-2-amino-3-((1-(hydroxymethyl)cyclohex-3-en-1-yl)methoxy)-1-(4-(trifluoromethyl)piperidin-1-yl)butan-1-one

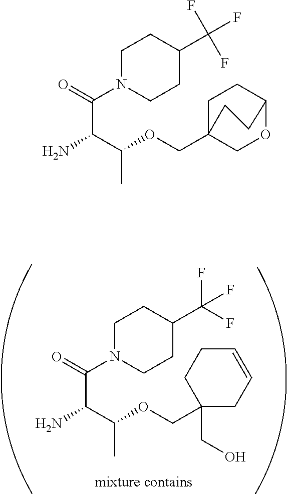

A mixture of (2S,3R)-3-((2-oxabicyclo[2.2.2]octan-4-yl)methoxy)-2-amino-1-(4-(trifluoromethyl)piperidin-1-yl)butan-1-one and (2S,3R)-2-amino-3-((1-(hydroxymethyl)cyclohex-3-en-1-yl)methoxy)-1-(4-(trifluoromethyl)piperidin-1-yl)butan-1-one (ratio˜1/1) was synthesized from 2-methyl 1-(4-nitrobenzyl) (2S,3S)-3-methylaziridine-1,2-dicarboxylate according to the procedures outlined for (2S,3R)-3-((2-oxabicyclo[2.2.2]octan-4-yl)methoxy)-2-amino-1-((S)-3-((trifluoromethoxy)methyl)piperidin-1-yl)butan-1-one using the appropriate commercially available reagents and/or intermediates described elsewhere. LCMS m/z=379.2 [M+H]⁺. ¹H NMR (400 MHz, DMSO-d₆): δ 5.63-5.50 (m, 1H), 3.68-3.43 (m, 5H), 3.27-2.91 (m, 4H), 2.03-1.95 (m, 1H), 1.94-1.70 (m, 6H), 1.69-1.47 (m, 4H), 1.46-1.32 (m, 4H).

### Synthesis of (S)-6-(1-benzyl-1H-pyrazole-4-carbonyl)-N-((2S,3R)-3-(cyclohexylmethoxy)-1-(methylamino)-1-oxobutan-2-yl)-2-(oxazol-2-yl)-2,6-diazaspiro[3.4]octane-8-carboxamide I-2

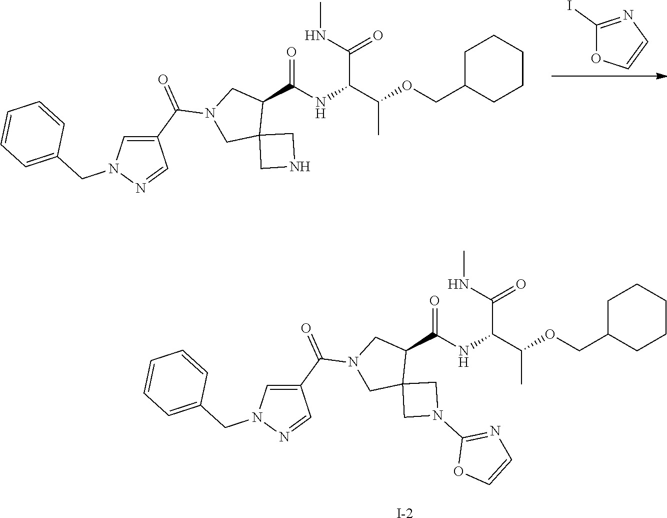

To a solution of (S)-6-(1-benzyl-1H-pyrazole-4-carbonyl)-N-((2S,3R)-3-(cyclohexylmethoxy)-1-(methylamino)-1-oxobutan-2-yl)-2,6-diazaspiro[3.4]octane-8-carboxamide (169 mg, 0.31 mmol) in DMSO was added 2-iodooxazole (40 mg, 0.21 mmol), L-proline (5 mg, 0.04 mmol), and potassium carbonate (85 mg, 0.62 mmol). The reaction was heated at 90° C. overnight. The reaction was diluted with water and extracted with EtOAc. The combined organic layers were concentrated, and the residue obtained was purified by prep-TLC (eluent: DCM/MeOH=20/1, v/v) followed by prep-HPLC to afford (S)-6-(1-benzyl-1H-pyrazole-4-carbonyl)-N-((2S,3R)-3-(cyclohexylmethoxy)-1-(methylamino)-1-oxobutan-2-yl)-2-(oxazol-2-yl)-2,6-diazaspiro[3.4]octane-8-carboxamide (5 mg, 4%) as a white solid. LCMS m/z=618.4 [M+H]⁺. ¹H NMR (400 MHz, CDCl₃): δ 7.85-7.81 (m, 2H), 7.39-7.31 (m, 3H), 7.26-7.21 (m, 3H), 7.03-6.90 (m, 1H), 6.82 (s, 1H), 6.64-6.53 (m, 1H), 5.30 (s, 2H), 4.56-4.43 (m, 1H), 4.31-3.73 (m, 9H), 3.37 (d, J=6.4 Hz, 2H), 3.18-3.09 (m, 1H), 2.84 (d, J=4.7 Hz, 3H), 1.62-1.49 (m, 2H), 1.33-1.10 (m, 4H), 1.04-0.88 (m, 5H).

### Synthesis of (8S)—N-(2-hydroxy-2-methyl-1-(6-(tetrahydro-2H-pyran-4-yl) 239yridine-2-yl) propyl)-2-(oxazol-2-yl)-6-(thiazole-5-carbonyl)-2,6-diazaspiro[3.4]octane-8-carboxamide I-5

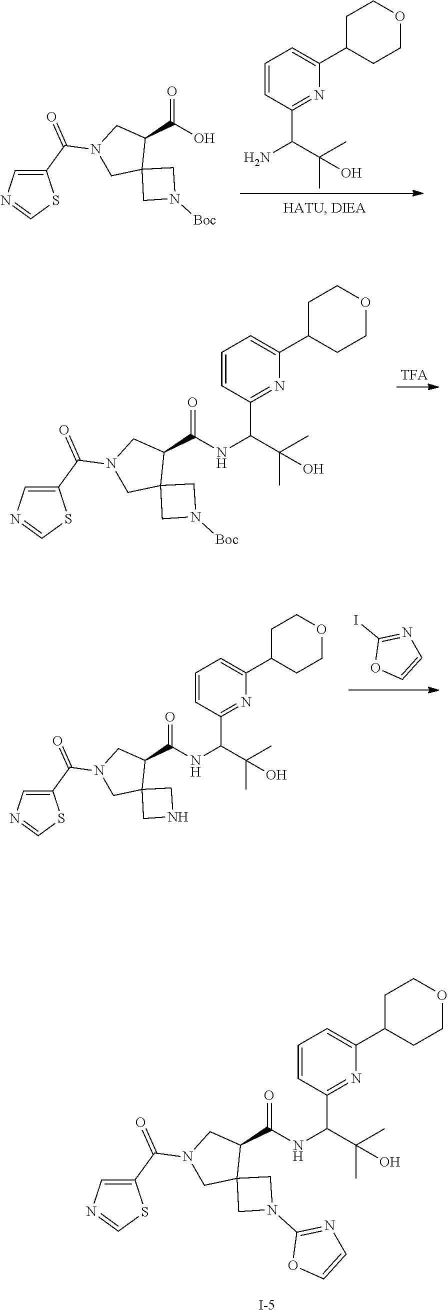

Step 1: tert-butyl (8S)-8-((2-hydroxy-2-methyl-1-(6-(tetrahydro-2H-pyran-4-yl) 240yridine-2-yl) propyl)carbamoyl)-6-(thiazole-5-carbonyl)-2,6-diazaspiro[3.4]octane-2-carboxylate: To a solution of 1-amino-2-methyl-1-(6-(tetrahydro-2H-pyran-4-yl) 240yridine-2-yl) propan-2-ol (136 mg, 0.54 mmol) in DCM (4 mL) was added HATU (205 mg, 0.54 mmol) and DIPEA (279 mg, 2.16 mmol). The mixture was stirred at room temperature for 30 minutes. (S)-2-(tert-butoxycarbonyl)-6-(thiazole-5-carbonyl)-2,6-diazaspiro[3.4]octane-8-carboxylic acid (200 mg, 0.54 mmol) was added, and stirring was continued for another 2 hours. The mixture was diluted with water (15 mL) and extracted with DCM (30 mL×3). The combined organic layers were washed with brine, dried over Na₂SO₄, filtered, and concentrated. The residue obtained was purified by prep-TLC (eluent: Pet. DCM/MeOH=15:1) to afford tert-butyl (8S)-8-((2-hydroxy-2-methyl-1-(6-(tetrahydro-2H-pyran-4-yl) 240yridine-2-yl) propyl)carbamoyl)-6-(thiazole-5-carbonyl)-2,6-diazaspiro[3.4]octane-2-carboxylate (235 mg, 72%) as a yellow oil. LCMS m/z=600.3 [M+H]⁺. ¹H NMR (400 MHz, CDCl₃): δ 8.91 (d, J=3.2 Hz, 1H), 8.23 (d, J=8.0 Hz, 1H), 7.66 (m, 1H), 7.22-7.13 (m, 2H), 7.04-6.95 (m, 1H), 4.93-4.77 (m, 1H), 4.13-4.05 (m, 3H), 3.96-3.86 (m, 3H), 3.75-3.70 (m, 3H), 3.59-3.51 (m, 3H), 3.19-3.17 (m, 1H), 3.07-3.01 (m, 1H), 1.70-1.67 (m, 4H), 1.44 (s, 9H), 1.39-1.33 (m, 7H).

Step 2: (8S)—N-(2-hydroxy-2-methyl-1-(6-(tetrahydro-2H-pyran-4-yl)240yridine-2-yl) propyl)-6-(thiazole-5-carbonyl)-2,6-diazaspiro[3.4]octane-8-carboxamide: To a solution of tert-butyl (8S)-8-((2-hydroxy-2-methyl-1-(6-(tetrahydro-2H-pyran-4-yl)240yridine-2-yl) propyl)carbamoyl)-6-(thiazole-5-carbonyl)-2,6-diazaspiro[3.4]octane-2-carboxylate (230 mg, 0.38 mmol) in DCM (3 mL) was added TFA (1.5 mL). The reaction mixture was stirred at room temperature for 2 hours. The solvent was removed under vacuum, and the crude residue was purified by SCX-column to afford (8S)—N-(2-hydroxy-2-methyl-1-(6-(tetrahydro-2H-pyran-4-yl)241yridine-2-yl) propyl)-6-(thiazole-5-carbonyl)-2,6-diazaspiro[3.4]octane-8-carboxamide (75 mg, 39%) as a white solid. LCMS m/z=500.3 [M+H]⁺.

Step 3: (85)-N-(2-hydroxy-2-methyl-1-(6-(tetrahydro-2H-pyran-4-yl)241yridine-2-yl) propyl)-2-(oxazol-2-yl)-6-(thiazole-5-carbonyl)-2,6-diazaspiro[3.4]octane-8-carboxamide: To a solution of (8S)—N-(2-hydroxy-2-methyl-1-(6-(tetrahydro-2H-pyran-4-yl) 241 yridine-2-yl) propyl)-6-(thiazole-5-carbonyl)-2,6-diazaspiro[3.4]octane-8-carboxamide (75 mg, 0.15 mmol) in THF was added 2-iodooxazole (35 mg, 0.18 mmol). The reaction was heated at 70° C. overnight, and then the solvent was removed under vacuum. The residue obtained was purified by prep-HPLC followed by prep-TLC (eluent: DCM/MeOH=10:1, v/v) to afford (8S)—N-(2-hydroxy-2-methyl-1-(6-(tetrahydro-2H-pyran-4-yl)241yridine-2-yl) propyl)-2-(oxazol-2-yl)-6-(thiazole-5-carbonyl)-2,6-diazaspiro[3.4]octane-8-carboxamide (9 mg, 11%) as a white solid. LCMS m/z=567.3 [M+H]⁺. ¹H NMR (400 MHz, DMSO-d₆) δ 9.28-9.19 (m, 1H), 8.61-8.50 (m, 1H), 8.41-8.27 (m, 1H), 7.71-7.52 (m, 2H), 7.25-7.10 (m, 2H), 6.83 (d, J=14.2 Hz, 1H), 5.14-4.99 (m, 1H), 4.90-4.76 (m, 1H), 4.32-3.83 (m, 8H), 3.81-3.52 (m, 3H), 3.50-3.39 (m, 2H), 3.00-2.85 (m, 1H), 1.86-1.67 (m, 4H), 1.13-0.98 (m, 6H).

### Synthesis of (8-(5-((3,4-dichlorophenyl)difluoromethyl)-1,3,4-oxadiazol-2-yl)-2-(241yridine-2-yl)-2,6-diazaspiro[3.4]octan-6-yl) (thiazol-5-yl)methanone I-58

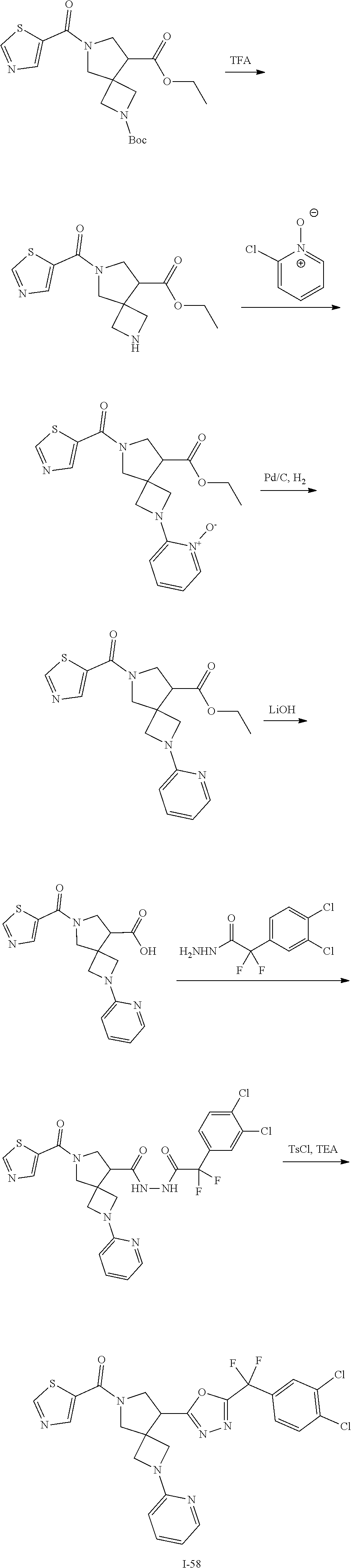

Step 1: ethyl 6-(thiazole-5-carbonyl)-2,6-diazaspiro[3.4]octane-8-carboxylate: To a solution of 2-(tert-butyl) 8-ethyl 6-(thiazole-5-carbonyl)-2,6-diazaspiro[3.4]octane-2,8-dicarboxylate (500 mg, 1.26 mmol) in DCM (5 mL) was added TFA (4 mL). The reaction was stirred at room temperature for 2 hours. The solvent was removed under vacuum to afford ethyl 6-(thiazole-5-carbonyl)-2,6-diazaspiro[3.4]octane-8-carboxylate (373 mg, 100%), which was used directly in the next step. LCMS m/z=296.1 [M+H]⁺.

Step 2: 2-(8-(ethoxycarbonyl)-6-(thiazole-5-carbonyl)-2,6-diazaspiro[3.4]octan-2-yl)pyridine 1-oxide: To a solution of ethyl 6-(thiazole-5-carbonyl)-2,6-diazaspiro[3.4]octane-8-carboxylate (373 mg, 1.26 mmol) in CH₃CN (5 mL) was added 2-chloropyridine 1-oxide (326 mg, 2.52 mmol) and Na₂CO₃ (400 mg, 3.78 mmol). The reaction mixture was heated at 100° C. for 4 hours in a microwave reactor. The solvent was removed under reduced pressure, and the residue obtained was purified by prep-TLC (eluent: DCM/MeOH-10:1) to afford 2-(8-(ethoxycarbonyl)-6-(thiazole-5-carbonyl)-2,6-diazaspiro[3.4]octan-2-yl)pyridine 1-oxide (230 mg, 47%) as a white solid LCMS m/z=389.1 [M+H]⁺. ¹H NMR (400 MHz, DMSO-d₆): δ 9.26 (s, 1H), 8.39 (d, J=13.4 Hz, 1H), 7.95 (d, J=5.8 Hz, 1H), 7.18 (t, J=7.2 Hz, 1H), 6.75 (t, J=6.6 Hz, 1H), 6.61 (d, J=8.2 Hz, 1H), 4.32-3.95 (m, 6H), 3.80 (dd, J=32.2, 5.0 Hz, 1H), 3.44 (m, 1H), 2.25 (t, J=6.6 Hz, 1H), 1.76 (p, J=6.0 Hz, 1H), 1.23 (d, J=4.4 Hz, 1H), 1.14 (m, 2H).

Step 3: ethyl 2-(pyridin-2-yl)-6-(thiazole-5-carbonyl)-2,6-diazaspiro[3.4]octane-8-carboxylate: To a solution of 2-(8-(ethoxy carbonyl)-6-(thiazole-5-carbonyl)-2,6-diazaspiro[3.4]octan-2-yl)pyridine 1-oxide (320 mg, 0.82 mmol) in EtOH (3 mL) was added 10% Pd/C (170 mg). The reaction was stirred under a H₂ atmosphere for 24 hours. The catalyst was removed by filtration through Celite, and the filtrate was concentrated. The residue obtained was purified by prep-TLC (eluent: DCM/MeOH-10:1) to afford ethyl 2-(pyridin-2-yl)-6-(thiazole-5-carbonyl)-2,6-diazaspiro[3.4]octane-8-carboxylate (270 mg, 88%) as a white solid. LCMS m/z=373.2 [M+H]⁺. ¹H NMR (DMSO, 400 MHz): δ 9.26 (d, J=3.4 Hz, 1H), 8.39 (d, J=12.2 Hz, 1H), 8.06 (d, J=5.2 Hz, 1H), 7.50 (t, J=7.2 Hz, 1H), 6.65 (q, J=5.4 Hz, 1H), 6.40 (t, J=7.0 Hz, 1H), 4.21-3.81 (m, 9H), 3.77 (d, J=6.6 Hz, 1H), 1.11-1.02 (m, 4H).

Step 4: 2-(pyridin-2-yl)-6-(thiazole-5-carbonyl)-2,6-diazaspiro[3.4]octane-8-carboxylic acid: To a solution of ethyl 2-(pyridin-2-yl)-6-(thiazole-5-carbonyl)-2,6-diazaspiro[3.4]octane-8-carboxylate (230 mg, 0.618 mmol) in a mixture of THF, MeOH, and water (4 mL/1 mL/1 mL) was added LiOH (30 mg, 1.23 mmol). The reaction was stirred at room temperature for 2 hours. The mixture was diluted with water (50 mL) and extracted with EtOAc (80 mL). The aqueous layer was collected, acidified with 1M HCl to PH˜2, and extracted with EtOAc (100 mL×2). The combined organic layers were washed with brine, dried over Na₂SO₄, filtered, and concentrated to afford 2-(pyridin-2-yl)-6-(thiazole-5-carbonyl)-2,6-diazaspiro[3.4]octane-8-carboxylic acid (80 mg, 38%) as a white solid, which was used directly in the next step. LCMS m/z=345.1 [M+H]⁺. ¹H NMR (DMSO, 400 MHz): δ 9.25 (d, J=5.4 Hz, 1H), 8.44-8.27 (m, 1H), 8.05 (d, J=5.2 Hz, 1H), 7.53 (q, J=6.8 Hz, 1H), 7.44-7.31 (m, 1H), 6.66 (q, J=5.6 Hz, 1H), 6.43 (t, J=7.2 Hz, 1H), 4.14 (d, J=3.0 Hz, 1H), 4.05 (m, 2H), 3.96 (d, J=8.4 Hz, 2H), 3.85 (s, 1H), 3.78-3.67 (m, 2H), 3.62 (d, J=6.8 Hz, 1H).

Step 5: N′-(2-(3,4-dichlorophenyl)-2,2-difluoroacetyl)-2-(pyridin-2-yl)-6-(thiazole-5-carbonyl)-2,6-diazaspiro[3.4]octane-8-carbohydrazide: To a solution of 2-(pyridin-2-yl)-6-(thiazole-5-carbonyl)-2,6-diazaspiro[3.4]octane-8-carboxylic acid (30 mg, 0.09 mmol) in DMF (2 mL) was added 2-(3,4-dichlorophenyl)-2,2-difluoroacetohydrazide (24 mg, 0.09 mmol), EDCl (21 mg, 0.11 mmol), HOBt (18 mg, 0.14 mmol), and DIPEA (45 mg, 0.36 mmol). The reaction was stirred at room temperature for 14 hours, and then the solvent was removed under reduced pressure. The residue was purified by prep-TLC (eluent: DCM/MeOH=12:1) to afford N′-(2-(3,4-dichlorophenyl)-2,2-difluoroacetyl)-2-(pyridin-2-yl)-6-(thiazole-5-carbonyl)-2,6-diazaspiro[3.4]octane-8-carbohydrazide (25 mg, 48%) as a yellow solid. LCMS m/z=581.0 [M+H]⁺.

Step 6: (8-(5-((3,4-dichlorophenyl)difluoromethyl)-1,3,4-oxadiazol-2-yl)-2-(pyridin-2-yl)-2,6-diazaspiro[3.4]octan-6-yl) (thiazol-5-yl)methanone (1-58): To a solution of N-(2-(3,4-dichlorophenyl)-2,2-difluoroacetyl)-2-(pyridin-2-yl)-6-(thiazole-5-carbonyl)-2,6-diazaspiro[3.4]octane-8-carbohydrazide (20 mg, 0.03 mmol) in DCM (5 mL) was added TEA (17 mg, 0.17 mmol) and TsCl (20 mg, 0.10 mmol). The reaction was stirred at room temperature for 14 hours, and then the solvent was removed under reduced pressure. The residue was purified by prep-TLC (eluent: DCM/MeOH-15:1) to afford (8-(5-((3,4-dichlorophenyl)difluoromethyl)-1,3,4-oxadiazol-2-yl)-2-(pyridin-2-yl)-2,6-diazaspiro[3.4]octan-6-yl) (thiazol-5-yl)methanone I-58 (5 mg, 26%) as a white solid. LCMS m/z=563.1 [M+H]⁺: ¹H NMR (DMSO, 400 MHz): δ 9.27 (s, 1H), 8.40 (d, J=9.2 Hz, 1H), 8.05 (s, 1H), 7.97 (d, J=7.8 Hz, 1H), 7.78 (d, J=8.4 Hz, 1H), 7.64 (d, J=8.0 Hz, 1H), 7.50 (s, 1H), 6.66 (d, J=6.6 Hz, 1H), 6.34 (d, J=8.2 Hz, 1H), 4.24 (dd, J=20.5, 9.3 Hz, 3H), 4.03 (t, J=11.6 Hz, 3H), 3.91 (t, J=13.4 Hz, 3H).

### Synthesis of (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indol-4-yl)-2,6-diazaspiro[3.4]octane-8-carboxamide (I-14)

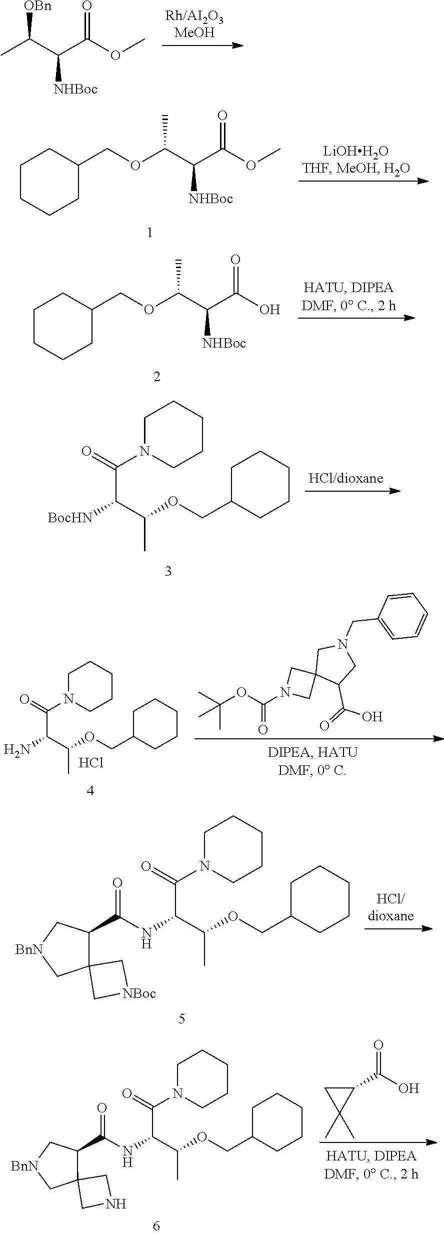

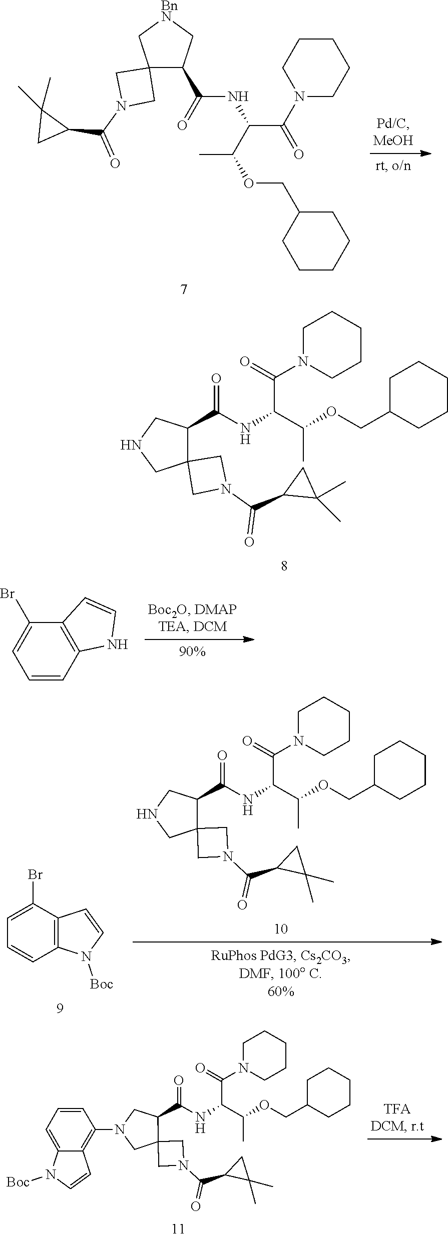 [SMILES: C[C@@H](OCC1CCCCC1)[C@H](NC(=O)[C@@H]1CN(Cc2ccccc2)CC12CN(C(=O)[C@H]1CC1(C)C)C2)C(=O)N1CCCCC1 ; C[C@@H](OCC1CCCCC1)[C@H](NC(=O)[C@@H]1CNCC12CN(C(=O)[C@H]1CC1(C)C)C2)C(=O)N1CCCCC1 ; Brc1cccc2[nH]ccc12 ; CC(C)(C)OC(=O)n1ccc2c(Br)cccc21 ; C[C@@H](OCC1CCCCC1)[C@H](NC(=O)[C@@H]1CNCC12CN(C(=O)[C@H]1CC1(C)C)C2)C(=O)N1CCCCC1 ; C[C@@H](OCC1CCCCC1)[C@H](NC(=O)[C@@H]1CN(c2cccc3c2ccn3C(=O)OC(C)(C)C)CC12CN(C(=O)[C@H]1CC1(C)C)C2)C(=O)N1CCCCC1]

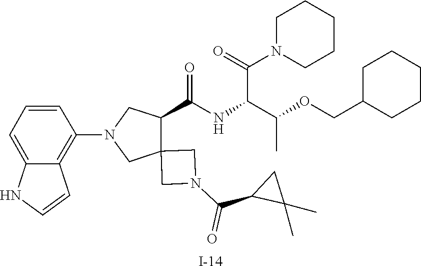

Step 1: (2S,3R)-methyl 2-((tert-butoxycarbonyl)amino)-3-(cyclohexylmethoxy) butanoate (1): To a solution of (2S,3R)-methyl 3-(benzyloxy)-2-((tert-butoxycarbonyl)amino) butanoate (0.960 g, 2.97 mmol) in methanol (30 mL) was added Rh/Al₂O₃ (0.300 g). The resulting mixture was stirred at 45° C. under hydrogen atmosphere (H₂ balloon) for 16 hours. Rh/Al₂O₃ was removed through filtration and washed with methanol (20 mL×2). The combined organic solution was concentrated under reduced pressure to give a crude residue, which was purified by silica gel column chromatography using a 10-20% ethyl acetate in hexane gradient to afford (2S,3R)-methyl 2-((tert-butoxycarbonyl)amino)-3-(cyclohexylmethoxy)butanoate 1 (0.767 g, 78% yield) as a colorless oil. ¹HNMR (400 MHz, CDCl₃): δ 5.21 (d, J=9.6 Hz, 1H), 4.27-4.24 (m, 1H), 3.98-3.93 (m, 1H), 3.73 (s, 3H), 3.32-3.29 (m, 1H), 3.03-2.99 (m, 1H), 1.68-1.66 (m, 4H), 1.46-1.42 (m, 11H), 1.23-1.14-(m, 6H), 0.90-0.83 (m, 2H).

Step 2: (2S,3R)-2-((tert-butoxycarbonyl)amino)-3-(cyclohexylmethoxy)butanoic acid (2): To a solution of (2S,3R)-methyl 2-((tert-butoxycarbonyl)amino)-3-(cyclohexylmethoxy) butanoate 1 (0.500 g, 1.52 mmol) in a mixture of tetrahydrofuran (8 mL)-methanol (2 mL)-water (2 mL) at 0° C. was added lithium hydroxide monohydrate (0.128 g, 3.04 mmol). The resulting mixture was stirred at room temperature overnight. The reaction mixture was acidified to pH of 4-5 with hydrochloric acid (2.0 N, 50.8 mL, 101.6 mmol). The precipitate was collected through filtration and dried under vacuum to afford (2S,3R)-2-((tert-butoxycarbonyl)amino)-3-(cyclohexylmethoxy)butanoic acid 2 (0.470 g, 98% yield) as a colorless oil. ¹H NMR (400 MHz, CDCl₃): δ 5.32 (d, J=6.8 Hz, 1H), 4.37 (s, 1H), 4.02 (s, 1H), 3.42 (t, J=8.8 Hz, 1H), 3.29 (t, J=7.2 Hz, 1H), 1.72-1.67 (m, 5H), 1.46 (s, 9H), 1.22-1.13 (m, 6H), 0.96-0.90 (m, 2H).

Step 3: tert-Butyl ((2S,3R)-3-(cyclohexylmethoxy)-1-oxo-1-(piperidin-1-yl)butan-2-yl)carbamate (3): To a solution of (2S,3R)-2-((tert-butoxy carbonyl)amino)-3-(cyclohexylmethoxy)butanoic acid 2 (0.470 g, crude), piperidine (0.140 g, 1.64 mmol), and N-ethyl-N-isopropylpropan-2-amine (0.5 mL) in N,N-dimethylformamide (3 mL) at 0° C. was added HATU (2-(7-aza-1H-benzotriazole-1-yl)-1,1,3,3-tetramethyluronium hexafluorophosphate (0.680 g, 1.79 mmol). The resulting mixture was stirred at 0° C. for 1 hour. TLC showed the reaction was complete. The mixture was poured into saturated ammonium chloride solution (15 mL) and extracted with ethyl acetate (20 mL). The organic layer was collected, washed with brine (15 mL), dried over anhydrous sodium sulfate, and concentrated under reduced pressure to give a crude residue, which was purified by column chromatography using a 10% ethyl acetate in hexane gradient to afford tert-butyl ((2S,3R)-3-(cyclohexylmethoxy)-1-oxo-1-(piperidin-1-yl)butan-2-yl)carbamate 3 (0.510 g, 88% yield) as a colorless oil. ¹H NMR (400 MHz, CDCl₃): δ 5.60 (d, J=8.2 Hz, 1H), 4.67 (dd, J=8.4, 3.8 Hz, 1H), 3.69-3.65 (m, 1H), 3.60-3.55 (m, 2H), 3.49-3.42 (m, 2H), 3.32 (dd, J=9.2, 6.4 Hz, 1H), 3.20-3.16 (m, 1H), 1.71-1.59 (m, 10H), 1.43 (s, 9H), 1.24-1.15 (m, 3H), 1.12 (d, J=6.4 Hz, 3H), 0.93-0.84 (m, 3H).

Step 4: tert-Butyl (S)-6-benzyl-8-(((2S,3R)-3-(cyclohexylmethoxy)-1-oxo-1-(piperidin-1-yl)butan-2-yl)carbamoyl)-2,6-diazaspiro[3.4]octane-2-carboxylate (5): A mixture of tert-butyl ((2S,3R)-3-(cyclohexylmethoxy)-1-oxo-1-(piperidin-1-yl)butan-2-yl)carbamate 3 (0.510 g, 1.49 mmol) in hydrogen chloride solution in dioxane (4 M, 2 mL) was stirred at room temperature for 2 hours. The reaction mixture was concentrated in vacuo to give crude 4 which was used in the coupling reaction of the next step without further purification. To a solution of (2S,3R)-2-amino-3-(cyclohexylmethoxy)-1-(piperidin-1-yl)butan-1-one 4 (0.445 g, 1.40 mmol), 6-benzyl-2-(tert-butoxy carbonyl)-2,6-diazaspiro[3.4]octane-8-carboxylic acid (0.484 g. 1.40 mmol), and N-ethyl-N-isopropylpropan-2-amine (1 mL) in N,N-dimethylformamide (3 mL) at 0° C. was added HATU (2-(7-aza-1H-benzotriazole-1-yl)-1,1,3,3-tetramethyluronium hexafluorophosphate) (0.585 g, 1.54 mmol). The resulting mixture was stirred at 0° C. for 1 hour. The reaction mixture was poured into saturated ammonium chloride solution (25 mL) and extracted with ethyl acetate (15 mL×2). The combined organic phases were washed with brine (20) mL), dried over anhydrous sodium sulfate, and concentrated under reduced pressure to give a crude residue, which was purified by silica gel column chromatography using a mixture of dichloromethane-ethyl acetate-hexane (2:2:1, v/v/v) gradient to afford tert-butyl (S)-6-benzyl-8-(((2S,3R)-3-(cyclohexylmethoxy)-1-oxo-1-(piperidin-1-yl)butan-2-yl)carbamoyl)-2,6-diazaspiro[3.4]octane-2-carboxylate 5 (0.615 g, 71% yield) as a yellow oil. MS: [MH]⁺ 611.1.

Step 5: (S)-6-benzyl-N-((2S,3R)-3-(cyclohexylmethoxy)-1-oxo-1-(piperidin-1-yl) butan-2-yl)-2,6-diazaspiro[3.4]octane-8-carboxamide (6): A mixture of tert-butyl (S)-6-benzyl-8-(((2S,3R)-3-(cyclohexylmethoxy)-1-oxo-1-(piperidin-1-yl)butan-2-yl)carbamoyl)-2,6-diazaspiro[3.4]octane-2-carboxylate 5 (0) 615 g, 1.01 mmol) in hydrogen chloride solution in dioxane (4 M, 3 mL) was stirred at room temperature overnight. The reaction mixture was concentrated under reduced pressure to give a crude residue, which was taken up into water (10) mL) and basified to pH 8 with aq. NaOH solution (1.0 M). The aqueous layer was extracted with ethyl acetate (10) mL×2). The combined organic layers were washed with brine (20 mL), dried over anhydrous sodium sulfate, and concentrated under reduced pressure to give a crude residue, which was purified by silica gel column chromatography using a 2.5% methanol in dichloromethane gradient to afford (S)-6-benzyl-N-((2S,3R)-3-(cyclohexylmethoxy)-1-oxo-1-(piperidin-1-yl)butan-2-yl)-2,6-diazaspiro[3.4]octane-8-carboxamide 6 (415 mg, 58% yield) as a colorless oil. MS: [MH]⁺ 511.2.

Step 6: (S)-6-benzyl-N-((2S,3R)-3-(cyclohexylmethoxy)-1-oxo-1-(piperidin-1-yl) butan-2-yl)-2-((S)-2,2-dimethylcyclopropane-1-carbonyl)-2,6-diazaspiro[3.4]octane-8-carboxamide (7): To a solution of (S)-6-benzyl-N-((2S,3R)-3-(cyclohexylmethoxy)-1-oxo-1-(piperidin-1-yl)butan-2-yl)-2,6-diazaspiro[3.4]octane-8-carboxamide 6 (0.315 g, 0.62 mmol), (S)-2,2-dimethylcyclopropanecarboxylic acid (0.071 g, 0.62 mmol), and N-ethyl-N-isopropylpropan-2-amine (0.240 g, 1.86 mmol) in N,N-dimethylformamide (2 mL) at (° C.) was added HATU (2-(7-aza-1H-benzotriazole-1-yl)-1,1,3,3-tetramethyluronium hexafluorophosphate (0.259 g, 0.68 mmol). The resulting mixture was stirred at room temperature under N₂ for 2 hours. The reaction mixture was poured into saturated ammonium chloride solution (15 mL) and extracted with ethyl acetate (20) mL). The combined organic phases were washed with brine (15 mL), dried over anhydrous sodium sulfate, and concentrated under reduced pressure to give a crude residue, which was purified by silica gel column chromatography using a 3% methanol in dichloromethane gradient to afford (S)-6-benzyl-N-((2S,3R)-3-(cyclohexylmethoxy)-1-oxo-1-(piperidin-1-yl) butan-2-yl)-2-((S)-2,2-dimethylcyclopropane-1-carbonyl)-2,6-diazaspiro[3.4]octane-8-carboxamide 7 (0.340 g, 90% yield) as a colorless oil. MS: [MH]⁺ 607.8.

Step 7: N-((2S,3R)-3-(cyclohexylmethoxy)-1-oxo-1-(piperidin-1-yl)butan-2-yl)-2-((S′)-2,2-dimethylcyclopropanecarbonyl)-2,6-diazaspiro[3.4]octane-8-carboxamide (8): To a solution of (S)-6-benzyl-N-((2S,3R)-3-(cyclohexylmethoxy)-1-oxo-1-(piperidin-1-yl)butan-2-yl)-2-((S)-2,2-dimethylcyclopropane-1-carbonyl)-2,6-diazaspiro[3.4]octane-8-carboxamide 7 (0.340 g, 0.56 mmol) in MeOH (20 mL) was added Pd/C (10%, 44 mg). The mixture was stirred at 40° C.) under H₂ atmosphere overnight. The reaction mixture was filtered, and the filter cake was washed with DCM (10 mL×3). The combined filtrates were concentrated under reduced pressure to give a crude residue, which was purified by column chromatography using a 5% methanol in dichloromethane containing 1% NH₃·H₂O gradient to afford N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide 8 (0.290 g, 99% yield) as a white solid. MS: [MH]⁺ 517.8.

Step 8: tert-Butyl 4-bromo-1H-indole-1-carboxylate (9): To a solution of 4-bromo-1H-indole (1 g, 5.1 mmol), di-tert-butyl dicarbonate (1.67 g, 7.65 mmol), and triethylamine (1.03 g. 10.20 mmol) in dichloromethane (10) mL) was added 4-dimethylaminopyridine (125 mg, 1.02 mmol). The resulting mixture was stirred at room temperature under nitrogen atmosphere for 40 minutes. The reaction mixture was poured into water (8 mL) and extracted with dichloromethane (12 mL). The combined organic layers were washed with water (8 mL×2) and brine (8 mL), dried over anhydrous sodium sulfate, and concentrated to give a crude residue, which was purified by silica gel column chromatography using a 0.5% ethyl acetate in hexane gradient to afford tert-butyl 4-bromo-1/1-indole-1-carboxylate 9 (1.35 g, 90%) as a colorless oil. ¹H NMR (400 MHz, DMSO-d₆): δ 8.07 (d, J=4.2 Hz, 1H), 7.79 (d, J=1.8 Hz, 1H), 7.47 (d, J=3.8 Hz, 1H), 7.27 (t, J=8.2 Hz, 1H), 6.66 (d, J=1.8 Hz, 1H), 1.63 (s, 9H).

Step 9: tert-Butyl 4-((S)-8-(((2S,3R)-3-(cyclohexylmethoxy)-1-oxo-1-(piperidin-1-yl) butan-2-yl)carbamoyl)-2-((S)-2,2-dimethylcyclopropane-1-carbonyl)-2,6-diazaspiro[3.4]octan-6-yl)-1H-indole-1-carboxylate (11): To a solution of (S)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide 8 (0.090 g, 0.17 mmol), tert-butyl 4-bromo-1H-indole-1-carboxylate 9 (0.067 g, 0.23 mmol), and cesium carbonate (0.113 g, 0.35 mmol) in N,N-dimethylformamide (1.5 mL) was added RuPhos-Pd-G3 (0.029 g, 0.035 mmol) under nitrogen atmosphere. The mixture was stirred at 100° C. overnight. The reaction mixture was poured into water (5 mL) and extracted with ethyl acetate (8 mL). The organic layer was washed with water (5 mL×2) and brine (5 mL), dried over anhydrous sodium sulfate, and concentrated to give a crude residue, which was purified by silica gel column chromatography using a 10%-50% ethyl acetate in dichloromethane gradient to afford tert-butyl 4-((S)-8-(((2S,3R)-3-(cyclohexylmethoxy)-1-oxo-1-(piperidin-1-yl)butan-2-yl)carbamoyl)-2-((S)-2,2-dimethylcyclopropane-1-carbonyl)-2,6-diazaspiro[3.4]octan-6-yl)-1H-indole-1-carboxylate 11 (0.077 g, 62% yield) as a green solid. MS: [MH]⁺ 732.5.

Step 10: (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indol-4-yl)-2,6-diazaspiro[3.4]octane-8-carboxamide (1-14): To a solution of tert-butyl 4-((S)-8-(((2S,3R)-3-(cyclohexylmethoxy)-1-oxo-1-(piperidin-1-yl)butan-2-yl)carbamoyl)-2-((S)-2,2-dimethylcyclopropane-1-carbonyl)-2,6-diazaspiro[3.4]octan-6-yl)-1H-indole-1-carboxylate 11 (0.072 g, 0.098 mmol) in dichloromethane (0.5 mL) was added trifluoroacetic acid (0.5 mL). The resulting mixture was stirred at room temperature for 2 hours. The reaction mixture was basified with saturated aqueous sodium bicarbonate solution (3 mL), and extracted with dichloromethane (5 mL×3). The combined organic layers were dried over anhydrous sodium sulfate and concentrated to give a crude residue, which was purified by prep-TLC using a 3% methanol in dichloromethane gradient to afford (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indol-4-yl)-2,6-diazaspiro[3.4]octane-8-carboxamide I-14 (0.028 g, 37% yield) as a green solid. ¹H NMR (400 MHz, CD₃OD): δ 8.22-8.14 (m, 1H), 7.11 (s, 1H), 6.97 (t, J=8.0 Hz, 1H), 6.88 (d, J=4.0 Hz, 1H), 6.68 (s, 1H), 6.23 (d, J=3.8 Hz, 1H), 4.96-4.93 (m, 1H), 4.44-4.39 (m, 1H), 4.31-4.18 (m, 2H), 4.11-3.79 (m, 6H), 3.75-3.70 (m, 1H), 3.56 (s, 4H), 3.37 (s, 1H), 3.30 (d, J=3.8 Hz, 1H), 3.19-3.12 (m, 1H), 1.67-1.52 (m, 11H), 1.20-1.07 (m, 13H), 0.92-0.74-(m, 4H). MS: [MH]⁺ 632.6.

The following compounds were prepared in a manner analogous to the procedures described above for (S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indol-4-yl)-2,6-diazaspiro[3.4]octane-8-carboxamide (I-14):

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indazol-4-yl)-2,6-diazaspiro[3.4]octane-8-carboxamide I-13 (0.025 g, 20% yield) as a green solid. ¹HNMR (400 MHz. CD₃OD): δ 8.21 (s. 1H), 7.19 (t, J=7.8 Hz, 1H), 6.81 (d, J=8.4 Hz, 1H), 6.11 (d, J=8.4 Hz, 1H), 4.97-4.93 (m, 1H). 4.43 (t, J=9.8 Hz, 1H), 4.35-4.19 (m, 2H), 4.15-3.99 (m, 3H), 3.97-3.87 (m, 3H), 3.76-3.70 (m, 1H), 3.61-3.38 (m, 6H), 3.20-3.14 (m, 1H), 1.74-1.64 (m, 6H), 1.58-1.42 (m, 7H), 1.19-1.15 (m, 11H), 1.07-1.04 (m, 1H), 0.92-0.85 (m, 2H), 0.81-0.77 (m, 1H). MS: [MH]⁺ 633.3.

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-pyrazolo[4,3-c]pyridin-4-yl)-2,6-diazaspiro[3.4]octane-8-carboxamide I-9 (0.040 g, 66% yield) as a white solid. ¹HNMR (400 MHz, CD₃OD): δ 8.36 (s, 1H), 7.72 (d, J=6.4 Hz, 1H), 6.84 (d, J=6.4 Hz, 1H), 4.96-4.93 (m, 1H), 4.47-4.01 (m, 8H), 3.78-3.73 (m, 1H), 3.58-3.51 (m, 5H), 3.38-3.33 (m, 1H), 3.23-3.17 (m, 1H), 1.76-1.44 (m, 13H), 1.56-1.24 (m, 11H), 1.08-1.05 (m, 1H), 0.97-0.89 (m, 3H), 0.83-0.78 (m, 1H). MS: [MH]⁺635.0.

(S)-6-(benzo[d]thiazol-7-yl)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide 1-7 (0.026 g, 42% yield) as a yellow solid. ¹HNMR (400 MHz, CD₃OD): δ 9.14 (s. 1H), 7.49 (d, J=7.6 Hz, 1H), 7.40) (t, J=8.0 Hz, 1H), 6.72-6.70) (m, 1H), 4.95-4.92 (m, 1H), 4.42 (d, J=8.8 Hz, 1H), 4.32-4.06 (m, 3H), 4.01-3.85 (m, 5H), 3.75-3.72 (m, 1H), 3.54-3.42 (m, 5H), 3.36-3.34 (m, 1H), 3.20-3.14 (m, 1H), 1.70-1.42 (m, 13H), 1.18-1.14 (m, 11H), 1.07-1.04 (m, 1H). 0.93-0.88 (m, 2H), 0.79-0.76 (m, 1H). MS: [MH]⁺ 650.4.

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(thiazolo[5,4-c]pyridin-4-yl)-2,6-diazaspiro[3.4]octane-8-carboxamide I-15 (0.040 g, 31% yield) as a yellow solid. ¹HNMR (400 MH/CD₃OD): δ 9.36 (s, 1H), 8.05 (d, J=5.6 Hz, 1H), 7.28 (d, J=5.6 Hz, 1H), 4.96-4.93 (m, 1H), 4.45-4.40 (m, 1H). 4.34-4.16 (m, 3H), 4.15-4.07 (m, 3H), 4.01-3.96 (m, 1H), 3.70-3.77 (m, 1H), 3.43-3.56 (m, 5H). 3.32-3.36 (m, 1H), 3.17-3.22 (m, 1H), 1.75-1.46 (m, 13H), 1.26-1.21 (m, 2H), 1.18-1.14 (m, 10H). 1.07-1.04 (m, 1H), 0.96-0.90 (m, 2H), 0.81-0.76 (m, 1H). MS: [MH]⁺ 651.7.

(S)-N-((2S,3R)-3-(cyclohexylmethoxy)-1-oxo-1-(piperidin-1-yl)butan-2-yl)-2-((S)-2,2-dimethylcyclopropane-1-carbonyl)-6-(1H-pyrazolo[3,4-c]pyridin-7-yl)-2,6-diazaspiro[3.4]octane-8-carboxamide 2,2,2-trifluoroacetate I-12 (60) mg, 61% yield) as an off-white solid. ¹HNMR (400 MHz CD₃OD): δ 8.37 (s, 1H), 7.23 (d, J=6.4 Hz, 1H), 7.14 (d, J=6.8 Hz, 1H), 4.99-4.93 (m, 1H), 4.89-4.82 (m, 1H), 4.75-3.92 (m, 7H), 3.82-3.44 (m, 6H), 3.40-3.34 (m, 1H), 3.25-3.17 (m, 1H), 1.81-1.31 (m, 13H), 1.31-1.13 (m, 11H), 1.11-1.04 (m, 1H), 0.99-0.88 (m, 2H), 0.85-0.78 (m, 1H). MS: [MH]⁺634.9.

6-(benzo[d]thiazol-4-yl)-N-((2S,3R)-3-(cyclohexylmethoxy)-1-oxo-1-(piperidin-1-yl)butan-2-yl)-2-((S)-2,2-dimethylcyclopropanecarbonyl)-2,6-diazaspiro[3.4]octane-8-carboxamide 1-8 (33 mg, 36% yield) as a white solid. ¹HNMR (400 MHz. CD₃OD): δ 8.89 (d, J=2.8 Hz, 1H), 7.34 (d, J=7.8 Hz, 1H), 7.27 (t, J=7.8 Hz, 1H), 6.65 (dd. J=7.8, 1.8 Hz, 1H). 4.94-4.91 (m, 1H), 4.42 (t, J=8.8 Hz, 1H), 4.32-3.95 (m, 8H), 3.75-3.69 (m, 1H), 3.53-3.48 (m, 4H), 3.42-3.33 (m, 2H), 3.20-3.13 (m, 1H), 1.70-1.41 (m, 13H), 1.29-1.15 (m, 12H), 1.07-1.04 (m, 1H), 0.94-0.88 (m, 2H), 0.81-0.76 (m, 1H). MS: [MH]⁺ 650.3.

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(1H-indazol-7-yl)-2,6-diazaspiro[3.4]octane-8-carboxamide I-11 (28 mg, 37% yield) as a green solid. ¹H NMR (400 MHz. CDCl₃): δ 8.70 (d, J=4.4 Hz, 1H), 8.46-8.44 (m, 1H), 8.06 (s, 1H), 7.41 (d, J=4.0 Hz, 1H), 7.07 (t, J=7.6 Hz, 1H). 6.83-6.78 (m, 1H), 4.97 (d, J=4.0 Hz, 1H), 4.42-4.25 (m, 2H), 4.19-4.11 (m, 2H), 4.04-3.96 (m, 2H), 3.85-3.80 (m, 1H), 3.70-3.67 (m, 2H), 3.60-3.50 (m, 3H), 3.45-3.35 (m, 2H), 3.16-3.09 (m, 2H), 3.02-2.94 (m, 1H), 1.66-1.57 (m, 7H), 1.34-1.20 (m, 6H), 1.15-1.08 (m, 9H), 0.94-0.86 (m, 3H), 0.74-0.71 (m, 1H), 0.64-0.50 (m, 2H). MS: [MH]⁺ 633.6.

(S)-N-((2S,3R)-3-(cyclohexylmethoxy)-1-oxo-1-(piperidin-1-yl)butan-2-yl)-2-((S)-2,2-dimethylcyclopropanecarbonyl)-6-(thiazolo[4,5-c]pyridin-4-yl)-2,6-diazaspiro[3.4]octane-8-carboxamide I-10 (22 mg, 29% yield) as a yellow solid. ¹HNMR (400 MHz. CD₃OD): δ 8.98 (s. 1H), 7.93 (d, J=5.6 Hz, 1H), 7.26 (d, J=5.6 Hz, 1H), 4.96-4.93 (m, 1H), 4.07-4.47 (m, 7H), 3.95-3.99 (m, 1H), 3.71-3.77 (m, 1H), 3.34-3.59 (m, 6H), 3.17-3.22 (m, 1H), 1.60-1.76 (m, 6H), 1.40-1.56 (m, 7H), 1.14-1.27 (m, 11H), 1.04-1.07 (m, 1H), 0.88-0.96 (m, 2H), 0.76-0.81 (m, 1H). MS: [MH]⁺ 651.7.

### Synthesis of 2-methyl 1-(4-nitrophenyl)methyl (2S,3S)-3-methylaziridine-1,2-dicarboxylate

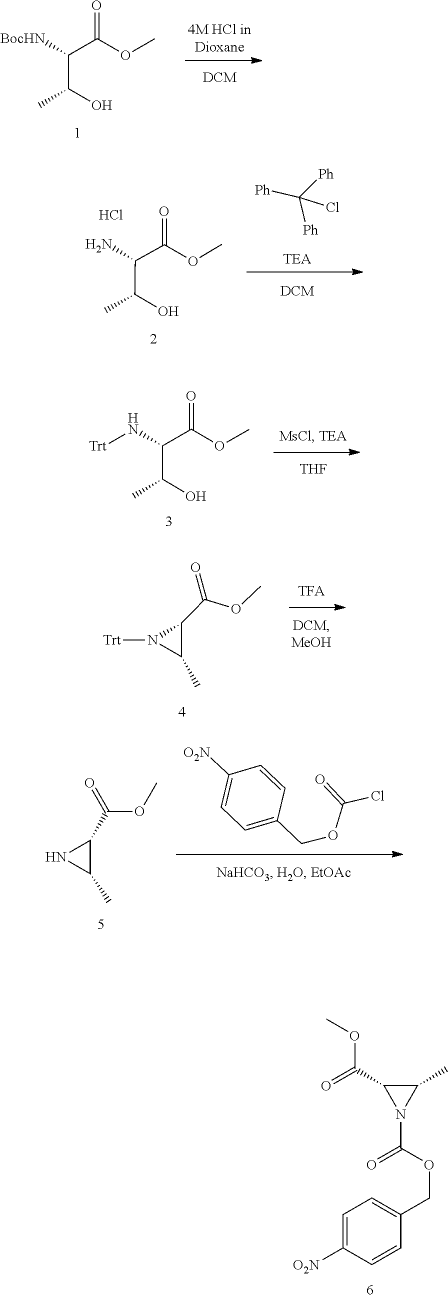

Methyl L-threoninate (2): To a stirred solution of methyl (tert-butoxycarbonyl)-L-threoninate 1 (20.0 g, 85.78 mmol) in DCM (100 mL) was added HCl (4M) in dioxane (40 mL) at 0° C. The mixture was stirred at room temperature for 2 hours. [Another identical batch of reagents was reacted in the same manner, and the respective reaction mixtures were worked up together]. Combined reaction mixtures were concentrated under reduced pressure. The obtained crude product was purified by trituration using n-pentane, to afford methyl L-threoninate 2 (45.0 g, quantitative; crude). MS: [MH]⁺ 134.2.

Methyl trityl-L-threoninate (3): To a stirred solution of methyl L-threoninate 2 (14.5 g, 109.02 mmol) in DCM (100 mL) were added TEA (55.13 g, 544.82 mmol) and (chloromethanetriyl)tribenzene (36.36 g, 130.75 mmol) sequentially at 0° C. under nitrogen. The reaction mixture was stirred for 30 minutes at the same temperature. [Two identical batches with 5.0 g and 12.5 g of methyl L-threoninate 2 were reacted in the same manner, and the respective reaction mixtures were combined prior to work-up]. Combined reaction mixtures were slowly poured into water (300 mL) and extracted with DCM (300 mL×3). Combined organic extracts were washed with brine (200 mL), dried over anhydrous Na₂SO₄, and concentrated in vacuo. The crude product was purified by silica gel column chromatography using ethyl acetate-hexane=0:1-+2:3 as gradient, to afford methyl trityl-L-threoninate 3 (48.0 g, 53%) as a yellow sticky compound. 1H-NMR (400 MHz, DMSO-d₆): δ 7.43-7.42 (d, J=7.6 Hz, 6H), 7.29-7.26 (t, J=7.2 Hz, 6H), 7.21-7.17 (t, J=7.2 Hz, 3H), 5.04-5.03 (d, J=4.4 Hz, 1H), 3.96-3.91 (m, 1H), 3.22-3.16 (m, 1H), 3.02 (s, 3H), 2.69-2.67 (d, J=10.0 Hz, 1H), 1.99 (s, 3H), 1.09-1.07 (d, J=6.4 Hz, 3H). MS: [MH]⁺ not supported.

Methyl (2S,3S)-3-methyl-1-tritylaziridine-2-carboxylate (4): To a stirred solution of methyl trityl-L-threoninate 3 (35.0 g, 93.28 mmol) in THF (300 mL) was added TEA (26.7 mL, 186.57 mmol) at 0° C. The reaction mixture was stirred for 20 minutes. Mesyl chloride (8.9 mL, 111.94 mmol) was then added dropwise into the reaction mixture at the same temperature over a period of 10 minutes. The resulting mixture was stirred at 80° C. for 24 hours. The reaction mixture was cooled to room temperature, diluted with water (50 mL), and extracted with ethyl acetate (50) mL×3). Combined organic extracts were washed with brine (50 mL), dried over anhydrous Na₂SO₄, and concentrated in vacuo. The crude product was purified by silica gel column chromatography, using ethyl acetate-hexane=0:1→0.8 as gradient, to afford methyl (2S,3S)-3-methyl-1-tritylaziridine-2-carboxylate 4 (22.0 g, 66%) as a white solid. 1H-NMR (400 MHz, DMSO-d₆): δ 7.44-7.42 (d, J=7.6 Hz, 6H), 7.34-7.30 (t, J=7.2 Hz, 6H), 7.28-7.19 (m, 3H), 3.67 (s, 3H), 1.73-1.71 (d, J=6.4 Hz, 1H), 1.59-1.53 (m, 1H), 1.29-1.28 (d, J=5.2 Hz, 3H). MS: [MH]⁺ not supported.

2-Methyl 1-(4-nitrobenzyl) (2S,3S)-3-methylaziridine-1,2-dicarboxylate (6): To a stirred suspension of methyl (2S,3S)-3-methyl-1-tritylaziridine-2-carboxylate 4 (2.0 g, 5.60 mmol) in DCM-MeOH mixture (1:3; 20 mL) was added TFA (10.0 mL) at 0° C. The resulting reaction mixture was stirred at room temperature for 2 hours. The reaction mixture was concentrated under reduced pressure until all methanol was distilled off. The obtained crude was diluted with water (30 mL) and extracted with diethyl ether (30 mL×3). Collected aqueous layers were basified (pH˜9) with the addition of solid NaHCO₃ and diluted with ethyl acetate (30 mL). To the stirred biphasic solution was added 4-nitrobenzylchloroformate (1.80 g, 8.403 mmol) slowly at 0° C. The reaction mixture was stirred at room temperature for 16 hours. The reaction mixture was diluted with water (30 mL) and extracted with ethyl acetate (50 mL×2). Combined organic extracts were washed with brine (30 mL), dried over anhydrous Na₂SO₄, and concentrated in vacuo. The crude product was purified by silica gel column chromatography, using ethyl acetate-hexane=0:1→3:7 as gradient, to afford 2-methyl 1-(4-nitrobenzyl) (2S,3S)-3-methylaziridine-1,2-dicarboxylate 6 (1.0 g, 62%) as a white solid. 1H-NMR (400 MHz, DMSO-d₆): δ 8.25-8.23 (d, J=8.0 Hz, 2H), 7.65-7.63 (d, J=8.0 Hz, 2H), 5.24 (s, 2H), 3.73-3.69 (d, J=14.8 Hz, 3H), 3.43-3.41 (d, J=6.8 Hz, 1H), 3.05-3.01 (m, 1H), 1.21-1.19 (d, J=5.6 Hz, 3H). MS: [MH]⁺ not supported.

### Synthesis of (S)-N-((2R,3R)-3-((2-oxabicyclo[2.2.2]octan-4-yl)methoxy)-1-((tetrahydro-2H-pyran-4-yl)oxy)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-57A and (R)—N-((2R,3R)-3-((2-oxabicyclo[2.2.2]octan-4-yl)methoxy)-1-((tetrahydro-2H-pyran-4-yl)oxy)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-57B

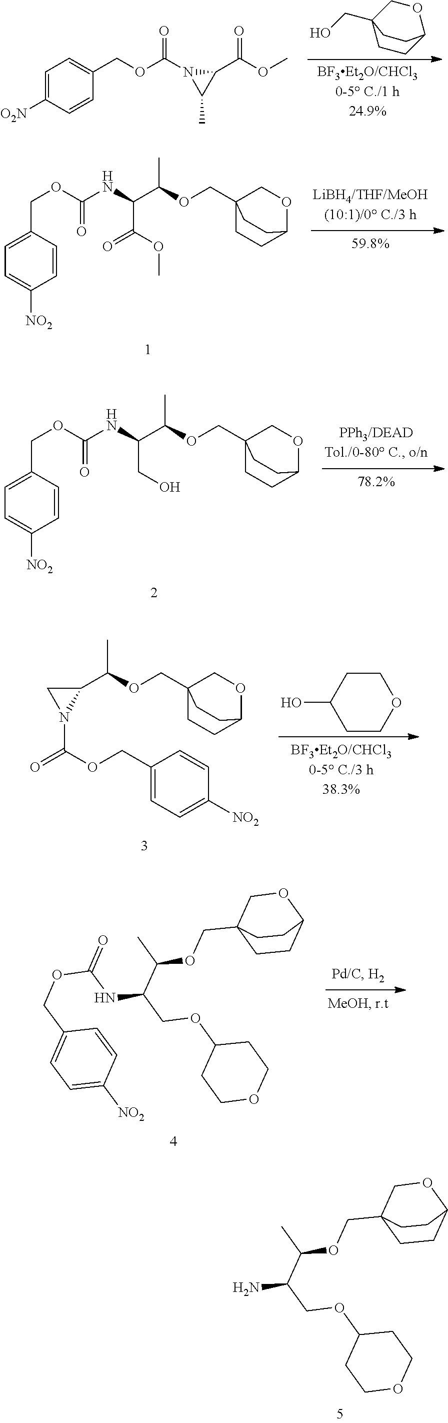

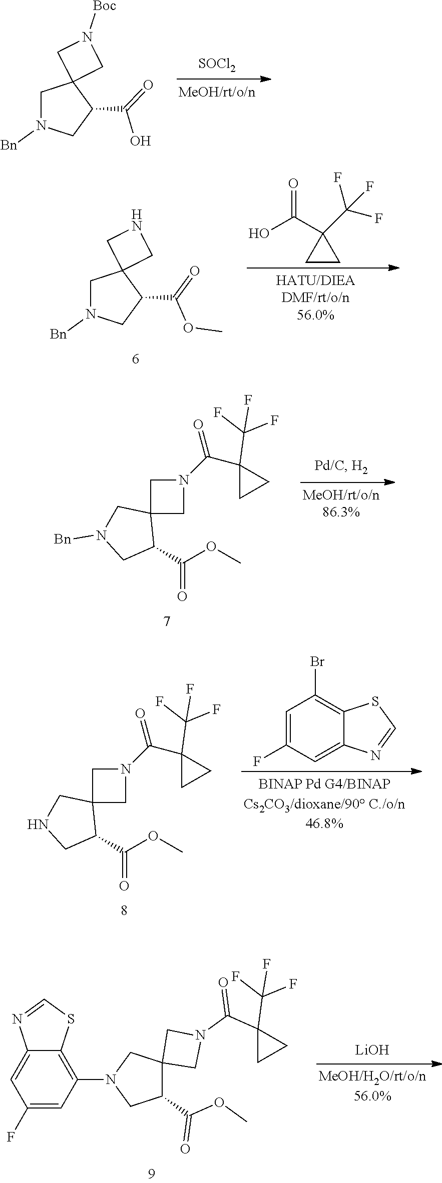

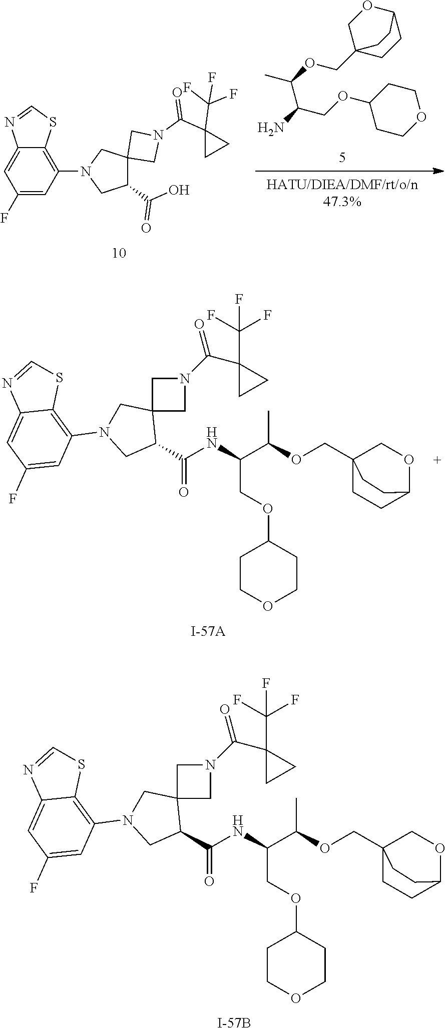

Step 1: Methyl (2S,3R)-2-({[(4-nitrophenyl)methoxy]carbonyl}amino)-3-{2-oxabicyclo[2.2.2]octan-4-ylmethoxy}butanoate: A solution of 2-methyl 1-(4-nitrophenyl)methyl (2S,3S)-3-methylaziridine-1,2-dicarboxylate (10 g, 34.01 mmol, 1.0 equiv). 2-oxabicyclo[2.2.2]octan-4-ylmethanol (4.83 g, 34.01 mmol, 1.0 equiv), and BF₃·Et₂O (965 mg, 6.80 mmol, 0.2 equiv) in chloroform (100 mL) was stirred for 1 hour at 0° C. under nitrogen atmosphere. The reaction was monitored by LCMS. The resulting mixture was concentrated under reduced pressure. The residue was purified by silica gel column chromatography, eluting with petroleum ether/ethyl acetate (1:1) to afford methyl (2S,3R)-2-({[(4-nitrophenyl) methoxy]carbonyl}amino)-3-{2-oxabicyclo[2.2.2]octan-4-ylmethoxy}butanoate (3.7 g, 24.9%) as a yellow oil. LCMS (ESI, m/z)=437.3 [M+H]⁺.

Step 2: (4-Nitrophenyl)methyl N-[(2R,3R)-1-hydroxy-3-{2-oxabicyclo[2.2.2]octan-4-ylmethoxy}butan-2-yl]carbamate: Methyl (2S,3R)-2-({[(4-nitrophenyl)methoxy]carbonyl}amino)-3-{2-oxabicyclo[2.2.2]octan-4-ylmethoxy}butanoate (5 g, 11.467 mmol, 1.0 equiv) was dissolved in MeOH (2 mL) and THF (8.5 mL). Next, LiBH₄ (11.5 mL, 2.0 equiv, 2 M in THF) was added dropwise at 0° C. The reaction was stirred for 3 h at 0° C. under nitrogen atmosphere. The reaction was monitored by LCMS. The reaction was quenched with water at 0° C. and extracted with EtOAc (3×30 mL). The combined organic layers were washed with brine (1×30 mL) and dried over anhydrous Na₂SO₄. After filtration, the filtrate was concentrated under reduced pressure to give a residue. The residue was purified by silica gel column chromatography, eluting with petroleum ether/ethyl acetate (1:1) to afford (4-nitrophenyl)methyl N-[(2R,3R)-1-hydroxy-3-{2-oxabicyclo[2.2.2]octan-4-ylmethoxy}butan-2-yl]carbamate (2.8 g, 59.8%) as a yellow oil. LCMS (ESI, m/z)=409.3 [M+H]⁺.

Step 3: (4-Nitrophenyl)methyl (2R)-2-[(1R)-1-{2-oxabicyclo[2.2.2]octan-4-ylmethoxy}ethyl]aziridine-1-carboxylate: A solution of (4-nitrophenyl)methyl N-[(2R,3R)-1-hydroxy-3-{2-oxabicyclo[2.2.2]octan-4-ylmethoxy}butan-2-yl]carbamate (2 g, 4.902 mmol, 1.0 equiv) and PPh₃ (1.80 g, 6.862 mmol, 1.4 equiv) in toluene (20 mL) was stirred for 10 minutes at room temperature under nitrogen atmosphere. Then DEAD (1.2 g, 5.382 mmol, 1.4 equiv) was added dropwise at 0° C. The resulting mixture was stirred overnight at 80° C. under nitrogen atmosphere. The reaction was monitored by LCMS. The resulting mixture was concentrated under reduced pressure. The residue was purified by silica gel column chromatography, eluting with petroleum ether/ethyl acetate (1:1) to afford (4-nitrophenyl)methyl (2R)-2-[(1R)-1-{2-oxabicyclo[2.2.2]octan-4-ylmethoxy}ethyl]aziridine-1-carboxylate (1.5 g, 78.2%) as a yellow solid. LCMS (ESI, m/z)=391.4 [M+H]⁺.

Step 4: N-[(2R,3R)-3-{2-oxabicyclo[2.2.2]octan-4-ylmethoxy}-1-(oxan-4-yloxy)butan-2-yl]carbamate: (4-Nitrophenyl)methyl (2R)-2-[(1R)-1-{2-oxabicyclo[2.2.2]octan-4-ylmethoxy}ethyl]aziridine-1-carboxylate (3 g, 7.684 mmol, 1.0 equiv) and oxan-4-ol (7.85 g, 76.840 mmol, 10 equiv) was dissolved in CHCl₃ (30 mL). Next, a solution of BF₃·Et₂O (327.2 mg, 2.305 mmol, 0.3 equiv) in CHCl₃ (2 mL) was added dropwise at 0° C. under nitrogen atmosphere. The resulting mixture was stirred for 3 hours at 0° C. under nitrogen atmosphere. The reaction was monitored by LCMS. The reaction mixture was quenched with water (100 mL) at 0° C. and extracted with DCM (3×50 mL). The combined organic phase was washed with water (4×60 mL) and brine (1×50) mL) and dried over anhydrous Na₂SO₄. After filtration, the filtrate was concentrated under reduced pressure. The residue was purified by silica gel column chromatography, eluting with CH₂Cl/ethyl acetate (3:1) to afford (4-nitrophenyl)methyl N-[(2R,3R)-3-{2-oxabicyclo[2.2.2]octan-4-ylmethoxy}-1-(oxan-4-yloxy)butan-2-yl]carbamate (1.45 g, 38.3%) as an off-white semi-solid. LCMS (ESI, m/z)=493.4 [M+H]⁺.

Step 5: (2R,3R)-3-((2-oxabicyclo[2.2.2]octan-4-yl)methoxy)-1-((tetrahydro-2H-pyran-4-yl)oxy)butan-2-amine: (4-Nitrophenyl)methyl N-[(2R,3R)-3-{2-oxabicyclo[2.2.2]octan-4-ylmethoxy}-1-(oxan-4-yloxy)butan-2-yl]carbamate (1.45 g, 2.944 mmol, 1.0 equiv) was dissolved in MeOH (2 mL). Next, Pd/C (1.4 g) was added under nitrogen atmosphere. The reaction was stirred for 3 hours at room temperature under H₂ atmosphere. The reaction was monitored by LCMS. The resulting mixture was filtered, and the filtrate was concentrated under reduced pressure to afford crude (2R,3R)-3-{2-oxabicyclo[2.2.2]octan-4-ylmethoxy}-1-(oxan-4-yloxy) butan-2-amine (1.1 g, crude) as a colorless oil. The crude product was purified by protection with benzyloxycarbonyl (Cbz) followed by removal of the protecting group with Pd/C to afford the pure product (310 mg, 33.6%). LCMS (ESI, m/z)=314.3 [M+H]⁺. ¹H NMR (400 MHz, Methanol-d₄): δ 3.91-3.86 (m, 2H), 3.75-3.70 (m, 3H), 3.54-3.29 (m, 6H), 3.26 (d, J=12.0 Hz, 1H), 2.97 (d, J=8.0 Hz, 1H), 2.77-2.72 (m, 1H), 2.04-1.87 (m, 4H), 1.70-1.64 (m, 4H), 1.57-1.48 (m, 4H), 1.11 (d, J=4.0 Hz, 3H).

Step 6: Methyl (8S)-6-benzyl-2,6-diazaspiro[3.4]octane-8-carboxylate: To a solution of (8S)-6-benzyl-2-(tert-butoxy carbonyl)-2,6-diazaspiro[3.4]octane-8-carboxylic acid (3.7 g, 10.680 mmol, 1.0 equiv) in MeOH (30 mL) was added thionyl chloride (3 mL) at (° C.) under nitrogen atmosphere. The mixture was stirred overnight at room temperature. The reaction was monitored by LCMS. The mixture was concentrated under reduced pressure to afford the crude product, methyl (85)-6-benzyl-2,6-diazaspiro[3.4]octane-8-carboxylate (2.9 g), as a yellow solid, which was used in the next step directly without further purification. LCMS (ESI, m/z)=261.3 [M+H]⁺.

Step 7: Methyl (8S)-6-benzyl-2-[1-(trifluoromethyl)cyclopropanecarbonyl]-2,6-diazaspiro[3.4]octane-8-carboxylate: A solution of 1-(trifluoromethyl)cyclopropane-1-carboxylic acid (1.65 g, 10.680 mmol, 1.0 equiv) and HATU (6.09 g, 16.02 mmol. 1.5 equiv) in DMF (20 mL) was stirred for 5 min at 0° C.) Next, DIEA (5.59 mL, 32.04 mmol, 3.0 equiv) and methyl (85)-6-benzyl-2,6-diazaspiro[3.4]octane-8-carboxylate (2.9 g, crude) in DMF (10 mL) were added dropwise at 0° C. The mixture was stirred for 3 hours at room temperature. The reaction was monitored by LCMS. The reaction mixture was quenched by the addition of water and extracted with DCM (3×100 mL). The combined organic phase was washed with brine (60) mL) and dried over anhydrous Na₂SO₄. After filtration, the filtrate was concentrated under reduced pressure. The residue was purified by silica gel column chromatography, eluting with CH₃CN/DCM (1:9) to afford methyl (85)-6-benzyl-2-[1-(trifluoromethyl)cyclopropanecarbonyl]-2,6-diazaspiro[3.4]octane-8-carboxylate (2.37 g, 56.0%) as a brown oil. LCMS (ESI, m/z)=397.0 [M+H]⁺.

Step 8: Methyl (8S)-2-[1-(trifluoromethyl)cyclopropanecarbonyl]-2,6-diazaspiro[3.4]octane-8-carboxylate: Methyl (85)-6-benzyl-2-[1-(trifluoromethyl)cyclopropanecarbonyl]-2,6-diazaspiro[3.4]octane-8-carboxylate (2.37 g, 5.985 mmol, 1.0 equiv) and Pd/C (2.4 g) in methanol (30) mL) were stirred overnight at room temperature under H₂ atmosphere. The resulting mixture was filtered, and the filter cake was washed with MeOH (4×20 mL). The filtrate was concentrated under reduced pressure. The residue was purified by silica gel column chromatography, eluting with CH₂Cl₂/MeOH (1:1) to afford methyl (88)-2-[1-(trifluoromethyl)cyclopropanecarbonyl]-2,6-diazaspiro[3.4]octane-8-carboxylate (1.58 g, 86.3%) as a light-yellow oil. LCMS (ESI, m/z)=307.1 [M+H]⁺.

Step 9: Methyl (8S)-6-(5-fluoro-1,3-benzothiazol-7-yl)-2-[1-(trifluoromethyl)cyclopropanecarbonyl]-2,6-diazaspiro[3.4]octane-8-carboxylate: A solution of methyl (8S)-2-[1-(trifluoromethyl)cyclopropanecarbonyl]-2,6-diazaspiro[3.4]octane-8-carboxylate (500) mg, 1.632 mmol, 1.0 equiv), 7-bromo-5-fluoro-1,3-benzothiazole (378.8 mg, 1.632 mmol, 1.0 equiv). Cs₂CO₃ (1063.7 mg, 3.264 mmol. 2.0 equiv). BINAP (101.6 mg, 0.163 mmol, 0.1 equiv), and BINAP Pd G4 (164.3 mg, 0.163 mmol, 0.1 equiv) in 1,4-dioxane (8 mL) was stirred at 90° C. overnight. The reaction was monitored by LCMS. The reaction mixture was allowed to cool down to room temperature, and water was added. The mixture was extracted with ethyl acetate (3×50 mL). The combined organic phase was washed with brine (30 mL) and dried over anhydrous Na₂SO₄. After filtration, the filtrate was concentrated under reduced pressure. The residue was purified by silica gel column chromatography, eluting with CH₂Cl₂/MeOH (1:1) to afford methyl (8S)-6-(5-fluoro-1,3-benzothiazol-7-yl)-2-[1-(trifluoromethyl)cyclopropanecarbonyl]-2,6-diazaspiro[3.4]octane-8-carboxylate (350) mg, 46.8%) as a brown solid. LCMS (ESI, m/z)=458.4 [M+H]⁺.

Step 10: (8S)-6-(5-fluoro-1,3-benzothiazol-7-yl)-2-[1-(trifluoromethyl)cyclopropanecarbonyl]-2,6-diazaspiro[3.4]octane-8-carboxylic acid: To a stirred solution of methyl (8S)-6-(5-fluoro-1,3-benzothiazol-7-yl)-2-[1-(trifluoromethyl)cyclopropanecarbonyl]-2,6-diazaspiro[3.4]octane-8-carboxylate (350) mg, 0.765 mmol, 1.0 equiv) in MeOH (6 mL) was added a solution of LiOH (36.7 mg, 1.530 mmol. 2.0 equiv) in H₂O (6 mL) at (° C.) The mixture was stirred overnight at room temperature. The reaction was monitored by LCMS. The mixture was concentrated under reduced pressure. The residue was purified by reversed-phase flash chromatography with the following conditions (column, C18 silica gel; mobile phase. MeCN in water (0.1% FA). 10% to 50% gradient in 10 min; detector, UV 220 nm) to afford (85)-6-(5-fluoro-1,3-benzothiazol-7-yl)-2-[1-(trifluoromethyl)cyclopropanecarbonyl]-2,6-diazaspiro[3.4]octane-8-carboxylic acid (190 mg, 56.0%) as a white solid. LCMS (ESI, m/z)=444.2 [M+H]⁺.

Step 11: (S)-N-((2R,3R)-3-((2-oxabicyclo[2.2.2]octan-4-yl)methoxy)-1-((tetrahydro-2H-pyran-4-yl)oxy)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-64A and (R)-N-((2R,3R)-3-((2-oxabicyclo[2.2.2]octan-4-yl)methoxy)-1-((tetrahydro-2H-pyran-4-yl)oxy) butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide I-64B: A solution of (8S)-6-(5-fluoro-1,3-benzothiazol-7-yl)-2-[1-(trifluoromethyl)cyclopropanecarbonyl]-2,6-diazaspiro[3.4]octane-8-carboxylic acid (190 mg, 0.428 mmol, 1.0 equiv). DIEA (224 uL, 1.284 mmol. 3.0 equiv). (2R,3R)-3-{2-oxabicyclo[2.2.2]octan-4-ylmethoxy}-1-(oxan-4-yloxy)butan-2-amine (134.3 mg, 0.428 mmol, 1.0 equiv), and HATU (244.4 mg, 0.642 mmol, 1.5 equiv) in DMF (4 mL) was stirred overnight at room temperature under nitrogen atmosphere. The reaction was monitored by LCMS. The reaction mixture was quenched with water and extracted with ethyl acetate (3×50 mL). The combined organic phase was washed with brine (30 mL) and dried over Na₂SO₄. After filtration, the filtrate was concentrated under reduced pressure. The residue was purified by prep-HPLC (Column: XBridge Prep OBD C18 Column, 30×150 mm, 5 μm: Mobile Phase A: water (0.05% NH₃·H₂O), Mobile Phase B: ACN; Flow rate: 60 mL/min; Gradient: 37% B to 50% B in 10 min, 50% B: Wave Length: 220/254 nm; RT1 (min): 11.18/11.6) to afford the (S)-N-((2R,3R)-3-((2-oxabicyclo[2.2.2]octan-4-yl)methoxy)-1-((tetrahydro-2H-pyran-4-yl)oxy)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide (I-64A) (36.1 mg, major isomer) as a white solid and (R)—N-((2R,3R)-3-((2-oxabicyclo[2.2.2]octan-4-yl)methoxy)-1-((tetrahydro-2H-pyran-4-yl)oxy)butan-2-yl)-6-(5-fluorobenzo[d]thiazol-7-yl)-2-(1-(trifluoromethyl)cyclopropane-1-carbonyl)-2,6-diazaspiro[3.4]octane-8-carboxamide (I-64B) (17.7 mg, minor isomer) as a white solid. I-64A: LCMS (ESI, m/z)=739.25 [M+H]⁺: ¹H NMR (400 MHz, Methanol-d₄) δ 9.19 (s, 1H), 7.13 (dd, J=8.0 Hz, 4.0 Hz, 1H), 6.44 (d, J=12.0 Hz, 1H), 4.62-4.58 (m, 1H), 4.39-4.22 (m, 2H), 4.15-3.95 (m, 4H), 3.93-3.75 (m, 4H), 3.75-3.72 (m, 3H), 3.61-3.55 (m, 2H), 3.52-3.45 (m, 2H), 3.41-3.34 (m, 3H), 3.24 (d, J=8.0 Hz, 1H), 2.97 (d, J=8.0 Hz, 1H), 2.04-1.91 (m, 2H), 1.85-1.81 (m, 2H), 1.71-1.59 (m, 4H), 1.54-1.36 (m, 4H), 1.28-1.25 (m, 4H), 1.07 (d, J=8.0 Hz, 3H). I-64B: LCMS (ESI, m/z)=739.25 [M+H]′: ¹H NMR (400 MHz, Methanol-d₄) δ 9.19 (s, 1H), 7.13 (dd, J=12.0, 4.0 Hz, 1H), 6.46 (dd, J=12.0 Hz, 4.0 Hz, 1H), 4.66 (s, 1H), 4.38-4.27 (m, 2H), 4.15-3.80 (m, 8H), 3.74-3.67 (m, 3H), 3.61-3.49 (m, 4H), 3.48-3.38 (m, 2H), 3.40-3.38 (m, 1H), 3.21 (d, J=8.0 Hz, 1H), 2.95 (d, J=8.0 Hz, 1H), 1.99-1.86 (m, 4H), 1.731.36 (m, 9H), 1.28-1.25 (m, 5H), 1.07 (d, J=8.0 Hz, 3H).

The compounds listed in Table 8 were synthesized according to the procedures described for the syntheses of compounds I-2 and I-5 using the appropriate commercially available reagents and/or intermediates described elsewhere.

TABLE 8

| Example | | | |
|---|---|---|---|
| Compound | | | |
| Number | Compound | ¹HNMR | LCMS |
| 200 | 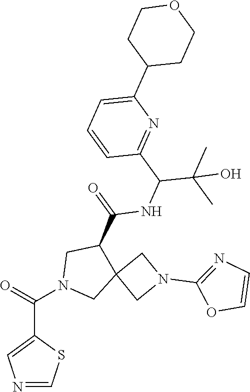 [SMILES: CC(C)(O)C(NC(=O)[C@@H]1CN(C(=O)c2cncs2)CC12CN(c1ncco1)C2)c1cccc(C2CCOCC2)n1] | ¹H NMR (400 MHz, DMSO-d6) δ 9.23 (t, J = 7.4 Hz, 1H), 8.61- 8.50 (m, 1H), 8.41- 8.27 (m, 1H), 7.71- 7.52 (m, 2H), 7.25- 7.10 (m, 2H), 6.88- 6.78 (m, 1H), 5.14- 4.99 (m, 1H), 4.90- 4.76 (m, 1H), 4.32- 3.83 (m, 8H), 3.81- 3.52 (m, 3H), 3.50- 3.39 (m, 2H), 3.00- 2.85 (m, 1H), 1.86- 1.67 (m, 4H), 1.13- 0.98 (m, 6H). | m/z = 567.3 [M + H]⁺ |
| 201 | 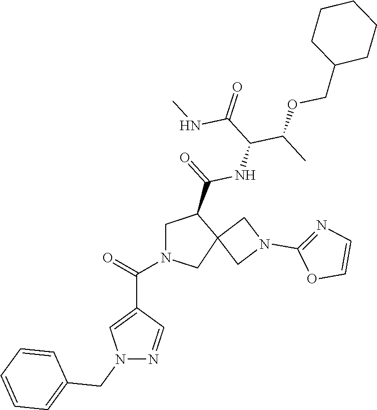 [SMILES: CNC(=O)[C@@H](NC(=O)[C@@H]1CN(C(=O)c2cnn(Cc3ccccc3)c2)CC12CN(c1ncco1)C2)[C@@H](C)OCC1CCCCC1] | ¹H NMR (400 MHz, Chloroform-d) δ 7.83 (d, J = 2.4 Hz, 2H), 7.39-7.31 (m, 3H), 7.26-7.21 (m, 3H), 7.03-6.90 (m, 1H), 6.82 (s, 1H), 6.64- 6.53 (m, 1H), 5.30 (s, 2H), 4.49 (s, 1H), 4.31- 3.73 (m, 9H), 3.37 (d, J = 6.4 Hz, 2H), 3.18- 3.09 (m, 1H), 2.84 (d, J = 4.8 Hz, 3H), 1.62- 1.49 (m, 2H), 1.33- 1.10 (m, 4H), 1.04- 0.88 (m, 5H). | m/z = 618.4 [M + H]⁺ |
| 202 (I-1) | 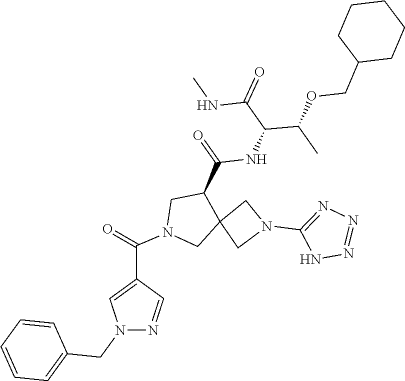 [SMILES: CNC(=O)[C@@H](NC(=O)[C@@H]1CN(C(=O)c2cnn(Cc3ccccc3)c2)CC12CN(c1nnn[nH]1)C2)[C@@H](C)OCC1CCCCC1] | ¹H NMR (400 MHz, DMSO-d6) δ 8.36 (d, J = 15.4 Hz, 1H), 8.21- 8.13 (m, 1H), 7.82 (d, J = 14.8 Hz, 1H), 7.77- 7.68 (m, 1H), 7.38- 7.22 (m, 5H), 5.35 (s, 2H), 4.27-3.61 (m, 10H), 3.57-3.44 (m, 1H), 3.23-3.16 (m, 1H), 3.11-3.05 (m, 1H), 2.61-2.54 (m, 3H), 1.67-1.56 (m, 5H), 1.46-1.35 (m, 1H), 1.21-1.07 (m, 3H), 0.94 (dd, J = 15.0, 6.2 Hz, 3H), 0.87-0.74 (m, 2H). | m/z = 619.5 [M + H]⁺ |

### tert-Butyl (S)-6-benzyl-8-((R)-2-oxo-4-phenyloxazolidine-3-carbonyl)-2,6-diazaspiro[3.4]octane-2-carboxylate (Intermediate 11) and tert-butyl (R)-6-benzyl-8-((R)-2-oxo-4-phenyloxazolidine-3-carbonyl)-2,6-diazaspiro[3.4]octane-2-carboxylate (Intermediate 12)

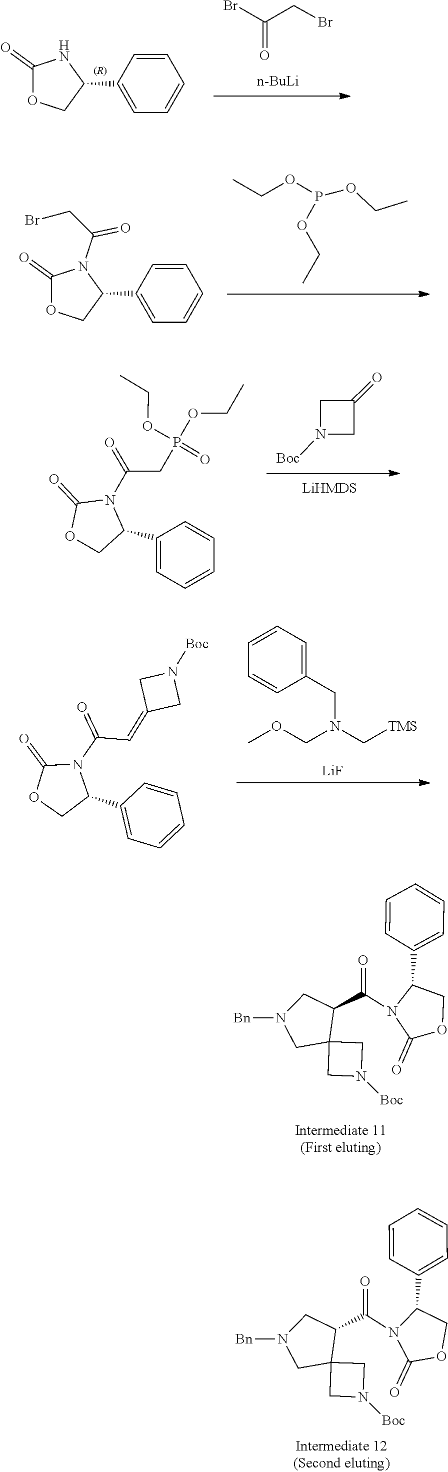

Step 1: (R)-3-(2-bromoacetyl)-4-phenyloxazolidin-2-one: To a solution of (R)-4-phenyloxazolidin-2-one (10 g, 61 mmol) in anhydrous THF (100 mL) at −78° C. under a N₂ atmosphere was added n-BuLi (2.5 M n-BuLi in hexanes, 27 mL, 67 mmol) dropwise. The reaction mixture was stirred at −78° C. for 0.5 hour after which 2-bromoacetyl bromide (5.6 mL, 64 mmol) was added. The reaction was allowed to warm to room temperature and stirred for an additional 2 hours. The mixture was diluted with EtOAc (100 mL), quenched with saturated NH₄Cl (100 mL), extracted with EtOAc (100 mL×2), dried over Na₂SO₄, filtered, and concentrated. The residue was purified by column chromatography on silica gel (eluent: petroleum ether/EtOAc=10/1 to 3/1) to afford (R)-3-(2-bromoacetyl)-4-phenyloxazolidin-2-one (9 g, 52%) as a yellow oil. ¹H NMR (400 MHz, DMSO-d₆): δ 7.42-7.37 (m, 2H), 7.36-7.30 (m, 3H), 5.52-5.46 (m, 1H), 4.83-4.75 (m, 2H), 4.56-4.50 (m, 1H), 4.24-4.18 (m, 1H).

Step 2: Diethyl (R)-(2-oxo-2-(2-oxo-4-phenyloxazolidin-3-yl)ethyl)phosphonate: A mixture of (R)-3-(2-bromoacetyl)-4-phenyloxazolidin-2-one (10 g, 35 mmol) in triethyl phosphite (29 g, 175 mmol) was heated at 50° C. for 18 hours. The excess triethyl phosphite was removed under vacuum at 70° C. to afford diethyl (R)-(2-oxo-2-(2-oxo-4-phenyloxazolidin-3-yl)ethyl)phosphonate (12 g crude. 99%) as a yellow oil. LCMS m/z=342.1 [M+H]⁺: ¹H NMR (400 MHz, CDCl₃): δ 7.41-7.29 (m, 5H), 5.55-5.48 (m, 1H), 4.78-4.70 (m, 1H), 4.20-4.14 (m, 1H), 4.03-3.96 (m, 5H), 3.60-3.48 (m, 1H), 1.27-1.15 (m, 8H).

Step 3: tert-Butyl (R)-3-(2-oxo-2-(2-oxo-4-phenyloxazolidin-3-yl)ethylidene) azetidine-1-carboxylate: To a solution of diethyl (R)-(2-oxo-2-(2-oxo-4-phenyloxazolidin-3-yl)ethyl)phosphonate (10 g, 29 mmol) in anhydrous THF (100 mL) at (° C.) under N₂ atmosphere was added LiHMDS (1.0 M in THF. 29 mL, 29 mmol) dropwise. The reaction mixture was stirred at 0° C. for 30 minutes after which tert-butyl 3-oxoaretidine-1-carboxylate (21.68 g, 127 mmol) was added. The reaction was warmed to room temperature and stirred for 1 hour. The reaction was diluted with EtOAc (200 mL), and the organic layer was washed with saturated NH₄Cl (100 mL), dried over Na₂SO₄, filtered, and concentrated. The residue was purified by column chromatography on silica gel (eluent: petroleum ether/EtOAc=10/1) to afford tert-butyl (R)-3-(2-oxo-2-(2-oxo-4-phenyloxazolidin-3-yl)ethylidene) azetidine-1-carboxylate (10 g, 95%) as a yellow solid. LCMS m/z=492.0 [M+H]⁺: ¹H NMR (400 MHz, DMSO-d₆): δ 7.41-7.36 (m, 2H), 7.34-7.28 (m, 3H), 7.20-7.15 (m, 1H), 5.53-5.46 (m, 1H), 4.79-4.73 (m, 1H), 4.61 (s. 4H). 4.21-4.14 (m, 1H), 1.37 (s, 9H).

Step 4: tert-Butyl (S)-6-benzyl-8-((R)-2-oxo-4-phenyloxazolidine-3-carbonyl)-2,6-diazaspiro[3.4]octane-2-carboxylate (Intermediate 11) and tert-butyl (R)-6-benzyl-8-((R)-2-oxo-4-phenyloxazolidine-3-carbonyl)-2,6-diazaspiro[3.4]octane-2-carboxylate (Intermediate 12): A mixture of tert-butyl (R)-3-(2-oxo-2-(2-oxo-4-phenyloxazolidin-3-yl)ethylidene) azetidine-1-carboxylate (10 g, 27.90 mmol). N-benzyl-1-methoxy-N-((trimethylsilyl)methyl)methanamine (8.61 g, 36.27 mmol), and LiF (2.17 g, 83.71 mmol) in acetonitrile (100 mL) was heated at 80° C. for 16 hours. After cooling to room temperature, the mixture was diluted with water (100 mL) and extracted with EtOAc (100 mL×3). The combined organic layers were washed with brine, dried over Na₂SO₁, filtered, and concentrated. The mixture was purified by column chromatography on silica gel (eluent: petroleum ether/EtOAc=6/1) to afford tert-butyl (S)-6-benzyl-8-((R)-2-oxo-4-phenyloxazolidine-3-carbonyl)-2,6-diazaspiro[3.4]octane-2-carboxylate (Intermediate 11) (8 g. 58%) as a yellow solid as the first eluting isomer. Further elution provided tert-butyl (R)-6-benzyl-8-((R)-2-oxo-4-phenyloxazolidine-3-carbonyl)-2,6-diazaspiro[3.4]octane-2-carboxylate (Intermediate 12) (4 g, 29%). Intermediate 11: LCMS m/z=492 [M+H]⁺; ¹H NMR (400 MHz, DMSO-d₆): δ 7.41-7.22 (m, 10H), 5.49-5.43 (m, 1H), 4.75-4.69 (m, 1H), 4.23-4.11 (m, 2H), 3.93-3.85 (m, 1H), 3.79 (s, 1H), 3.65-3.58 (m, 2H), 3.56-3.51 (m, 2H), 3.24-3.15 (m, 1H), 2.94-2.87 (m, 1H), 2.57-2.52 (m, 1H), 2.35-2.27 (m, 1H), 1.36 (s, 9H). Intermediate 12: LCMS m/z=492 [M+H]⁺: ¹H NMR (400 MHz, DMSO-d₆): δ 7.37-7.24 (m, 10H), 5.47-5.41 (m, 1H), 4.76-4.69 (m, 1H), 4.35-4.28 (m, 1H), 4.21-4.14 (m, 1H), 4.00-3.92 (m, 1H), 3.66-3.60 (m, 1H), 3.59-3.55 (m, 2H), 3.36 (s, 2H), 3.19-3.15 (m, 1H), 3.06-2.98 (m, 1H), 2.94-2.88 (m, 1H), 2.55 (s, 1H), 1.36 (s, 9H).

### Synthesis of (S)-6-(1-benzyl-1H-pyrazole-4-carbonyl)-N-((2S,3R)-3-(cyclohexylmethoxy)-1-(methylamino)-1-oxobutan-2-yl)-2,6-diazaspiro[3.4]octane-8-carboxamide

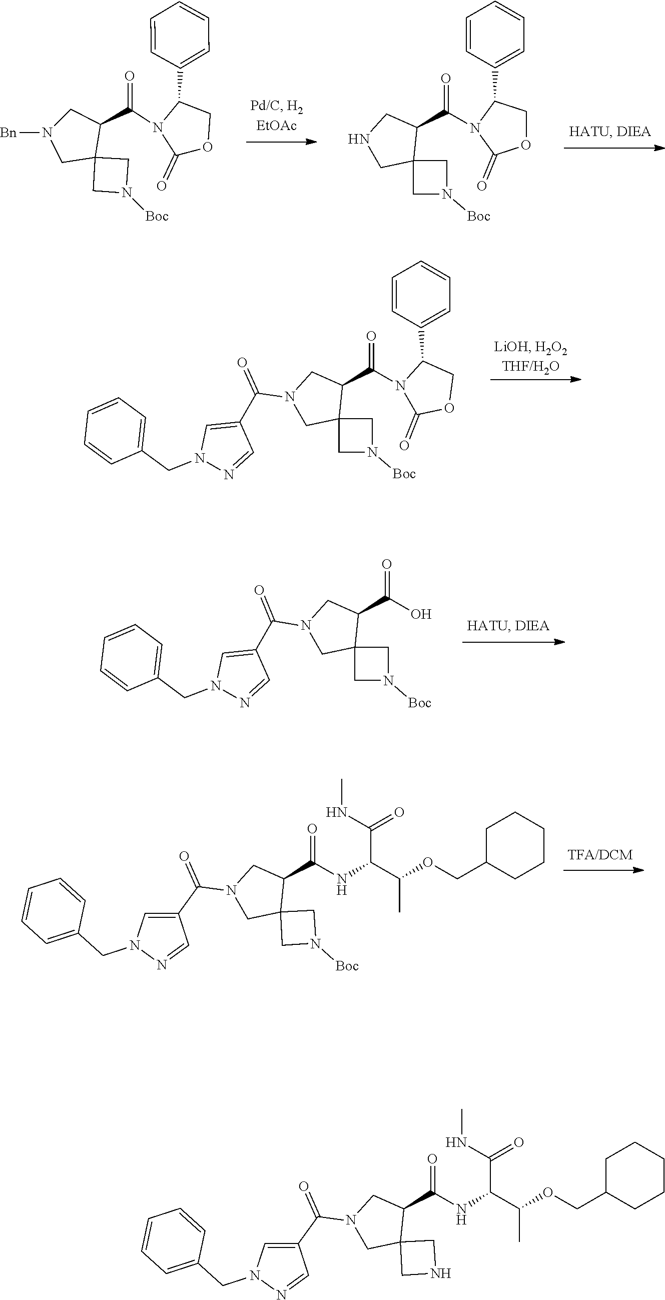 [SMILES: CC(C)(C)OC(=O)N1CC2(CN(Cc3ccccc3)C[C@H]2C(=O)N2C(=O)OC[C@H]2c2ccccc2)C1 ; CC(C)(C)OC(=O)N1CC2(CNC[C@H]2C(=O)N2C(=O)OC[C@H]2c2ccccc2)C1 ; CC(C)(C)OC(=O)N1CC2(C1)CN(C(=O)c1cnn(Cc3ccccc3)c1)C[C@H]2C(=O)N1C(=O)OC[C@H]1c1ccccc1 ; CC(C)(C)OC(=O)N1CC2(C1)CN(C(=O)c1cnn(Cc3ccccc3)c1)C[C@H]2C(=O)O ; CNC(=O)[C@@H](NC(=O)[C@@H]1CN(C(=O)c2cnn(Cc3ccccc3)c2)CC12CN(C(=O)OC(C)(C)C)C2)[C@@H](C)OCC1CCCCC1 ; CNC(=O)[C@@H](NC(=O)[C@@H]1CN(C(=O)c2cnn(Cc3ccccc3)c2)CC12CNC2)[C@@H](C)OCC1CCCCC1]

Step 1: tert-Butyl (S)-8-((R)-2-oxo-4-phenyloxazolidine-3-carbonyl)-2,6-diazaspiro[3.4]octane-2-carboxylate: To a solution of tert-butyl (S)-6-benzyl-8-((R)-2-oxo-4-phenyloxazolidine-3-carbonyl)-2,6-diazaspiro[3.4]octane-2-carboxylate (Intermediate 11) (1 g, 2.0 mmol) in EtOAc (10 mL) was added 10% Pd/C (300 mg). The reaction mixture was stirred under a H₂ atmosphere for 48 hours. The mixture was filtered and concentrated to afford crude tert-butyl (S)-8-((R)-2-oxo-4-phenyloxazolidine-3-carbonyl)-2,6-diazaspiro[3.4]octane-2-carboxylate (800 mg, 100%), which was used directly in the next step without further purification. LCMS m/z=402.2 [M+H]⁺.

Step 2: tert-Butyl (S)-6-(1-benzyl-1H-pyrazole-4-carbonyl)-8-((R)-2-oxo-4-phenyloxazolidine-3-carbonyl)-2,6-diazaspiro[3.4]octane-2-carboxylate: To a solution of 1-benzyl-1H-pyrazole-4-carboxylic acid (2.2 g, 11.0 mmol) in DCM (40 mL) was added HATU (4.79 g, 12.6 mmol), and the mixture was stirred at room temperature for 30 minutes. tert-Butyl (S)-8-((R)-2-oxo-4-phenyloxazolidine-3-carbonyl)-2,6-diazaspiro[3.4]octane-2-carboxylate (4.2 g, 10.5 mmol) and DIPEA (2.03 g, 15.75 mmol) were added and the reaction stirred at room temperature for an additional 2 hours. The mixture was diluted with water (100 mL) and extracted with EtOAc (150 mL×3). The combined organic layers were washed with brine, dried over Na₂SO₄, filtered, and concentrated. The residue obtained was purified by column chromatography on silica gel (eluent: DCM/MeOH=30/1) to afford tert-butyl (S)-6-(1-benzyl-1H-pyrazole-4-carbonyl)-8-((R)-2-oxo-4-phenyloxazolidine-3-carbonyl)-2,6-diazaspiro[3.4]octane-2-carboxylate (4.7 g, 77%) as a yellow solid. LCMS m/z=586.3 [M+H]⁺.

Step 3: (S)-6-(1-Benzyl-1H-pyrazole-4-carbonyl)-2-(tert-butoxycarbonyl)-2,6-diazaspiro[3.4]octane-8-carboxylic acid: To a solution of tert-butyl (S)-6-(1-benzyl-1H-pyrazole-4-carbonyl)-8-((R)-2-oxo-4-phenyloxazolidine-3-carbonyl)-2,6-diazaspiro[3.4]octane-2-carboxylate (100 mg, 0.17 mmol) in a mixture of THE and H₂O (3 mL/0.25 mL) at 0° C.) was added a solution of lithium hydroxide monohydrate (10 mg, 0.43 mmol) in water (0.25 mL) and 30% H₂O₂ (12 mg, 0.34 mmol) in water (0.25 mL). The reaction mixture was stirred at (° C.) for 1 hour and then diluted with water (15 mL) and extracted with EtOAc (30 mL). The aqueous layer was collected and acidified with HCl (1M) to pH˜2 and extracted with EtOAc (80 mL×3). The combined organic layers were washed with brine, dried over Na₂SO₄, filtered, and concentrated to afford crude (S)-6-(1-benzyl-1H-pyrazole-4-carbonyl)-2-(tert-butoxy carbonyl)-2,6-diazaspiro[3.4]octane-8-carboxylic acid (50 mg, 66%) as a white solid, which was used directly in the next step. LCMS m/z=441.2 [M+H]⁺.

Step 4: tert-Butyl (S)-6-(1-benzyl-1H-pyrazole-4-carbonyl)-8-(((2S,3R)-3-(cyclohexylmethoxy)-1-(methylamino)-1-oxobutan-2-yl)carbamoyl)-2,6-diazaspiro[3.4]octane-2-carboxylate: To a solution of (S)-6-(1-benzyl-1H-pyrazole-4-carbonyl)-2-(tert-butoxycarbonyl)-2,6-diazaspiro[3.4]octane-8-carboxylic acid (100 mg, 0.227 mmol) in DCM (2 mL) was added HATU (103 mg, 0.272 mmol), and the mixture was stirred at room temperature for 30 minutes. (2S,3R)-2-Amino-3-(cyclohexylmethoxy)-N-methylbutanamide hydrochloride (52 mg, 0.227 mmol) and DIPEA (117 mg, 0.908 mmol) were added and the reaction stirred at room temperature for an additional 2 hours. The mixture was diluted with water (30 mL) and extracted with EtOAc (50 mL×3). The combined organic layers were washed with brine, dried over Na₂SO₄, filtered, and concentrated. The residue obtained was purified by column chromatography on silica gel (eluent: DCM/MeOH=30/1) to afford tert-butyl (S)-6-(1-benzyl-1H-pyrazole-4-carbonyl)-8-(((2S,3R)-3-(cyclohexylmethoxy)-1-(methylamino)-1-oxobutan-2-yl) carbamoyl)-2,6-diazaspiro[3.4]octane-2-carboxylate (114 mg, 78%) as a yellow solid. LCMS m/z=651.4 [M+H]⁺.

Step 5: (S)-6-(1-benzyl-1H-pyrazole-4-carbonyl)-N-((2S,3R)-3-(cyclohexylmethoxy)-1-(methylamino)-1-oxobutan-2-yl)-2,6-diazaspiro[3.4]octane-8-carboxamide: To a solution of (S)-6-(1-benzyl-1H-pyrazole-4-carbonyl)-8-(((2S,3R)-3-(cyclohexylmethoxy)-1-(methylamino)-1-oxobutan-2-yl)carbamoyl)-2,6-diazaspiro[3.4]octane-2-carboxylate (50 mg, 0.077 mmol) in DCM (2 mL) was added TFA (1 mL). The reaction mixture was stirred at room temperature for 1 hour. The solvent was removed under vacuum to afford (S)-6-(1-benzyl-1H-pyrazole-4-carbonyl)-N-((2S,3R)-3-(cyclohexylmethoxy)-1-(methylamino)-1-oxobutan-2-yl)-2,6-diazaspiro[3.4]octane-8-carboxamide (42 mg, 100%), which was used directly in the next step. LCMS m/z=551.3 [M+H]⁺.

### Synthesis of (S)-6-(1-Benzyl-1H-pyrazole-4-carbonyl)-N-((2S,3R)-3-(cyclohexylmethoxy)-1-(methylamino)-1-oxobutan-2-yl)-2-(1H-tetrazol-5-yl)-2,6-diazaspiro[3.4]octane-8-carboxamide I-1

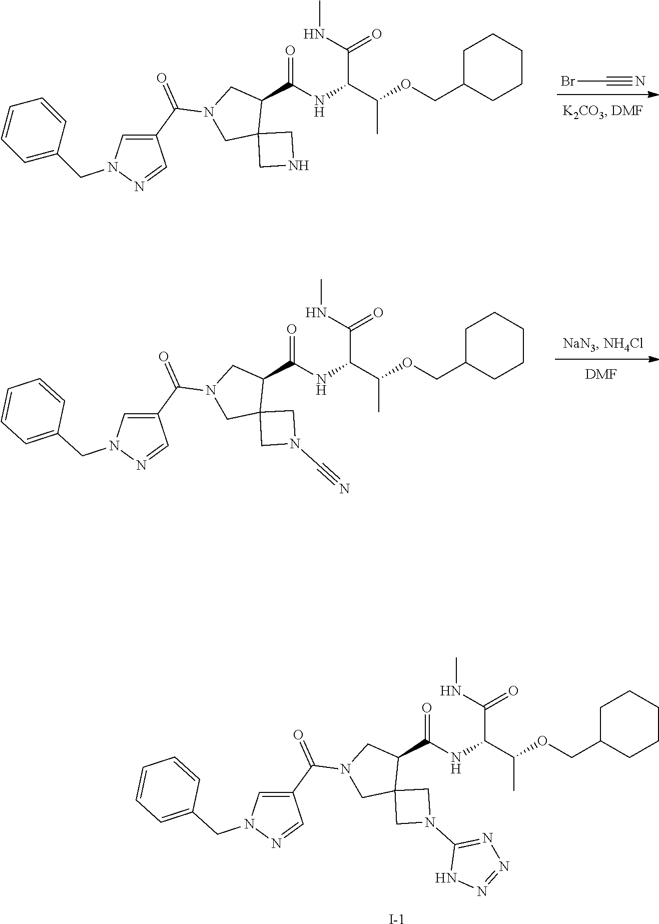

Step 1: (S)-6-(1-benzyl-1H-pyrazole-4-carbonyl)-2-cyano-N-((2S,3R)-3-(cyclohexylmethoxy)-1-(methylamino)-1-oxobutan-2-yl)-2,6-diazaspiro[3.4]octane-8-carboxamide: To a solution of (S)-6-(1-benzyl-1H-pyrazole-4-carbonyl)-N-((2S,3R)-3-(cyclohexylmethoxy)-1-(methylamino)-1-oxobutan-2-yl)-2,6-diazaspiro[3.4]octane-8-carboxamide (211 mg, 0.38 mmol) in DMF (6 mL) was added K₂CO₃ (210 mg, 1.52 mmol) and cyanogenbromide (41 mg, 0.38 mmol). The mixture was stirred at room temperature for 3 hours. The mixture was diluted with water (50 mL) and the precipitate that formed collected by filtration to afford crude (S)-6-(1-benzyl-1H-pyrazole-4-carbonyl)-2-cyano-N-((2S,3R)-3-(cyclohexylmethoxy)-1-(methylamino)-1-oxobutan-2-yl)-2,6-diazaspiro[3.4]octane-8-carboxamide (170 mg, 27%) as a yellow solid. LCMS m/z=576.4 [M+H]⁺.

Step 2: (S)-6-(1-benzyl-1H-pyrazole-4-carbonyl)-N-((2S,3R)-3-(cyclohexylmethoxy)-1-(methylamino)-1-oxobutan-2-yl)-2-(1H-tetrazol-5-yl)-2,6-diazaspiro[3.4]octane-8-carboxamide: To a solution of (S)-6-(1-benzyl-1H-pyrazole-4-carbonyl)-2-cyano-N-((2S,3R)-3-(cyclohexylmethoxy)-1-(methylamino)-1-oxobutan-2-yl)-2,6-diazaspiro[3.4]octane-8-carboxamide (80 mg, 0.14 mmol) in DMF (3 mL) was added NH₄Cl (30 mg, 0.56 mmol) and NaN₃ (18 mg, 0.28 mmol). The reaction mixture was stirred at 110° C. for 5 hours. The reaction mixture was purified by prep-HPLC to afford (S)-6-(1-benzyl-1H-pyrazole-4-carbonyl)-N-((2S,3R)-3-(cyclohexylmethoxy)-1-(methylamino)-1-oxobutan-2-yl)-2-(1H-tetrazol-5-yl)-2,6-diazaspiro[3.4]octane-8-carboxamide 1-1 (19.7 mg, 22%) as a white solid. LCMS m/z=619.5 [M+H]⁺: ¹H NMR (400 MHz, DMSO-d₆): δ 8.36 (d, J=15.4 Hz, 1H), 8.21-8.13 (m, 1H), 7.82 (d, J=14.8 Hz, 1H), 7.77-7.68 (m, 1H), 7.38-7.22 (m, 5H), 5.35 (s, 2H), 4.27-3.61 (m, 10H), 3.57-3.44 (m, 1H), 3.23-3.16 (m, 1H), 3.11-3.05 (m, 1H), 2.61-2.54 (m, 3H), 1.67-1.56 (m, 5H), 1.46-1.35 (m, 1H), 1.21-1.07 (m, 3H), 0.94 (dd, J=15.0, 6.2 Hz, 3H), 0.87-0.74 (m, 2H).

### Synthesis of 4-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoic acid I-47

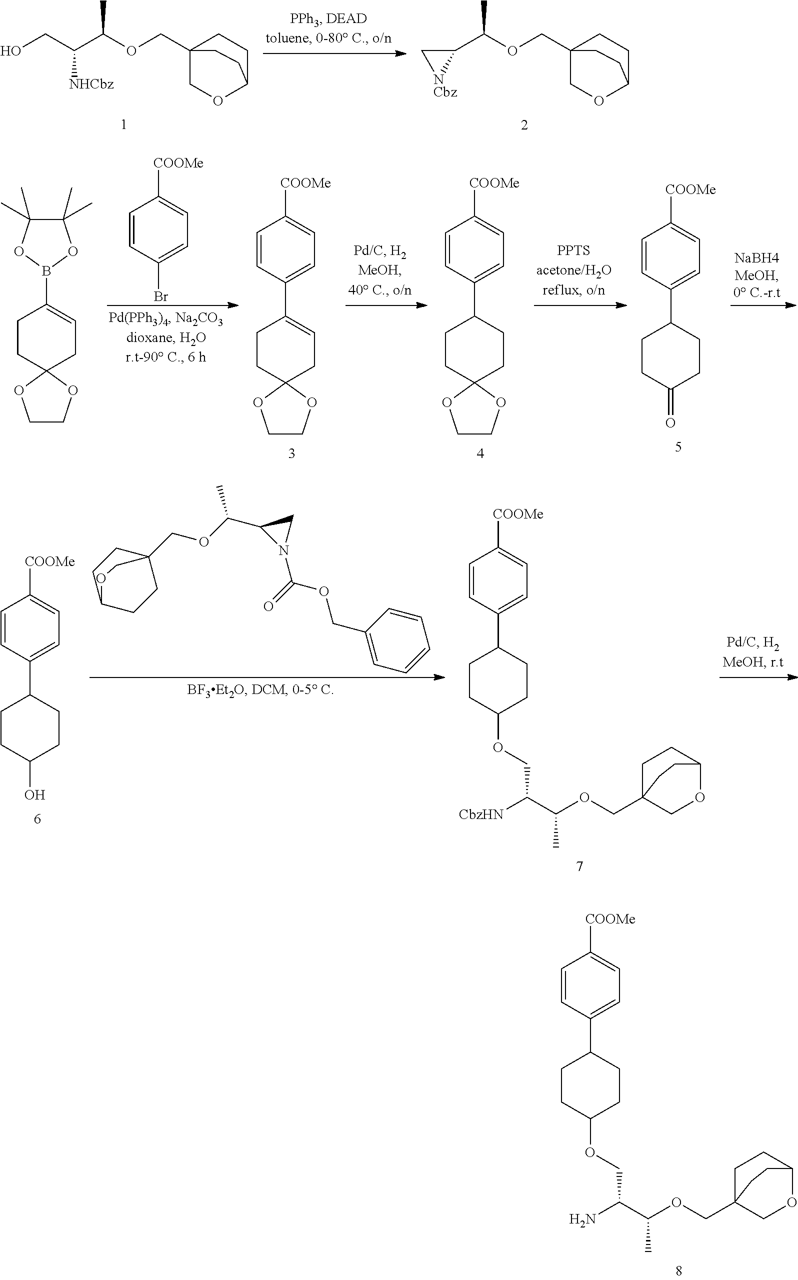

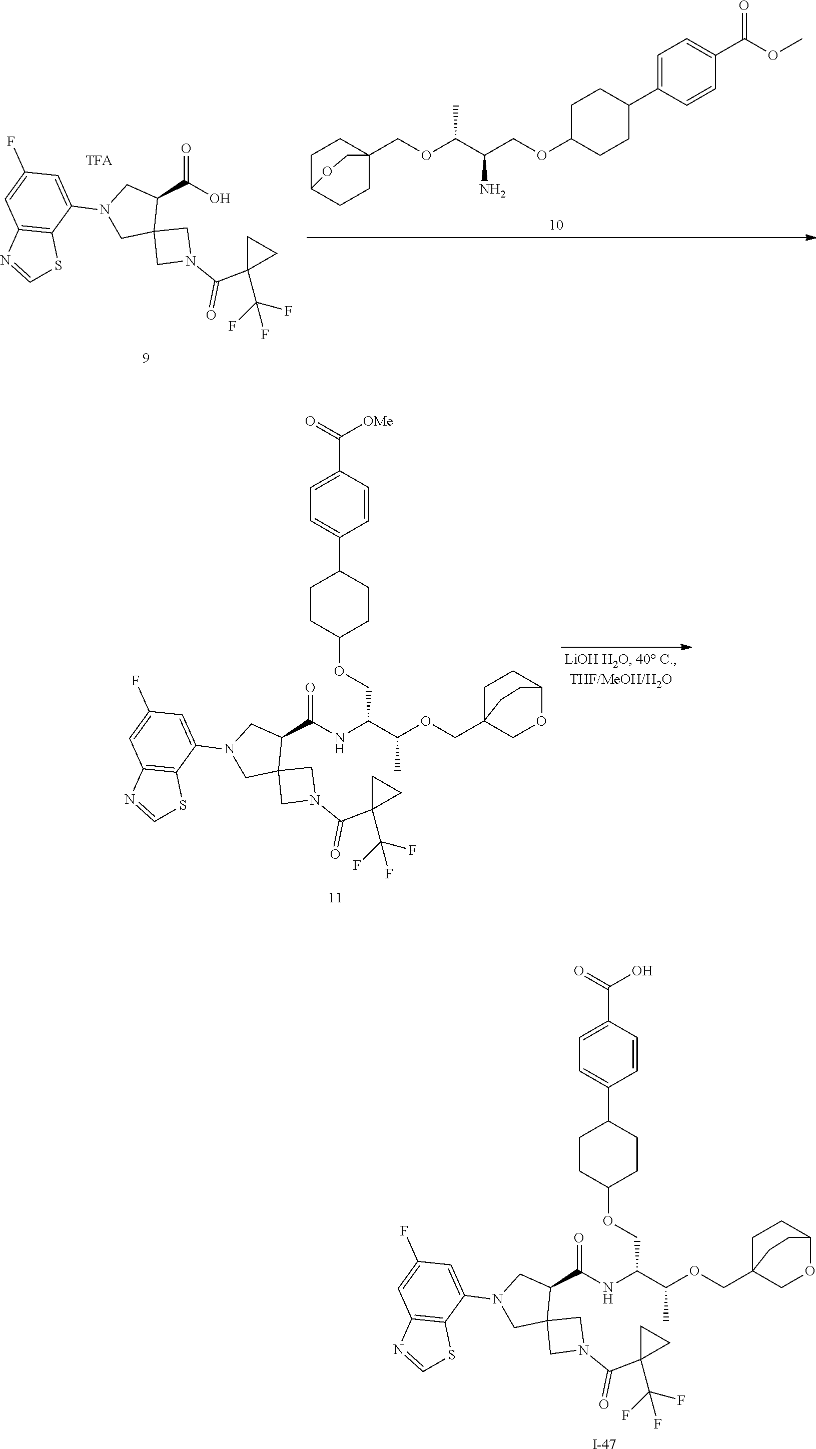

Step 1: (R)-Benzyl 2-((R)-1-(2-oxabicyclo[2.2.2]octan-4-ylmethoxy)ethyl)aziridine-1-carboxylate (2): To a solution of benzyl (2R,3R)-3-(2-oxabicyclo[2.2.2]octan-4-ylmethoxy)-1-hydroxybutan-2-yl)carbamate 1 (0.200 g, 0.550 mmol) and triphenylphosphine (0.202 g, 0.770 mmol) in toluene (3 mL) at 0° C. under nitrogen atmosphere was added diethyl azodicarboxylate (0.134 g, 0.770 mmol). The resulting mixture was stirred at 80° C. under nitrogen overnight. The reaction mixture was concentrated under reduced pressure to give a crude residue, which was purified by silica gel flash column chromatography using a 14% ethyl acetate in hexane gradient to afford (R)-benzyl 2-((R)-1-(2-oxabicyclo[2.2.2]octan-4-ylmethoxy)ethyl) aziridine-1-carboxylate 2 (0.135 g, 64% yield) as a colorless oil. MS: [MH]⁺ 346.50.

Step 2: Methyl 4-(1,4-dioxaspiro[4.5]dec-7-en-8-yl)benzoate (3): To a mixture of methyl 4-bromobenzoate (1.305 g, 6.10 mmol), 4,4,5,5-tetramethyl-2-(1,4-dioxaspiro[4.5]dec-7-en-8-yl)-1,3,2-dioxaborolane (1.947 g, 7.316 mmol), and sodium carbonate (3.231 g, 30.485 mmol) in a mixture of 1,4-dioxane (30 mL) and water (15 mL) was added tetrakis(triphenylphosphine) palladium (0.352 g, 0.305 mmol) under nitrogen atmosphere. The resulting mixture was stirred at 90° C. under nitrogen atmosphere for 6 hours. The reaction mixture was poured into water (10 mL) and extracted with ethyl acetate (30 mL×2). The combined organic layers were washed with brine (30 mL), dried over anhydrous sodium sulfate, and concentrated to give a crude residue, which was purified by silica gel column chromatography using a 12.5% ethyl acetate in hexane gradient to afford methyl 4-(1,4-dioxaspiro[4.5]dec-7-en-8-yl)benzoate 3 (1.593 g, 95%) as a yellow oil. MS: [MH]⁺ 275.20.

Step 3: Methyl 4-(1,4-dioxaspiro[4.5]decan-8-yl)benzoate (4): To a solution of methyl 4-(1,4-dioxaspiro[4.5]dec-7-en-8-yl)benzoate 3 (1.593 g, 5.79 mmol) in ethyl acetate (70 mL) was added palladium (10% on carbon, 0.650 g). The mixture was stirred at 40° C. under hydrogen atmosphere overnight. The catalyst was removed by filtration, and the filtrate was concentrated to give a crude residue, which was purified by silica gel flash column chromatography using a hexane-ethyl acetate-dichloromethane (3:1:1 v/v/v) gradient to afford methyl 4-(1,4-dioxaspiro[4.5]decan-8-yl)benzoate 4 (1.523 g, 95% yield) as a colorless oil. MS: [MH]⁺ 277.20.

Step 4: Methyl 4-(4-oxocyclohexyl)benzoate (5): A mixture of methyl 4-(1,4-dioxaspiro[4.5]decan-8-yl)benzoate 4 (1.523 g, 5.50 mmol) and pyridinium p-toluenesulfonate (1.272 g, 5.06 mmol) in a mixture of acetone (12 mL) and water (12 mL) was refluxed overnight. The reaction mixture was concentrated in vacuo. The residue was taken up in ethyl acetate (25 mL), washed with brine (30 mL), dried over anhydrous sodium sulfate, and concentrated to give methyl 4-(4-oxocyclohexyl)benzoate 5 (1.166 g, 91%) as a white solid, which was used in the next step without further purification. MS: [MH]⁺ 233.30.

Step 5: trans-Methyl 4-(4-hydroxycyclohexyl)benzoate (6): To a solution of methyl 4-(4-oxocyclohexyl)benzoate 5 (1.166 g, 5.00 mmol) in methanol (15 mL) at 0° C. was added sodium borohydride (0.193 g, 16.552 mmol). The resulting mixture was stirred at room temperature for 1.5 hours. The reaction mixture was poured into water (10 mL) and extracted with ethyl acetate (20 mL×3). The combined organic layers were washed with brine (30 mL), dried over anhydrous sodium sulfate, and concentrated to give a crude residue, which was purified by silica gel flash column chromatography using a hexane-ethyl acetate-dichloromethane (1:1:1, v/v/v) gradient to afford trans-methyl 4-(4-hydroxy cyclohexyl)benzoate 6 (0.580 g, 49%) as a white solid. MS: [MH]⁺ 235.20.

Step 6: Methyl 4-(4-((2R,3R)-3-(2-oxabicyclo[2.2.2]octan-4-ylmethoxy)-2-(((benzyloxy)carbonyl)amino)butoxy)cyclohexyl)benzoate (7): To a solution of (R)-benzyl 2-((R)-1-(2-oxabicyclo[2.2.2]octan-4-ylmethoxy)ethyl) aziridine-1-carboxylate (0.670 g, 1.94 mmol) and trans-methyl 4-(4-hydroxycyclohexyl)benzoate 6 (0.477 g, 2.04 mmol) in chloroform (30 mL) at 0° C. was slowly added boron trifluoride etherate (0.193 g, 1.360 mmol). The resulting mixture was stirred at 0-5° C. under nitrogen atmosphere for 1 hour. The reaction mixture was concentrated to give a crude residue, which was purified by silica gel flash column chromatography using a hexane-ethyl acetate-dichloromethane (3:1:1, v/v/v) gradient to afford methyl 4-(4-((2R,3R)-3-(2-oxabicyclo[2.2.2]octan-4-ylmethoxy)-2-(((benzyloxy)carbonyl)amino)butoxy)cyclohexyl)benzoate 7 (0.157 g, 14% yield) as a colorless oil. MS: [MH]⁺ 580.65

Step 7: trans-Methyl 4-(4-((2R,3R)-3-(2-oxabicyclo[2.2.2]octan-4-ylmethoxy)-2-aminobutoxy)cyclohexyl)benzoate (8): To a solution of methyl 4-(4-((2R,3R)-3-(2-oxabicyclo[2.2.2]octan-4-ylmethoxy)-2-(((benzyloxy)carbonyl)amino)butoxy)cyclohexyl)benzoate 7 (0.190 g, 0.328 mmol) in methanol (5 mL) was added Pd/C (10%, 0.020 g). The mixture was stirred at room temperature under hydrogen overnight. The reaction mixture was filtered to remove the catalyst, and the filtrate was concentrated to afford trans-methyl 4-(4-((2R,3R)-3-(2-oxabicyclo [2.2.2]octan-4-ylmethoxy)-2-aminobutoxy)cyclohexyl)benzoate 8 (0.120 g, 80% yield) as a white solid, which was used in the next step without further purification. MS: [MH]⁺ 447.00.

Step 8: Methyl 4-(4-((2R,3R)-3-((2-oxabicyclo[2.2.2]octan-4-yl)methoxy)-2-((S)-6-(5 fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoate 11: To a solution of methyl 4-(4-((2R,3R)-3-(2-oxabicyclo[2.2.2]octan-4-ylmethoxy)-2-aminobutoxy)cyclohexyl)benzoate 10 (0.064 g, 0.14 mmol), (S)-6-(5-fluorobenzo[d]thiazol-7-yl)-2-(1-(trifluoromethyl)cyclopropane-1-carbonyl)-2,6-diazaspiro[3.4]octane-8-carboxylic acid 9 (0.080 g, 0.15 mmol), and N-ethyl-N-isopropylpropan-2-amine (0.056 g, 0.43 mmol) in N,N-dimethylformamide (2 mL) at 0)−5° C. was added (2-(7-aza-1H-benzotriazole-1-yl)-1,1,3,3-tetramethyluronium hexafluorophosphate) (0.066 g, 0.17 mmol). The resulting mixture was stirred at room temperature under nitrogen for 1 hour. The reaction mixture was poured into aqueous ammonium chloride solution (4 mL) and extracted with ethyl acetate (10 mL). The organic layer was washed with water (8 mL×3) and brine (8 mL), dried over anhydrous sodium sulfate, and concentrated under reduced pressure to give a crude residue, which was purified by prep-TLC using a 3.3% methanol in dichloromethane gradient to afford methyl 4-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoate 11 (0.110 g, 88% yield) as a white solid. MS: [MH]⁺ 871.9.

Step 9:4-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoic acid (1-47): To a solution of methyl 4-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoate 11 (0.110 g, 0.13 mmol) in a mixture of tetrahydrofuran (4 mL), methanol (2 mL), and water (1 mL) was added lithium hydroxide monohydrate (0.053 g, 1.30 mmol). The resulting mixture was stirred at 40° C. for 3 hours. The reaction mixture was concentrated. The residue was acidified to pH 6 with diluted hydrochloric acid (1.0 N), and extracted with dichloromethane (6 mL×3). The combined organic layers were dried over anhydrous sodium sulfate and concentrated under reduced pressure to give a crude residue, which was purified by prep-TLC using a 5% methanol in dichloromethane gradient to afford 4-(4-((2R,3R)-3-((2-oxabicyclo[2.2.2]octan-4-yl)methoxy)-2-((S)-6-(5-fluorobenzo[d]thiazol-7-yl)-2-(1-(trifluoromethyl)cyclopropane-1-carbonyl)-2,6-diazaspiro[3.4]octane-8-carboxamido)butoxy)cyclohexyl)benzoic acid I-47 (0.080 g, yield 74%) as a white solid. ¹H NMR (400 MHz, CD₃OD): δ 9.20 (s, 1H), 8.04-7.81 (m, 3H), 7.35-7.25 (m, 2H), 7.16-7.10 (m, 1H), 6.52-6.41 (m, 1H), 4.70-4.28 (m, 2H), 4.15-3.82 (m, 6H), 3.78-3.69 (m, 3H), 3.66-3.47 (m, 3H), 3.43-3.37 (m, 1H), 3.27-3.19 (m, 1H), 3.02-2.94 (m, 1H), 2.67-2.41 (m, 1H), 2.25-1.87 (m, 5H), 1.85-1.35 (m, 10H), 1.34-1.16 (m, 6H), 1.11-1.04 (m, 3H). MS: [MH]⁺ 857.75.

### Example 2: Caliper Assay

Inhibition of CDK2/Cyclin E1 activity in the presence of compounds of the present disclosure was evaluated using a Caliper LabChip® EZ Reader mobility shift assay. In the assay, activated CDK2/Cyclin E1 catalyzes the phosphorylation of a fluorescently tagged peptide 5-FAM-QSPKKG-CONH2 (PerkinElmer, FL Peptide 18) which induces a difference in capillary electrophoresis mobility. The peptide substrate and product were measured, and the conversion ratio was used to determine the inhibition (as % activity and IC₅₀ values) of CDK2/Cyclin E1. Reactions contained 50 mM HEPES pH 7.5, 10 mM MgCl₂, 1 mM EDTA, 2 mM DTT, 0.01% Brij35, 0.5 mg/mL BSA, 0.1% DMSO, 2.5 nM CDK2/Cyclin E1 (14-475), 100 μM ATP, and 1.5 μM fluorescent peptide substrate.

Dose titrations of inhibitors in 100% DMSO were combined with 3.25 nM CDK2/Cyclin E1 (14-475) and 130 μM of ATP in reaction buffer. The mixtures were incubated for 30 minutes before the addition of fluorescent peptide substrate to initiate the kinase reaction. The final conditions were 2.5 nM CDK2/Cyclin E1 (14-475), 100 μM ATP, and 1.5 μM fluorescent peptide. The reactions were stopped after 100 minutes with the addition of EDTA (6 mM final EDTA concentration). The stopped reactions were analyzed on a Caliper LabChip® EZ Reader II. The conversion ratios were normalized to yield % activity, plotted against compound concentration, and fit to a four-parameter equation to determine the IC₅₀ for each compound.

The results of the Caliper Assay are reported in Table 9, below. Compounds with an IC₅₀ less than or equal to 0.01 μM are designated as “A.” Compounds with an IC₅₀ greater than 0.01 μM and less than or equal to 0.1 μM are designated as “B.” Compounds with an IC₅₀ greater than 0.1 μM and less than or equal to 1.0 μM are designated as “C.” Compounds with an IC₅₀ greater than 1.0 μM and less than or equal to 10.0 μM are designated as “D.” Compounds with an IC₅₀ greater than 10.0 μM are designated as “E.”

### Example 3: BrdU Cell Proliferation Assay

A BrdU assay was used as a measure of proliferation based on the DNA replication process of proliferating cells. BrdU, a pyrimidine analog, is added to the cell culture and incorporated into the DNA of proliferating cells. The presence of the BrdU analog was then measured through a colorimetric ELISA. After fixation and permeabilization of cells, peroxidase-conjugated antibody recognizing BrdU is added and allowed to incubate, followed by thorough washing to remove unbound antibody. In order to quantify the amount of bound antibody, peroxidase substrate is added and produces a color that can be measured at 450 nm.

On day −1, Kuramochi cells (Sekisui XenoTech JCRB0098) were seeded at 2,000 cells/well in columns 2-12 of a 96 well plate (Corning, CLS3596) in 150 uL media and allowed to adhere overnight at 37 degree with 5% CO₂. In order to assess specificity of the compounds, Kuramochi RB^{KO} cells were also plated and treated, as RB^{KO} cells were not expected to show a proliferative response to CCNE/CDK specific inhibitors.

On day 0, the source plate was prepared by adding 10 mM compounds and performing 3-fold serial dilutions for a 4-point dose response of each compound. Using a multichannel pipette, 2 μL of the contents of the source plate were stamped into an intermediate plate with 500 μL of RPMI 1640 Media, GlutaMAX Supplement (Life Technologies. 61870127) in each well of a Nunc 96 DeepWell™ plate, non-treated 96 DeepWell plate, 2 mL/well, sterile, natural. 60/cs (Sigma Z717274) and mixed thoroughly. 50 uL from row A of this intermediate plate were added to rows A-H of one plate of previously seeded Kuramochi cells, and each subsequent row of the intermediate plate was added to a full plate of cells.

On day 4, the plates were developed using the BrdU ELISA Cell Proliferation Assay according to manufacturer's instructions (Roche, 11647229001). Briefly, BrdU was diluted 1:100 in Gibco®. Opti-MEM® and 20 μL/well was added, shaken for 10 minutes at 350 rpm, and then returned to the incubator for 1 hour. Following incubation, the medium was discarded, and the cells were fixed by adding 200 μL of Fix/Denature solution. The anti-BrdU peroxidase antibody was diluted 1:1000 in OptiMEM, added at 100 μL/well, and incubated while shaking (350 rpm) for one hour. Three washes with PBS were performed to remove any unbound antibody, followed by the addition of 100 μL of substrate solution to each well. μL/well of 1M sulfuric acid solution was then added to halt the reaction, and plates were read out using an Envision spectrophotometer (Perkin Elmer) set to read 450 nm absorbance. Background absorbance values from empty wells were subtracted from all samples and then normalized to DMSO treated wells.

The results of the BrdU cell proliferation assay are reported in Table 9, below. Compounds with an IC₅₀ less than or equal to 0.5 μM are designated as “A.” Compounds with an IC₅₀ greater than 0.5 μM and less than or equal to 5.0 μM are designated as “B.” Compounds with an IC₅₀ greater than 5.0 μM and less than or equal to 10.0 μM are designated as “C.” Compounds with an IC₅₀ greater than 10.0 μM are designated as “D.”

### Example 4: IncuCyte® Cell Proliferation Assay

IncuCyte® assay was used to measure the effect of disclosed compounds on cell proliferation. Fluorescent microscopy images of cells were taken immediately after compound treatment and 72 hours later. Image analysis software was used to obtain cell counts as a function of compound concentration. Kuramochi cells labeled with mApple-H2B were seeded on 384-well assay-ready plates. Plates were placed in an IncuCyte® (Sartorius) and scanned at 0 and 72 hours. IncuCyte® software was used to count the number of fluorescent nuclei in each well. The fold change in cell count from 0 to 72 hours in wells treated with increasing compounds concentrations (10 pts, 1/2 log dilution, 20 μM top concentration) was normalized to DMSO control wells. The normalized cell counts were fit with dose response curves and a GI50 was calculated.

The results of the IncuCyte® cell proliferation assay are reported in Table 9, below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 μM and less than or equal to 20.0 μM are designated as “C”. Compounds with an IC₅₀ greater than 20.0 μM are designated as “D”.

### Example 5: ADPGLO (CDK2/E1-37C)

Inhibition of CDK2/Cyclin E1 activity by the presence of small molecules was evaluated using ADP-Glo Luminescent Kinase Assay (Promega). Activated CDK2/Cyclin E1 was incubated with its substrate Histone H1 (SignalChem H10-54N) in the kinase reaction buffer (100 μM ATP, 50 mM HEPES pH 7.5, 10 mM MgCl2, 1 mM EDTA, 2 mM DTT, 0.01% Brij35, 0.5 mg/mL BSA). Luminescence was recorded with an Envision plate reader (PerkinElmer).

Dose titrations of inhibitors in 100% DMSO were combined with 0.36 nM CDK2/Cyclin E1 in reaction buffer. The mixtures were incubated for 60 minutes at 37° C. before the addition of ATP and Histone H1 substrate to initiate the kinase reaction. The final conditions were 0.18 nM CDK2/Cyclin E1, 100 μM ATP, and 1.5 μM Histone H₁. The reactions were incubated at 37° C. for 90 minutes before being stopped with the addition of ADP-Glo reagent. This mixture was incubated at room temperature for 60 minutes before Kinase Detection Solution is added to generate luminescence. The stopped reactions were analyzed on an Envision plate reader. The conversion ratios were normalized to yield % activity, plotted against compound concentration, and fit to a four-parameter equation to determine the IC₅₀ for each compound.

The results of the ADPGLO assay are reported in Table 9, below. Compounds with an IC₅₀ less than or equal to 0.5 μM are designated as “A”. Compounds with an IC₅₀ greater than 0. μM and less than or equal to 5.0 μM are designated as “B”. Compounds with an IC₅₀ greater than 5.0 μM and less than or equal to 10.0 μM are designated as “C”. Compounds with an IC₅₀ greater than 10.0 μM are designated as “D”.

Entries noted in Table 9 as either “first isomer” or “second isomer” show the assay data for an isolated stereoisomer of said compound, wherein the specific stereochemistry is believed to be one of the stereoisomers designated as “A” or “B” in Table 9.

TABLE 9
Assay Results

| | Caliper | | IncuCyte | Pharmaron |
|---|---|---|---|---|
| | Assay | BrdU - | KURAM | ADPGLO |
| | (CDK2/ | KURAM | OCHI | (CDK2/ |
| | CCNE1): | OCHI.1: | RB-WT: | E1-37C): |
| Cmpd | IC50 | IC50 | IC50 | IC50 |
| # | (μM) | (μM) | (μM) | (μM) |
| I-1 | E | D | D | |
| I-2 | C | D | D | |
| I-3 | E | D | D | |
| I-4 | D | D | D | |
| I-5 | E | | D | |
| I-6 | E | | D | |
| I-7 | C | C | D | |
| I-8 | E | C | D | |
| I-9 | E | D | D | |
| I-10 | E | D | C | |
| I-11 | | B | B | |
| I-12 | E | D | | |
| I-13 | D | | C | |
| I-14 | E | D | B | |
| I-15 | C | | C | |
| I-16 | E | | | |
| I-16 | E | | | |
| I-17 | E | | D | |
| I-17 | E | | D | |
| 2^{nd} | | | | |
| I-18 | B | | B | |
| I-18 | E | | D | |
| I-19 | D | | D | |
| I-20 | C | | C | |
| I-21 | E | | C | |
| I-22 | D | | D | |
| I-23 | D | | D | |
| I-24 | E | | | |
| I-25 | C | | D | |
| I-25 | C | | D | |
| I-26 | C | | D | |
| I-27 | D | | D | |
| I-28 | E | | B | |
| I-29 | B | | C | |
| I-29 | B | | C | |
| I-30 | B | | B | |
| I-30 | B | | B | |
| I-31 | E | | | |
| I-32 | E | | D | |
| I-33 | A | | B | |
| I-34 | C | | | |
| 1-35 | B | | D | |
| I-36 | B | | D | |
| I-37 | C | | D | |
| I-38 | | | C | |
| I-44 | | | D | B |
| I-45 | | | D | D |
| I-46 | | | D | D |
| I-47 | | | C | D |
| 1-48 | | | B | B |
| I-49 | | | D | D |
| I-50 | | | D | C |
| I-51 | | | D | C |
| I-52 | | | C | C |
| I-53 | | | D | D |

1. A compound, wherein the compound is of Formula I: 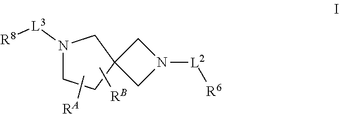
or a pharmaceutically acceptable salt thereof, wherein:
R^{A} is 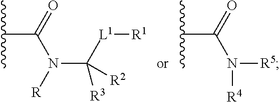
R^{B} is a hydrogen, an optionally substituted C₁₋₆ aliphatic group, —OR, —NR₂, or a halogen;
L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
R² is hydrogen, an optionally substituted C₁₋₆ aliphatic group, —C₁₋₆ alkylene-OR—C₁₋₃ alkylene-O—C₁₋₃ alkylene-R—C(O)OR, —C(O)NR₂, or an optionally substituted cyclic group selected from phenyl and a 5-6 membered monocyclic heteroaromatic ring (having 1-4 heteroatoms independently selected from nitrogen, oxygen, and sulfur); and
R³ is hydrogen; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and
R⁵ is hydrogen; or
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a covalent bond or a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a covalent bond or a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur), two R groups on the same nitrogen atom are taken together with the nitrogen atom to form an optionally substituted 4-7 membered saturated, partially unsaturated, or heteroaryl ring (having 0-3 heteroatoms, in addition to the nitrogen, independently selected from nitrogen, oxygen, and sulfur), or the two R groups on the same nitrogen atom are taken together with the nitrogen atom to form an optionally substituted 5-12 membered saturated or partially unsaturated bicyclic ring that is optionally bridged bicyclic or spirocyclic (having 0-3 heteroatoms, in addition to the nitrogen, independently selected from nitrogen, oxygen, and sulfur);
wherein one or both of L² and L³ is a covalent bond.

2. The compound of claim 1, wherein R^{A} is

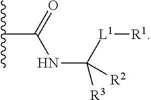

3. (canceled)

4. The compound of claim 1, wherein L¹ is a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

5. The compound of claim 1, wherein L¹ is an optionally substituted straight or branched C₁₋₄ alkylene chain, wherein 1-2 methylene units of L¹ are independently replaced by —O—, —NR—, —S—, —C(O)O—, —C(O)—, —S(O)₂—, or —NRC(O)—.

6. The compound of claim 1, wherein L¹ is an optionally substituted straight or branched C₁₋₄ alkylene chain, wherein 1-2 methylene units of L¹ are independently replaced by —O—, —NR—, —C(O)O—, or —NRC(O)—.

7. The compound of claim 1, wherein L¹ is a covalent bond,

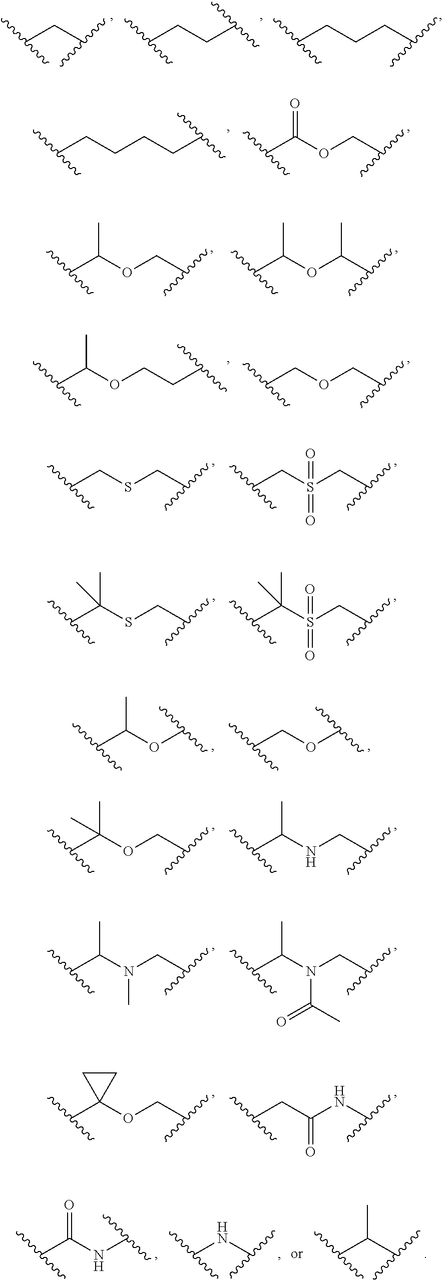

8. The compound of claim 1, wherein L¹ is

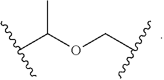 [SMILES: *COC(*)C]

9-31. (canceled)

32. The compound of claim 1, wherein the R^{A} is a substituent of Table 2.

33-53. (canceled)

54. The compound of claim 1, wherein the compound of Formula I is a compound of Formula II: 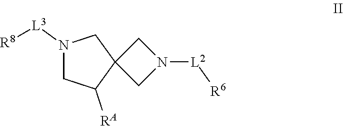
or a pharmaceutically acceptable salt thereof.

55. The compound of claim 1, wherein the compound of Formula I is a compound of Formula IIIa: 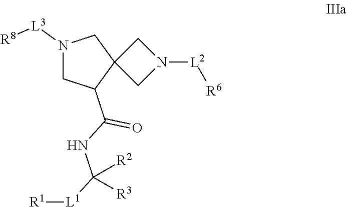
or a pharmaceutically acceptable salt thereof.

56. The compound of claim 1, wherein the compound of Formula I is a compound of Formula IVa: 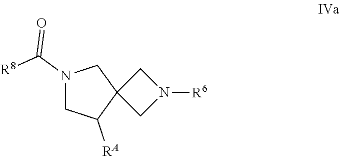
or a pharmaceutically acceptable salt thereof.

57. (canceled)

58. The compound of claim 1, wherein the compound of Formula I is a compound of Formula IVc: 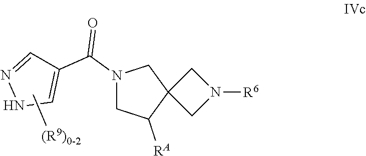
or a pharmaceutically acceptable salt thereof.

59. (canceled)

60. The compound of claim 1, wherein the compound of Formula I is a compound of Formula VIa: 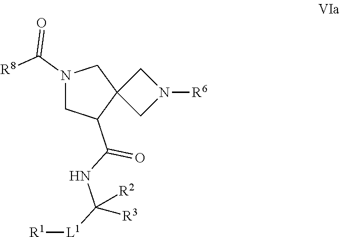
or a pharmaceutically acceptable salt thereof.

61. (canceled)

62. The compound of claim 1, wherein the compound of Formula I is a compound of Formula VIc: 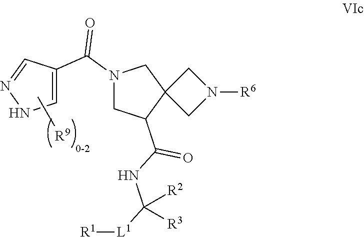
or a pharmaceutically acceptable salt thereof.

63. The compound of claim 1, wherein the compound of Formula I is a compound of Formula VIIa: 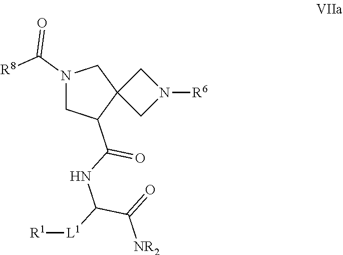
or a pharmaceutically acceptable salt thereof.

64. (canceled)

65. The compound of claim 1, wherein the compound of Formula I is a compound of Formula VIIc: 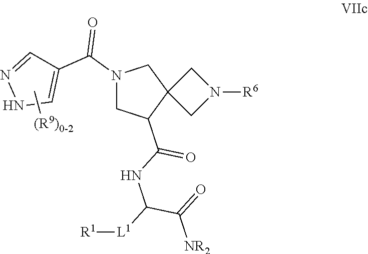
or a pharmaceutically acceptable salt thereof.

66-70. (canceled)

71. The compound of claim 1, wherein the compound is one of those in Table 7 or a pharmaceutically acceptable salt thereof.

72-74. (canceled)

75. A method of treating a disease or disorder associated with CDK2 activity in a patient comprising administering to the patient in need thereof a compound of claim 1, wherein the disease or disorder associated with CDK2 activity is a cancer selected from the group consisting of breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

76-83. (canceled)
